SEQUENCE LISTING (229 sequences)

SEQ ID NO: 1 - RNA, 47 nt, synthetic construct
  source 1..47: mol_type = other RNA
gggaaataagagagaaaagaagagtaagaagaaatataagagccacc

SEQ ID NO: 2 - RNA, 1209 nt, synthetic construct
  source 1..1209: mol_type = other RNA
atgagcgacgagggtccgggcacaggacccggtaacgggttgggcgaaaagggtgacactagcgggcccgaaggttccggcggatctggcccgcaacgcagaggtggtgataaccacggtcgcggtagaggcagaggccgtggccggggtggcggaaggcccggtgcccctggagggtcagggtctggcccaaggcaccgtgatggggtgcgtcgcccccagaaacgcccgtcctgtatcggctgcaagggcacgcatggcggaacgggaggtggcgcaggagccggaggtgcaagaggtcgcggggggtcaggggggagaggtagagggggatcaggggggcggagagggcggggtcgagaaagagcaagaggtggctccagagagcgcgctcggggtcgcgggcgaggccggggtgaaaaacggccccggagcccatcctcgcaatcatcaagctcgggatcgccacctcggcggccgccccccggcaggcgtccctttttccacccagtgggcgaagccgactacttcgagtaccaccaggagggaggaccggatggtgagccggatgttcctcccggcgccatcgaacaaggtcctgccgatgatccaggcgaggggccctcaacaggcccccgagggcagggggacgggggacgtcggaagaaaggaggctggtttggcaagcaccgaggacaggggggtagcaacccgaaatttgagaacatcgccgaggggctcagggctctcctagcccgtagccacgtcgaaagaactaccgatgagggcacctgggttgccggcgtgttcgtgtacgggggtagcaagacctctctatacaaccttcggagggggactgcacttgctatcccccaatgccgcctcacaccactgtctcgcctgccattcggcatggcccccggcccggggccacagcctgggcccctacgggaatctattgtttgctattttatggtgttcctgcagacccatatcttcgcagaagtcctgaaagacgccatcaaagacttagttatgactaaacccgcacccacctgcaacattcgcgtgaccgtatgcagctttgatgatggcgtcgatctgcccccatggttcccccctatggtcgagggcgccgccgccgaaggggacgacggggatgacggggatgaaggcggggacggcgatgagggcgaggagggccaggaa

SEQ ID NO: 3 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 4 - protein, 403 aa, synthetic construct
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGGGAGAGGARGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 5 - RNA, 1209 nt, synthetic construct
  source 1..1209: mol_type = other RNA
atgagtgacgagggacctgggactggccccgggaacggtctgggtgagaaaggtgacacgtctggtcctgaaggatctggcggttcagggccgcagcgcagaggaggagacaaccacggtagaggacgtggacgcgggagaggccgagggggcggaagacctggtgcaccgggcggttccggctcggggccccgccacagggatggggtgcggagaccacagaagcgcccctcttgcatcggctgcaagggaacccatgggggtaccggagggggagctggtgcaggtggcgcccgtggtaggggtggcagcggggggcgtggaagaggtggttcaggcggacggaggggacgcggacgtgaacgtgcccggggaggatcccgggagagggcacggggccgcggcagaggcagaggggagaagagacctagatcccctagctctcaatccagcagcagtgggtcccccccgaggcgaccgcctccaggtaggcgccctttcttccaccccgttggcgaggcagattacttcgaatatcatcaagagggggggcccgatggcgagccggacgtaccacccggtgctatcgagcaaggtccggctgacgatcctggtgaagggccaagtaccggacctagggggcagggcgacggtggtaggcgtaaaaaaggagggtggttcggcaaacatcgcggccagggagggagcaaccccaagtttgaaaatatcgccgaagggctacgagctttgctcgcgaggtcccatgtggaacgaactacagacgagggcacctgggtggctggtgtcttcgtttatggcgggagcaagacctccctgtataatctaaggcggggcacagccctggccataccgcagtgtcgcctgacacccctgtctcgcctgccttttggcatggcgccagggccaggaccacaacctgggcccctgcgcgaaagtattgtgtgttacttcatggtcttcctgcagacacatattttcgcagaagtcctaaaggatgcaataaaggatctcgtaatgacgaagcccgccccaacgtgtaatattagggttacggtctgtagtttcgatgacggggtagatctccccccctggttcccacctatggtcgagggtgccgctgcggagggggacgatggcgacgacggcgatgaaggcggggatggcgacgagggggaggagggacaggaa

SEQ ID NO: 6 - RNA, 1209 nt, synthetic construct
  source 1..1209: mol_type = other RNA
atgtcagacgagggcccaggcacgggccctggaaacggactgggagagaagggggatacctcagggccagagggtagtgggggtagcgggcctcagagaagggggggcgacaatcacggtcggggaagagggagaggtaggggtagaggaggaggccgtcccggggctcctggcggttccgggtccggccctcgccacagagatggcgtgaggagacctcagaagcgtccgtcctgtattgggtgcaaggggacccacggtggcaccggtggcggagctggggcaggaggtgctagaggaagaggcggttcaggtggaaggggtcgcggtggttcaggaggacggcgcggccgggggagagagcgggcaagaggcggcagcagggagcgggcaagaggacgagggaggggccgaggcgagaagcgacctaggtcgcctagttcacagagctccagttctgggtcgcccccccggcggccgcctcctgggcgaagaccctttttccaccccgtgggggaggctgactacttcgagtaccaccaggagggcggcccagacggagagcccgatgttccgcctggggccattgaacaaggccctgcagatgacccaggggaaggaccatcaaccggccctaggggacagggcgatggggggagacggaaaaagggagggtggttcgggaagcaccgcggccagggggggtctaatccgaaattcgaaaacattgccgagggcctccgtgctttgttggctagatcacacgtggaaagaaccacggatgaaggcacgtgggtggcgggcgtcttcgtgtatggtgggagtaagacctccctgtataacctcaggcgtggcacagcccttgccatcccacagtgcaggctcacaccccttagcaggcttccctttggcatggctcctggtcccggcccccagcccggtccactgcgagaatccattgtatgttacttcatggttttcttgcagacacatatcttcgccgaggtgctgaaagacgccattaaagacctggtgatgacaaagcctgcccccacgtgcaatatccgtgtgaccgtctgctcctttgatgacggagtcgacctgcctccctggtttcccccaatggtcgagggcgctgcggcggaaggggacgacggcgatgatggtgacgagggcggtgatggcgacgaaggtgaagagggtcaggag

SEQ ID NO: 7 - RNA, 1209 nt, synthetic construct
  source 1..1209: mol_type = other RNA
atgagcgacgagggtccaggcacaggcccggggaacgggttgggagaaaaaggggacacttccggtccagaaggctccggggggagcggacctcagagaagaggtggtgataatcacgggagagggagagggcggggcaggggacgtgggggcggtagacctggggcgccggggggttccggctccggaccacggcatagggatggtgtgcgaaggccgcagaagcggccgtcttgtataggctgcaaaggcactcacggtggtaccggggggggagcaggagcaggaggcgctcgaggtcggggcgggtccggcggcagaggccgcggtggttctgggggaaggaggggaagagggagagaacgcgcccgcggaggctctcgcgaacgggccagagggcgggggagggggcgcggggagaaaaggcccaggtcgccaagtagtcagtcctccagtagcggcagcccaccccgcagacctcctccaggacgacgacctttctttcatcccgtcggcgaggccgattactttgagtaccatcaggaaggggggcctgatggagaaccggatgttcctcctggtgctatcgaacagggccccgccgacgatccaggcgagggccccagtacgggccctagaggccagggagatggaggtagaaggaagaaggggggatggtttggcaagcacagagggcagggaggtagtaatcccaaatttgagaatattgccgagggtctgcgggcacttctggcccggtctcatgttgagagaacgactgatgagggaacatgggtggctggcgtctttgtgtatggcggtagtaaaaccagcctctacaatttacggcgtgggacagcgctagctattccgcaatgccggctgacccctttgtctaggctgccctttggtatggcccccggccccggcccacagcctgggcctttgcgggaaagcatcgtctgctactttatggtgttcctccagacccacatctttgcagaggtgcttaaggacgccataaaggacctggttatgaccaaacccgcccctacctgcaatatccgcgtcacggtgtgctccttcgacgacggggttgacttgcctccatggttcccacccatggtagagggcgctgctgccgagggggatgatggagacgacggagacgagggcggcgatggcgatgaaggcgaagaaggccaggaa

SEQ ID NO: 8 - RNA, 1209 nt, synthetic construct
  source 1..1209: mol_type = other RNA
atgtctgacgaaggcccgggcacaggcccaggcaacggattgggcgaaaaaggtgatacaagcgggccagaagggagcgggggctctgggccccagagacgggggggtgacaatcacggccgaggaaggggaaggggcaggggaagaggcggaggacggcctggcgctcctggaggctctggctcaggtccaagacatagggatggggtcagaagaccacagaagagacctagctgtattggatgcaagggtacacacggtggcacgggcggcggtgctggcgctgggggcgcgagaggacgtggaggttcaggcgggagaggtcgaggaggctctggaggccgcagggggagaggacgagagagggcccggggggggtcccgggaaagagctagagggaggggacgcgggcggggggaaaagcggcctcgctctccttcttctcaatcttcctcctctggttctccgccccgacgccccccaccgggcaggaggccattttttcaccctgtcggcgaggcagactacttcgagtaccaccaagaaggaggcccagacggcgagcctgatgtgccgccaggtgccatcgagcaggggccagctgatgacccgggggagggacctagcacggggccccgcggtcagggcgacggagggaggagaaaaaaaggtgggtggtttggcaagcatagagggcagggtggctcaaacccaaagtttgaaaacatcgcagaaggtcttcgggctctgctggcccgctctcatgtagagcgcactactgacgaaggcacctgggtggccggggtgttcgtgtacggaggatctaagaccagtctgtacaacctccggagggggacagctctcgctatcccccagtgtaggctcacccctttgagtcgccttccgttcggaatggctcctggcccgggcccccagcccgggccactaagagagagcatcgtatgctactttatggtatttttgcaaacccacatcttcgctgaagtgcttaaggacgccatcaaagacctggtgatgacaaaaccagctccaacatgcaatattagggtaaccgtatgctccttcgatgatggcgtcgaccttccaccttggttccccccaatggtggagggagccgcagcagaaggagacgatggtgatgatggagacgaaggcggggatggcgacgaaggagaggagggacaggaa

SEQ ID NO: 9 - RNA, 726 nt, synthetic construct
  source 1..726: mol_type = other RNA
atgggcagaagacccttctttcaccccgtgggagaagccgactatttcgagtaccaccaggagggcggtcctgacggcgagcccgacgtgcctccaggcgccatcgaacagggccccgctgacgatcctggcgaggggccttccacaggccctcgcggccagggagatgggggaaggcggaagaaggggggctggtttggcaagcataggggccagggcggctccaatcccaagttcgagaatatcgccgaggggctccgggctctgctggcccggtcgcacgtcgaacgtaccactgacgaggggacgtgggtggccggcgtctttgtgtacggaggatccaagacctccctgtacaacctgagaagggggaccgcccttgccatccctcagtgcagactcacccccctgagcaggctcccctttgggatggctcctgggccaggcccccagcccgggcccctcagggaaagcatcgtgtgttactttatggtgtttctgcagacacacatttttgcagaagttctgaaagatgcaatcaaggatctggtgatgaccaagcctgcacctacgtgcaatatcagggtgacagtatgctccttcgacgacggtgtggacctccccccctggttcccacccatggtcgagggggccgccgccgagggagacgatggcgacgacggtgatgaggggggagacggagatgaaggcgaggaggggcaggag

SEQ ID NO: 10 - protein, 242 aa, synthetic construct
MGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 11 - RNA, 726 nt, synthetic construct
  source 1..726: mol_type = other RNA
atgggacggagaccattcttccaccccgtgggagaagccgactacttcgagtaccaccaggagggcggtccagacggcgaacccgacgtccctcctggagccatcgagcagggccccgccgacgaccctggcgagggccctagcaccggacctagaggacaaggcgacggcgggaggagaaagaaggggggctggttcgggaagcaccgcgggcaaggcggcagcaacccaaagttcgagaacatcgccgaggggttgcgggccctgctcgcccgttcccacgtggagcggacaaccgacgagggaacctgggttgccggggtatttgtgtacggcggcagtaagaccagcctgtataacctcagaagaggaacagccctggccatcccacagtgcaggctgacacccctctcccggctgccatttggaatggcacccgggcctggaccccagcctggcccactgcgcgagtctatcgtgtgttactttatggtcttcctgcaaacccacatcttcgccgaggttttaaaggatgccatcaaagacctcgtgatgacaaagcccgctcccacctgcaatatcagagtgaccgtatgcagctttgacgacggcgtggacctgcctccctggttccctcctatggtggagggcgccgccgcagaaggggacgacggtgacgatggcgatgagggaggggatggagacgaaggcgaggagggccaggag

SEQ ID NO: 12 - RNA, 726 nt, synthetic construct
  source 1..726: mol_type = other RNA
atgggccgcagacccttctttcaccccgtgggagaggccgactattttgagtaccaccaggagggcgggcccgacggcgaacctgacgtgccacctggcgccattgaacaaggcccagccgacgaccctggggagggccctagcacagggcccaggggccagggagacgggggtaggcgtaagaagggaggctggttcggtaagcaccgcggccagggtggcagcaaccctaaattcgagaatattgccgaaggtctgcgagccctcttggccagaagccacgtggaacgcactactgatgagggcacctgggtcgccggagtgttcgtgtacggcggaagcaaaaccagtctgtacaacctgcgccggggaaccgctcttgccatcccccagtgtcgcctgaccccattgtccagactgccatttggcatggcccccggccccggcccccagcccggccccctgagggagagcatcgtgtgctacttcatggtgttcctgcagacccacatattcgctgaggtgctgaaggacgccattaaggacctggtgatgaccaagcccgcccctacctgcaacatccgcgtcaccgtctgtagtttcgatgacggcgtggatctcccaccatggtttcctccaatggtggagggcgctgctgccgagggggacgacggagacgacggagacgagggcggggacggggacgaaggcgaagaggggcaagag

SEQ ID NO: 13 - RNA, 726 nt, synthetic construct
  source 1..726: mol_type = other RNA
atgggacggagacctttctttcaccccgtaggtgaagccgactactttgaataccaccaggagggcggcccagacggcgagcccgacgttccccccggggctatcgagcaggggcctgcggacgacccaggagagggacctagcactggaccccgaggtcagggcgatggggggcgaagaaagaagggcggctggttcggcaaacataggggccagggcgggtctaacccaaagttcgagaacatagccgagggcctgagagccctgctggcaagaagccacgtggagcgaaccacagatgaaggcacctgggtggccggcgtgttcgtgtacgggggcagcaagacctccctgtataacctcagaagagggaccgccctggccatcccccagtgccgactgacccccctgagcaggctgcccttcggaatggcccctggtcccggtccccaaccgggcccgcttcgggagtccatagtgtgctacttcatggtgttcctgcagacacacatcttcgccgaggtgctgaaggatgcaatcaaggacctggtgatgaccaagccagctcccacctgcaacatccgggtgaccgtgtgcagctttgatgacggcgtggacctgccaccctggttcccgcctatggtggagggcgctgcagccgagggcgacgacggggacgacggcgacgagggcggagatggcgacgagggcgaggagggccaggag

SEQ ID NO: 14 - RNA, 726 nt, synthetic construct
  source 1..726: mol_type = other RNA
atgggcagacggcccttcttccaccccgtgggcgaagcagactattttgagtatcaccaggaggggggccccgacggcgagccagacgtcccacctggtgcaatcgagcaaggtcccgccgacgaccctggggagggccccagcaccggccccagaggccagggggacgggggcaggagaaaaaagggtggctggttcggcaagcacagagggcagggggggagcaacccaaagttcgagaacatcgccgagggcttgagagcactgctggccagaagccacgtggagagaaccaccgatgagggaacctgggttgctggcgtgttcgtctacggcggcagcaagaccagcctgtacaaccttagaagaggcaccgccctggccatcccccagtgccgcctgacaccgctgagcagactccccttcggcatggctcctggccccggcccccagcccgggcccctgagagagagcatcgtgtgctacttcatggtgtttcttcagacgcacatcttcgctgaggtcctgaaagacgccattaaggacctggtgatgacaaaaccagctccgacctgcaacatcagggtcactgtctgtagcttcgacgatggcgtggatctccctccctggttccccccaatggtggagggcgctgccgctgagggggacgacggcgacgacggggatgaaggcggcgacggcgacgagggggaagagggccaggag

SEQ ID NO: 15 - RNA, 1158 nt, synthetic construct
  source 1..1158: mol_type = other RNA
atggagcacgacctggagagggggccaccaggccccaggcgacccccaagaggaccaccactgagctccagcctgggactggccctcctgctgttgctcctcgctttactgttttggctgtatatagtcatgtctgactggacaggcggggcattattggtgctctacagctttgcactgatgctcattatcattattctgattatctttatttttagacgggacttgctctgtccattaggcgcgctgtgtattttgctgctgatgatcacactgctgctgattgcgctgtggaacctccacggtcaggccctttttctcggcatcgtgctgtttatctttgggtgcctcttggtgcttgggatttggatctaccttctcgaaatgttatggcggctcggcgctacaatctggcagctcctggccttcttcttagcattttttctggatctgatcctgctgataatagccctgtacctacaacagaactggtggactctgctggttgacctgttatggctgctcctgttcctcgccattctcatttggatgtactaccacgggcagcgtcattcagacgaacaccaccacgatgatagcctgccgcaccctcaacaagctacggatgactccggacacgagtccgatagtaactccaacgagggacgccatcacttgctggtctctggggctggcgacgggcctccactgtgtagccaaaatctgggagcccccggcgggggccctgataatgggcctcaggatcctgataacacggatgataacggcccacaggatccagataataccgacgataatggacctcatgatcctcttccacaggatcccgacaatacagatgacaatgggccgcaggatccagacaatactgacgataatggcccacatgatcccctgccacacagcccctcagactcagcgggaaatgatggcgggcccccacagttaaccgaggaagtggaaaacaaagggggagatcagggtcctcccctcatgacagatggcgggggcggccacagtcatgatagtgggcacgggggtggcgatccacatctccccacactgctgctaggcagctccggtagcgggggagatgatgacgatccccacggccctgtgcagttgagctattacgac

SEQ ID NO: 16 - protein, 386 aa, synthetic construct
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 17 - RNA, 1158 nt, synthetic construct
  source 1..1158: mol_type = other RNA
atggagcacgacctggagcgcgggcctccaggaccccgacggccgcccagggggccaccgctgtcatccagtctgggccttgctctgttgctgttactccttgccctgctattctggttgtacatcgtcatgagcgattggacagggggtgctctgctggtgttgtacagctttgcgctgatgttgattattatcatcctgattatctttatatttcgaagggacctcctttgtcccctgggagcgctgtgcatactgctcttaatgatcacgttgctcctcattgctttatggaatctgcacggacaggctctattcctcggcatcgttttattcattttcgggtgcctccttgtgctgggaatctggatctatctgctagagatgctgtggcggctcggcgccaccatttggcagcttctggcatttttcctagcgttttttctggatctcatcctacttattatcgccttgtatttgcagcagaactggtggactctcctggtggatctcctgtggctcctcctgttcctcgccatattgatatggatgtactaccacggacagcgccacagcgacgagcaccaccacgatgatagtctgcctcaccctcaacaggctacagatgactcaggccacgaaagcgattccaactcaaatgaaggccggcaccacttgttggtgtccggagccggcgacgggccaccactctgcagccagaatctgggcgctcccggcggaggtccagacaatgggccacaggaccctgataacacggatgataacggaccgcaggatcctgacaatacagatgacaacggcccgcatgatccactgcctcaggatcctgataatactgacgataacggaccccaggatcctgataacaccgacgacaacgggcctcacgatcccctaccccattccccatctgactctgctggcaacgatggcggcccaccgcagctgaccgaagaggtagaaaacaagggcggggatcagggccctccactgatgacagacggcggcgggggacactcccatgattcgggccatgggggaggggatcctcaccttcccacactcttgctgggaagtagcgggtccggcggggacgatgatgatccccatggtccagtccagttgtcatattacgat

SEQ ID NO: 18 - RNA, 1158 nt, synthetic construct
  source 1..1158: mol_type = other RNA
atggagcacgaccttgagcggggtccaccaggcccacggcgccctccgcgcgggcctcctttgtcatcctccctgggactggcactgctgctgttgcttctggccctgctgttctggctgtacatcgttatgtctgattggacaggaggagctctgctggttctttactctttcgcgctgatgctgatcatcatcatcttgatcattttcattttcagacgagacctcctttgtcctctcggagccctgtgtatcctcctgctgatgataacccttctgctgattgctttgtggaatctgcatgggcaagctctgtttcttggaattgtcttatttatttttgggtgtctgctggtgttgggaatctggatctatcttcttgaaatgctgtggcggctgggcgccacaatctggcagctactcgcctttttcctagctttctttttggatctgattctgctgattattgctctctacctgcagcagaattggtggaccctcttagtcgatctgctgtggctgttgctctttctcgccatcctgatttggatgtactatcatgggcagcgccatagtgatgaacatcatcacgatgattccctgccacatcctcagcaggccacagatgattcaggccacgaatcagacagcaatagcaatgagggccgccaccacctgctggtttcaggggccggtgacggccctccattgtgcagtcagaacctgggagccccggggggtggacctgataacgggccgcaggatcctgacaatactgatgacaacggaccccaggatccagataacactgacgacaatggcccccatgaccccctaccccaggatcccgacaatacagacgacaacggaccgcaggaccccgacaacactgatgataatgggccacacgatcctctgcctcactctccatcagactctgccggaaatgacggtggaccgccacagttaactgaagaagtcgaaaacaagggcggcgatcaggggccgccgctcatgacagatggcgggggaggtcacagccacgatagcggacacggaggaggtgaccctcatctgcctacacttctgttgggtagctctgggagtgggggggatgacgacgaccctcacggacctgttcagctctcatactacgac

SEQ ID NO: 19 - RNA, 1158 nt, synthetic construct
  source 1..1158: mol_type = other RNA
atggaacacgatcttgaacgggggccgccgggaccgaggaggccgccacgggggccccccctgagcagtagcttggggcttgcgctgcttctcctgctgttggccctcctgttttggctgtatattgtgatgtccgactggaccggcggcgccctgctcgtcctgtatagctttgctttgatgctgataattattatattgatcatctttattttccggcgtgatttgctctgtcctcttggtgccctttgtatcctgctgttaatgatcactctgctcctcattgccctatggaacctccacggacaagccctgtttcttggcatcgttctctttatattcggatgcctcttagtgcttgggatctggatatacctgttggaaatgctgtggaggcttggagccacgatatggcaattattggcattctttctggccttcttcctggatctgatcctgttgataattgctctgtacctgcagcagaactggtggacactacttgttgacctgctctggctactcctgttccttgcgatcctgatttggatgtactatcacggacagagacactccgacgagcatcatcacgacgattccttaccacaccctcagcaggcgacagatgattctggccatgagagcgattccaacagcaacgagggccggcaccatttgctggtgtcgggggctggagacgggcctccgttatgtagccaaaacttgggcgcgcccggcggcgggcccgataacggcccgcaggacccggacaataccgacgataatgggcctcaagatcccgacaatactgatgacaatggaccacacgatcctctgccacaggatcccgacaacacagatgacaatgggccgcaagaccccgacaatacagacgataatggcccccacgacccactaccacattctccctctgacagtgcggggaatgatggtggtcccccacaactgaccgaggaggtggagaataaggggggggaccaaggtccacccctcatgactgatggaggaggaggacactcgcacgattcaggtcacggcgggggggaccctcacttgcccactctgctgctgggcagcagcggttctgggggcgacgatgatgatccccacggcccggtgcagctaagttattacgac

SEQ ID NO: 20 - RNA, 1158 nt, synthetic construct
  source 1..1158: mol_type = other RNA
atggagcacgatttagagaggggcccacctggccccaggcgcccacctagaggcccacccttgtcgtcgtcattagggctggcattgctcctgctcctcctggccttgctgttctggctttacatcgtgatgagcgactggactggcggggccctgctggtcctctatagctttgcactgatgctgattataatcatattgatcatcttcatcttccgacgggatttactttgccccctgggggcattatgtattctattgctgatgataacacttctgcttattgctttatggaatctacacggtcaggctctttttctgggaatcgtgctctttatctttggatgccttctggtactaggaatttggatatacctcttagaaatgctgtggcggctcggcgcaaccatctggcaactccttgcgttttttctcgcgtttttcctcgacctgattctgctaataatcgccttataccttcagcagaattggtggacactcctggtggacctgctgtggctcctgctattccttgcaatcctcatttggatgtattaccatggccagaggcactctgatgagcaccaccacgatgatagtctacctcatccacagcaggctacggacgattcagggcatgagtcagattctaattccaacgaaggaagacaccatctgttggtgtcaggggccggagacggcccccctctgtgtagccagaacctcggggcaccagggggaggtcctgacaatgggccgcaagaccctgacaatactgatgataatggcccccaagatcctgacaataccgatgacaacggcccgcacgaccccctgccgcaggatccggataacaccgacgacaatggaccccaggaccccgataacacggacgataacggccctcacgatcccttaccccattctccgtccgactctgcagggaacgacgggggccctcctcagttaaccgaagaggtcgaaaataaggggggagaccagggccccccattaatgacagacgggggcggcggtcacagtcacgactcaggtcacggcggcggggacccccatttgcctacactgctgttagggtcttctggctcaggcggggacgatgatgatccacacggtccggtgcagctgtcatattatgat

SEQ ID NO: 21 - RNA, 1491 nt, synthetic construct
  source 1..1491: mol_type = other RNA
atgggtagtcttgagatggtaccaatgggcgcaggtccaccgagccctggaggcgatcctgacgggtacgacgggggaaataactcgcaatatccctcagcctcaggctcatcgggtaatactccgacccctccaaacgacgaggaaagggaatccaatgaggaaccgcctccaccatatgaggatccgtactgggggaacggtgataggcacagtgactaccagccacttgggacacaagaccaaagcctgtacctcggacttcagcacgatggtaacgacggtttgccgcctcccccttatagccctagagatgatagctcccagcatatatacgaggaggctggtaggggctccatgaatccagtatgcttgcccgttatcgtagccccctacctcttctggcttgcggcgatcgcagctagctgttttacagcttccgtgagcacagtcgtaaccgcgacaggactggcactgtctctactcctcttggcagcggttgccagcagctacgccgccgctcagcggaaactgctcacccctgttacagtcttaactgcagttgtgaccttcttcgccatttgtttgacatggaggatcgaagatcctccatttaatagtctcctgtttgcactgctggccgctgcgggcgggctacaagggatatatgtgctagtaatgctcgtcctacttattctggcctataggcggaggtggcggcgcctgacagtgtgcggagggataatgttcctagcctgtgtgctcgtgctcattgtggacgctgttctacagttgtctcctctgctgggtgcagtgacggtggtctcaatgaccctgctgttacttgccttcgtgctttggttgtcatctccagggggactgggcacactgggcgccgccctgttaaccctggcggccgctctggcactcctggcctcactgattctgggaactctgaatctcaccactatgtttcttctgatgttattgtggactttggtggtgttgctgatttgcagctcctgcagttcttgccccctatccaaaatattactggcgcggctgttcctttatgccctcgccttactgctgctcgctagcgccttaattgctgggggctccattcttcagaccaactttaaatccctgtccagcactgaattcatccccaaccttttttgcatgctgctcctaatcgtggctgggattctgtttattctggccattttgaccgagtggggcagtggcaacaggacctacggccctgtgttcatgtgtctgggggggctcttaacgatggtcgctggcgctgtctggctcacggtgatgagtaacactttgctcagcgcgtggatcctgacagccggctttctcatttttctgattgggttcgctttgtttggagtcattcggtgttgccgctattgctgttactattgtttgacattggagtccgaggagaggccacccaccccctaccgaaatacggta

SEQ ID NO: 22 - protein, 497 aa, synthetic construct
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 23 - RNA, 1491 nt, synthetic construct
  source 1..1491: mol_type = other RNA
atgggatccttggaaatggtgcccatgggagccggacccccctcaccagggggagacccagacggatacgacgggggaaacaattctcagtatccgagcgcgtccggcagctcaggaaacacacccaccccccccaacgacgaggagagagaatccaacgaggagcctcctcccccatacgaagacccttactgggggaatggagaccgtcattctgactatcaacctctaggcactcaggaccagtctctttacctagggttacaacacgatggtaatgatgggttgcccccgccgccctatagtccccgggacgacagtagtcagcatatatatgaggaagccggcagaggttctatgaatcccgtttgtctaccggtcattgtcgccccctatttattctggcttgcagccatcgccgcttcgtgcttcaccgcctccgtgtccacggtggtgacagctacagggctggcactctctctgctactcctggctgctgtggccagctcctacgcagcggctcagcggaagcttctcactcctgtaactgtcctcaccgcggtagtgaccttttttgccatatgcctgacatggaggattgaggacccaccctttaattctctgctgtttgcgcttttggccgctgccggaggactgcaaggtatttatgtgctggtaatgttggttctacttattctcgcataccggcgacggtggcggaggctgactgtatgcggggggattatgttcctggcgtgtgttctggttttgatcgtcgatgccgtgctgcagttgtcaccgctgctcggggcggtcaccgttgtgagtatgacactgcttctgctggctttcgtgctctggctatcttcccccggggggctggggaccttgggcgctgcgcttctgactctggcagctgctctggccctgctggcctctcttattcttggcacactgaatttgactaccatgttcctcttgatgttgctgtggaccttagttgtgctgctgatctgttcttcctgtagcagctgtccactgtccaagattctcctggccaggttgtttctgtatgccctagcgctgctgctgttagcttctgctctgatagccggcggcagtatcctgcaaaccaacttcaagagcctctccagcacagaattcatccctaatctcttttgcatgctactgctcatcgtggcaggcatactgttcattcttgctatcctgactgaatggggctctggcaatagaacatacgggccagtgttcatgtgcttgggcggcctgttgacaatggttgctggagccgtgtggctgacggtaatgtccaacactctgctgagcgcatggatcttaacagccggattcctgatctttttaatcggattcgcactcttcggagtgatccggtgctgtaggtattgttgttattactgcctgacactcgagtcggaagagaggccccctacaccttaccggaatacagtt

SEQ ID NO: 24 - RNA, 1491 nt, synthetic construct
  source 1..1491: mol_type = other RNA
atggggtcgttggagatggtccccatgggcgctggtcctccaagccccggcggggacccggacggatacgacgggggcaataatagtcagtatccgagtgcaagcggaagcagtgggaatacgcctacaccaccaaacgacgaggagcgggagtctaacgaggagcctccacccccttacgaggatccttactgggggaacggagatcgacattctgactaccagcctttagggacacaggatcagtctctctatctcggactgcagcatgatggcaacgacggactgcccccaccaccttattctccacgcgacgacagctcacagcacatatacgaagaggccgggcgcgggtctatgaacccggtttgcctgccagtcatcgtcgcaccctacctcttttggctggcggccattgccgcgtcctgtttcacagcctcagtgtcgactgtggtaaccgcaacagggctcgcactcagcctactgctgctcgctgccgtcgcgtcctcttatgctgctgcccagcgcaaactgctcaccccagttacggtgctcactgctgttgttacctttttcgctatttgtctcacctggagaatcgaagacccgccgttcaacagtctgttatttgccctcttggctgcagcaggcggccttcagggcatctatgtcctggtgatgctggtcctgctcatcctggcttacaggagaagatggcgtcggctgaccgtgtgtggtggcatcatgtttctcgcgtgtgttctggtgctcatcgtcgatgccgtgctacagctgagcccactgcttggagccgtgacagtcgttagcatgacactcctgctattggctttcgtgctctggctgagtagcccaggtgggctcggcacacttggtgccgcgcttttaacactggccgcggccctcgcactgctggcctctctaattctcggaacattgaacctgacaaccatgttcctcctgatgctcctctggacactggtggtactgttaatctgtagctcttgctcatcatgcccactgagcaagatactcctggcgagacttttcctatacgccctggccctgctgctgttggcatcggctctgatcgctgggggaagcatccttcaaacaaatttcaagagccttagcagtactgagtttatacctaacctgttctgtatgcttctgctgatagtcgctgggatactttttattctggcaatcctaacagagtgggggtcaggtaacaggacctatgggcctgtttttatgtgcctcggaggcctcctcaccatggttgccggcgcagtttggttgacagtgatgtccaacaccctactgagtgcttggatactgacggccgggttcctgattttcctaataggatttgccctgtttggggttatccggtgctgccgttactgctgctactattgtttgacccttgagtctgaagaaaggccgcccaccccctacagaaacactgta

SEQ ID NO: 25 - RNA, 1491 nt, synthetic construct
  source 1..1491: mol_type = other RNA
atggggtcattggagatggtgcctatgggggccggcccacccagcccagggggcgatcctgacggatacgacgggggcaataattcacaatatcctagcgccagcggatcttctgggaatacccccacaccacctaacgacgaggaacgggagtcaaacgaggaacccccaccgccgtacgaggacccatactgggggaacggggatcgacactcagactaccagcctctcggaacgcaggatcaaagcctgtacctgggactgcagcacgatggtaacgatggactgccaccacctccttacagtccaagggatgactccagccagcacatttatgaagaagccggccgaggtagtatgaatccagtgtgtctgcccgtgattgtcgccccttatctcttttggctggccgccattgccgccagctgtttcaccgcgtcggtttcaaccgtagtgaccgccacaggcctggctctcagcttgttactgttggccgctgtggcgagctcctacgccgccgcccagcgcaagctacttacccctgtgacagtgctgacggcggtggttacattcttcgccatttgcctgacctggcgaattgaggatccaccattcaattccttattgtttgccctcctcgccgctgctggagggctccagggtatctacgtgctggtgatgctcgttttgctgatattggcttaccggcggcgctggcgccgccttaccgtctgcggaggaatcatgtttttggcatgcgtgctcgttctcattgtggatgctgttttgcagctgtcacccctgttgggtgccgtgacagttgtaagtatgaccctgctcctgctcgcattcgtgctctggctgagctctcccggggggctgggcactctcggagcagctctgctgacactagccgcagcactagcccttctggccagtcttatcctcggaacgctgaacctgacgaccatgttcctcctgatgttgctctggaccctcgtagtgctgttaatatgctctagctgtagctcctgccctctgagcaagattctccttgctaggctgttcctatacgccctcgctctcctattgctggcgagcgctctcatcgccggtgggagcatcctgcagactaacttcaagtccctgagttcaacagaattcattcccaacctcttttgtatgctgctgcttatcgtggccggcatcctctttatcctcgccatcttgactgagtgggggtccggaaatcgcacatatggcccagttttcatgtgtctggggggcctgctcacgatggtggccggcgcagtgtggctgacggtaatgtcaaacactttgctctctgcgtggatcttgaccgcaggcttcctgatctttttgatcggcttcgccctgttcggtgtcatccggtgctgcagatactgctgctactattgcctcacactcgaatccgaagagaggcctcccacaccttaccgaaatacagtg

SEQ ID NO: 26 - RNA, 1491 nt, synthetic construct
  source 1..1491: mol_type = other RNA
atggggtctttagaaatggtccccatgggtgccggcccaccctcgccaggaggcgatccagacggctacgacgggggaaacaacagccagtaccccagcgccagcggtagcagcggcaacaccccgaccccaccaaacgacgaagagcgggagagcaatgaggagcccccacccccctatgaggacccttactggggaaatggcgatcgacacagcgattaccagcccctgggcacacaggatcagagcctgtacctgggcttgcagcatgatggtaacgacgggcttccgcccccaccatactcacccagagacgattcaagtcagcacatctacgaggaggctggtaggggtagtatgaatcctgtgtgtctccctgtgatcgtcgccccctacctattttggttagctgcgatcgctgcctcctgcttcaccgctagcgtgtcaacagtggttacggcaaccgggctggcgttaagtctgcttctgcttgcggcagtggcatcttcttatgcagcggcccagcggaagcttttgacccctgtgacagtactgaccgcagtggttacgttttttgcgatttgcttgacgtggagaatagaggatcccccttttaactcactgctctttgcactgctcgcggctgctgggggcctgcaggggatttacgtcctcgttatgctggtgctcctgatcttagcctacagaaggagatggcgccgactgacagtatgcggggggattatgtttttggcatgtgtactcgtattgatcgtggatgccgtcttgcagctttcccccctgctgggcgcagtcaccgtcgtgagtatgactctgctgttactggccttcgtactgtggcttagcagccctggaggactaggcacgctgggcgccgctttattgaccctggcagccgccctggctctgctggcctccctgatactggggacattaaatttaacaactatgtttctgctgatgctgctgtggactctagttgtgctcttgatatgttcctcctgttccagctgccctctgagcaagatcctgttggctcggctattcctgtacgcgctggctctcctcctgctagcttcagcgctaattgcaggcgggtctattctccagacgaattttaagagcctcagtagcactgagtttatcccgaatctgttctgcatgttgctattgatcgtcgcaggcattctcttcattctggccatacttacagagtggggtagcggcaaccggacttatggccctgtgttcatgtgcttggggggccttctcacaatggtggcaggggcggtatggctgaccgtgatgtctaacaccttgttatcagcctggatccttaccgccggcttcctaatttttctgatcggcttcgccctcttcggagtaatacgatgctgtcggtactgctgctactattgccttactttggagagcgaagagcggcctcccactccctatagaaacaccgtt

SEQ ID NO: 27 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgtttttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttttctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacccccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttttgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaaaacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacccccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacccccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaaaccaggcgaagttaacgtgacaaaaggaacgcccccccagaacgcaaccagtcctcaggcccccagcgggcagaaaactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccccctacaagccagccacgcttctccaaccttagtatgctcgttctccagtgggccagccttgctgttctgaccctcctcctgctgctcgtgatggccgattgcgcctttaggagaaacctcagcactagccacacgtacaccacgcccccctacgatgacgccgagacttacgtc

SEQ ID NO: 28 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 29 - RNA, 2595 nt, synthetic construct
  source 1..2595: mol_type = other RNA
atggaggctgctctattggtgtgccaatacaccattcagtcgctgatccacttgactggggaggatcccggatttttcaacgtggagattccggaattccctttttaccccacctgcaacgtgtgcactgccgacgtaaacgtgaccataaactttgacgtcggtgggaagaagcaccagctggacctggacttcgggcagttaacaccccacacaaaggcagtttaccagcccagaggagccttcggcggatcggagaatgcaacaaatcttttcctgctagaactgctcggcgccggcgagttggccctgacaatgagaagcaagaaacttcctatcaacgtaaccacaggggaggagcagcaggtcagcctcgaaagtgttgacgtctatttccaggatgtctttgggactatgtggtgccatcacgccgaaatgcagaatcccgtgtacctcattcccgagacagtaccctatatcaagtgggacaactgcaactctactaatattacagccgtggtgcgcgctcagggacttgatgtaacattgcctttgtctttgcccacaagcgctcaggattctaatttctctgtaaagacggagatgctggggaatgaaatcgacatagaatgtattatggaagacggtgaaatcagtcaggtgctaccgggagacaacaaattcaatattacatgttctggctatgagagccacgtgccgagcgggggcatcctgacttcgacttctcccgtcgccacacccataccaggcacaggctacgcttatagtctgaggctgactcccaggccagtatcccgatttctgggtaataattccatcctttatgtgttttactccggtaacggccccaaagcttcggggggcgactattgtatccagtctaatatagtgttctccgatgagattcccgcgagtcaggatatgcccaccaacaccaccgacatcacgtatgttggggataacgctacctatagtgtgccgatggtgacatccgaggacgcaaactctcctaacgtaaccgtgaccgccttttgggcatggccaaataataccgaaaccgattttaaatgcaagtggaccctgacatccgggactccctccggatgcgaaaatatttctggagcttttgccagcaatcggacctttgatatcactgtgagtggtttaggcaccgccccaaaaacactgatcataaccaggacagccacaaatgcgaccacaactacacacaaagttatatttagtaaagcacctgagtccaccacaacttctccgaccctcaacactaccggcttcgctgatccgaataccaccacaggtcttccatcttccacccacgttccaactaatcttacggcgcctgctagcaccggtccaaccgtgtccaccgccgatgtgactagccccacgccggctggaacaacatcaggggccagccctgtaaccccaagcccttcaccctgggacaatggtaccgagagtaaggcgcccgacatgacctcatcaacttctcctgttacaactccaacaccaaatgccactagtcctacccccgcagtcactaccccaacgcctaatgcaacgtcaccaactcctgcagtgaccacaccaacacccaatgctacttcgccgaccctgggcaagaccagcccaaccagtgccgtgacgacaccgactccaaatgcgaccggacctactgtcggtgagacaagcccccaggcaaacgcgaccaaccacaccctcggcgggacttctccaacacccgtcgtaacgtcccaaccaaaaaatgctacatcggccgtgactaccggacagcataatatcacctcaagctccacgtcgtcaatgagcctgcggccgtcaagtaatccagagactctgtcgccatctacctcagataactcaactagccacatgcctctactcactagtgcccaccccaccggcggggagaacatcacgcaggtaacaccagcgtctatcagtactcaccatgtgagtaccagctcccccgctcctcgaccgggaaccacgagccaggccagcggaccgggtaatagtagcacttctactaaaccgggagaggtcaatgtgactaaggggactcccccccaaaacgctaccagcccacaggctccctcagggcagaagaccgctgttccaacagttacatctactgggggcaaggcaaatagcacaaccggtggcaagcatactactgggcacggtgcccgcacaagcaccgagcctacaacggactatgggggggattctaccacaccacgtccccggtataacgccaccacgtatcttccgcctagcacatcaagcaaactcagacctcgctggacatttacatcacccccagttaccactgctcaggcaaccgtcccggtgcctcctacttcacaaccacgtttcagcaacttgagcatgctggtcctgcagtgggcttccctcgccgtgctgacattactgttattgctggtgatggccgattgcgcgtttcgcaggaacctgtctaccagccacacatacacaacgcccccgtatgacgacgcagagacttatgtg

SEQ ID NO: 30 - RNA, 2595 nt, synthetic construct
  source 1..2595: mol_type = other RNA
atggaagccgctcttctggtctgccagtatacaatccagtcgctgattcatctcaccggcgaggatccaggtttctttaacgtggagattccagaattccctttctatccaacttgtaacgtgtgtactgccgacgttaacgttactattaactttgacgtgggcggaaagaagcaccagctggatcttgatttcggccagctaacacctcataccaaggccgtctaccaaccacgcggggcatttggcggttctgagaatgctacaaacctgttccttttggaactcctcggagcaggggaactggcacttacaatgaggagtaagaagctgcccatcaacgtcaccacgggggaagaacagcaggtgtccctggagtccgttgatgtgtatttccaggacgttttcgggacaatgtggtgtcatcatgccgaaatgcagaatccggtgtatctcattcctgagaccgtgccctatattaaatgggataactgtaattcgacaaacataactgccgtagtgcgtgcccagggtctggatgtaactttgcctctctcccttcccacttctgcccaggactccaatttttcagtgaagaccgaaatgcttgggaacgagatcgacatcgagtgtatcatggaggatggggagatcagtcaggtgttgcctggtgacaataagtttaatataacatgttccggctatgaatcacatgttccaagcggaggtattctcacaagtacatctcccgtagccacgccgattccgggaaccggatacgcctattcacttcggctgactcctcggccagtgtcacgattcctgggcaataactcaattctgtacgtcttttacagcggcaacggcccaaaggccagtggaggcgattactgtatccagtctaacatcgtattttcagatgagattccagcaagccaagacatgccaactaatactacagacattacatacgtcggtgataacgccacttattcagtccctatggtcacatccgaggacgcgaactcacccaatgttaccgttaccgcattctgggcatggcccaataatacggaaacagacttcaaatgtaagtggaccctgaccagtggcaccccttctggctgcgaaaatataagcggggcatttgctagcaacagaacattcgatattactgtgtctggcctggggaccgcccccaaaacactcattataacgcgaacagctacgaacgctaccacaaccacacataaagtgattttcagcaaggctccagaatcaactacaaccagtccgacccttaataccacaggcttcgcagacccgaacacaacgacgggactgccctcctcaacgcatgtgccgactaatctgactgctcccgcaagtaccggcccaacagtctccacagcagacgtcacctctccgactcccgccggcactacatctggagcctcaccggtgactcctagtccttccccctgggacaatgggaccgagtctaaagccccggacatgacgagttctacctcaccagttacaaccccaacacctaatgccacaagccctacgccagccgtgaccacccctacccccaatgccaccagcccaacacccgctgttacaacgccaacccccaacgctacttctccaacgctaggaaaaacatcacccacctcagctgtaaccacccccacaccaaacgctaccgggccaaccgttggcgaaacctccccccaggccaatgcgacaaaccacacccttggggggacttctcccactcctgtggtgacttcacaaccgaagaacgcgacgtcggccgttacgaccgggcagcacaatatcacctcgtcctccacgtcgtccatgtccttgcggccttccagtaacccagagacactgtctcctagtaccagtgacaacagcacctctcacatgccattactgacttccgcccacccaaccggcggagagaacatcactcaggtgacccccgcatctatttctacccaccacgtgagcactagcagcccggcaccaagaccagggaccacctcccaggctagcggcccgggcaactcctccactagcacgaaaccaggagaggtgaacgtgaccaagggcacaccgccacagaatgcaacttccccacaggccccctccggccagaaaactgctgtacctacggtcactagcactggcggaaaagcgaattcaaccaccggtggtaagcataccaccggacacggcgctcggactagcacagagccaaccactgattacgggggtgactcaacgaccccgaggcccaggtacaacgcaaccacctatctcccaccatccacatcttccaagctcaggcctcgttggactttcacctccccgcctgtgactacagcgcaggccacagtgccagtgcctccaacgtcacagccgcggtttagcaatctctccatgctggtcctgcagtgggcatctctggcagtcctgacactgcttcttttgctcgtgatggccgattgtgcgtttagacgcaacctttcaacatcccacacatatacaactccaccctatgatgacgcagagacttatgtg

SEQ ID NO: 31 - RNA, 2595 nt, synthetic construct
  source 1..2595: mol_type = other RNA
atggaagccgccctattagtctgccagtacaccattcaaagcctcatccacttgactggggaggatcctgggttctttaacgtggaaatcccggaattccctttctacccaacttgcaacgtgtgcacggctgacgtcaacgtgactatcaacttcgatgtgggggggaagaaacatcaacttgatctggactttggccagcttactccccacaccaaggccgtgtaccagccccgcggcgcattcggtgggagcgagaacgcgacaaatctattcttgctggaactgctgggcgcaggagagctcgccctgacgatgcgatctaaaaagcttcccatcaacgttaccactggcgaggagcagcaggtgtccctcgaaagcgtcgacgtctatttccaagacgtcttcggaacaatgtggtgccaccacgctgagatgcagaaccccgtatatcttattcccgaaaccgtgccatacatcaagtgggacaactgcaactcaactaacatcactgcagtagtgcgcgcccagggactggacgtcactttgccactgtcactgcccacatcagcccaagacagcaacttcagcgtcaagacagaaatgctgggcaacgaaatcgacatcgaatgcataatggaggatggggaaatttcccaggtcctccccggtgataacaagtttaatattacttgttctggatatgaatcccatgtgccgtctggtggtatactgacgagtacatcgccagtggctaccccaatccccgggacaggctatgcctacagcctgagactgacgccaagaccagtttccaggtttctggggaataactctatcctgtatgtgttttattctggtaacggacccaaggcatccggcggtgattattgtatccagagcaatatagtgttctctgatgagattccggcctcccaggatatgccaaccaacacgacagatatcacctatgtcggggacaacgccacatatagcgtgccaatggtgaccagcgaagatgccaattctcccaacgtcacagtgacagccttttgggcgtggcctaataacacagaaactgactttaaatgcaaatggactctaacctcaggcaccccatctggctgcgagaacataagcggcgcgtttgcatcgaacagaacttttgatattacagtatctgggttgggcactgcccccaaaactctaataattacacggacggccactaacgccaccacgacgactcacaaggtgatcttcagcaaagctcccgaatctactactacctcgccgacactgaatacgacaggattcgccgacccaaatactacgaccggccttcccagctcgacacatgtgcctacaaacttaacggcccccgccagtaccggccccaccgtgagcaccgctgacgtcacctctcctacccctgccgggactacaagtggtgcttccccagttactcccagccctagcccttgggacaacggcactgagagcaaagctccggacatgaccagcagtaccagtccagtgacaactccgacacccaatgcgacgtccccaactcccgccgtgaccacgccaacaccaaatgccacgtcacctactccagcggtcaccaccccaacgccaaacgctactagtcccactctaggcaagacatctcccacctccgctgtcaccacccctacacccaatgctaccggtcccacggtgggtgagactagcccacaggccaacgcaactaatcatacattggggggcacaagcccaacccccgtcgtcactagccagcctaaaaacgccacaagcgctgtaactaccggccagcacaatataacaagttccagtactagcagtatgtctctgagacccagctcaaatcctgaaaccctctccccctctacgtcggataactccacgtcccacatgcctctactcacaagcgcgcaccctaccgggggagaaaatatcactcaggttaccccagcttctatctcgactcaccatgtctctacttcttccccagcgccacggcccggcactaccagccaggctagcgggccaggtaattcctccactagcactaaacctggcgaagtcaacgttaccaagggaacacccccccagaatgcaacctccccccaagctccctctggacagaaaacagccgtccctaccgtcacaagcaccggtgggaaggcgaattctacgacaggcgggaagcacactaccggacacggagcaaggacatccactgagcccacgaccgactacggtggcgactccaccacccccagacccaggtacaatgcgactacctacctgcccccttccacttccagcaaactgaggcctcgttggactttcacttctcccccagtcaccaccgctcaggctaccgtgcctgtcccgccgacctctcagccccggttttcaaatttgagtatgctcgtgctgcagtgggccagcctggcagtgcttaccctcctgctcttgctggttatggctgactgcgccttccgacgtaacttatccaccagtcatacttacactacacccccttatgacgacgcggagacctacgtg

SEQ ID NO: 32 - RNA, 2595 nt, synthetic construct
  source 1..2595: mol_type = other RNA
atggaagccgccttactggtctgccagtacactattcagtccctcatccatctcacaggcgaggacccggggttcttcaacgtcgaaatccccgaattcccattctatcccacctgtaacgtctgcacagccgacgttaacgttacaatcaatttcgacgtgggcgggaaaaaacaccagctggacctcgactttgggcagctgacgccccacactaaagccgtttaccagcccaggggtgcattcggtggatctgaaaatgctacaaacctctttctgcttgagctgcttggtgctggagagctggccctgacaatgcgcagcaaaaagttgccaatcaatgtgaccacaggtgaagaacagcaggtgtccctagaatccgtggatgtgtacttccaggacgttttcggcacaatgtggtgccatcacgcagagatgcaaaatccggtgtaccttatccccgaaacagtgccgtacattaaatgggataattgtaactccacaaacatcacggcagtggtgcgcgctcaggggctggacgtgacactccccctttccttaccaacaagtgctcaagattccaacttttctgtgaaaaccgagatgctgggcaacgagattgatattgaatgcatcatggaggatggcgagattagccaggtgttgcccggcgacaataaatttaatattacctgttccggctatgagtcacacgtgccaagtggaggcattctcacgagcacctctcccgttgcaacacccattcctgggacagggtacgcatatagtctcaggctgacaccgagaccggtatcgcgcttcttaggtaacaattccatcttgtatgtgttttacagcggaaacgggccaaaggcctcaggaggagactactgcatccagagcaatatagtttttagcgacgagatccctgcctcacaagacatgcccaccaacaccaccgacatcacgtacgtcggtgataatgccacttacagcgtgcctatggtgacctctgaagacgcaaattcacccaatgtcacggttaccgcattctgggcttggccaaataacactgaaaccgacttcaagtgtaaatggaccctaaccagcggcaccccatcagggtgcgagaatatcagcggggccttcgccagcaaccggacattcgacatcaccgtgtcaggactagggactgctccaaaaacattaattattacacggacagcaactaacgctacgactacgactcacaaagtgatatttagcaaagcccccgaatcgactactacgagcccgaccctaaacaccaccggtttcgccgatcccaacacaactactggtctcccatcttcgacccatgttccaacgaatctgacagctccagcaagtacggggcccactgtgagtacagccgacgttactagcccaactcccgcgggtacgactagtggcgcgtctccagtgaccccttcgccttctccgtgggataatggaaccgagtcaaaggctcctgatatgaccagctccaccagtcccgttacaacgccaacccccaatgcaacgagtccaactccggcggttaccactccgacaccaaacgccacctcccccacgcctgcagttactacgcccacccctaatgctacgtcacccactctgggaaagacctcaccaacctctgccgtgactactcctacacctaatgccacaggacccacagttggggaaaccagcccacaggctaacgcaactaatcatacgcttggggggacctctcccacgcccgtggtcacttcacagcctaagaatgccacaagcgctgtgactacaggccagcataatataaccagcagttcaaccagtagcatgtccctgcgtccttcatccaatcctgagactctgagccccagcactagtgacaactcaacttctcacatgccgttgctgacctccgcgcatcccacgggcggtgagaatatcacacaggtgacacccgcgtctattagcacacatcacgtgagtacgtcctcccccgcaccccgcccaggcacaacctcacaagcatcaggtccggggaacagcagcacaagtaccaagcctggcgaagtgaatgtaaccaagggcacgcccccgcagaacgccacatcccctcaagccccatccggccagaagaccgctgtacccaccgtgacatcaactggggggaaagcgaactcaactaccggggggaagcatacaacagggcacggtgccagaacttctacagagccaaccacggactacggaggggacagcacaacgcccagaccgcggtacaatgcgaccacatacctgcctccctcaacttcgagtaagctgcgacccagatggacctttacttctccccctgtcaccaccgctcaagccaccgtcccggttccacctacttcccagcctagattctccaatctgagcatgcttgtcctacagtgggcatcactcgcggtactaaccttgctgctactgttggtgatggcggactgcgcgtttcgccgcaacttatccacctcccacacgtacacaacacctccctatgatgacgctgagacttacgtc

SEQ ID NO: 33 - RNA, 669 nt, synthetic construct
  source 1..669: mol_type = other RNA
atggtgagcttcaagcaggtgagagtgcccctgttcaccgccatcgccctggtgatcgtgctgctgctggcctacttcctgccccccagagtgagaggcggcggcagagtggccgccgccgccatcacctgggtgcccaagcccaacgtggaggtgtggcccgtggaccccccaccccccgtgaacttcaacaagaccgccgagcaggagtacggcgacaaggaggtgaagctgccccactggacccccaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccacccccgacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagc

SEQ ID NO: 34 - protein, 223 aa, synthetic construct
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 35 - RNA, 411 nt, synthetic construct
  source 1..411: mol_type = other RNA
atgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggc

SEQ ID NO: 36 - protein, 137 aa, synthetic construct
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 37 - RNA, 2118 nt, synthetic construct
  source 1..2118: mol_type = other RNA
atgcagttgttgtgcgtgttctgcctcgtgttactctgggaggtgggcgccgccagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctg

SEQ ID NO: 38 - protein, 706 aa, synthetic construct
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 39 - RNA, 1158 nt, synthetic construct
  source 1..1158: mol_type = other RNA
atggaacacgaccttgagaggggcccaccgggcccgcgacggccacctcgaggacctcccctctcctcttccctaggccttgctctccttctcctcctcttggcgctactgttttggctgtacatcgttatgagtgactggactggaggagccctccttgtcctctattcctttgctctcatgcttataattataattttgatcatctttatcttcagaagagaccttctctgtccacttggagccctttgtatactcctactgatgatcaccctcctgctcatcgctctctggaatttgcacggacaggcattgttccttggaattgtgctgttcatcttcgggtgcttacttgtcttaggtatctggatctacttattggagatgctctggcgacttggtgccaccatctggcagcttttggccttcttcctagccttcttcctagacctcatcctgctcattattgctctctatctacaacagaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggaccaccactctgctctcagaacctaggcgcacctggaggtggtcctgacaatggcccacaggaccctgacaacacggatgataatggcccacaggaccctgataacactgatgacaatggcccacatgacccgctgcctcaagacccagacaatactgatgacaatggcccacaggaccctgacaacactgatgacaatggcccgcacgacccgttacctcatagccctagcgactctgctggaaatgatggaggccctccacaattgacggaagaggttgagaacaaaggaggtgaccagggcccgcctttgatgacagacggaggcggcggtcatagtcatgattccggccatggcggcggtgatccacaccttcctacgctgctgttgggttcttctggttccggtggagatgatgacgacccgcacggcccagttcagctaagctactatgac

SEQ ID NO: 40 - RNA, 1491 nt, synthetic construct
  source 1..1491: mol_type = other RNA
atggggtccctagaaatggtgccaatgggcgcgggtcctcctagccccggcggggatccggatgggtacgatggcggaaacaactcccaatatccatctgcttctggctcttctgggaacacgcccaccccaccgaacgatgaggaacgtgaatctaatgaagagccaccaccgccttatgaggacccatattggggcaatggcgaccgtcactcggactatcaaccactaggaacccaagatcaaagtctgtacttgggattgcaacacgacgggaatgacgggctccctccacctccctactctccacgggatgactcatctcaacacatatacgaagaagcgggcagaggaagtatgaatccagtatgcctgcctgtaattgttgcgccctacctcttttggctggcggctattgccgcctcgtgtttcacggcctcagttagtaccgttgtgaccgccaccggcttggccctctcacttctactcttggcagcagtggccagctcatatgccgctgcacaaaggaaactgctgacaccggtgacagtgcttactgcggttgtcactttctttgcaatttgcctaacatggaggattgaggacccaccttttaattctcttctgtttgcattgctggccgcagctggcggactacaaggcatttacgttctggtgatgcttgtgctcctgatactagcgtacagaaggagatggcgccgtttgactgtttgtggcggcatcatgttcttggcatgtgtacttgtcctcatcgtcgacgctgttttgcagctgagtcccctccttggagctgtaactgtggtttccatgacgctgctgctactggctttcgtcctctggctctcttcgccaggtggcctaggtactcttggtgcagcccttttaacattggcagcagctctggcactgctagcgtcactgattttgggcacacttaacttgactacaatgttccttctcatgctcctatggacacttgtggttctcctgatttgctcttcgtgctcttcatgtccactgagcaagatccttctggcacgactgttcctatatgctctcgcactcttgttgctagcctccgcgctaatcgctggtggcagtattttgcaaacaaacttcaagagtttaagcagcactgaatttatacccaatttgttctgcatgttattactgattgtcgctggcatactcttcattcttgctatcctgaccgaatggggcagtggaaatagaacatacggtccagtctttatgtgcctcggtggcctgctcaccatggtagccggcgctgtgtggctgacggtgatgtctaacacgcttttgtctgcctggattcttacagcaggattcctgattttcctcattggctttgccctctttggggtcattagatgctgccgctactgctgctactactgccttacactggaaagtgaggagcgcccaccgaccccatatcgcaacactgta

SEQ ID NO: 41 - RNA, 726 nt, synthetic construct
  source 1..726: mol_type = other RNA
atgggcagaagacccttctttcaccccgtgggagaagccgactatttcgagtaccaccaggagggcggtcctgacggcgagcccgacgtgcctccaggcgccatcgaacagggccccgctgacgatcctggcgaggggccttccacaggccctcgcggccagggagatggcggaaggcggaagaagggcggctggtttggcaagcataggggccagggcggctccaatcccaagttcgagaatatcgccgaggggctccgggctctgctggcccggtcgcacgtcgaacgtaccactgacgaggggacgtgggtggccggcgtctttgtgtacggaggatccaagacctccctgtacaacctgagaaggggcaccgcccttgccatccctcagtgcagactcacgcccctgagcaggctcccctttgggatggctcctgggccaggcccgcagcccgggcccctcagggaaagcatcgtgtgttactttatggtgtttctgcagacacacatctttgcagaagttctgaaagatgcaatcaaggatctggtgatgaccaagcctgcacctacgtgcaatatcagggtgacagtatgctccttcgacgacggtgtggacctcccgccctggttcccacccatggtcgagggtgccgccgccgagggagacgatggcgacgacggtgatgagggtggagacggagatgaaggcgaggaggggcaggag

SEQ ID NO: 42 - RNA, 2832 nt, synthetic construct
  source 1..2832: mol_type = other RNA
atggacaaggacaggcccggccctcccgccttggacgacaacatggaggaggaggtgcccagcaccagcgtggtgcaggagcaggtgagcgccggcgactgggagaacgtgttaatcgagttgagcgacagcagcagcgagaaggaggccgaggacgcccacctggagcccgcccagaagggcaccaagaggaagagggtggaccacgacgctggcggatctgcccctgccaggcccatgctgccaccccagcccgacctgcccggcagggaggccatcctgaggaggttcccactggacctgaggaccctgctgcaggccatcggcgccgccgccaccaggatcgacaccagggccatcgaccagttcttcggcagccagatcagcaacaccgagatgtacatcatgtacgccatggccatcagacaagcaattagggatcgacgcagaaatcctgccagcaggagggaccaggccaagtggaggctgcagaccctggccgccggctggcccatgggctaccaggcctacagcagctggatgtacagctacaccgaccaccagaccactccgaccttcgtgcacctccaggctaccctgggctgcactggcggtcgcaggtgccacgtgaccttctcagcgggcaccttcaagctgcccaggtgcacacctggcgacaggcagtggctgtacgtgcagagcagcgtgggcaacattgttcagagttgcaaccccaggtacagcatcttcttcgactacatggcgattcacaggagcctgaccaagatctgggaggaagtcctgacccctgaccagagggtgagcttcatggagttcctgggcttcctgcagaggaccgacctgagctacatcaagagcttcgtgagcgacgccctgggcaccaccagcatccagactccttggatcgacgataaccctagcaccgaaaccgcccaggcctggaacgccggtttcctgaggggcagggcctacggcatcgacctgctccggaccgagggcgagcacgtggagggcgccaccggcgagactagggaggagagcgaggacactgagagcgacggcgacgacgaggatctgccgtgcatcgtgagcaggggcggccccaaggtgaagaggcctcccatcttcatccggaggcttcacaggctgctgctgatgagggccggcaagcgtactgaacagggcaaggaggtactggagaaggccaggggcagcacatatggcacccctagaccgcctgtgcccaagccgcgtcctgaagtcccgcagagtgatgagacagcgactagccatggcagtgctcaggtgcccgagccacccaccatccatctcgccgcacagggcatggcctacccactgcacgagcagcacggcatggcgccctgccccgtggcccaggctcctccaaccccgcttcctccagtatcccctggtgaccagttgccaggcgtgttcagcgacggtagggtggcctgcgcgccagttcctgcaccagcaggcccaattgtgaggccctgggagccctccctaacccaggcggcaggtcaggccttcgctccagttagaccacagcacatgcccgtggaaccggtgccagtgcctacggtggcgctggagaggcccgtgtacccaaagcctgtgcgacctgcgcctcctaagatcgccatgcaaggcccgggcgagactagcggtatcaggcgggccagggagaggtggcgcccggcaccgtggactcccaacccaccaaggtcacctagtcagatgagcgtgcgtgataggctggcacggcttcgggcagaggctcaggtaaagcaggccagtgtggaggtgcagccaccacagctgactcaggtgtccccgcagcagcccatggagggcccactggtgccggaacagcagatgttccccggcgccccgttcagccaggtggccgacgtggttagggctcctggcgtgcctgctatgcagcctcagtacttcgacctaccgctgatccagcccatcagtcaaggtgccccagtggccccactgagggctagcatgggcccggtccctcctgttccagccacccagcctcaatatttcgacattcctctgaccgagcctataaaccagggcgcgtctgcggcccacttcctgcctcaacagccaatggaaggcccgctggtcccagaacagtggatgttccctggagctgccctgagccaatcagtgaggccgggagttgcccagagccagtatttcgatctccctttgacacagcctatcaatcacggagcacctgccgctcacttccttcaccagcctccgatggaaggtccttgggttccagagcaatggatgttccaaggtgcgccaccatctcagggcacagacgtggtccagcatcagctggacgcgcttggctacactctacacggcctgaaccacccgggcgttccagtgagtccagccgtgaaccagtaccatctctctcaggctgccttcggcctgcccattgacgaggacgagagcggcgagggcagcgataccagcgagccctgcgaagccctggatctttccatccacggccgaccttgcccacaggctccagagtggcccgtacaggaagaaggcggccaggacgccaccgaggtcctggacttgtccatacatggcagaccgcgccctaggaccccggagtggccagttcagggtgaaggtggtcagaacgtgaccggcccggaaacccgaagggtggtcgtgtccgctgttgtccacatgtgtcaggatgacgaattcccagatctgcaagatccacccgacgaggcc

SEQ ID NO: 43 - protein, 944 aa, synthetic construct
MDKDRPGPPALDDNMEEEVPSTSVVQEQVSAGDWENVLIELSDSSSEKEAEDAHLEPAQKGTKRKRVDHDAGGSAPARPMLPPQPDLPGREAILRRFPLDLRTLLQAIGAAATRIDTRAIDQFFGSQISNTEMYIMYAMAIRQAIRDRRRNPASRRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGPKVKRPPIFIRRLHRLLLMRAGKRTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQAFAPVRPQHMPVEPVPVPTVALERPVYPKPVRPAPPKIAMQGPGETSGIRRARERWRPAPWTPNPPRSPSQMSVRDRLARLRAEAQVKQASVEVQPPQLTQVSPQQPMEGPLVPEQQMFPGAPFSQVADVVRAPGVPAMQPQYFDLPLIQPISQGAPVAPLRASMGPVPPVPATQPQYFDIPLTEPINQGASAAHFLPQQPMEGPLVPEQWMFPGAALSQSVRPGVAQSQYFDLPLTQPINHGAPAAHFLHQPPMEGPWVPEQWMFQGAPPSQGTDVVQHQLDALGYTLHGLNHPGVPVSPAVNQYHLSQAAFGLPIDEDESGEGSDTSEPCEALDLSIHGRPCPQAPEWPVQEEGGQDATEVLDLSIHGRPRPRTPEWPVQGEGGQNVTGPETRRVVVSAVVHMCQDDEFPDLQDPPDEA

SEQ ID NO: 44 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcacttacgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 45 - protein, 865 aa, synthetic construct
MEAALLVCQYTIQSLIHLTGEDPGFFNVEIPEFPFYPTCNVCTYDVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 46 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacattcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 47 - protein, 865 aa, synthetic construct
MEAALLVCQYTIQSLIHLTGEDPGFFNVEIPEFPFYPTCNVCTADVNVTFNFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 48 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatactcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 49 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEILGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 50 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacctggaaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 51 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTWKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 52 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcacttacgacgtgaacgtgacattcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatactcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacctggaaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 53 - protein, 865 aa, synthetic construct
MEAALLVCQYTIQSLIHLTGEDPGFFNVEIPEFPFYPTCNVCTYDVNVTFNFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEILGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTWKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 54 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcacttacgacgtgaacgtgacattcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattacggctgtggtgagggcccagggcttggacgtgactctgcccttatctctacctacttctgcccaagactccaatttcgcggtcaagaccgagatactcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttgcggtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccgcgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgacctgggatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 55 - protein, 865 aa, synthetic construct
MEAALLVCQYTIQSLIHLTGEDPGFFNVEIPEFPFYPTCNVCTYDVNVTFNFDVGGKKHQLDLDFGQLTPHTKAVYQPRGAFGGSENAANLFLLELLGAGELALTMRSKKLPINVTTGEEQQVSLESVDVYFQDVFGTMWCHHAEMQNPVYLIPETVPYIKWDNCNSANITAVVRAQGLDVTLPLSLPTSAQDSNFAVKTEILGNEIDIECIMEDGEISQVLPGDNKFNIACSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVAVTAFWAWPNNTETDFKCKWTLTSGTPSGCENISGAFASNRTFDITVSGLGTAPKTLIITRTATNAATTTHKVIFSKAPESTTTSPTLNTTGFADPNTTTGLPSSTHVPTNLTAPASTGPTVSTWDVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 56 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtggcgatcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttgcgaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattgcggctgtggtgagggcccagggcttggacgtgactctgcccttatctctacctacttctgcccaagactccaatttcgcggtcaagaccgagatgctcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatgcgatcttatacgtgttttatagcggaaacggccctaaagcgtccggaggggactattgtattcagagtaatatcgttttctctgatgagattcctgccagtcaggacatgccgacaaacacagcggatattacctacgtgggcgacaatgccgcgtattcagtgcccatggtcacgagcgaggacgccaattcaccaaatgttgcggtaacagctttctgggcctggccaaataacgcggagactgacttcaaatgtaagtggactttgacctctggaactccgtcgggttgcgagaatatcgcgggggcctttgcttccaacagggcgttcgacatcactgtctcagggctggggacagcaccgaagacattaatcataacacggaccgccaccaacgccgcgactacaacccataaggtgatcttttccaaggcacctgagtccaccactacctccccgactcttaacactgcgggcttcgctgatcccaacaccgcgacggggttgcctagctcgacacatgtgccgacgaacctggcggcccctgcatcgaccgggcccacagtttcgaccgccgatgtgacatcaccaacgcccgcaggtacaacctcaggcgccagcccagtgaccccttccccaagcccctgggataatggagcggagtccaaggctcctgatatgacttcctctaccagccccgtgactacacccactcccaacgcagcgagcccaaccccagctgtgacgacgcccaccccgaacgcggcgtctcccacacctgctgtgacaaccccaacccctaacgccgcgagccctaccctaggtaagaccagtccgactagcgccgttacaacccctacccctaacgcagcgggcccgaccgtgggcgagacttccccgcaagccaatgcggcgaatcacgcgttgggcgggacctctcctacaccagtcgttacatctcagcctaagaacgctgcgtccgctgtcactaccggacagcacaacatcgcgagctcaagcacgagttccatgagcttgcggccgagctcaaatcccgagaccctatcaccatccacatcagacaacagtgcgtcacacatgccactcttgacgagcgctcaccccaccggcggcgagaacatcgcgcaggtgactccggcgtctatttccacccaccacgtcagcacgtcgtctcccgcaccgagaccagggacgacttctcaggccagtgggcctggcaactccgcgacaagcacaaagccaggcgaagttaacgtggcgaagggaacgcctccccagaacgcagcgagtcctcaggcgcccagcgggcagaagactgcggtgccaactgtgaccagcaccggtggcaaggccaactcagcgactggaggcaagcatacgacggggcacggcgcccggacctccactgaacccacgaccgattacggaggtgacagcacaacaccgcggccacgatataatgccgcgacttatctgccaccatccacaagctccaagctgcggccacggtggaccttcacaagcccacccgtgacgactgcccaagcgacggtgccagtgccacctacaagccagccacgcttctccaaccttgcgatgctcgttctccagtgggccagccttgctgttctgaccctcctcctgctgctcgtgatggccgattgcgcctttaggagaaacctcgcgactagccacacgtacaccacgccgccctacgatgacgccgagacttacgtc

SEQ ID NO: 57 - protein, 865 aa, synthetic construct
MEAALLVCQYTIQSLIHLTGEDPGFFNVEIPEFPFYPTCNVCTADVNVAINFDVGGKKHQLDLDFGQLTPHTKAVYQPRGAFGGSENAANLFLLELLGAGELALTMRSKKLPINVATGEEQQVSLESVDVYFQDVFGTMWCHHAEMQNPVYLIPETVPYIKWDNCNSANIAAVVRAQGLDVTLPLSLPTSAQDSNFAVKTEMLGNEIDIECIMEDGEISQVLPGDNKFNIACSGYESHVPSGGILTSTSPVATPIPGTGYAYSLRLTPRPVSRFLGNNAILYVFYSGNGPKASGGDYCIQSNIVFSDEIPASQDMPTNTADITYVGDNAAYSVPMVTSEDANSPNVAVTAFWAWPNNAETDFKCKWTLTSGTPSGCENIAGAFASNRAFDITVSGLGTAPKTLIITRTATNAATTTHKVIFSKAPESTTTSPTLNTAGFADPNTATGLPSSTHVPTNLAAPASTGPTVSTADVTSPTPAGTTSGASPVTPSPSPWDNGAESKAPDMTSSTSPVTTPTPNAASPTPAVTTPTPNAASPTPAVTTPTPNAASPTLGKTSPTSAVTTPTPNAAGPTVGETSPQANAANHALGGTSPTPVVTSQPKNAASAVTTGQHNIASSSTSSMSLRPSSNPETLSPSTSDNSASHMPLLTSAHPTGGENIAQVTPASISTHHVSTSSPAPRPGTTSQASGPGNSATSTKPGEVNVAKGTPPQNAASPQAPSGQKTAVPTVTSTGGKANSATGGKHTTGHGARTSTEPTTDYGGDSTTPRPRYNAATYLPPSTSSKLRPRWTFTSPPVTTAQATVPVPPTSQPRFSNLAMLVLQWASLAVLTLLLLLVMADCAFRRNLATSHTYTTPPYDDAETYV

SEQ ID NO: 58 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtggcgatcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttgcgaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattgcggctgtggtgagggcccagggcttggacgtgactctgcccttatctctacctacttctgcccaagactccaatttcgcggtcaagaccgagatgctcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatgcgatcttatacgtgttttatagcggaaacggccctaaagcgtccggaggggactattgtattcagagtaatatcgttttctctgatgagattcctgccagtcaggacatgccgacaaacacagcggatattacctacgtgggcgacaatgccgcgtattcagtgcccatggtcacgagcgaggacgccaattcaccaaatgttgcggtaacagctttctgggcctggccaaataacgcggagactgacttcaaatgtaagtggactttgacctctggaactccgtcgggttgcgagaatatcgcgggggcctttgcttccaacagggcgttcgacatcactgtctcagggctggggacagcaccgaagacattaatcataacacggaccgccaccaacgccgcgactacaacccataaggtgatcttttccaaggcacctgagtccaccactacctccccgactcttaacactgcgggcttcgctgatcccaacaccgcgacggggttgcctagctcgacacatgtgccgacgaacctggcggcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 59 - protein, 865 aa, synthetic construct
MEAALLVCQYTIQSLIHLTGEDPGFFNVEIPEFPFYPTCNVCTADVNVAINFDVGGKKHQLDLDFGQLTPHTKAVYQPRGAFGGSENAANLFLLELLGAGELALTMRSKKLPINVATGEEQQVSLESVDVYFQDVFGTMWCHHAEMQNPVYLIPETVPYIKWDNCNSANIAAVVRAQGLDVTLPLSLPTSAQDSNFAVKTEMLGNEIDIECIMEDGEISQVLPGDNKFNIACSGYESHVPSGGILTSTSPVATPIPGTGYAYSLRLTPRPVSRFLGNNAILYVFYSGNGPKASGGDYCIQSNIVFSDEIPASQDMPTNTADITYVGDNAAYSVPMVTSEDANSPNVAVTAFWAWPNNAETDFKCKWTLTSGTPSGCENIAGAFASNRAFDITVSGLGTAPKTLIITRTATNAATTTHKVIFSKAPESTTTSPTLNTAGFADPNTATGLPSSTHVPTNLAAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 60 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 61 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENAANLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 62 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 63 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSANITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 64 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttcgcggtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 65 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFAVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 66 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 67 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNIACSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 68 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttgcggtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 69 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccgcgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 70 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNAATTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 71 - RNA, 669 nt, synthetic construct; identical to SEQ ID NO: 33
  source 1..669: mol_type = other RNA

SEQ ID NO: 72 - RNA, 2832 nt, synthetic construct; identical to SEQ ID NO: 42
  source 1..2832: mol_type = other RNA

SEQ ID NO: 73 - RNA, 726 nt, synthetic construct; identical to SEQ ID NO: 41
  source 1..726: mol_type = other RNA

SEQ ID NO: 74 - RNA, 1158 nt, synthetic construct; identical to SEQ ID NO: 39
  source 1..1158: mol_type = other RNA

SEQ ID NO: 75 - RNA, 1491 nt, synthetic construct; identical to SEQ ID NO: 40
  source 1..1491: mol_type = other RNA

SEQ ID NO: 76 - RNA, 1491 nt, synthetic construct; identical to SEQ ID NO: 40
  source 1..1491: mol_type = other RNA

SEQ ID NO: 77 - RNA, 2595 nt, synthetic construct
  source 1..2595: mol_type = other RNA
atggaagccgctcttctggtctgccagtatacaatccagtcgctgattcatctcaccggcgaggatccaggtttctttaacgtggagattccagaattccctttctatccaacttgtaacgtgtgtactgccgacgttaacgttactattaactttgacgtgggcggaaagaagcaccagctggatcttgatttcggccagctaacacctcataccaaggccgtctaccaaccacgcggggcatttggcggttctgagaatgctacaaacctgttccttttggaactcctcggagcaggggaactggcacttacaatgaggagtaagaagctgcccatcaacgtcaccacgggtgaagaacagcaggtgtccctggagtccgttgatgtgtatttccaggacgttttcgggacaatgtggtgtcatcatgccgaaatgcagaatccggtgtatctcattcctgagaccgtgccctatattaaatgggataactgtaattcgacaaacataactgccgtagtgcgtgcccagggtctggatgtaactttgcctctctcccttcccacttctgcccaggactccaatttctcagtgaagaccgaaatgcttgggaacgagatcgacatcgagtgtatcatggaggatggggagatcagtcaggtgttgcctggtgacaataagtttaatataacatgttccggctatgaatcacatgttccaagcggaggtattctcacaagtacatctcccgtagccacgccgattccgggaaccggatacgcctattcacttcggctgactcctcggccagtgtcacgattcctgggcaataactcaattctgtacgtcttttacagcggcaacggcccaaaggccagtggaggcgattactgtatccagtctaacatcgtattttcagatgagattccagcaagccaagacatgccaactaatactacagacattacatacgtcggtgataacgccacttattcagtccctatggtcacatccgaggacgcgaactcacccaatgttaccgttaccgcattctgggcatggcccaataatacggaaacagacttcaaatgtaagtggaccctgaccagtggcaccccttctggctgcgagaatataagcggggcatttgctagcaacagaacattcgatattactgtgtctggcctggggaccgcgcccaagacactcattataacgcgaacagctacgaacgctaccacaaccacacataaagtgattttcagcaaggctccagaatcaactacaaccagtccgacccttaataccacaggcttcgcagacccgaacacaacgacgggactgccctcctcaacgcatgtgccgactaatctgactgctcccgcaagtaccggcccaacagtctccacagcagacgtcacctctccgactcccgccggcactacatctggagcctcaccggtgactcctagtccttcgccctgggacaatgggaccgagtctaaagccccggacatgacgagttctacctcaccagttacaaccccaacacctaatgccacaagccctacgccagccgtgaccacccctacgcccaatgccaccagcccaacacccgctgttacaacgccaacgcccaacgctacttctccaacgctaggaaagacatcacccacctcagctgtaaccacgcccacaccaaacgctaccgggccaaccgttggcgaaacctcgccccaggccaatgcgacaaaccacacccttggcgggacttctcccactcctgtggtgacttcacaaccgaagaacgcgacgtcggccgttacgaccgggcagcacaatatcacctcgtcctccacgtcgtccatgtccttgcggccttccagtaacccagagacactgtctcctagtaccagtgacaacagcacctctcacatgccattactgacttccgcccacccaaccggcggagagaacatcactcaggtgacgcccgcatctatttctacccaccacgtgagcactagcagcccggcaccaagaccagggaccacctcccaggctagcggcccgggcaactcctccactagcacgaaaccaggagaggtgaacgtgaccaagggcacaccgccacagaatgcaacttccccacaggcgccctccggccagaagactgctgtacctacggtcactagcactggcggaaaggcgaattcaaccaccggtggtaagcataccaccggacacggcgctcggactagcacagagccaaccactgattacggaggtgactcaacgaccccgaggcccaggtacaacgcaaccacctatctcccaccatccacatcttccaagctcaggcctcgttggactttcacctccccgcctgtgactacagcgcaggccacagtgccagtgcctccaacgtcacagccgcggtttagcaatctctccatgctggtcctgcagtgggcatctctggcagtcctgacactgcttcttttgctcgtgatggccgattgtgcgtttagacgcaacctttcaacatcccacacatatacaactccaccctatgatgacgcagagacttatgtg

SEQ ID NO: 78 - protein, 865 aa, synthetic construct; identical to SEQ ID NO: 28

SEQ ID NO: 79 - RNA, 2118 nt, synthetic construct; identical to SEQ ID NO: 37
  source 1..2118: mol_type = other RNA

SEQ ID NO: 80 - RNA, 411 nt, synthetic construct; identical to SEQ ID NO: 35
  source 1..411: mol_type = other RNA

SEQ ID NO: 81 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 82 - RNA, 1029 nt, synthetic construct
  source 1..1029: mol_type = other RNA
atgagtgactggacaggaggagccctccttgtcctctattcctttgctctcatgcttataattatcatcctgataatctttatcttcagacgggaccttctctgtccacttggagccctttgtatactcctattgatgatcaccctcctcctcatcgctctctggaatttgcacggacaggcattgttccttggaattgtgctgttcatcttcgggtgcttacttgtcttaggtatctggatctacttattggagatgctctggcgacttggtgccaccatctggcagcttttggccttcttcctagcgttcttcctcgacctcattctactcattattgctctctatctacaacagaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggaccaccactctgctctcagaacctaggcgcacctggaggtggtcctgacaatggcccacaggaccctgacaacacggatgataacggtccacaagacccagataacactgacgacaatggaccgcatgacccgctgcctcaggatcctgataacacagacgacaacggcccacaagatccggacaataccgacgataacggaccgcacgacccgttacctcatagccctagcgactctgctggaaatgatggaggccctccacaattgacggaagaggttgagaacaaaggaggtgaccagggcccgcctttgatgacagacggaggcggcggtcatagtcatgattccggccatggcggaggagatccacaccttcctacgctgctgttgggttcttctggttccggtggagatgatgacgacccgcacggcccagttcagctaagctactatgac

SEQ ID NO: 83 - protein, 343 aa, synthetic construct
MSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 84 - RNA, 2118 nt, synthetic construct; identical to SEQ ID NO: 37
  source 1..2118: mol_type = other RNA

SEQ ID NO: 85 - RNA, 411 nt, synthetic construct; identical to SEQ ID NO: 35
  source 1..411: mol_type = other RNA

SEQ ID NO: 86 - RNA, 669 nt, synthetic construct; identical to SEQ ID NO: 33
  source 1..669: mol_type = other RNA

SEQ ID NO: 87 - RNA, 1881 nt, synthetic construct
  source 1..1881: mol_type = other RNA
atggacagcaagggcagcagccagaagggaagcaggttgttattgcttttagtggtgagcaacttactcttaccccagggcgtggtggaggcagcactcctcgtctgccagtatacaatccagtcgctcattcatttgacaggcgaggaccccggcttctttaacgtggaaatcccagagttccctttctatccaacctgtaacgtttgcacagccgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagatccctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctggcagcggcagcgcaggctctgcaagcggcggcgcctccagcggctccggaggtgccagcatgcagatctacgagggcaagctgaccgccgagggcctgaggttcggcatcgtggccagcaggttcaaccacgccctggtggacaggctggtggaaggcgccatcgacgccatcgtgaggcacggcggcagggaggaggacatcaccctggtgcgggtgcccggcagctgggagatccccgtggccgccggcgagctggccaggaaggaagatattgatgcagtgatcgccatcggcgtgctgatcaggggcgccaccccacacttcgactacatcgccagcgaagtgagcaagggactggcggacctgagcctggagctgaggaagcccatcaccttcggcgtgatcaccgccgacaccctggagcaggccatcgagagggccggcaccaagcacggcaacaagggatgggaggccgccctgagcgctatcgaaatggccaacctcttcaagagcctgagg

SEQ ID NO: 88 - protein, 627 aa, synthetic construct
MDSKGSSQKGSRLLLLLVVSNLLLPQGVVEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGSAGSASGGASSGSGGASMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 89 - RNA, 2097 nt, synthetic construct
  source 1..2097: mol_type = other RNA
atggacagcaagggcagcagccagaagggcagtaggctccttttgctacttgtggtgagcaacctcttgttaccccagggcgtggtggaggcagcactcctcgtctgccagtatacaatccagtcgctcattcatttgacaggcgaggaccccggcttctttaacgtggaaatcccagagttccctttctatccaacctgtaacgtttgcacggcagacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtagtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattccagccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctggcagcggcagcgctggctccgctagcggcggcgcctctagcggatcaggaggcgcatccatggagaacattgcatctggcctgctgggacctctactggtgctgcaggccggattcttcctgctgaccaagatcctgaccatccctcagagcctggacagctggtggaccagcctgaacttcctgggcggcaccccagtgtgcctgggccagaacagccagagccagatctcctcccacagccccacctgctgtccgcccatctgccccggctaccgttggatgtgcctgaggaggttcatcatcttcctgtgcatactcctgttgtgtctaattttcctgctcgttctgctcgactaccagggcatgctgccggtgtgtcctctgatccccggctcctccaccaccagcaccggcccctgcaggacctgcaccacaccagcacaaggtaccagcatgttccccagctgttgctgtaccaagcccaccgacggcaactgcacctgcatccccatccccagcagctgggccttcgccaagtacctgtgggagtgggccagcgtgaggttcagctggctgtcacttcttgcgccattcgtgcagtggttcgtgggcctgagccctactgtgtggctgtcggtaatctggatgatgtggttctggggccccagcctgtacaacatcctgtcaccgttcatgcctctcctaccgctgttcttctgcctgtgggtgtacatc

SEQ ID NO: 90 - protein, 699 aa, synthetic construct
MDSKGSSQKGSRLLLLLVVSNLLLPQGVVEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGSAGSASGGASSGSGGASMENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCRTCTTPAQGTSMFPSCCCTKPTDGNCTCIPIPSSWAFAKYLWEWASVRFSWLSLLAPFVQWFVGLSPTVWLSVIWMMWFWGPSLYNILSPFMPLLPLFFCLWVYI

SEQ ID NO: 91 - RNA, 2403 nt, synthetic construct
  source 1..2403: mol_type = other RNA
atggacagcaagggcagcagccagaagggatccaggctattgttactattagtggtgagcaacctcctcttgccccagggcgtggtggacatcgacccctacaaggagttcggcgccaccgtggagttgttaagcttcctccccagcgacttcttccccagcgtgagggacctgctggacaccgccgccgccctgtacagggaggccctggagagccccgagcactgctcgcctcaccacaccgccctgaggcaggccatcctgtgctggggcgagctgatgaccctggccacctgggtgggcaacaacctggaggaccccgccagccgcgacctcgttgtgaactacgtgaacaccaacatgggcctgaagatcaggcagctgctgtggttccacatcagctgcctgaccttcggcagggagacagtgctggagtacctggtgagcttcggcgtgtggatcaggactccacccgcctacaggcctcccaacgcgccaatcctgagcaccctgcccgagacaacagtggtgggcagcggcagcgcaggttccgccagcggcggcgcatccagtggctctggaggagcttctgacattgacccatataaggaattcggtgctacagtagagctactgtctttcttgcctagtgatttcttcccgagtgtaagggatctcctagatacagccgctgctctatatagagaggcactggaatctccagagcactgttccccacaccataccgctctccggcaggcaatcctgtgttggggtgaacttatgacactggcaacctgggtcggtaacaacggatcaggtgaggcggctctgctggtctgccagtatacaatccagtcgctcattcatctgacaggcgaagatccaggtttctttaacgtggaaatcccagagttccctttctatccaacctgtaacgtttgtacagctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgccttctggtggcatactcacaagtacttctcccgtagccacgcccattcccggaaccggatatgcgtatagtctgcgtctgaccccacggcctgtgtccagattcctgggtaacaatagtatcttatacgtgttttatagcggaaacggccctaaagcgtccggaggggactattgtattcagagtaatatcgttttctctgatgaaattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctggatccggactggtggtcaactatgtgaatacaaatatgggcctcaagattaggcagctcctatggttccatatttcctgcttaactttcggtagggaaactgtcctggaatatctcgttagcttcggagtgtggattcggactcctcctgcctacagaccacctaacgctccaattctgtcaactctcccagagacgactgtggtcagaaggaggggcaggagccccagacggcggacaccaagccctcgccgaagaagaagccaaagcccgcggcgccggagatctcagtccagagagagccagtgc

SEQ ID NO: 92 - protein, 801 aa, synthetic construct
MDSKGSSQKGSRLLLLLVVSNLLLPQGVVDIDPYKEFGATVELLSFLPSDFFPSVRDLLDTAAALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVGSGSAGSASGGASSGSGGASDIDPYKEFGATVELLSFLPSDFFPSVRDLLDTAAALYREALESPEHCSPHHTALRQAILCWGELMTLATWVGNNGSGEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGLVVNYVNTNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 93 - RNA, 2538 nt, synthetic construct
  source 1..2538: mol_type = other RNA
atggacagcaagggcagcagccagaagggtagcaggctactcttgctcctagtggtgagcaacttgcttcttccccagggcgtggtggaggcagcattgcttgtctgccagtatacaatccagtcgctcattcatctaacaggcgaggaccccggcttctttaacgtggaaatcccagagttccctttctatccaacctgtaacgtttgcactgccgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgccttccggcggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattccagccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaatactactactgggttgcctagctcgacacatgtgccgacgaacctgactgcccctgcatcgaccgggcccacagtttcgaccgccgatgtgacaagccctacgcccgcaggtacaacctcaggcgccagcccagtgaccccttccccaagcccctgggacaacggaacggagtccaaggctcctgatatgacttcctctaccagccccgtgactacacccactcccaacgcaactagcccaaccccagctgtgacgacgcccaccccgaacgcgacatctcccacacctgctgtgacaacccctacccctaacgccactagccctaccctaggtaagaccagtccgactagcgccgttactacaccgacccctaatgcaaccggcccgactgtcggcgagacttccccgcaagccaatgcgacaaatcacacattgggcgggacctctcctacaccagtcgttacatctcagcctaagaacgctacctccgctgtcactaccggccagcacaacatcacaagctcaagcacgagttccatgagcttgcggccgagctcaaatcccgagacactatcaccatccacatcagacaacagtacttcacacatgccactcttgacgagcgctcaccccaccggcggcgagaacattacccaggtgactccggcgtctatttccacccaccacgtcagcacgtcgtctcccgcaccacggccagggacgacttctcaggccagcggcccaggaaactcctctacaagcacaaagccaggcgaagttaacgtcaccaagggaacgcctccccagaacgcaacctccccacaggctcccagcgggcagaagactgcggtgccaactgtgaccagcaccggtggcaaggccaactcaacaactggaggcaagcatacgacggggcacggcgcccggacctccactgaacccacgaccgattacggaggtgatagtaccaccccgcggccacgatataatgccaccacttatctgccacctagcacttcctccaagctccggccacggtggaccttcacaagcccacccgtgacgacagcccaggcgacggtgccagtcccacctacaagccagccacgcttctccaaccttagt

SEQ ID NO: 94 - protein, 846 aa, synthetic construct
MDSKGSSQKGSRLLLLLVVSNLLLPQGVVEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLS

SEQ ID NO: 95 - RNA, 3057 nt, synthetic construct
  source 1..3057: mol_type = other RNA
atggactcgaagggatctagtcagaaaggctctaggctcctactcctcttggtggtgagcaacttactccttccccagggcgtggtgagagcagtgggcgttttcttagcaatctgcctcgtgacaatctttgtgttgccgacctggggcaactgggcctacccctgctgccacgtgacccagctgagagctcaacacctgcttgccctggagaacatcagcgacatctaccttgtaagcaaccagacatgcgacggcttcagtctggccagcctgaacagccccaagaacggctcaaaccagctagtgatcagccggtgcgctaacggtctgaacgttgtgagcttcttcatcagcatcttgaagagaagtagtagcgcgctgaccggccacctgagagagctgctgaccactctcgagacactctatggcagcttcagcgtggaggacctgttcggcgcaaacctgaaccgctacgcctggcacagaggcggcggaagcggtagcgctggcagcgccagcggaggcgcttcatccggatccggcggcgcaagcagcctttctgaggtgaagctccacttggacatcgaaggccacgccagccattacaccataccatggaccgagctgatggcaaaggtgcccggccttagcccagaagccctgtggcgagaggccaacgttaccgaagacttggctagtatgttaaacaggtacaaacttatctacaagactagcggcaccctgggcatcgcattagccgagcccgtggacatccccgccgttagcgagggcagcatgcaggtggacgccagcaaggttcaccctggtgtaattagtggcctcaacagcccagcctgtatgttgagcgccccactggagaagcagctgttttactatatcgggaccatgttgcccaatacccggccacacagttacgtgttctaccagctgcggtgtcacctgagctacgtggccctgtctatcaacggcgacaagttccagtacaccggcgccatgaccagcaagttcctgatgggcacctacaagcgggtcaccgagaagggcgacgagcacgtgctgtcacttgtgttcggtaagaccaaggacctgcccgaccttcggggccccttttcataccctagtttgaccagcgcccaaagtggcgactacagccttgtgatagtgaccaccttcgtgcattacgccaacttccacaactacttcgtgcccaacctgaaggacatgttcagccgggcggtaaccatgacggccgcttcttatgcccggtatgtgctgcagaagctggtcctgctggagatgaagggcgggtgccgggagcccgagttggacaccgaaaccctgaccaccatgttcgaggttagcgtggccttcttcaaggtgggtcacgcggtgggcgaaaccggcaatggatgcgtggacttacgctggctggccaagagtttctttgagcttaccgtgctgaaggatatcatcggcatctgctacggcgccaccgtaaagggaatgcagagctatgggctggagagacttgccgccatgctcatggctaccgtgaagatggaggagctgggacacctgacaacagagaagcaggagtacgccctgagactggccaccgtgggctaccctaaggccggcgtgtacagtggactcataggcggagctaccagcgtgctcctatccgcttataacagacaccccttatttcagcccctgcacacagtgatgcgggagactttattcatcggatctcacgtcgtactgcgggagttgcggctgaacgtaaccacccagggccctaatttagcactttatcagctgctcagtactgccctgtgcagcgcacttgagatcggcgaggtactcagaggcctggccctcggtacggagagtggcctgttttctccatgctaccttagcctgcggtttgatctgaccagagacaagttgctgagcatggctcctcaagaggcgacccttgaccaggcagccgtgtcaaacgccgtggacggattcctgggcagactgtctctggaacgggaagaccgggatgcttggcaccttcctgcctataagtgtgtggatcggctggataaggtcctgatgataatccctctgattaacgttactttcatcatctctagcgatcgggaggtccgagggtccgccctctacgaggccagcaccacctatctgagcagcagcctcttcttgtctcctgtgattatgaacaagtgcagccagggtgcagtagccggcgagccccggcagatcccgaagatccagaacttcactcggacccagaagtcttgtatcttctgcggcttcgcccttctgagttacgacgagaaggagggcctggagactacaacttacatcacaagccaggaggtgcagaacagcatcctgtcatctaattactttgacttcgacaacctgcacgttcattatctactgctaacaactaacggtaccgtcatggaaatcgcaggactgtatgaggagcgggccggcagcgggagcgcgggctctgccagcggtggtgccagcagcggcagtggaggtgcctccatgcagatttacgaaggaaagctgaccgccgaaggcctgaggttcggcatcgtggcttccagattcaaccacgcactggtggacaggctggtggagggcgccatcgacgcaatcgtgcgccacggcggcagggaggaggacatcaccctcgtaagagtccctggtagctgggaaattcccgtggccgcgggcgagctggctaggaaggaggatatcgacgccgtcatcgccatcggcgttctcatccggggtgccacaccacatttcgactacatcgcaagcgaggtttcaaagggactcgccgacttaagtctggagctgaggaagcccatcacctttggcgtgatcaccgccgacaccctggagcaggccatcgagcgcgccggcaccaagcacgggaacaagggctgggaggcagccttatcagccatcgaaatggcgaatctgttcaagagcctgcgg

SEQ ID NO: 96 - protein, 1019 aa, synthetic construct
MDSKGSSQKGSRLLLLLVVSNLLLPQGVVRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGGGSGSAGSASGGASSGSGGASSLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAGSGSAGSASGGASSGSGGASMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 97 - RNA, 2784 nt, synthetic construct
  source 1..2784: mol_type = other RNA
atggactctaagggcagcagccagaagggcagtaggctcctacttctcctcgtggtgagcaacttattgcttccccagggagtggtgcagactccggaacagcccgctccgcccgctaccaccgtccagcctaccgccacgcgccagcagacttccttcccgtttcgcgtctgcgagctctcctctcacggcgatctcttccgcttctcctccgatattcagtgcccctccttcggcacccgcgagaaccataccgagggcctcctcatggtctttaaggacaacatcatcccttactccttcaaggtacgctcctacacaaagatagtcaccaacatcctcatctataatggatggtacgctgactctgttaccaatcgccacgaggagaagttctccgttgattcctacgagactgaccagatggataccatctaccagtgctacaatgccgtcaagatgaccaaggacggcctgacccgcgtctatgtcgaccgcgacggcgttaacataactgtcaacctcaagcctacaggcggcctggccaatggcgtcagacgctacgcctcccagaccgagctctacgacgctcctggctggctgatctggacctaccggacccgcaccactgtcaattgcctcattaccgacatgatggccaagtccaatagcccattcgatttcttcgtaaccaccaccggccagaccgtcgagatgtcgccattctatgacggcaagaataaggaaactttccacgagagagccgatagcttccacgtccgcacgaactacaagattgtcgactacgacaatcgcggcaccaatccacagggcgagcgccgcgccttcctggacaagggcacctacaccctctcctggaaactcgagaacagaaccgcctactgtcccctccagcactggcagacttttgattccaccatcgccactgagactggcaagagtatccatttcgtcaccgacgaaggaacatcttcattcgtcacaaataccactgtgggcattgagctccccgacgccttcaagtgcatcgaggaacaggtcaacaagacaatgcacgagaagtacgaggccgtgcaggaccggtataccaagggccaggaggccattacatacttcatcaccagcggtggattgcttctcgcctggcttccactcacacctcgcagtctggccacagttaagaacctcactgagttgaccactccaacatcgtcaccaccttcttcacctagccctcctgctccttccgccgcccgcggctccactcctgccgctgtcttaaggggctcgggcagcgccggcagcgcatcgggaggggcgagttcagggtcaggcggtgcctccgcaggcaatgctaccacaccagtgccaccaaccgcccctggcaagtccttaggtaccctgaacaatccagcgacagttcagatccagttcgcttacgattcactcaggcgccagatcaaccgcatgcttggtgaccttgcccgggcctggtgcctcgaacagaagcgccagaacatggtacttagggaacttaccaagattaatcctactacagtcatgtcctctatctacggcaaggccgttgctgccaagcgcctgggcgacgtcatcagcgtgtcgcagtgcgtcccagtcaaccaggctaccgttaccctcaggaagtccatgagagtgccaggctccgaaacaatgtgctattcccgccctctggtttcattttccttcatcaacgacacaaagacctacgagggccagttaggaactgacaatgagatattcttgactaagaagatgactgaggtatgccaggcaacttctcaatactacttccagtctggaaatgaaatccacgtatataacgactatcatcactttaagactatcgaactcgacggaattgctacactccaaacattcatctcactgaatacctcccttatcgagaacatcgacttcgcatccctcgagctgtatagcagagacgagcagagagccagcaacgtgttcgacctggagggtatctttcgcgagtacaacttccaggcacagaatatagccggcctccgtaaggacctggataacgccgtgtccaatggccgcaaccagttcgtcgacggtcttggcgaactcatggactctctgggatccgtggggcaatccatcacaaacttagtctctactgtcggtggactattcagcagcctggttagcggatttatctctttctttaagaacccaggcagtggttctgccggctcagccagcggcggcgctagtagcggcagcggaggcgcctcaatgcagatctatgagggtaagttgacagccgaaggcttgaggtttggcatcgtagccagccggttcaaccacgccctggtggacaggctggttgagggtgccattgacgccattgtgaggcacggcggcagggaggaggacattaccctggtcagagtccccggatcatgggagatccccgtggccgccggggagctggccaggaaggaagacatcgacgctgtgatcgccatcggcgtgctgatccgaggagccacaccccatttcgactacatcgccagtgaggttagcaaaggcctagcggacctgagcctggagctgcgcaagcccatcaccttcggcgtcattactgccgacaccttggaacaggccatcgaaagggccggtacaaagcacggcaacaagggctgggaggcagcccttagcgccatcgagatggcaaacctgtttaagtccttgagg

SEQ ID NO: 98 - protein, 928 aa, synthetic construct
MDSKGSSQKGSRLLLLLVVSNLLLPQGVVQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRGSGSAGSASGGASSGSGGASAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGSGSAGSASGGASSGSGGASMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 99 - RNA, 2595 nt, synthetic construct; identical to SEQ ID NO: 44
  source 1..2595: mol_type = other RNA

SEQ ID NO: 100 - RNA, 2595 nt, synthetic construct; identical to SEQ ID NO: 54
  source 1..2595: mol_type = other RNA

SEQ ID NO: 101 - RNA, 2595 nt, synthetic construct; identical to SEQ ID NO: 64
  source 1..2595: mol_type = other RNA

SEQ ID NO: 102 - RNA, 2595 nt, synthetic construct; identical to SEQ ID NO: 68
  source 1..2595: mol_type = other RNA

SEQ ID NO: 103 - RNA, 2595 nt, synthetic construct; identical to SEQ ID NO: 69
  source 1..2595: mol_type = other RNA

SEQ ID NO: 104 - RNA, 57 nt, synthetic construct
  source 1..57: mol_type = other RNA
gggaaataagagagaaaagaagagtaagaagaaatataagaccccggcgccgccacc

SEQ ID NO: 105 - RNA, 2118 nt, synthetic construct; identical to SEQ ID NO: 37
  source 1..2118: mol_type = other RNA

SEQ ID NO: 106 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 107 - RNA, 411 nt, synthetic construct; identical to SEQ ID NO: 35
  source 1..411: mol_type = other RNA

SEQ ID NO: 108 - RNA, 669 nt, synthetic construct
  source 1..669: mol_type = other RNA
atggtgagcttcaagcaggtgagagtgcccctgttcaccgccatcgccctggtgatcgtgctgctgctggcctacttcctgccacccagagtgagaggcggcggcagagtggccgccgccgccatcacctgggtgcccaagcccaacgtggaggtgtggcccgtggacccgccacctcccgtgaacttcaacaagaccgccgagcaggagtacggcgacaaggaggtgaagctgccccactggacgccgaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccaccccagacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagc

SEQ ID NO: 109 - RNA, 2595 nt, synthetic construct; identical to SEQ ID NO: 81
  source 1..2595: mol_type = other RNA

SEQ ID NO: 110 - RNA, 1881 nt, synthetic construct
  source 1..1881: mol_type = other RNA
atggacagcaagggcagcagccagaagggatcgaggctattattgttacttgtggtgagcaacttgctcctgcctcagggcgtggtgggcgaggccgccttgttggtgtgccagtacaccatccagagcctcatccacctcaccggcgaggaccccggcttcttcaacgtggagatccccgagttccccttctaccccacctgcaacgtgtgcaccgccgacgtgaacgtgaccatcaacttcgacgtgggcggcaagaagcaccagctggacctggacttcggccagctgaccccgcacaccaaggccgtgtaccagcccaggggcgccttcggcggcagcgagaacgccaccaacctgttcctgctggagctgctgggcgccggcgagctggccctgaccatgaggtccaagaagctgcccatcaatgtgaccacaggagaggagcagcaggtgagcctggagagcgtggacgtgtacttccaggacgtgttcggcaccatgtggtgccaccacgccgagatgcagaacccagtttacttgattcctgagacagtgccctacatcaagtgggacaactgcaacagcaccaacatcaccgccgtggtgagggcccagggcctggacgtgaccctgcccctgagcctgcccaccagcgcccaggatagcaacttcagcgtgaagaccgaaatgctgggcaacgagatcgacatcgagtgcatcatggaggacggcgagatcagccaggtgctgcccggcgacaacaagttcaacattacctgcagcggctacgagagccacgtgcccagcggcggcatcctgacctcaacttcacccgtggccactccgatccccggcaccggctacgcctacagcctgcgtctgacacctaggcccgtgagcaggttcctgggcaataacagcatcctgtacgtgttctacagcggcaacggccccaaggcttccggcggcgactactgtattcaatcaaacatcgtgttcagcgacgagataccagccagccaggacatgcctactaacaccaccgacatcacctatgtgggagacaatgctacttacagcgtgcccatggtgaccagcgaggacgccaacagcccaaatgtcaccgtgaccgccttctgggcctggcccaacaacactgagacagacttcaagtgcaagtggaccctcacatccggcacccctagcggatgcgagaacatcagcggagcgttcgccagcaacaggaccttcgatataacagtgagcggcctgggcaccgctcctaagaccctgatcatcaccaggacggccacaaatgcaactactactacacacaaggtgatcttcagcaaggctccggaatcacaagtgaggcagcagttctcaaaggatatcgagaagcttctgaacgagcaagttaacaaggaaatgcagagcagtaatctctacatgagcatgagcagctggtgctacacccactccctggacggagcaggcctcttcctgttcgaccacgcagccgaggagtacgagcacgctaagaagttgatcattttcttgaacgagaacaacgtgcccgtgcagctaacgtcaatcagcgcacctgagcacaagttcgagggcctgacccagatcttccagaaggcctacgaacacgaacagcacatctccgagagcatcaacaatattgtggatcacgctatcaagtccaaggaccacgctaccttcaacttcctgcagtggtacgtggccgagcaacatgaggaggaggtgctgttcaaggacatcctggacaagatcgagctgatcggtaatgagaatcacggcctgtacctggccgaccagtacgtgaagggcatcgccaagagccggaagtca

SEQ ID NO: 111 - protein, 627 aa, synthetic construct
MDSKGSSQKGSRLLLLLVVSNLLLPQGVVGEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 112 - RNA, 1884 nt, synthetic construct
  source 1..1884: mol_type = other RNA
atggacagcaagggcagcagccagaagggctcccggttgttgctcttgctcgtggtgagcaacttgttgttgcctcagggcgtggtgggcgaggccgccttgttggtgtgccagtacaccatccagagcctcatccacctcaccggcgaggaccctggcttcttcaacgtggagatccctgagttccctttctaccctacctgcaacgtgtgcaccgccgacgtgaacgtgaccatcaacttcgacgtgggcggcaagaagcaccagctggacctggacttcggccagctgacccctcacaccaaggccgtgtaccagcctagaggcgccttcggcggcagcgagaacgccaccaacctgttcctgctggagctgctgggcgccggcgagctggccctgaccatgagaagtaagaagctgcctatcaacgtcactactggcgaggagcagcaggtgagcctggagagcgtggacgtgtacttccaggacgtgttcggcaccatgtggtgccaccacgccgagatgcagaaccctgtgtacctgatcccagaaaccgtgccttacatcaagtgggacaactgcaacagcaccaacatcaccgctgtggtgagagcccagggcctggacgtgaccctgcctctgagcctgcctaccagcgcccaagacagtaacttcagcgtgaagacggagatgctgggcaacgagatcgacatcgagtgcatcatggaggacggcgagatcagccaggtgctgcctggcgacaacaagttcaacataacatgcagcggctacgagagccacgtgcctagcggcggcatcctgacctccaccagccctgtggccacccctatccctggcaccggctacgcctacagcctgagactcacgccaagacctgtgagcagattcttaggaaacaacagcatcctgtacgtgttctacagcggcaacggccctaaggcctccggcggtgactactgtatccagtctaacatcgtgttcagcgacgaaatcccagccagccaggacatgcctacaaacaccaccgacatcacttatgtcggcgacaatgcgacatacagcgtgcctatggtgaccagcgaggacgccaacagccctaacgtaaccgtgaccgccttctgggcctggcctaacaacaccgaaaccgacttcaagtgcaagtggactttaactagtggcaccccttctggctgcgagaacatcagcggcgcattcgccagcaacagaaccttcgatatcaccgtgagcggcctgggcaccgcccctaagaccctgatcatcaccagaaccgcaactaatgcaactacaacaacccacaaggtgatcttcagcaaggcccctggctccggctctgccggcagtggcagcgcaggcagtggatccgccggttcaggttccgccatgcagatctacgagggcaagctgaccgccgagggcctgagattcggcatcgtggccagcaggttcaaccacgccctggtggacagactggtggagggcgccatcgacgccatcgtgagacacggaggaagagaggaggatataacactggtcagagtgcctggatcatgggaaatccctgtcgccgctggtgagctggcaagaaaggaggacatcgatgcggtgatcgccatcggcgtgctgatccgcggtgcaactccacacttcgactacatcgcctctgaagtgtcaaagggccttgcggacctgtccctcgaactcagaaagcctatcacattcggagtgattactgcagataccctggagcaggccatcgagagagccggcaccaagcacggcaacaagggctgggaagccgcgctgtctgccatagaaatggcaaaccttttcaagtcccttaga

SEQ ID NO: 113 - protein, 628 aa, synthetic construct
MDSKGSSQKGSRLLLLLVVSNLLLPQGVVGEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPGSGSAGSGSAGSGSAGSGSAMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 114 - protein, 865 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVAVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 115 - protein, 30 aa, synthetic construct
MDSKGSSQKGSRLLLLLVVSNLLLPQGVVG

SEQ ID NO: 116 - protein, 18 aa, synthetic construct
MDWTWILFLVAAATRVHS

SEQ ID NO: 117 - protein, 20 aa, synthetic construct
METPAQLLFLLLLWLPDTTG

SEQ ID NO: 118 - protein, 24 aa, synthetic construct
MLGSNSGQRVVFTILLLLVAPAYS

SEQ ID NO: 119 - protein, 17 aa, synthetic construct
MKCLLYLAFLFIGVNCA

SEQ ID NO: 120 - protein, 15 aa, synthetic construct
MWLVSLAIVTACAGA

SEQ ID NO: 121 - skipped

SEQ ID NO: 122 - RNA, 11 nt, synthetic construct
  source 1..11: mol_type = other RNA
gggatcctacc

SEQ ID NO: 123 - skipped

SEQ ID NO: 124 - RNA, 2595 nt, synthetic construct
  source 1..2595: mol_type = other RNA
atggaagccgccctattagtctgccagtacaccattcaaagcctcatccacttgactggggaggatcctgggttctttaacgtggaaatcccggaattccctttctacccaacttgcaacgtgtgcacggctgacgtcaacgtgactatcaacttcgatgtgggagggaagaaacatcaacttgatctggactttggccagcttactccccacaccaaggccgtgtaccagccccgcggcgcattcggtgggagcgagaacgcgacaaatctattcttgctggaactgctgggcgcaggagagctcgccctgacgatgcgatctaagaagcttcccatcaacgttaccactggcgaggagcagcaggtgtccctcgaaagcgtcgacgtctatttccaagacgtcttcggaacaatgtggtgccaccacgctgagatgcagaaccccgtatatcttattcccgaaaccgtgccatacatcaagtgggacaactgcaactcaactaacatcactgcagtagtgcgcgcccagggactggacgtcactttgccactgtcactgcccacatcagcccaagacagcaacttcagcgtcaagacagaaatgctgggcaacgaaatcgacatcgaatgcataatggaggatggggaaatttcccaggtcctccccggtgataacaagtttaatattacttgttctggatatgaatcccatgtgccgtctggtggtatactgacgagtacatcgccagtggctaccccaatccccgggacaggctatgcctacagcctgagactgacgccaagaccagtttccaggtttctggggaataactctatcctgtatgtgttttattctggtaacggacccaaggcatccggcggtgattattgtatccagagcaatatagtgttctctgatgagattccggcctcccaggatatgccaaccaacacgacagatatcacctatgtcggggacaacgccacatatagcgtgccaatggtgaccagcgaagatgccaattctcccaacgtcacagtgacagccttttgggcgtggcctaataacacagaaactgactttaaatgcaaatggactctaacctcaggcaccccatctggctgcgagaacataagcggcgcgtttgcatcgaacagaacttttgatattacagtatctgggttgggcactgcgcccaagactctaataattacacggacggccactaacgccaccacgacgactcacaaggtgatcttcagcaaagctcccgaatctactactacctcgccgacactgaatacgacaggattcgccgacccaaatactacgaccggccttcccagctcgacacatgtgcctacaaacttaacggcgcccgccagtaccggccccaccgtgagcaccgctgacgtcacctctcctacccctgccgggactacaagtggtgcttccccagttactcccagccctagcccttgggacaacggcactgagagcaaagctccggacatgaccagcagtaccagtccagtgacaactccgacacccaatgcgacgtccccaactcccgccgtgaccacgccaacaccaaatgccacgtcacctactccagcggtcaccaccccaacgccaaacgctactagtcccactctaggcaagacatctcccacctccgctgtcaccacccctacacccaatgctaccggtcccacggtgggtgagactagcccacaggccaacgcaactaatcatacattgggtggcacaagcccaacgcccgtcgtcactagccagcctaagaacgccacaagcgctgtaactaccggccagcacaatataacaagttccagtactagcagtatgtctctgagacccagctcaaatcctgaaaccctctcgccctctacgtcggataactccacgtcccacatgcctctactcacaagcgcgcaccctaccggaggagagaatatcactcaggttaccccagcttctatctcgactcaccatgtctctacttcttccccagcgccacggcccggcactaccagccaggctagcgggccaggtaattcctccactagcactaaacctggcgaagtcaacgttaccaagggaacacctccccagaatgcaacctcgccccaagctccctctggacagaagacagccgtccctaccgtcacaagcaccggtgggaaggcgaattctacgacaggcgggaagcacactaccggacacggagcaaggacatccactgagcccacgaccgactacggtggcgactccaccacgcccagacccaggtacaatgcgactacctacctgccgccttccacttccagcaaactgaggcctcgttggactttcacttctccaccagtcaccaccgctcaggctaccgtgcctgtcccgccgacctctcagccccggttttcaaatttgagtatgctcgtgctgcagtgggccagcctggcagtgcttaccctcctgctcttgctggttatggctgactgcgccttccgacgtaacttatccaccagtcatacttacactacacctccttatgacgacgcggagacctacgtg

SEQ ID NO: 125 - protein, 865 aa, synthetic construct; identical to SEQ ID NO: 28

SEQ ID NO: 126 - RNA, 2595 nt, synthetic construct
  source 1..2595: mol_type = other RNA
atggaagccgccttactggtctgccagtacactattcagtccctcatccatctcacaggcgaggacccggggttcttcaacgtcgaaatccccgaattcccattctatcccacctgtaacgtctgcacagccgacgttaacgttacaatcaatttcgacgtgggcgggaagaaacaccagctggacctcgactttgggcagctgacgccccacactaaagccgtttaccagcccaggggtgcattcggtggatctgagaatgctacaaacctctttctgcttgagctgcttggtgctggagagctggccctgacaatgcgcagcaagaagttgccaatcaatgtgaccacaggtgaagaacagcaggtgtccctagaatccgtggatgtgtacttccaggacgttttcggcacaatgtggtgccatcacgcagagatgcagaatccggtgtaccttatccccgaaacagtgccgtacattaaatgggataattgtaactccacaaacatcacggcagtggtgcgcgctcaggggctggacgtgacactcccgctttccttaccaacaagtgctcaagattccaacttttctgtgaagaccgagatgctgggcaacgagattgatattgaatgcatcatggaggatggcgagattagccaggtgttgcccggcgacaataaatttaatattacctgttccggctatgagtcacacgtgccaagtggaggcattctcacgagcacctctcccgttgcaacacccattcctgggacagggtacgcatatagtctcaggctgacaccgagaccggtatcgcgcttcttaggtaacaattccatcttgtatgtgttttacagcggaaacgggccaaaggcctcaggaggagactactgcatccagagcaatatagtctttagcgacgagatccctgcctcacaagacatgcccaccaacaccaccgacatcacgtacgtcggtgataatgccacttacagcgtgcctatggtgacctctgaagacgcaaattcacccaatgtcacggttaccgcattctgggcttggccaaataacactgaaaccgacttcaagtgtaaatggaccctaaccagcggcaccccatcagggtgcgagaatatcagcggggccttcgccagcaaccggacattcgacatcaccgtgtcaggactagggactgctccaaagacattaattattacacggacagcaactaacgctacgactacgactcacaaagtgatatttagcaaagcgcccgaatcgactactacgagcccgaccctaaacaccaccggtttcgccgatcccaacacaactactggtctcccatcttcgacccatgttccaacgaatctgacagctccagcaagtacggggcccactgtgagtacagccgacgttactagcccaactcccgcgggtacgactagtggcgcgtctccagtgaccccttcgccttctccgtgggataatggaaccgagtcaaaggctcctgatatgaccagctccaccagtcccgttacaacgccaacgcccaatgcaacgagtccaactccggcggttaccactccgacaccaaacgccacctcgcccacgcctgcagttactacgcccacccctaatgctacgtcacccactctgggaaagacctcaccaacctctgccgtgactactcctacacctaatgccacaggacccacagttggggaaaccagcccacaggctaacgcaactaatcatacgcttggcgggacctctcccacgcccgtggtcacttcacagcctaagaatgccacaagcgctgtgactacaggccagcataatataaccagcagttcaaccagtagcatgtccctgcgtccttcatccaatcctgagactctgagccccagcactagtgacaactcaacttctcacatgccgttgctgacctccgcgcatcccacgggcggtgagaatatcacacaggtgacacccgcgtctattagcacacatcacgtgagtacgtcctcgcccgcaccccgcccaggcacaacctcacaagcatcaggtccggggaatagcagcacaagtaccaagcctggcgaagtgaatgtaaccaagggcacgccaccgcagaacgccacatcccctcaagccccatccggccagaagaccgctgtacccaccgtgacatcaactggggggaaagcgaactcaactaccggtgggaagcatacaacagggcacggtgccagaacttctacagagccaaccacggactacggaggggacagcacaacgcccagaccgcggtacaatgcgaccacatacctgcctccctcaacttcgagtaagctgcgacccagatggacctttacttctcctcctgtcaccaccgctcaagccaccgtcccggttccacctacttcccagcctagattctccaatctgagcatgcttgtcctacagtgggcatcactcgcggtactaaccttgctgctactgttggtgatggcggactgcgcgtttcgccgcaacttatccacctcccacacgtacacaacacctccctatgatgacgctgagacttacgtc

SEQ ID NO: 127 - protein, 865 aa, synthetic construct; identical to SEQ ID NO: 28

SEQ ID NO: 128 - RNA, 2571 nt, synthetic construct
  source 1..2571: mol_type = other RNA
atgacccggcggcgggtgttaagcgtggtggtgctcttagccgccttagcctgccggcttggcgcccagactccggagcagcccgctcctcccgccaccaccgtccagcccaccgccacccgccagcagacctccttccccttccgcgtctgcgagctctcctcccacggcgacctcttccgcttctcctccgacatccagtgcccctccttcggcacccgcgagaaccacaccgagggcctcctcatggtcttcaaggacaacatcatcccctactccttcaaggtccgctcctacaccaagatcgtcaccaacatcctcatctacaacggctggtacgccgactctgtgactaatcgccacgaggagaagttctccgtcgactcctacgagactgaccagatggacaccatctaccagtgctacaacgccgtcaagatgaccaaggacggcctcacccgcgtctacgtcgaccgcgacggcgtcaacatcaccgtcaacctcaagcctacaggcggcctcgccaacggcgtccgccgctacgcctcccagaccgagctctacgacgcccctggctggctcatctggacctaccgcaccaggaccactgtcaattgcctcatcaccgacatgatggccaagtccaactcccctttcgacttcttcgtgaccaccacaggccagaccgtcgagatgtcgccattctatgacggcaagaacaaggagactttccacgagagagcggactctttccatgtccgcaccaactacaagattgtcgactacgacaaccgcggcaccaatccacagggcgagcgccgcgccttcctcgacaagggcacctacaccctctcctggaagctcgagaaccgcaccgcctactgtcctctccagcactggcagaccttcgattccaccatcgccaccgagactggcaagtccatccatttcgtcaccgacgaaggaacatcttcattcgtcacaaatacaaccgtgggcatcgagctccccgacgccttcaagtgcatcgaggagcaggtcaacaagaccatgcacgagaagtacgaggccgtccaggaccggtatacaaagggccaggaggccatcacctacttcatcaccagcggaggtctgcttctcgcctggcttccactcacacctcgctccctggccacagtgaagaacctcactgagttgaccaccccaacaagttccccacctagtagtcctagccctcctgctccttccgccgcccgcggctccacgcctgccgccgtcctgcggagacggagaagggacgccggcaacgctaccactcctgtgccgccgacagcccctggaaagtccctaggtaccctcaacaacccagcgacagttcagatccagttcgcctacgactctctcaggcgccagatcaaccgcatgcttggtgaccttgcccgcgcctggtgcctcgagcagaagcgccagaacatggtgcttcgtgaactaacaaagattaatcctactacagtgatgtcctccatctacggcaaggccgtcgccgccaagcgcctcggcgacgtcatctccgtctcccagtgcgtcccagtgaaccaggctaccgtgaccctcaggaagtccatgagagtgccaggctccgagacaatgtgctactcccgcccactcgtctccttctccttcatcaacgacacaaagacctacgagggccagttaggcactgacaacgagatcttcttgactaagaagatgactgaggtatgccaggcaacttctcaatactacttccagtctggaaatgagatccacgtatataacgactaccaccacttcaagactattgaactcgacggaattgccaccctccaaacattcatctcactgaatacctccctcatcgagaacatcgacttcgcctccctcgagctgtatagcagagacgagcagcgcgcctccaacgtcttcgacctcgagggcatcttccgcgagtacaacttccaggcacagaacatagccggcctccgtaaggatctggacaatgccgtgtccaacggccgcaaccagttcgtcgacggtttgggtgaactcatggactctctgggctccgtcggccagtccataactaacttagtctctacggtgggaggcctattcagcagcctggtgagcggcttcatctctttcttcaagaaccccttcggcggcatgctcatcctcgtcctcgtcgccggcgtcgtcatactggtgatctcactcacaaggaggacgcgccaaatgtcccagcagccagtgcagatgctctacccaggcatagacgagctcgctcagcagcacgcgtcgggtgagggaccaggcatcaatcctatctccaagactgagctgcaagccattatgctcgccctccacgagcagaaccaggaacagaagcgggccgcccagcgagctgccggcccctccgtcgccagtagggcactacaagccgcccgggaccgcttcccaggcctgagaaggaggagataccacgacccagagacagccgctgccctccttggcgaagcagaaaccgagttc

SEQ ID NO: 129 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 130 - RNA, 1375 nt, synthetic construct
  source 1..1375: mol_type = other RNA
gggaaataagagagaaaagaagagtaagaagaaatataagagccaccatgagcgacgagggtccgggcacaggacccggtaacgggttgggcgaaaagggtgacactagcgggcccgaaggttccggcggatctggcccgcaacgcagaggtggtgataaccacggtcgcggtagaggcagaggccgtggccggggtggcggaaggcccggtgcccctggagggtcagggtctggcccaaggcaccgtgatggggtgcgtcgcccccagaaacgcccgtcctgtatcggctgcaagggcacgcatggcggaacgggaggtggcgcaggagccggaggtgcaagaggtcgcggggggtcaggggggagaggtagagggggatcaggggggcggagagggcggggtcgagaaagagcaagaggtggctccagagagcgcgctcggggtcgcgggcgaggccggggtgaaaaacggccccggagcccatcctcgcaatcatcaagctcgggatcgccacctcggcggccgccccccggcaggcgtccctttttccacccagtgggcgaagccgactacttcgagtaccaccaggagggaggaccggatggtgagccggatgttcctcccggcgccatcgaacaaggtcctgccgatgatccaggcgaggggccctcaacaggcccccgagggcagggggacgggggacgtcggaagaaaggaggctggtttggcaagcaccgaggacaggggggtagcaacccgaaatttgagaacatcgccgaggggctcagggctctcctagcccgtagccacgtcgaaagaactaccgatgagggcacctgggttgccggcgtgttcgtgtacgggggtagcaagacctctctatacaaccttcggagggggactgcacttgctatcccccaatgccgcctcacaccactgtctcgcctgccattcggcatggcccccggcccggggccacagcctgggcccctacgggaatctattgtttgctattttatggtgttcctgcagacccatatcttcgcagaagtcctgaaagacgccatcaaagacttagttatgactaaacccgcacccacctgcaacattcgcgtgaccgtatgcagctttgatgatggcgtcgatctgcccccatggttcccccctatggtcgagggcgccgccgccgaaggggacgacggggatgacggggatgaaggcggggacggcgatgagggcgaggagggccaggaatgataataggctggagcctcggtggccatgcttcttgccccttgggcctccccccagcccctcctccccttcctgcacccgtacccccgtggtctttgaataaagtctgagtgggcggc

SEQ ID NO: 131 - RNA, 1375 nt, synthetic construct
  source 1..1375: mol_type = other RNA
gggaaataagagagaaaagaagagtaagaagaaatataagagccaccatgagtgacgagggacctgggactggccccgggaacggtctgggtgagaaaggtgacacgtctggtcctgaaggatctggcggttcagggccgcagcgcagaggaggagacaaccacggtagaggacgtggacgcgggagaggccgagggggcggaagacctggtgcaccgggcggttccggctcggggccccgccacagggatggggtgcggagaccacagaagcgcccctcttgcatcggctgcaagggaacccatgggggtaccggagggggagctggtgcaggtggcgcccgtggtaggggtggcagcggggggcgtggaagaggtggttcaggcggacggaggggacgcggacgtgaacgtgcccggggaggatcccgggagagggcacggggccgcggcagaggcagaggggagaagagacctagatcccctagctctcaatccagcagcagtgggtcccccccgaggcgaccgcctccaggtaggcgccctttcttccaccccgttggcgaggcagattacttcgaatatcatcaagagggggggcccgatggcgagccggacgtaccacccggtgctatcgagcaaggtccggctgacgatcctggtgaagggccaagtaccggacctagggggcagggcgacggtggtaggcgtaaaaaaggagggtggttcggcaaacatcgcggccagggagggagcaaccccaagtttgaaaatatcgccgaagggctacgagctttgctcgcgaggtcccatgtggaacgaactacagacgagggcacctgggtggctggtgtcttcgtttatggcgggagcaagacctccctgtataatctaaggcggggcacagccctggccataccgcagtgtcgcctgacacccctgtctcgcctgccttttggcatggcgccagggccaggaccacaacctgggcccctgcgcgaaagtattgtgtgttacttcatggtcttcctgcagacacatattttcgcagaagtcctaaaggatgcaataaaggatctcgtaatgacgaagcccgccccaacgtgtaatattagggttacggtctgtagtttcgatgacggggtagatctccccccctggttcccacctatggtcgagggtgccgctgcggagggggacgatggcgacgacggcgatgaaggcggggatggcgacgagggggaggagggacaggaatgataataggctggagcctcggtggccatgcttcttgccccttgggcctccccccagcccctcctccccttcctgcacccgtacccccgtggtctttgaataaagtctgagtgggcggc

SEQ ID NO: 132 - RNA, 1375 nt, synthetic construct
  source 1..1375: mol_type = other RNA
gggaaataagagagaaaagaagagtaagaagaaatataagagccaccatgtcagacgagggcccaggcacgggccctggaaacggactgggagagaagggggatacctcagggccagagggtagtgggggtagcgggcctcagagaagggggggcgacaatcacggtcggggaagagggagaggtaggggtagaggaggaggccgtcccggggctcctggcggttccgggtccggccctcgccacagagatggcgtgaggagacctcagaagcgtccgtcctgtattgggtgcaaggggacccacggtggcaccggtggcggagctggggcaggaggtgctagaggaagaggcggttcaggtggaaggggtcgcggtggttcaggaggacggcgcggccgggggagagagcgggcaagaggcggcagcagggagcgggcaagaggacgagggaggggccgaggcgagaagcgacctaggtcgcctagttcacagagctccagttctgggtcgcccccccggcggccgcctcctgggcgaagaccctttttccaccccgtgggggaggctgactacttcgagtaccaccaggagggcggcccagacggagagcccgatgttccgcctggggccattgaacaaggccctgcagatgacccaggggaaggaccatcaaccggccctaggggacagggcgatggggggagacggaaaaagggagggtggttcgggaagcaccgcggccagggggggtctaatccgaaattcgaaaacattgccgagggcctccgtgctttgttggctagatcacacgtggaaagaaccacggatgaaggcacgtgggtggcgggcgtcttcgtgtatggtgggagtaagacctccctgtataacctcaggcgtggcacagcccttgccatcccacagtgcaggctcacaccccttagcaggcttccctttggcatggctcctggtcccggcccccagcccggtccactgcgagaatccattgtatgttacttcatggttttcttgcagacacatatcttcgccgaggtgctgaaagacgccattaaagacctggtgatgacaaagcctgcccccacgtgcaatatccgtgtgaccgtctgctcctttgatgacggagtcgacctgcctccctggtttcccccaatggtcgagggcgctgcggcggaaggggacgacggcgatgatggtgacgagggcggtgatggcgacgaaggtgaagagggtcaggagtgataataggctggagcctcggtggccatgcttcttgccccttgggcctccccccagcccctcctccccttcctgcacccgtacccccgtggtctttgaataaagtctgagtgggcggc

SEQ ID NO: 133 - RNA, 1375 nt, synthetic construct
  source 1..1375: mol_type = other RNA
gggaaataagagagaaaagaagagtaagaagaaatataagagccaccatgagcgacgagggtccaggcacaggcccggggaacgggttgggagaaaaaggggacacttccggtccagaaggctccggggggagcggacctcagagaagaggtggtgataatcacgggagagggagagggcggggcaggggacgtgggggcggtagacctggggcgccggggggttccggctccggaccacggcatagggatggtgtgcgaaggccgcagaagcggccgtcttgtataggctgcaaaggcactcacggtggtaccggggggggagcaggagcaggaggcgctcgaggtcggggcgggtccggcggcagaggccgcggtggttctgggggaaggaggggaagagggagagaacgcgcccgcggaggctctcgcgaacgggccagagggcgggggagggggcgcggggagaaaaggcccaggtcgccaagtagtcagtcctccagtagcggcagcccaccccgcagacctcctccaggacgacgacctttctttcatcccgtcggcgaggccgattactttgagtaccatcaggaaggggggcctgatggagaaccggatgttcctcctggtgctatcgaacagggccccgccgacgatccaggcgagggccccagtacgggccctagaggccagggagatggaggtagaaggaagaaggggggatggtttggcaagcacagagggcagggaggtagtaatcccaaatttgagaatattgccgagggtctgcgggcacttctggcccggtctcatgttgagagaacgactgatgagggaacatgggtggctggcgtctttgtgtatggcggtagtaaaaccagcctctacaatttacggcgtgggacagcgctagctattccgcaatgccggctgacccctttgtctaggctgccctttggtatggcccccggccccggcccacagcctgggcctttgcgggaaagcatcgtctgctactttatggtgttcctccagacccacatctttgcagaggtgcttaaggacgccataaaggacctggttatgaccaaacccgcccctacctgcaatatccgcgtcacggtgtgctccttcgacgacggggttgacttgcctccatggttcccacccatggtagagggcgctgctgccgagggggatgatggagacgacggagacgagggcggcgatggcgatgaaggcgaagaaggccaggaatgataataggctggagcctcggtggccatgcttcttgccccttgggcctccccccagcccctcctccccttcctgcacccgtacccccgtggtctttgaataaagtctgagtgggcggc

SEQ ID NO: 134 - RNA, 1375 nt, synthetic construct
  source 1..1375: mol_type = other RNA
gggaaataagagagaaaagaagagtaagaagaaatataagagccaccatgtctgacgaaggcccgggcacaggcccaggcaacggattgggcgaaaaaggtgatacaagcgggccagaagggagcgggggctctgggccccagagacgggggggtgacaatcacggccgaggaaggggaaggggcaggggaagaggcggaggacggcctggcgctcctggaggctctggctcaggtccaagacatagggatggggtcagaagaccacagaagagacctagctgtattggatgcaagggtacacacggtggcacgggcggcggtgctggcgctgggggcgcgagaggacgtggaggttcaggcgggagaggtcgaggaggctctggaggccgcagggggagaggacgagagagggcccggggggggtcccgggaaagagctagagggaggggacgcgggcggggggaaaagcggcctcgctctccttcttctcaatcttcctcctctggttctccgccccgacgccccccaccgggcaggaggccattttttcaccctgtcggcgaggcagactacttcgagtaccaccaagaaggaggcccagacggcgagcctgatgtgccgccaggtgccatcgagcaggggccagctgatgacccgggggagggacctagcacggggccccgcggtcagggcgacggagggaggagaaaaaaaggtgggtggtttggcaagcatagagggcagggtggctcaaacccaaagtttgaaaacatcgcagaaggtcttcgggctctgctggcccgctctcatgtagagcgcactactgacgaaggcacctgggtggccggggtgttcgtgtacggaggatctaagaccagtctgtacaacctccggagggggacagctctcgctatcccccagtgtaggctcacccctttgagtcgccttccgttcggaatggctcctggcccgggcccccagcccgggccactaagagagagcatcgtatgctactttatggtatttttgcaaacccacatcttcgctgaagtgcttaaggacgccatcaaagacctggtgatgacaaaaccagctccaacatgcaatattagggtaaccgtatgctccttcgatgatggcgtcgaccttccaccttggttccccccaatggtggagggagccgcagcagaaggagacgatggtgatgatggagacgaaggcggggatggcgacgaaggagaggagggacaggaatgataataggctggagcctcggtggccatgcttcttgccccttgggcctccccccagcccctcctccccttcctgcacccgtacccccgtggtctttgaataaagtctgagtgggcggc

SEQ ID NO: 135 - RNA, 892 nt, synthetic construct
  source 1..892: mol_type = other RNA
gggaaataagagagaaaagaagagtaagaagaaatataagagccaccatgggcagaagacccttctttcaccccgtgggagaagccgactatttcgagtaccaccaggagggcggtcctgacggcgagcccgacgtgcctccaggcgccatcgaacagggccccgctgacgatcctggcgaggggccttccacaggccctcgcggccagggagatgggggaaggcggaagaaggggggctggtttggcaagcataggggccagggcggctccaatcccaagttcgagaatatcgccgaggggctccgggctctgctggcccggtcgcacgtcgaacgtaccactgacgaggggacgtgggtggccggcgtctttgtgtacggaggatccaagacctccctgtacaacctgagaagggggaccgcccttgccatccctcagtgcagactcacccccctgagcaggctcccctttgggatggctcctgggccaggcccccagcccgggcccctcagggaaagcatcgtgtgttactttatggtgtttctgcagacacacatttttgcagaagttctgaaagatgcaatcaaggatctggtgatgaccaagcctgcacctacgtgcaatatcagggtgacagtatgctccttcgacgacggtgtggacctccccccctggttcccacccatggtcgagggggccgccgccgagggagacgatggcgacgacggtgatgaggggggagacggagatgaaggcgaggaggggcaggagtgataataggctggagcctcggtggccatgcttcttgccccttgggcctccccccagcccctcctccccttcctgcacccgtacccccgtggtctttgaataaagtctgagtgggcggc

SEQ ID NO: 136 - RNA, 892 nt, synthetic construct
  source 1..892: mol_type = other RNA
gggaaataagagagaaaagaagagtaagaagaaatataagagccaccatgggacggagaccattcttccaccccgtgggagaagccgactacttcgagtaccaccaggagggcggtccagacggcgaacccgacgtccctcctggagccatcgagcagggccccgccgacgaccctggcgagggccctagcaccggacctagaggacaaggcgacggcgggaggagaaagaaggggggctggttcgggaagcaccgcgggcaaggcggcagcaacccaaagttcgagaacatcgccgaggggttgcgggccctgctcgcccgttcccacgtggagcggacaaccgacgagggaacctgggttgccggggtatttgtgtacggcggcagtaagaccagcctgtataacctcagaagaggaacagccctggccatcccacagtgcaggctgacacccctctcccggctgccatttggaatggcacccgggcctggaccccagcctggcccactgcgcgagtctatcgtgtgttactttatggtcttcctgcaaacccacatcttcgccgaggttttaaaggatgccatcaaagacctcgtgatgacaaagcccgctcccacctgcaatatcagagtgaccgtatgcagctttgacgacggcgtggacctgcctccctggttccctcctatggtggagggcgccgccgcagaaggggacgacggtgacgatggcgatgagggaggggatggagacgaaggcgaggagggccaggagtgataataggctggagcctcggtggccatgcttcttgccccttgggcctccccccagcccctcctccccttcctgcacccgtacccccgtggtctttgaataaagtctgagtgggcggc

SEQ ID NO: 137 - RNA, 892 nt, synthetic construct
  source 1..892: mol_type = other RNA
gggaaataagagagaaaagaagagtaagaagaaatataagagccaccatgggccgcagacccttctttcaccccgtgggagaggccgactattttgagtaccaccaggagggcgggcccgacggcgaacctgacgtgccacctggcgccattgaacaaggcccagccgacgaccctggggagggccctagcacagggcccaggggccagggagacgggggtaggcgtaagaagggaggctggttcggtaagcaccgcggccagggtggcagcaaccctaaattcgagaatattgccgaaggtctgcgagccctcttggccagaagccacgtggaacgcactactgatgagggcacctgggtcgccggagtgttcgtgtacggcggaagcaaaaccagtctgtacaacctgcgccggggaaccgctcttgccatcccccagtgtcgcctgaccccattgtccagactgccatttggcatggcccccggccccggcccccagcccggccccctgagggagagcatcgtgtgctacttcatggtgttcctgcagacccacatattcgctgaggtgctgaaggacgccattaaggacctggtgatgaccaagcccgcccctacctgcaacatccgcgtcaccgtctgtagtttcgatgacggcgtggatctcccaccatggtttcctccaatggtggagggcgctgctgccgagggggacgacggagacgacggagacgagggcggggacggggacgaaggcgaagaggggcaagagtgataataggctggagcctcggtggccatgcttcttgccccttgggcctccccccagcccctcctccccttcctgcacccgtacccccgtggtctttgaataaagtctgagtgggcggc

SEQ ID NO: 138 - RNA, 892 nt, synthetic construct
  source 1..892: mol_type = other RNA
gggaaataagagagaaaagaagagtaagaagaaatataagagccaccatgggacggagacctttctttcaccccgtaggtgaagccgactactttgaataccaccaggagggcggcccagacggcgagcccgacgttccccccggggctatcgagcaggggcctgcggacgacccaggagagggacctagcactggaccccgaggtcagggcgatggggggcgaagaaagaagggcggctggttcggcaaacataggggccagggcgggtctaacccaaagttcgagaacatagccgagggcctgagagccctgctggcaagaagccacgtggagcgaaccacagatgaaggcacctgggtggccggcgtgttcgtgtacgggggcagcaagacctccctgtataacctcagaagagggaccgccctggccatcccccagtgccgactgacccccctgagcaggctgcccttcggaatggcccctggtcccggtccccaaccgggcccgcttcgggagtccatagtgtgctacttcatggtgttcctgcagacacacatcttcgccgaggtgctgaaggatgcaatcaaggacctggtgatgaccaagccagctcccacctgcaacatccgggtgaccgtgtgcagctttgatgacggcgtggacctgccaccctggttcccgcctatggtggagggcgctgcagccgagggcgacgacggggacgacggcgacgagggcggagatggcgacgagggcgaggagggccaggagtgataataggctggagcctcggtggccatgcttcttgccccttgggcctccccccagcccctcctccccttcctgcacccgtacccccgtggtctttgaataaagtctgagtgggcggc

SEQ ID NO: 139 - RNA, 892 nt, synthetic construct
  source 1..892: mol_type = other RNA
gggaaataagagagaaaagaagagtaagaagaaatataagagccaccatgggcagacggcccttcttccaccccgtgggcgaagcagactattttgagtatcaccaggaggggggccccgacggcgagccagacgtcccacctggtgcaatcgagcaaggtcccgccgacgaccctggggagggccccagcaccggccccagaggccagggggacgggggcaggagaaaaaagggtggctggttcggcaagcacagagggcagggggggagcaacccaaagttcgagaacatcgccgagggcttgagagcactgctggccagaagccacgtggagagaaccaccgatgagggaacctgggttgctggcgtgttcgtctacggcggcagcaagaccagcctgtacaaccttagaagaggcaccgccctggccatcccccagtgccgcctgacaccgctgagcagactccccttcggcatggctcctggccccggcccccagcccgggcccctgagagagagcatcgtgtgctacttcatggtgtttcttcagacgcacatcttcgctgaggtcctgaaagacgccattaaggacctggtgatgacaaaaccagctccgacctgcaacatcagggtcactgtctgtagcttcgacgatggcgtggatctccctccctggttccccccaatggtggagggcgctgccgctgagggggacgacggcgacgacggggatgaaggcggcgacggcgacgagggggaagagggccaggagtgataataggctggagcctcggtggccatgcttcttgccccttgggcctccccccagcccctcctccccttcctgcacccgtacccccgtggtctttgaataaagtctgagtgggcggc

SEQ ID NO: 140 - RNA, 1324 nt, synthetic construct
  source 1..1324: mol_type = other RNA
gggaaataagagagaaaagaagagtaagaagaaatataagagccaccatggagcacgacctggagagggggccaccaggccccaggcgacccccaagaggaccaccactgagctccagcctgggactggccctcctgctgttgctcctcgctttactgttttggctgtatatagtcatgtctgactggacaggcggggcattattggtgctctacagctttgcactgatgctcattatcattattctgattatctttatttttagacgggacttgctctgtccattaggcgcgctgtgtattttgctgctgatgatcacactgctgctgattgcgctgtggaacctccacggtcaggccctttttctcggcatcgtgctgtttatctttgggtgcctcttggtgcttgggatttggatctaccttctcgaaatgttatggcggctcggcgctacaatctggcagctcctggccttcttcttagcattttttctggatctgatcctgctgataatagccctgtacctacaacagaactggtggactctgctggttgacctgttatggctgctcctgttcctcgccattctcatttggatgtactaccacgggcagcgtcattcagacgaacaccaccacgatgatagcctgccgcaccctcaacaagctacggatgactccggacacgagtccgatagtaactccaacgagggacgccatcacttgctggtctctggggctggcgacgggcctccactgtgtagccaaaatctgggagcccccggcgggggccctgataatgggcctcaggatcctgataacacggatgataacggcccacaggatccagataataccgacgataatggacctcatgatcctcttccacaggatcccgacaatacagatgacaatgggccgcaggatccagacaatactgacgataatggcccacatgatcccctgccacacagcccctcagactcagcgggaaatgatggcgggcccccacagttaaccgaggaagtggaaaacaaagggggagatcagggtcctcccctcatgacagatggcgggggcggccacagtcatgatagtgggcacgggggtggcgatccacatctccccacactgctgctaggcagctccggtagcgggggagatgatgacgatccccacggccctgtgcagttgagctattacgactgataataggctggagcctcggtggccatgcttcttgccccttgggcctccccccagcccctcctccccttcctgcacccgtacccccgtggtctttgaataaagtctgagtgggcggc

SEQ ID NO: 141 - RNA, 1324 nt, synthetic construct
  source 1..1324: mol_type = other RNA
gggaaataagagagaaaagaagagtaagaagaaatataagagccaccatggagcacgacctggagcgcgggcctccaggaccccgacggccgcccagggggccaccgctgtcatccagtctgggccttgctctgttgctgttactccttgccctgctattctggttgtacatcgtcatgagcgattggacagggggtgctctgctggtgttgtacagctttgcgctgatgttgattattatcatcctgattatctttatatttcgaagggacctcctttgtcccctgggagcgctgtgcatactgctcttaatgatcacgttgctcctcattgctttatggaatctgcacggacaggctctattcctcggcatcgttttattcattttcgggtgcctccttgtgctgggaatctggatctatctgctagagatgctgtggcggctcggcgccaccatttggcagcttctggcatttttcctagcgttttttctggatctcatcctacttattatcgccttgtatttgcagcagaactggtggactctcctggtggatctcctgtggctcctcctgttcctcgccatattgatatggatgtactaccacggacagcgccacagcgacgagcaccaccacgatgatagtctgcctcaccctcaacaggctacagatgactcaggccacgaaagcgattccaactcaaatgaaggccggcaccacttgttggtgtccggagccggcgacgggccaccactctgcagccagaatctgggcgctcccggcggaggtccagacaatgggccacaggaccctgataacacggatgataacggaccgcaggatcctgacaatacagatgacaacggcccgcatgatccactgcctcaggatcctgataatactgacgataacggaccccaggatcctgataacaccgacgacaacgggcctcacgatcccctaccccattccccatctgactctgctggcaacgatggcggcccaccgcagctgaccgaagaggtagaaaacaagggcggggatcagggccctccactgatgacagacggcggcgggggacactcccatgattcgggccatgggggaggggatcctcaccttcccacactcttgctgggaagtagcgggtccggcggggacgatgatgatccccatggtccagtccagttgtcatattacgattgataataggctggagcctcggtggccatgcttcttgccccttgggcctccccccagcccctcctccccttcctgcacccgtacccccgtggtctttgaataaagtctgagtgggcggc

SEQ ID NO: 142 - RNA, 1324 nt, synthetic construct
  source 1..1324: mol_type = other RNA
gggaaataagagagaaaagaagagtaagaagaaatataagagccaccatggagcacgaccttgagcggggtccaccaggcccacggcgccctccgcgcgggcctcctttgtcatcctccctgggactggcactgctgctgttgcttctggccctgctgttctggctgtacatcgttatgtctgattggacaggaggagctctgctggttctttactctttcgcgctgatgctgatcatcatcatcttgatcattttcattttcagacgagacctcctttgtcctctcggagccctgtgtatcctcctgctgatgataacccttctgctgattgctttgtggaatctgcatgggcaagctctgtttcttggaattgtcttatttatttttgggtgtctgctggtgttgggaatctggatctatcttcttgaaatgctgtggcggctgggcgccacaatctggcagctactcgcctttttcctagctttctttttggatctgattctgctgattattgctctctacctgcagcagaattggtggaccctcttagtcgatctgctgtggctgttgctctttctcgccatcctgatttggatgtactatcatgggcagcgccatagtgatgaacatcatcacgatgattccctgccacatcctcagcaggccacagatgattcaggccacgaatcagacagcaatagcaatgagggccgccaccacctgctggtttcaggggccggtgacggccctccattgtgcagtcagaacctgggagccccggggggtggacctgataacgggccgcaggatcctgacaatactgatgacaacggaccccaggatccagataacactgacgacaatggcccccatgaccccctaccccaggatcccgacaatacagacgacaacggaccgcaggaccccgacaacactgatgataatgggccacacgatcctctgcctcactctccatcagactctgccggaaatgacggtggaccgccacagttaactgaagaagtcgaaaacaagggcggcgatcaggggccgccgctcatgacagatggcgggggaggtcacagccacgatagcggacacggaggaggtgaccctcatctgcctacacttctgttgggtagctctgggagtgggggggatgacgacgaccctcacggacctgttcagctctcatactacgactgataataggctggagcctcggtggccatgcttcttgccccttgggcctccccccagcccctcctccccttcctgcacccgtacccccgtggtctttgaataaagtctgagtgggcggc

SEQ ID NO: 143 - RNA, 1324 nt, synthetic construct
  source 1..1324: mol_type = other RNA
gggaaataagagagaaaagaagagtaagaagaaatataagagccaccatggaacacgatcttgaacgggggccgccgggaccgaggaggccgccacgggggccccccctgagcagtagcttggggcttgcgctgcttctcctgctgttggccctcctgttttggctgtatattgtgatgtccgactggaccggcggcgccctgctcgtcctgtatagctttgctttgatgctgataattattatattgatcatctttattttccggcgtgatttgctctgtcctcttggtgccctttgtatcctgctgttaatgatcactctgctcctcattgccctatggaacctccacggacaagccctgtttcttggcatcgttctctttatattcggatgcctcttagtgcttgggatctggatatacctgttggaaatgctgtggaggcttggagccacgatatggcaattattggcattctttctggccttcttcctggatctgatcctgttgataattgctctgtacctgcagcagaactggtggacactacttgttgacctgctctggctactcctgttccttgcgatcctgatttggatgtactatcacggacagagacactccgacgagcatcatcacgacgattccttaccacaccctcagcaggcgacagatgattctggccatgagagcgattccaacagcaacgagggccggcaccatttgctggtgtcgggggctggagacgggcctccgttatgtagccaaaacttgggcgcgcccggcggcgggcccgataacggcccgcaggacccggacaataccgacgataatgggcctcaagatcccgacaatactgatgacaatggaccacacgatcctctgccacaggatcccgacaacacagatgacaatgggccgcaagaccccgacaatacagacgataatggcccccacgacccactaccacattctccctctgacagtgcggggaatgatggtggtcccccacaactgaccgaggaggtggagaataaggggggggaccaaggtccacccctcatgactgatggaggaggaggacactcgcacgattcaggtcacggcgggggggaccctcacttgcccactctgctgctgggcagcagcggttctgggggcgacgatgatgatccccacggcccggtgcagctaagttattacgactgataataggctggagcctcggtggccatgcttcttgccccttgggcctccccccagcccctcctccccttcctgcacccgtacccccgtggtctttgaataaagtctgagtgggcggc

SEQ ID NO: 144 - RNA, 1324 nt, synthetic construct
  source 1..1324: mol_type = other RNA
gggaaataagagagaaaagaagagtaagaagaaatataagagccaccatggagcacgatttagagaggggcccacctggccccaggcgcccacctagaggcccacccttgtcgtcgtcattagggctggcattgctcctgctcctcctggccttgctgttctggctttacatcgtgatgagcgactggactggcggggccctgctggtcctctatagctttgcactgatgctgattataatcatattgatcatcttcatcttccgacgggatttactttgccccctgggggcattatgtattctattgctgatgataacacttctgcttattgctttatggaatctacacggtcaggctctttttctgggaatcgtgctctttatctttggatgccttctggtactaggaatttggatatacctcttagaaatgctgtggcggctcggcgcaaccatctggcaactccttgcgttttttctcgcgtttttcctcgacctgattctgctaataatcgccttataccttcagcagaattggtggacactcctggtggacctgctgtggctcctgctattccttgcaatcctcatttggatgtattaccatggccagaggcactctgatgagcaccaccacgatgatagtctacctcatccacagcaggctacggacgattcagggcatgagtcagattctaattccaacgaaggaagacaccatctgttggtgtcaggggccggagacggcccccctctgtgtagccagaacctcggggcaccagggggaggtcctgacaatgggccgcaagaccctgacaatactgatgataatggcccccaagatcctgacaataccgatgacaacggcccgcacgaccccctgccgcaggatccggataacaccgacgacaatggaccccaggaccccgataacacggacgataacggccctcacgatcccttaccccattctccgtccgactctgcagggaacgacgggggccctcctcagttaaccgaagaggtcgaaaataaggggggagaccagggccccccattaatgacagacgggggcggcggtcacagtcacgactcaggtcacggcggcggggacccccatttgcctacactgctgttagggtcttctggctcaggcggggacgatgatgatccacacggtccggtgcagctgtcatattatgattgataataggctggagcctcggtggccatgcttcttgccccttgggcctccccccagcccctcctccccttcctgcacccgtacccccgtggtctttgaataaagtctgagtgggcggc

SEQ ID NO: 145 - RNA, 1657 nt, synthetic construct
  source 1..1657: mol_type = other RNA
gggaaataagagagaaaagaagagtaagaagaaatataagagccaccatgggtagtcttgagatggtaccaatgggcgcaggtccaccgagccctggaggcgatcctgacgggtacgacgggggaaataactcgcaatatccctcagcctcaggctcatcgggtaatactccgacccctccaaacgacgaggaaagggaatccaatgaggaaccgcctccaccatatgaggatccgtactgggggaacggtgataggcacagtgactaccagccacttgggacacaagaccaaagcctgtacctcggacttcagcacgatggtaacgacggtttgccgcctcccccttatagccctagagatgatagctcccagcatatatacgaggaggctggtaggggctccatgaatccagtatgcttgcccgttatcgtagccccctacctcttctggcttgcggcgatcgcagctagctgttttacagcttccgtgagcacagtcgtaaccgcgacaggactggcactgtctctactcctcttggcagcggttgccagcagctacgccgccgctcagcggaaactgctcacccctgttacagtcttaactgcagttgtgaccttcttcgccatttgtttgacatggaggatcgaagatcctccatttaatagtctcctgtttgcactgctggccgctgcgggcgggctacaagggatatatgtgctagtaatgctcgtcctacttattctggcctataggcggaggtggcggcgcctgacagtgtgcggagggataatgttcctagcctgtgtgctcgtgctcattgtggacgctgttctacagttgtctcctctgctgggtgcagtgacggtggtctcaatgaccctgctgttacttgccttcgtgctttggttgtcatctccagggggactgggcacactgggcgccgccctgttaaccctggcggccgctctggcactcctggcctcactgattctgggaactctgaatctcaccactatgtttcttctgatgttattgtggactttggtggtgttgctgatttgcagctcctgcagttcttgccccctatccaaaatattactggcgcggctgttcctttatgccctcgccttactgctgctcgctagcgccttaattgctgggggctccattcttcagaccaactttaaatccctgtccagcactgaattcatccccaaccttttttgcatgctgctcctaatcgtggctgggattctgtttattctggccattttgaccgagtggggcagtggcaacaggacctacggccctgtgttcatgtgtctgggggggctcttaacgatggtcgctggcgctgtctggctcacggtgatgagtaacactttgctcagcgcgtggatcctgacagccggctttctcatttttctgattgggttcgctttgtttggagtcattcggtgttgccgctattgctgttactattgtttgacattggagtccgaggagaggccacccaccccctaccgaaatacggtatgataataggctggagcctcggtggccatgcttcttgccccttgggcctccccccagcccctcctccccttcctgcacccgtacccccgtggtctttgaataaagtctgagtgggcggc

SEQ ID NO: 146 - RNA, 1657 nt, synthetic construct
  source 1..1657: mol_type = other RNA
gggaaataagagagaaaagaagagtaagaagaaatataagagccaccatgggatccttggaaatggtgcccatgggagccggacccccctcaccagggggagacccagacggatacgacgggggaaacaattctcagtatccgagcgcgtccggcagctcaggaaacacacccaccccccccaacgacgaggagagagaatccaacgaggagcctcctcccccatacgaagacccttactgggggaatggagaccgtcattctgactatcaacctctaggcactcaggaccagtctctttacctagggttacaacacgatggtaatgatgggttgcccccgccgccctatagtccccgggacgacagtagtcagcatatatatgaggaagccggcagaggttctatgaatcccgtttgtctaccggtcattgtcgccccctatttattctggcttgcagccatcgccgcttcgtgcttcaccgcctccgtgtccacggtggtgacagctacagggctggcactctctctgctactcctggctgctgtggccagctcctacgcagcggctcagcggaagcttctcactcctgtaactgtcctcaccgcggtagtgaccttttttgccatatgcctgacatggaggattgaggacccaccctttaattctctgctgtttgcgcttttggccgctgccggaggactgcaaggtatttatgtgctggtaatgttggttctacttattctcgcataccggcgacggtggcggaggctgactgtatgcggggggattatgttcctggcgtgtgttctggttttgatcgtcgatgccgtgctgcagttgtcaccgctgctcggggcggtcaccgttgtgagtatgacactgcttctgctggctttcgtgctctggctatcttcccccggggggctggggaccttgggcgctgcgcttctgactctggcagctgctctggccctgctggcctctcttattcttggcacactgaatttgactaccatgttcctcttgatgttgctgtggaccttagttgtgctgctgatctgttcttcctgtagcagctgtccactgtccaagattctcctggccaggttgtttctgtatgccctagcgctgctgctgttagcttctgctctgatagccggcggcagtatcctgcaaaccaacttcaagagcctctccagcacagaattcatccctaatctcttttgcatgctactgctcatcgtggcaggcatactgttcattcttgctatcctgactgaatggggctctggcaatagaacatacgggccagtgttcatgtgcttgggcggcctgttgacaatggttgctggagccgtgtggctgacggtaatgtccaacactctgctgagcgcatggatcttaacagccggattcctgatctttttaatcggattcgcactcttcggagtgatccggtgctgtaggtattgttgttattactgcctgacactcgagtcggaagagaggccccctacaccttaccggaatacagtttgataataggctggagcctcggtggccatgcttcttgccccttgggcctccccccagcccctcctccccttcctgcacccgtacccccgtggtctttgaataaagtctgagtgggcggc

SEQ ID NO: 147 - RNA, 1657 nt, synthetic construct
  source 1..1657: mol_type = other RNA
gggaaataagagagaaaagaagagtaagaagaaatataagagccaccatggggtcgttggagatggtccccatgggcgctggtcctccaagccccggcggggacccggacggatacgacgggggcaataatagtcagtatccgagtgcaagcggaagcagtgggaatacgcctacaccaccaaacgacgaggagcgggagtctaacgaggagcctccacccccttacgaggatccttactgggggaacggagatcgacattctgactaccagcctttagggacacaggatcagtctctctatctcggactgcagcatgatggcaacgacggactgcccccaccaccttattctccacgcgacgacagctcacagcacatatacgaagaggccgggcgcgggtctatgaacccggtttgcctgccagtcatcgtcgcaccctacctcttttggctggcggccattgccgcgtcctgtttcacagcctcagtgtcgactgtggtaaccgcaacagggctcgcactcagcctactgctgctcgctgccgtcgcgtcctcttatgctgctgcccagcgcaaactgctcaccccagttacggtgctcactgctgttgttacctttttcgctatttgtctcacctggagaatcgaagacccgccgttcaacagtctgttatttgccctcttggctgcagcaggcggccttcagggcatctatgtcctggtgatgctggtcctgctcatcctggcttacaggagaagatggcgtcggctgaccgtgtgtggtggcatcatgtttctcgcgtgtgttctggtgctcatcgtcgatgccgtgctacagctgagcccactgcttggagccgtgacagtcgttagcatgacactcctgctattggctttcgtgctctggctgagtagcccaggtgggctcggcacacttggtgccgcgcttttaacactggccgcggccctcgcactgctggcctctctaattctcggaacattgaacctgacaaccatgttcctcctgatgctcctctggacactggtggtactgttaatctgtagctcttgctcatcatgcccactgagcaagatactcctggcgagacttttcctatacgccctggccctgctgctgttggcatcggctctgatcgctgggggaagcatccttcaaacaaatttcaagagccttagcagtactgagtttatacctaacctgttctgtatgcttctgctgatagtcgctgggatactttttattctggcaatcctaacagagtgggggtcaggtaacaggacctatgggcctgtttttatgtgcctcggaggcctcctcaccatggttgccggcgcagtttggttgacagtgatgtccaacaccctactgagtgcttggatactgacggccgggttcctgattttcctaataggatttgccctgtttggggttatccggtgctgccgttactgctgctactattgtttgacccttgagtctgaagaaaggccgcccaccccctacagaaacactgtatgataataggctggagcctcggtggccatgcttcttgccccttgggcctccccccagcccctcctccccttcctgcacccgtacccccgtggtctttgaataaagtctgagtgggcggc

SEQ ID NO: 148 - RNA, 1657 nt, synthetic construct
  source 1..1657: mol_type = other RNA
gggaaataagagagaaaagaagagtaagaagaaatataagagccaccatggggtcattggagatggtgcctatgggggccggcccacccagcccagggggcgatcctgacggatacgacgggggcaataattcacaatatcctagcgccagcggatcttctgggaatacccccacaccacctaacgacgaggaacgggagtcaaacgaggaacccccaccgccgtacgaggacccatactgggggaacggggatcgacactcagactaccagcctctcggaacgcaggatcaaagcctgtacctgggactgcagcacgatggtaacgatggactgccaccacctccttacagtccaagggatgactccagccagcacatttatgaagaagccggccgaggtagtatgaatccagtgtgtctgcccgtgattgtcgccccttatctcttttggctggccgccattgccgccagctgtttcaccgcgtcggtttcaaccgtagtgaccgccacaggcctggctctcagcttgttactgttggccgctgtggcgagctcctacgccgccgcccagcgcaagctacttacccctgtgacagtgctgacggcggtggttacattcttcgccatttgcctgacctggcgaattgaggatccaccattcaattccttattgtttgccctcctcgccgctgctggagggctccagggtatctacgtgctggtgatgctcgttttgctgatattggcttaccggcggcgctggcgccgccttaccgtctgcggaggaatcatgtttttggcatgcgtgctcgttctcattgtggatgctgttttgcagctgtcacccctgttgggtgccgtgacagttgtaagtatgaccctgctcctgctcgcattcgtgctctggctgagctctcccggggggctgggcactctcggagcagctctgctgacactagccgcagcactagcccttctggccagtcttatcctcggaacgctgaacctgacgaccatgttcctcctgatgttgctctggaccctcgtagtgctgttaatatgctctagctgtagctcctgccctctgagcaagattctccttgctaggctgttcctatacgccctcgctctcctattgctggcgagcgctctcatcgccggtgggagcatcctgcagactaacttcaagtccctgagttcaacagaattcattcccaacctcttttgtatgctgctgcttatcgtggccggcatcctctttatcctcgccatcttgactgagtgggggtccggaaatcgcacatatggcccagttttcatgtgtctggggggcctgctcacgatggtggccggcgcagtgtggctgacggtaatgtcaaacactttgctctctgcgtggatcttgaccgcaggcttcctgatctttttgatcggcttcgccctgttcggtgtcatccggtgctgcagatactgctgctactattgcctcacactcgaatccgaagagaggcctcccacaccttaccgaaatacagtgtgataataggctggagcctcggtggccatgcttcttgccccttgggcctccccccagcccctcctccccttcctgcacccgtacccccgtggtctttgaataaagtctgagtgggcggc

SEQ ID NO: 149 - RNA, 1657 nt, synthetic construct
  source 1..1657: mol_type = other RNA
gggaaataagagagaaaagaagagtaagaagaaatataagagccaccatggggtctttagaaatggtccccatgggtgccggcccaccctcgccaggaggcgatccagacggctacgacgggggaaacaacagccagtaccccagcgccagcggtagcagcggcaacaccccgaccccaccaaacgacgaagagcgggagagcaatgaggagcccccacccccctatgaggacccttactggggaaatggcgatcgacacagcgattaccagcccctgggcacacaggatcagagcctgtacctgggcttgcagcatgatggtaacgacgggcttccgcccccaccatactcacccagagacgattcaagtcagcacatctacgaggaggctggtaggggtagtatgaatcctgtgtgtctccctgtgatcgtcgccccctacctattttggttagctgcgatcgctgcctcctgcttcaccgctagcgtgtcaacagtggttacggcaaccgggctggcgttaagtctgcttctgcttgcggcagtggcatcttcttatgcagcggcccagcggaagcttttgacccctgtgacagtactgaccgcagtggttacgttttttgcgatttgcttgacgtggagaatagaggatcccccttttaactcactgctctttgcactgctcgcggctgctgggggcctgcaggggatttacgtcctcgttatgctggtgctcctgatcttagcctacagaaggagatggcgccgactgacagtatgcggggggattatgtttttggcatgtgtactcgtattgatcgtggatgccgtcttgcagctttcccccctgctgggcgcagtcaccgtcgtgagtatgactctgctgttactggccttcgtactgtggcttagcagccctggaggactaggcacgctgggcgccgctttattgaccctggcagccgccctggctctgctggcctccctgatactggggacattaaatttaacaactatgtttctgctgatgctgctgtggactctagttgtgctcttgatatgttcctcctgttccagctgccctctgagcaagatcctgttggctcggctattcctgtacgcgctggctctcctcctgctagcttcagcgctaattgcaggcgggtctattctccagacgaattttaagagcctcagtagcactgagtttatcccgaatctgttctgcatgttgctattgatcgtcgcaggcattctcttcattctggccatacttacagagtggggtagcggcaaccggacttatggccctgtgttcatgtgcttggggggccttctcacaatggtggcaggggcggtatggctgaccgtgatgtctaacaccttgttatcagcctggatccttaccgccggcttcctaatttttctgatcggcttcgccctcttcggagtaatacgatgctgtcggtactgctgctactattgccttactttggagagcgaagagcggcctcccactccctatagaaacaccgtttgataataggctggagcctcggtggccatgcttcttgccccttgggcctccccccagcccctcctccccttcctgcacccgtacccccgtggtctttgaataaagtctgagtgggcggc

SEQ ID NO: 150 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgtttttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttttctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacccccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttttgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaaaacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacccccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacccccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaaaccaggcgaagttaacgtgacaaaaggaacgcccccccagaacgcaaccagtcctcaggcccccagcgggcagaaaactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccccctacaagccagccacgcttctccaaccttagtatgctcgttctccagtgggccagccttgctgttctgaccctcctcctgctgctcgtgatggccgattgcgcctttaggagaaacctcagcactagccacacgtacaccacgcccccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 151 - RNA, 2761 nt, synthetic construct
  source 1..2761: mol_type = other RNA
gggaaataagagagaaaagaagagtaagaagaaatataagagccaccatggaggctgctctattggtgtgccaatacaccattcagtcgctgatccacttgactggggaggatcccggatttttcaacgtggagattccggaattccctttttaccccacctgcaacgtgtgcactgccgacgtaaacgtgaccataaactttgacgtcggtgggaagaagcaccagctggacctggacttcgggcagttaacaccccacacaaaggcagtttaccagcccagaggagccttcggcggatcggagaatgcaacaaatcttttcctgctagaactgctcggcgccggcgagttggccctgacaatgagaagcaagaaacttcctatcaacgtaaccacaggggaggagcagcaggtcagcctcgaaagtgttgacgtctatttccaggatgtctttgggactatgtggtgccatcacgccgaaatgcagaatcccgtgtacctcattcccgagacagtaccctatatcaagtgggacaactgcaactctactaatattacagccgtggtgcgcgctcagggacttgatgtaacattgcctttgtctttgcccacaagcgctcaggattctaatttctctgtaaagacggagatgctggggaatgaaatcgacatagaatgtattatggaagacggtgaaatcagtcaggtgctaccgggagacaacaaattcaatattacatgttctggctatgagagccacgtgccgagcgggggcatcctgacttcgacttctcccgtcgccacacccataccaggcacaggctacgcttatagtctgaggctgactcccaggccagtatcccgatttctgggtaataattccatcctttatgtgttttactccggtaacggccccaaagcttcggggggcgactattgtatccagtctaatatagtgttctccgatgagattcccgcgagtcaggatatgcccaccaacaccaccgacatcacgtatgttggggataacgctacctatagtgtgccgatggtgacatccgaggacgcaaactctcctaacgtaaccgtgaccgccttttgggcatggccaaataataccgaaaccgattttaaatgcaagtggaccctgacatccgggactccctccggatgcgaaaatatttctggagcttttgccagcaatcggacctttgatatcactgtgagtggtttaggcaccgccccaaaaacactgatcataaccaggacagccacaaatgcgaccacaactacacacaaagttatatttagtaaagcacctgagtccaccacaacttctccgaccctcaacactaccggcttcgctgatccgaataccaccacaggtcttccatcttccacccacgttccaactaatcttacggcgcctgctagcaccggtccaaccgtgtccaccgccgatgtgactagccccacgccggctggaacaacatcaggggccagccctgtaaccccaagcccttcaccctgggacaatggtaccgagagtaaggcgcccgacatgacctcatcaacttctcctgttacaactccaacaccaaatgccactagtcctacccccgcagtcactaccccaacgcctaatgcaacgtcaccaactcctgcagtgaccacaccaacacccaatgctacttcgccgaccctgggcaagaccagcccaaccagtgccgtgacgacaccgactccaaatgcgaccggacctactgtcggtgagacaagcccccaggcaaacgcgaccaaccacaccctcggcgggacttctccaacacccgtcgtaacgtcccaaccaaaaaatgctacatcggccgtgactaccggacagcataatatcacctcaagctccacgtcgtcaatgagcctgcggccgtcaagtaatccagagactctgtcgccatctacctcagataactcaactagccacatgcctctactcactagtgcccaccccaccggcggggagaacatcacgcaggtaacaccagcgtctatcagtactcaccatgtgagtaccagctcccccgctcctcgaccgggaaccacgagccaggccagcggaccgggtaatagtagcacttctactaaaccgggagaggtcaatgtgactaaggggactcccccccaaaacgctaccagcccacaggctccctcagggcagaagaccgctgttccaacagttacatctactgggggcaaggcaaatagcacaaccggtggcaagcatactactgggcacggtgcccgcacaagcaccgagcctacaacggactatgggggggattctaccacaccacgtccccggtataacgccaccacgtatcttccgcctagcacatcaagcaaactcagacctcgctggacatttacatcacccccagttaccactgctcaggcaaccgtcccggtgcctcctacttcacaaccacgtttcagcaacttgagcatgctggtcctgcagtgggcttccctcgccgtgctgacattactgttattgctggtgatggccgattgcgcgtttcgcaggaacctgtctaccagccacacatacacaacgcccccgtatgacgacgcagagacttatgtgtgataataggctggagcctcggtggccatgcttcttgccccttgggcctccccccagcccctcctccccttcctgcacccgtacccccgtggtctttgaataaagtctgagtgggcggc

SEQ ID NO: 152 - RNA, 2761 nt, synthetic construct
  source 1..2761: mol_type = other RNA
gggaaataagagagaaaagaagagtaagaagaaatataagagccaccatggaagccgctcttctggtctgccagtatacaatccagtcgctgattcatctcaccggcgaggatccaggtttctttaacgtggagattccagaattccctttctatccaacttgtaacgtgtgtactgccgacgttaacgttactattaactttgacgtgggcggaaagaagcaccagctggatcttgatttcggccagctaacacctcataccaaggccgtctaccaaccacgcggggcatttggcggttctgagaatgctacaaacctgttccttttggaactcctcggagcaggggaactggcacttacaatgaggagtaagaagctgcccatcaacgtcaccacgggggaagaacagcaggtgtccctggagtccgttgatgtgtatttccaggacgttttcgggacaatgtggtgtcatcatgccgaaatgcagaatccggtgtatctcattcctgagaccgtgccctatattaaatgggataactgtaattcgacaaacataactgccgtagtgcgtgcccagggtctggatgtaactttgcctctctcccttcccacttctgcccaggactccaatttttcagtgaagaccgaaatgcttgggaacgagatcgacatcgagtgtatcatggaggatggggagatcagtcaggtgttgcctggtgacaataagtttaatataacatgttccggctatgaatcacatgttccaagcggaggtattctcacaagtacatctcccgtagccacgccgattccgggaaccggatacgcctattcacttcggctgactcctcggccagtgtcacgattcctgggcaataactcaattctgtacgtcttttacagcggcaacggcccaaaggccagtggaggcgattactgtatccagtctaacatcgtattttcagatgagattccagcaagccaagacatgccaactaatactacagacattacatacgtcggtgataacgccacttattcagtccctatggtcacatccgaggacgcgaactcacccaatgttaccgttaccgcattctgggcatggcccaataatacggaaacagacttcaaatgtaagtggaccctgaccagtggcaccccttctggctgcgaaaatataagcggggcatttgctagcaacagaacattcgatattactgtgtctggcctggggaccgcccccaaaacactcattataacgcgaacagctacgaacgctaccacaaccacacataaagtgattttcagcaaggctccagaatcaactacaaccagtccgacccttaataccacaggcttcgcagacccgaacacaacgacgggactgccctcctcaacgcatgtgccgactaatctgactgctcccgcaagtaccggcccaacagtctccacagcagacgtcacctctccgactcccgccggcactacatctggagcctcaccggtgactcctagtccttccccctgggacaatgggaccgagtctaaagccccggacatgacgagttctacctcaccagttacaaccccaacacctaatgccacaagccctacgccagccgtgaccacccctacccccaatgccaccagcccaacacccgctgttacaacgccaacccccaacgctacttctccaacgctaggaaaaacatcacccacctcagctgtaaccacccccacaccaaacgctaccgggccaaccgttggcgaaacctccccccaggccaatgcgacaaaccacacccttggggggacttctcccactcctgtggtgacttcacaaccgaagaacgcgacgtcggccgttacgaccgggcagcacaatatcacctcgtcctccacgtcgtccatgtccttgcggccttccagtaacccagagacactgtctcctagtaccagtgacaacagcacctctcacatgccattactgacttccgcccacccaaccggcggagagaacatcactcaggtgacccccgcatctatttctacccaccacgtgagcactagcagcccggcaccaagaccagggaccacctcccaggctagcggcccgggcaactcctccactagcacgaaaccaggagaggtgaacgtgaccaagggcacaccgccacagaatgcaacttccccacaggccccctccggccagaaaactgctgtacctacggtcactagcactggcggaaaagcgaattcaaccaccggtggtaagcataccaccggacacggcgctcggactagcacagagccaaccactgattacgggggtgactcaacgaccccgaggcccaggtacaacgcaaccacctatctcccaccatccacatcttccaagctcaggcctcgttggactttcacctccccgcctgtgactacagcgcaggccacagtgccagtgcctccaacgtcacagccgcggtttagcaatctctccatgctggtcctgcagtgggcatctctggcagtcctgacactgcttcttttgctcgtgatggccgattgtgcgtttagacgcaacctttcaacatcccacacatatacaactccaccctatgatgacgcagagacttatgtgtgataataggctggagcctcggtggccatgcttcttgccccttgggcctccccccagcccctcctccccttcctgcacccgtacccccgtggtctttgaataaagtctgagtgggcggc

SEQ ID NO: 153 - RNA, 2761 nt, synthetic construct
  source 1..2761: mol_type = other RNA
gggaaataagagagaaaagaagagtaagaagaaatataagagccaccatggaagccgccctattagtctgccagtacaccattcaaagcctcatccacttgactggggaggatcctgggttctttaacgtggaaatcccggaattccctttctacccaacttgcaacgtgtgcacggctgacgtcaacgtgactatcaacttcgatgtgggggggaagaaacatcaacttgatctggactttggccagcttactccccacaccaaggccgtgtaccagccccgcggcgcattcggtgggagcgagaacgcgacaaatctattcttgctggaactgctgggcgcaggagagctcgccctgacgatgcgatctaaaaagcttcccatcaacgttaccactggcgaggagcagcaggtgtccctcgaaagcgtcgacgtctatttccaagacgtcttcggaacaatgtggtgccaccacgctgagatgcagaaccccgtatatcttattcccgaaaccgtgccatacatcaagtgggacaactgcaactcaactaacatcactgcagtagtgcgcgcccagggactggacgtcactttgccactgtcactgcccacatcagcccaagacagcaacttcagcgtcaagacagaaatgctgggcaacgaaatcgacatcgaatgcataatggaggatggggaaatttcccaggtcctccccggtgataacaagtttaatattacttgttctggatatgaatcccatgtgccgtctggtggtatactgacgagtacatcgccagtggctaccccaatccccgggacaggctatgcctacagcctgagactgacgccaagaccagtttccaggtttctggggaataactctatcctgtatgtgttttattctggtaacggacccaaggcatccggcggtgattattgtatccagagcaatatagtgttctctgatgagattccggcctcccaggatatgccaaccaacacgacagatatcacctatgtcggggacaacgccacatatagcgtgccaatggtgaccagcgaagatgccaattctcccaacgtcacagtgacagccttttgggcgtggcctaataacacagaaactgactttaaatgcaaatggactctaacctcaggcaccccatctggctgcgagaacataagcggcgcgtttgcatcgaacagaacttttgatattacagtatctgggttgggcactgcccccaaaactctaataattacacggacggccactaacgccaccacgacgactcacaaggtgatcttcagcaaagctcccgaatctactactacctcgccgacactgaatacgacaggattcgccgacccaaatactacgaccggccttcccagctcgacacatgtgcctacaaacttaacggcccccgccagtaccggccccaccgtgagcaccgctgacgtcacctctcctacccctgccgggactacaagtggtgcttccccagttactcccagccctagcccttgggacaacggcactgagagcaaagctccggacatgaccagcagtaccagtccagtgacaactccgacacccaatgcgacgtccccaactcccgccgtgaccacgccaacaccaaatgccacgtcacctactccagcggtcaccaccccaacgccaaacgctactagtcccactctaggcaagacatctcccacctccgctgtcaccacccctacacccaatgctaccggtcccacggtgggtgagactagcccacaggccaacgcaactaatcatacattggggggcacaagcccaacccccgtcgtcactagccagcctaaaaacgccacaagcgctgtaactaccggccagcacaatataacaagttccagtactagcagtatgtctctgagacccagctcaaatcctgaaaccctctccccctctacgtcggataactccacgtcccacatgcctctactcacaagcgcgcaccctaccgggggagaaaatatcactcaggttaccccagcttctatctcgactcaccatgtctctacttcttccccagcgccacggcccggcactaccagccaggctagcgggccaggtaattcctccactagcactaaacctggcgaagtcaacgttaccaagggaacacccccccagaatgcaacctccccccaagctccctctggacagaaaacagccgtccctaccgtcacaagcaccggtgggaaggcgaattctacgacaggcgggaagcacactaccggacacggagcaaggacatccactgagcccacgaccgactacggtggcgactccaccacccccagacccaggtacaatgcgactacctacctgcccccttccacttccagcaaactgaggcctcgttggactttcacttctcccccagtcaccaccgctcaggctaccgtgcctgtcccgccgacctctcagccccggttttcaaatttgagtatgctcgtgctgcagtgggccagcctggcagtgcttaccctcctgctcttgctggttatggctgactgcgccttccgacgtaacttatccaccagtcatacttacactacacccccttatgacgacgcggagacctacgtgtgataataggctggagcctcggtggccatgcttcttgccccttgggcctccccccagcccctcctccccttcctgcacccgtacccccgtggtctttgaataaagtctgagtgggcggc

SEQ ID NO: 154 - RNA, 2761 nt, synthetic construct
  source 1..2761: mol_type = other RNA
gggaaataagagagaaaagaagagtaagaagaaatataagagccaccatggaagccgccttactggtctgccagtacactattcagtccctcatccatctcacaggcgaggacccggggttcttcaacgtcgaaatccccgaattcccattctatcccacctgtaacgtctgcacagccgacgttaacgttacaatcaatttcgacgtgggcgggaaaaaacaccagctggacctcgactttgggcagctgacgccccacactaaagccgtttaccagcccaggggtgcattcggtggatctgaaaatgctacaaacctctttctgcttgagctgcttggtgctggagagctggccctgacaatgcgcagcaaaaagttgccaatcaatgtgaccacaggtgaagaacagcaggtgtccctagaatccgtggatgtgtacttccaggacgttttcggcacaatgtggtgccatcacgcagagatgcaaaatccggtgtaccttatccccgaaacagtgccgtacattaaatgggataattgtaactccacaaacatcacggcagtggtgcgcgctcaggggctggacgtgacactccccctttccttaccaacaagtgctcaagattccaacttttctgtgaaaaccgagatgctgggcaacgagattgatattgaatgcatcatggaggatggcgagattagccaggtgttgcccggcgacaataaatttaatattacctgttccggctatgagtcacacgtgccaagtggaggcattctcacgagcacctctcccgttgcaacacccattcctgggacagggtacgcatatagtctcaggctgacaccgagaccggtatcgcgcttcttaggtaacaattccatcttgtatgtgttttacagcggaaacgggccaaaggcctcaggaggagactactgcatccagagcaatatagtttttagcgacgagatccctgcctcacaagacatgcccaccaacaccaccgacatcacgtacgtcggtgataatgccacttacagcgtgcctatggtgacctctgaagacgcaaattcacccaatgtcacggttaccgcattctgggcttggccaaataacactgaaaccgacttcaagtgtaaatggaccctaaccagcggcaccccatcagggtgcgagaatatcagcggggccttcgccagcaaccggacattcgacatcaccgtgtcaggactagggactgctccaaaaacattaattattacacggacagcaactaacgctacgactacgactcacaaagtgatatttagcaaagcccccgaatcgactactacgagcccgaccctaaacaccaccggtttcgccgatcccaacacaactactggtctcccatcttcgacccatgttccaacgaatctgacagctccagcaagtacggggcccactgtgagtacagccgacgttactagcccaactcccgcgggtacgactagtggcgcgtctccagtgaccccttcgccttctccgtgggataatggaaccgagtcaaaggctcctgatatgaccagctccaccagtcccgttacaacgccaacccccaatgcaacgagtccaactccggcggttaccactccgacaccaaacgccacctcccccacgcctgcagttactacgcccacccctaatgctacgtcacccactctgggaaagacctcaccaacctctgccgtgactactcctacacctaatgccacaggacccacagttggggaaaccagcccacaggctaacgcaactaatcatacgcttggggggacctctcccacgcccgtggtcacttcacagcctaagaatgccacaagcgctgtgactacaggccagcataatataaccagcagttcaaccagtagcatgtccctgcgtccttcatccaatcctgagactctgagccccagcactagtgacaactcaacttctcacatgccgttgctgacctccgcgcatcccacgggcggtgagaatatcacacaggtgacacccgcgtctattagcacacatcacgtgagtacgtcctcccccgcaccccgcccaggcacaacctcacaagcatcaggtccggggaacagcagcacaagtaccaagcctggcgaagtgaatgtaaccaagggcacgcccccgcagaacgccacatcccctcaagccccatccggccagaagaccgctgtacccaccgtgacatcaactggggggaaagcgaactcaactaccggggggaagcatacaacagggcacggtgccagaacttctacagagccaaccacggactacggaggggacagcacaacgcccagaccgcggtacaatgcgaccacatacctgcctccctcaacttcgagtaagctgcgacccagatggacctttacttctccccctgtcaccaccgctcaagccaccgtcccggttccacctacttcccagcctagattctccaatctgagcatgcttgtcctacagtgggcatcactcgcggtactaaccttgctgctactgttggtgatggcggactgcgcgtttcgccgcaacttatccacctcccacacgtacacaacacctccctatgatgacgctgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 155 - RNA, 835 nt, synthetic construct
  source 1..835: mol_type = other RNA
gggaaataagagagaaaagaagagtaagaagaaatataagagccaccatggtgagcttcaagcaggtgagagtgcccctgttcaccgccatcgccctggtgatcgtgctgctgctggcctacttcctgccccccagagtgagaggcggcggcagagtggccgccgccgccatcacctgggtgcccaagcccaacgtggaggtgtggcccgtggaccccccaccccccgtgaacttcaacaagaccgccgagcaggagtacggcgacaaggaggtgaagctgccccactggacccccaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccacccccgacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagctgataataggctggagcctcggtggccatgcttcttgccccttgggcctccccccagcccctcctccccttcctgcacccgtacccccgtggtctttgaataaagtctgagtgggcggc

SEQ ID NO: 156 - RNA, 577 nt, synthetic construct
  source 1..577: mol_type = other RNA
gggaaataagagagaaaagaagagtaagaagaaatataagagccaccatgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggctgataataggctggagcctcggtggccatgcttcttgccccttgggcctccccccagcccctcctccccttcctgcacccgtacccccgtggtctttgaataaagtctgagtgggcggc

SEQ ID NO: 157 - RNA, 2284 nt, synthetic construct
  source 1..2284: mol_type = other RNA
gggaaataagagagaaaagaagagtaagaagaaatataagagccaccatgcagttgttgtgcgtgttctgcctcgtgttactctgggaggtgggcgccgccagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctgtgataataggctggagcctcggtggccatgcttcttgccccttgggcctccccccagcccctcctccccttcctgcacccgtacccccgtggtctttgaataaagtctgagtgggcggc

SEQ ID NO: 158 - RNA, 1324 nt, synthetic construct
  source 1..1324: mol_type = other RNA
gggaaataagagagaaaagaagagtaagaagaaatataagagccaccatggaacacgaccttgagaggggcccaccgggcccgcgacggccacctcgaggacctcccctctcctcttccctaggccttgctctccttctcctcctcttggcgctactgttttggctgtacatcgttatgagtgactggactggaggagccctccttgtcctctattcctttgctctcatgcttataattataattttgatcatctttatcttcagaagagaccttctctgtccacttggagccctttgtatactcctactgatgatcaccctcctgctcatcgctctctggaatttgcacggacaggcattgttccttggaattgtgctgttcatcttcgggtgcttacttgtcttaggtatctggatctacttattggagatgctctggcgacttggtgccaccatctggcagcttttggccttcttcctagccttcttcctagacctcatcctgctcattattgctctctatctacaacagaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggaccaccactctgctctcagaacctaggcgcacctggaggtggtcctgacaatggcccacaggaccctgacaacacggatgataatggcccacaggaccctgataacactgatgacaatggcccacatgacccgctgcctcaagacccagacaatactgatgacaatggcccacaggaccctgacaacactgatgacaatggcccgcacgacccgttacctcatagccctagcgactctgctggaaatgatggaggccctccacaattgacggaagaggttgagaacaaaggaggtgaccagggcccgcctttgatgacagacggaggcggcggtcatagtcatgattccggccatggcggcggtgatccacaccttcctacgctgctgttgggttcttctggttccggtggagatgatgacgacccgcacggcccagttcagctaagctactatgactgataataggctggagcctcggtggccatgcttcttgccccttgggcctccccccagcccctcctccccttcctgcacccgtacccccgtggtctttgaataaagtctgagtgggcggc

SEQ ID NO: 159 - RNA, 1657 nt, synthetic construct
  source 1..1657: mol_type = other RNA
gggaaataagagagaaaagaagagtaagaagaaatataagagccaccatggggtccctagaaatggtgccaatgggcgcgggtcctcctagccccggcggggatccggatgggtacgatggcggaaacaactcccaatatccatctgcttctggctcttctgggaacacgcccaccccaccgaacgatgaggaacgtgaatctaatgaagagccaccaccgccttatgaggacccatattggggcaatggcgaccgtcactcggactatcaaccactaggaacccaagatcaaagtctgtacttgggattgcaacacgacgggaatgacgggctccctccacctccctactctccacgggatgactcatctcaacacatatacgaagaagcgggcagaggaagtatgaatccagtatgcctgcctgtaattgttgcgccctacctcttttggctggcggctattgccgcctcgtgtttcacggcctcagttagtaccgttgtgaccgccaccggcttggccctctcacttctactcttggcagcagtggccagctcatatgccgctgcacaaaggaaactgctgacaccggtgacagtgcttactgcggttgtcactttctttgcaatttgcctaacatggaggattgaggacccaccttttaattctcttctgtttgcattgctggccgcagctggcggactacaaggcatttacgttctggtgatgcttgtgctcctgatactagcgtacagaaggagatggcgccgtttgactgtttgtggcggcatcatgttcttggcatgtgtacttgtcctcatcgtcgacgctgttttgcagctgagtcccctccttggagctgtaactgtggtttccatgacgctgctgctactggctttcgtcctctggctctcttcgccaggtggcctaggtactcttggtgcagcccttttaacattggcagcagctctggcactgctagcgtcactgattttgggcacacttaacttgactacaatgttccttctcatgctcctatggacacttgtggttctcctgatttgctcttcgtgctcttcatgtccactgagcaagatccttctggcacgactgttcctatatgctctcgcactcttgttgctagcctccgcgctaatcgctggtggcagtattttgcaaacaaacttcaagagtttaagcagcactgaatttatacccaatttgttctgcatgttattactgattgtcgctggcatactcttcattcttgctatcctgaccgaatggggcagtggaaatagaacatacggtccagtctttatgtgcctcggtggcctgctcaccatggtagccggcgctgtgtggctgacggtgatgtctaacacgcttttgtctgcctggattcttacagcaggattcctgattttcctcattggctttgccctctttggggtcattagatgctgccgctactgctgctactactgccttacactggaaagtgaggagcgcccaccgaccccatatcgcaacactgtatgataataggctggagcctcggtggccatgcttcttgccccttgggcctccccccagcccctcctccccttcctgcacccgtacccccgtggtctttgaataaagtctgagtgggcggc

SEQ ID NO: 160 - RNA, 892 nt, synthetic construct
  source 1..892: mol_type = other RNA
gggaaataagagagaaaagaagagtaagaagaaatataagagccaccatgggcagaagacccttctttcaccccgtgggagaagccgactatttcgagtaccaccaggagggcggtcctgacggcgagcccgacgtgcctccaggcgccatcgaacagggccccgctgacgatcctggcgaggggccttccacaggccctcgcggccagggagatggcggaaggcggaagaagggcggctggtttggcaagcataggggccagggcggctccaatcccaagttcgagaatatcgccgaggggctccgggctctgctggcccggtcgcacgtcgaacgtaccactgacgaggggacgtgggtggccggcgtctttgtgtacggaggatccaagacctccctgtacaacctgagaaggggcaccgcccttgccatccctcagtgcagactcacgcccctgagcaggctcccctttgggatggctcctgggccaggcccgcagcccgggcccctcagggaaagcatcgtgtgttactttatggtgtttctgcagacacacatctttgcagaagttctgaaagatgcaatcaaggatctggtgatgaccaagcctgcacctacgtgcaatatcagggtgacagtatgctccttcgacgacggtgtggacctcccgccctggttcccacccatggtcgagggtgccgccgccgagggagacgatggcgacgacggtgatgagggtggagacggagatgaaggcgaggaggggcaggagtgataataggctggagcctcggtggccatgcttcttgccccttgggcctccccccagcccctcctccccttcctgcacccgtacccccgtggtctttgaataaagtctgagtgggcggc

SEQ ID NO: 161 - RNA, 2998 nt, synthetic construct
  source 1..2998: mol_type = other RNA
gggaaataagagagaaaagaagagtaagaagaaatataagagccaccatggacaaggacaggcccggccctcccgccttggacgacaacatggaggaggaggtgcccagcaccagcgtggtgcaggagcaggtgagcgccggcgactgggagaacgtgttaatcgagttgagcgacagcagcagcgagaaggaggccgaggacgcccacctggagcccgcccagaagggcaccaagaggaagagggtggaccacgacgctggcggatctgcccctgccaggcccatgctgccaccccagcccgacctgcccggcagggaggccatcctgaggaggttcccactggacctgaggaccctgctgcaggccatcggcgccgccgccaccaggatcgacaccagggccatcgaccagttcttcggcagccagatcagcaacaccgagatgtacatcatgtacgccatggccatcagacaagcaattagggatcgacgcagaaatcctgccagcaggagggaccaggccaagtggaggctgcagaccctggccgccggctggcccatgggctaccaggcctacagcagctggatgtacagctacaccgaccaccagaccactccgaccttcgtgcacctccaggctaccctgggctgcactggcggtcgcaggtgccacgtgaccttctcagcgggcaccttcaagctgcccaggtgcacacctggcgacaggcagtggctgtacgtgcagagcagcgtgggcaacattgttcagagttgcaaccccaggtacagcatcttcttcgactacatggcgattcacaggagcctgaccaagatctgggaggaagtcctgacccctgaccagagggtgagcttcatggagttcctgggcttcctgcagaggaccgacctgagctacatcaagagcttcgtgagcgacgccctgggcaccaccagcatccagactccttggatcgacgataaccctagcaccgaaaccgcccaggcctggaacgccggtttcctgaggggcagggcctacggcatcgacctgctccggaccgagggcgagcacgtggagggcgccaccggcgagactagggaggagagcgaggacactgagagcgacggcgacgacgaggatctgccgtgcatcgtgagcaggggcggccccaaggtgaagaggcctcccatcttcatccggaggcttcacaggctgctgctgatgagggccggcaagcgtactgaacagggcaaggaggtactggagaaggccaggggcagcacatatggcacccctagaccgcctgtgcccaagccgcgtcctgaagtcccgcagagtgatgagacagcgactagccatggcagtgctcaggtgcccgagccacccaccatccatctcgccgcacagggcatggcctacccactgcacgagcagcacggcatggcgccctgccccgtggcccaggctcctccaaccccgcttcctccagtatcccctggtgaccagttgccaggcgtgttcagcgacggtagggtggcctgcgcgccagttcctgcaccagcaggcccaattgtgaggccctgggagccctccctaacccaggcggcaggtcaggccttcgctccagttagaccacagcacatgcccgtggaaccggtgccagtgcctacggtggcgctggagaggcccgtgtacccaaagcctgtgcgacctgcgcctcctaagatcgccatgcaaggcccgggcgagactagcggtatcaggcgggccagggagaggtggcgcccggcaccgtggactcccaacccaccaaggtcacctagtcagatgagcgtgcgtgataggctggcacggcttcgggcagaggctcaggtaaagcaggccagtgtggaggtgcagccaccacagctgactcaggtgtccccgcagcagcccatggagggcccactggtgccggaacagcagatgttccccggcgccccgttcagccaggtggccgacgtggttagggctcctggcgtgcctgctatgcagcctcagtacttcgacctaccgctgatccagcccatcagtcaaggtgccccagtggccccactgagggctagcatgggcccggtccctcctgttccagccacccagcctcaatatttcgacattcctctgaccgagcctataaaccagggcgcgtctgcggcccacttcctgcctcaacagccaatggaaggcccgctggtcccagaacagtggatgttccctggagctgccctgagccaatcagtgaggccgggagttgcccagagccagtatttcgatctccctttgacacagcctatcaatcacggagcacctgccgctcacttccttcaccagcctccgatggaaggtccttgggttccagagcaatggatgttccaaggtgcgccaccatctcagggcacagacgtggtccagcatcagctggacgcgcttggctacactctacacggcctgaaccacccgggcgttccagtgagtccagccgtgaaccagtaccatctctctcaggctgccttcggcctgcccattgacgaggacgagagcggcgagggcagcgataccagcgagccctgcgaagccctggatctttccatccacggccgaccttgcccacaggctccagagtggcccgtacaggaagaaggcggccaggacgccaccgaggtcctggacttgtccatacatggcagaccgcgccctaggaccccggagtggccagttcagggtgaaggtggtcagaacgtgaccggcccggaaacccgaagggtggtcgtgtccgctgttgtccacatgtgtcaggatgacgaattcccagatctgcaagatccacccgacgaggcctgataataggctggagcctcggtggccatgcttcttgccccttgggcctccccccagcccctcctccccttcctgcacccgtacccccgtggtctttgaataaagtctgagtgggcggc

SEQ ID NO: 162 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcacttacgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 163 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacattcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 164 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatactcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 165 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacctggaaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 166 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcacttacgacgtgaacgtgacattcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatactcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacctggaaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 167 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcacttacgacgtgaacgtgacattcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattacggctgtggtgagggcccagggcttggacgtgactctgcccttatctctacctacttctgcccaagactccaatttcgcggtcaagaccgagatactcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttgcggtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccgcgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgacctgggatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 168 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtggcgatcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttgcgaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattgcggctgtggtgagggcccagggcttggacgtgactctgcccttatctctacctacttctgcccaagactccaatttcgcggtcaagaccgagatgctcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatgcgatcttatacgtgttttatagcggaaacggccctaaagcgtccggaggggactattgtattcagagtaatatcgttttctctgatgagattcctgccagtcaggacatgccgacaaacacagcggatattacctacgtgggcgacaatgccgcgtattcagtgcccatggtcacgagcgaggacgccaattcaccaaatgttgcggtaacagctttctgggcctggccaaataacgcggagactgacttcaaatgtaagtggactttgacctctggaactccgtcgggttgcgagaatatcgcgggggcctttgcttccaacagggcgttcgacatcactgtctcagggctggggacagcaccgaagacattaatcataacacggaccgccaccaacgccgcgactacaacccataaggtgatcttttccaaggcacctgagtccaccactacctccccgactcttaacactgcgggcttcgctgatcccaacaccgcgacggggttgcctagctcgacacatgtgccgacgaacctggcggcccctgcatcgaccgggcccacagtttcgaccgccgatgtgacatcaccaacgcccgcaggtacaacctcaggcgccagcccagtgaccccttccccaagcccctgggataatggagcggagtccaaggctcctgatatgacttcctctaccagccccgtgactacacccactcccaacgcagcgagcccaaccccagctgtgacgacgcccaccccgaacgcggcgtctcccacacctgctgtgacaaccccaacccctaacgccgcgagccctaccctaggtaagaccagtccgactagcgccgttacaacccctacccctaacgcagcgggcccgaccgtgggcgagacttccccgcaagccaatgcggcgaatcacgcgttgggcgggacctctcctacaccagtcgttacatctcagcctaagaacgctgcgtccgctgtcactaccggacagcacaacatcgcgagctcaagcacgagttccatgagcttgcggccgagctcaaatcccgagaccctatcaccatccacatcagacaacagtgcgtcacacatgccactcttgacgagcgctcaccccaccggcggcgagaacatcgcgcaggtgactccggcgtctatttccacccaccacgtcagcacgtcgtctcccgcaccgagaccagggacgacttctcaggccagtgggcctggcaactccgcgacaagcacaaagccaggcgaagttaacgtggcgaagggaacgcctccccagaacgcagcgagtcctcaggcgcccagcgggcagaagactgcggtgccaactgtgaccagcaccggtggcaaggccaactcagcgactggaggcaagcatacgacggggcacggcgcccggacctccactgaacccacgaccgattacggaggtgacagcacaacaccgcggccacgatataatgccgcgacttatctgccaccatccacaagctccaagctgcggccacggtggaccttcacaagcccacccgtgacgactgcccaagcgacggtgccagtgccacctacaagccagccacgcttctccaaccttgcgatgctcgttctccagtgggccagccttgctgttctgaccctcctcctgctgctcgtgatggccgattgcgcctttaggagaaacctcgcgactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 169 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtggcgatcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttgcgaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattgcggctgtggtgagggcccagggcttggacgtgactctgcccttatctctacctacttctgcccaagactccaatttcgcggtcaagaccgagatgctcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatgcgatcttatacgtgttttatagcggaaacggccctaaagcgtccggaggggactattgtattcagagtaatatcgttttctctgatgagattcctgccagtcaggacatgccgacaaacacagcggatattacctacgtgggcgacaatgccgcgtattcagtgcccatggtcacgagcgaggacgccaattcaccaaatgttgcggtaacagctttctgggcctggccaaataacgcggagactgacttcaaatgtaagtggactttgacctctggaactccgtcgggttgcgagaatatcgcgggggcctttgcttccaacagggcgttcgacatcactgtctcagggctggggacagcaccgaagacattaatcataacacggaccgccaccaacgccgcgactacaacccataaggtgatcttttccaaggcacctgagtccaccactacctccccgactcttaacactgcgggcttcgctgatcccaacaccgcgacggggttgcctagctcgacacatgtgccgacgaacctggcggcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 170 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 171 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 172 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttcgcggtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 173 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 174 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttgcggtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 175 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccgcgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 176 - RNA, 835 nt, synthetic construct; identical to SEQ ID NO: 155
  source 1..835: mol_type = other RNA

SEQ ID NO: 177 - RNA, 2998 nt, synthetic construct; identical to SEQ ID NO: 161
  source 1..2998: mol_type = other RNA

SEQ ID NO: 178 - RNA, 892 nt, synthetic construct; identical to SEQ ID NO: 160
  source 1..892: mol_type = other RNA

SEQ ID NO: 179 - RNA, 1324 nt, synthetic construct; identical to SEQ ID NO: 158
  source 1..1324: mol_type = other RNA

SEQ ID NO: 180 - RNA, 1657 nt, synthetic construct; identical to SEQ ID NO: 159
  source 1..1657: mol_type = other RNA

SEQ ID NO: 181 - RNA, 1657 nt, synthetic construct; identical to SEQ ID NO: 159
  source 1..1657: mol_type = other RNA

SEQ ID NO: 182 - RNA, 2761 nt, synthetic construct
  source 1..2761: mol_type = other RNA
gggaaataagagagaaaagaagagtaagaagaaatataagagccaccatggaagccgctcttctggtctgccagtatacaatccagtcgctgattcatctcaccggcgaggatccaggtttctttaacgtggagattccagaattccctttctatccaacttgtaacgtgtgtactgccgacgttaacgttactattaactttgacgtgggcggaaagaagcaccagctggatcttgatttcggccagctaacacctcataccaaggccgtctaccaaccacgcggggcatttggcggttctgagaatgctacaaacctgttccttttggaactcctcggagcaggggaactggcacttacaatgaggagtaagaagctgcccatcaacgtcaccacgggtgaagaacagcaggtgtccctggagtccgttgatgtgtatttccaggacgttttcgggacaatgtggtgtcatcatgccgaaatgcagaatccggtgtatctcattcctgagaccgtgccctatattaaatgggataactgtaattcgacaaacataactgccgtagtgcgtgcccagggtctggatgtaactttgcctctctcccttcccacttctgcccaggactccaatttctcagtgaagaccgaaatgcttgggaacgagatcgacatcgagtgtatcatggaggatggggagatcagtcaggtgttgcctggtgacaataagtttaatataacatgttccggctatgaatcacatgttccaagcggaggtattctcacaagtacatctcccgtagccacgccgattccgggaaccggatacgcctattcacttcggctgactcctcggccagtgtcacgattcctgggcaataactcaattctgtacgtcttttacagcggcaacggcccaaaggccagtggaggcgattactgtatccagtctaacatcgtattttcagatgagattccagcaagccaagacatgccaactaatactacagacattacatacgtcggtgataacgccacttattcagtccctatggtcacatccgaggacgcgaactcacccaatgttaccgttaccgcattctgggcatggcccaataatacggaaacagacttcaaatgtaagtggaccctgaccagtggcaccccttctggctgcgagaatataagcggggcatttgctagcaacagaacattcgatattactgtgtctggcctggggaccgcgcccaagacactcattataacgcgaacagctacgaacgctaccacaaccacacataaagtgattttcagcaaggctccagaatcaactacaaccagtccgacccttaataccacaggcttcgcagacccgaacacaacgacgggactgccctcctcaacgcatgtgccgactaatctgactgctcccgcaagtaccggcccaacagtctccacagcagacgtcacctctccgactcccgccggcactacatctggagcctcaccggtgactcctagtccttcgccctgggacaatgggaccgagtctaaagccccggacatgacgagttctacctcaccagttacaaccccaacacctaatgccacaagccctacgccagccgtgaccacccctacgcccaatgccaccagcccaacacccgctgttacaacgccaacgcccaacgctacttctccaacgctaggaaagacatcacccacctcagctgtaaccacgcccacaccaaacgctaccgggccaaccgttggcgaaacctcgccccaggccaatgcgacaaaccacacccttggcgggacttctcccactcctgtggtgacttcacaaccgaagaacgcgacgtcggccgttacgaccgggcagcacaatatcacctcgtcctccacgtcgtccatgtccttgcggccttccagtaacccagagacactgtctcctagtaccagtgacaacagcacctctcacatgccattactgacttccgcccacccaaccggcggagagaacatcactcaggtgacgcccgcatctatttctacccaccacgtgagcactagcagcccggcaccaagaccagggaccacctcccaggctagcggcccgggcaactcctccactagcacgaaaccaggagaggtgaacgtgaccaagggcacaccgccacagaatgcaacttccccacaggcgccctccggccagaagactgctgtacctacggtcactagcactggcggaaaggcgaattcaaccaccggtggtaagcataccaccggacacggcgctcggactagcacagagccaaccactgattacggaggtgactcaacgaccccgaggcccaggtacaacgcaaccacctatctcccaccatccacatcttccaagctcaggcctcgttggactttcacctccccgcctgtgactacagcgcaggccacagtgccagtgcctccaacgtcacagccgcggtttagcaatctctccatgctggtcctgcagtgggcatctctggcagtcctgacactgcttcttttgctcgtgatggccgattgtgcgtttagacgcaacctttcaacatcccacacatatacaactccaccctatgatgacgcagagacttatgtgtgataataggctggagcctcggtggccatgcttcttgccccttgggcctccccccagcccctcctccccttcctgcacccgtacccccgtggtctttgaataaagtctgagtgggcggc

SEQ ID NO: 183 - RNA, 2284 nt, synthetic construct; identical to SEQ ID NO: 157
  source 1..2284: mol_type = other RNA

SEQ ID NO: 184 - RNA, 577 nt, synthetic construct; identical to SEQ ID NO: 156
  source 1..577: mol_type = other RNA

SEQ ID NO: 185 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 186 - RNA, 1195 nt, synthetic construct
  source 1..1195: mol_type = other RNA
gggaaataagagagaaaagaagagtaagaagaaatataagagccaccatgagtgactggacaggaggagccctccttgtcctctattcctttgctctcatgcttataattatcatcctgataatctttatcttcagacgggaccttctctgtccacttggagccctttgtatactcctattgatgatcaccctcctcctcatcgctctctggaatttgcacggacaggcattgttccttggaattgtgctgttcatcttcgggtgcttacttgtcttaggtatctggatctacttattggagatgctctggcgacttggtgccaccatctggcagcttttggccttcttcctagcgttcttcctcgacctcattctactcattattgctctctatctacaacagaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggaccaccactctgctctcagaacctaggcgcacctggaggtggtcctgacaatggcccacaggaccctgacaacacggatgataacggtccacaagacccagataacactgacgacaatggaccgcatgacccgctgcctcaggatcctgataacacagacgacaacggcccacaagatccggacaataccgacgataacggaccgcacgacccgttacctcatagccctagcgactctgctggaaatgatggaggccctccacaattgacggaagaggttgagaacaaaggaggtgaccagggcccgcctttgatgacagacggaggcggcggtcatagtcatgattccggccatggcggaggagatccacaccttcctacgctgctgttgggttcttctggttccggtggagatgatgacgacccgcacggcccagttcagctaagctactatgactgataataggctggagcctcggtggccatgcttcttgccccttgggcctccccccagcccctcctccccttcctgcacccgtacccccgtggtctttgaataaagtctgagtgggcggc

SEQ ID NO: 187 - RNA, 2284 nt, synthetic construct; identical to SEQ ID NO: 157
  source 1..2284: mol_type = other RNA

SEQ ID NO: 188 - RNA, 577 nt, synthetic construct; identical to SEQ ID NO: 156
  source 1..577: mol_type = other RNA

SEQ ID NO: 189 - RNA, 835 nt, synthetic construct; identical to SEQ ID NO: 155
  source 1..835: mol_type = other RNA

SEQ ID NO: 190 - RNA, 2047 nt, synthetic construct
  source 1..2047: mol_type = other RNA
gggaaataagagagaaaagaagagtaagaagaaatataagagccaccatggacagcaagggcagcagccagaagggaagcaggttgttattgcttttagtggtgagcaacttactcttaccccagggcgtggtggaggcagcactcctcgtctgccagtatacaatccagtcgctcattcatttgacaggcgaggaccccggcttctttaacgtggaaatcccagagttccctttctatccaacctgtaacgtttgcacagccgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagatccctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctggcagcggcagcgcaggctctgcaagcggcggcgcctccagcggctccggaggtgccagcatgcagatctacgagggcaagctgaccgccgagggcctgaggttcggcatcgtggccagcaggttcaaccacgccctggtggacaggctggtggaaggcgccatcgacgccatcgtgaggcacggcggcagggaggaggacatcaccctggtgcgggtgcccggcagctgggagatccccgtggccgccggcgagctggccaggaaggaagatattgatgcagtgatcgccatcggcgtgctgatcaggggcgccaccccacacttcgactacatcgccagcgaagtgagcaagggactggcggacctgagcctggagctgaggaagcccatcaccttcggcgtgatcaccgccgacaccctggagcaggccatcgagagggccggcaccaagcacggcaacaagggatgggaggccgccctgagcgctatcgaaatggccaacctcttcaagagcctgaggtgataataggctggagcctcggtggccatgcttcttgccccttgggcctccccccagcccctcctccccttcctgcacccgtacccccgtggtctttgaataaagtctgagtgggcggc

SEQ ID NO: 191 - RNA, 2263 nt, synthetic construct
  source 1..2263: mol_type = other RNA
gggaaataagagagaaaagaagagtaagaagaaatataagagccaccatggacagcaagggcagcagccagaagggcagtaggctccttttgctacttgtggtgagcaacctcttgttaccccagggcgtggtggaggcagcactcctcgtctgccagtatacaatccagtcgctcattcatttgacaggcgaggaccccggcttctttaacgtggaaatcccagagttccctttctatccaacctgtaacgtttgcacggcagacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtagtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattccagccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctggcagcggcagcgctggctccgctagcggcggcgcctctagcggatcaggaggcgcatccatggagaacattgcatctggcctgctgggacctctactggtgctgcaggccggattcttcctgctgaccaagatcctgaccatccctcagagcctggacagctggtggaccagcctgaacttcctgggcggcaccccagtgtgcctgggccagaacagccagagccagatctcctcccacagccccacctgctgtccgcccatctgccccggctaccgttggatgtgcctgaggaggttcatcatcttcctgtgcatactcctgttgtgtctaattttcctgctcgttctgctcgactaccagggcatgctgccggtgtgtcctctgatccccggctcctccaccaccagcaccggcccctgcaggacctgcaccacaccagcacaaggtaccagcatgttccccagctgttgctgtaccaagcccaccgacggcaactgcacctgcatccccatccccagcagctgggccttcgccaagtacctgtgggagtgggccagcgtgaggttcagctggctgtcacttcttgcgccattcgtgcagtggttcgtgggcctgagccctactgtgtggctgtcggtaatctggatgatgtggttctggggccccagcctgtacaacatcctgtcaccgttcatgcctctcctaccgctgttcttctgcctgtgggtgtacatctgataataggctggagcctcggtggccatgcttcttgccccttgggcctccccccagcccctcctccccttcctgcacccgtacccccgtggtctttgaataaagtctgagtgggcggc

SEQ ID NO: 192 - RNA, 2569 nt, synthetic construct
  source 1..2569: mol_type = other RNA
gggaaataagagagaaaagaagagtaagaagaaatataagagccaccatggacagcaagggcagcagccagaagggatccaggctattgttactattagtggtgagcaacctcctcttgccccagggcgtggtggacatcgacccctacaaggagttcggcgccaccgtggagttgttaagcttcctccccagcgacttcttccccagcgtgagggacctgctggacaccgccgccgccctgtacagggaggccctggagagccccgagcactgctcgcctcaccacaccgccctgaggcaggccatcctgtgctggggcgagctgatgaccctggccacctgggtgggcaacaacctggaggaccccgccagccgcgacctcgttgtgaactacgtgaacaccaacatgggcctgaagatcaggcagctgctgtggttccacatcagctgcctgaccttcggcagggagacagtgctggagtacctggtgagcttcggcgtgtggatcaggactccacccgcctacaggcctcccaacgcgccaatcctgagcaccctgcccgagacaacagtggtgggcagcggcagcgcaggttccgccagcggcggcgcatccagtggctctggaggagcttctgacattgacccatataaggaattcggtgctacagtagagctactgtctttcttgcctagtgatttcttcccgagtgtaagggatctcctagatacagccgctgctctatatagagaggcactggaatctccagagcactgttccccacaccataccgctctccggcaggcaatcctgtgttggggtgaacttatgacactggcaacctgggtcggtaacaacggatcaggtgaggcggctctgctggtctgccagtatacaatccagtcgctcattcatctgacaggcgaagatccaggtttctttaacgtggaaatcccagagttccctttctatccaacctgtaacgtttgtacagctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgccttctggtggcatactcacaagtacttctcccgtagccacgcccattcccggaaccggatatgcgtatagtctgcgtctgaccccacggcctgtgtccagattcctgggtaacaatagtatcttatacgtgttttatagcggaaacggccctaaagcgtccggaggggactattgtattcagagtaatatcgttttctctgatgaaattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctggatccggactggtggtcaactatgtgaatacaaatatgggcctcaagattaggcagctcctatggttccatatttcctgcttaactttcggtagggaaactgtcctggaatatctcgttagcttcggagtgtggattcggactcctcctgcctacagaccacctaacgctccaattctgtcaactctcccagagacgactgtggtcagaaggaggggcaggagccccagacggcggacaccaagccctcgccgaagaagaagccaaagcccgcggcgccggagatctcagtccagagagagccagtgctgataataggctggagcctcggtggccatgcttcttgccccttgggcctccccccagcccctcctccccttcctgcacccgtacccccgtggtctttgaataaagtctgagtgggcggc

SEQ ID NO: 193 - RNA, 2704 nt, synthetic construct
  source 1..2704: mol_type = other RNA
gggaaataagagagaaaagaagagtaagaagaaatataagagccaccatggacagcaagggcagcagccagaagggtagcaggctactcttgctcctagtggtgagcaacttgcttcttccccagggcgtggtggaggcagcattgcttgtctgccagtatacaatccagtcgctcattcatctaacaggcgaggaccccggcttctttaacgtggaaatcccagagttccctttctatccaacctgtaacgtttgcactgccgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgccttccggcggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattccagccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaatactactactgggttgcctagctcgacacatgtgccgacgaacctgactgcccctgcatcgaccgggcccacagtttcgaccgccgatgtgacaagccctacgcccgcaggtacaacctcaggcgccagcccagtgaccccttccccaagcccctgggacaacggaacggagtccaaggctcctgatatgacttcctctaccagccccgtgactacacccactcccaacgcaactagcccaaccccagctgtgacgacgcccaccccgaacgcgacatctcccacacctgctgtgacaacccctacccctaacgccactagccctaccctaggtaagaccagtccgactagcgccgttactacaccgacccctaatgcaaccggcccgactgtcggcgagacttccccgcaagccaatgcgacaaatcacacattgggcgggacctctcctacaccagtcgttacatctcagcctaagaacgctacctccgctgtcactaccggccagcacaacatcacaagctcaagcacgagttccatgagcttgcggccgagctcaaatcccgagacactatcaccatccacatcagacaacagtacttcacacatgccactcttgacgagcgctcaccccaccggcggcgagaacattacccaggtgactccggcgtctatttccacccaccacgtcagcacgtcgtctcccgcaccacggccagggacgacttctcaggccagcggcccaggaaactcctctacaagcacaaagccaggcgaagttaacgtcaccaagggaacgcctccccagaacgcaacctccccacaggctcccagcgggcagaagactgcggtgccaactgtgaccagcaccggtggcaaggccaactcaacaactggaggcaagcatacgacggggcacggcgcccggacctccactgaacccacgaccgattacggaggtgatagtaccaccccgcggccacgatataatgccaccacttatctgccacctagcacttcctccaagctccggccacggtggaccttcacaagcccacccgtgacgacagcccaggcgacggtgccagtcccacctacaagccagccacgcttctccaaccttagttgataataggctggagcctcggtggccatgcttcttgccccttgggcctccccccagcccctcctccccttcctgcacccgtacccccgtggtctttgaataaagtctgagtgggcggc

SEQ ID NO: 194 - RNA, 3223 nt, synthetic construct
  source 1..3223: mol_type = other RNA
gggaaataagagagaaaagaagagtaagaagaaatataagagccaccatggactcgaagggatctagtcagaaaggctctaggctcctactcctcttggtggtgagcaacttactccttccccagggcgtggtgagagcagtgggcgttttcttagcaatctgcctcgtgacaatctttgtgttgccgacctggggcaactgggcctacccctgctgccacgtgacccagctgagagctcaacacctgcttgccctggagaacatcagcgacatctaccttgtaagcaaccagacatgcgacggcttcagtctggccagcctgaacagccccaagaacggctcaaaccagctagtgatcagccggtgcgctaacggtctgaacgttgtgagcttcttcatcagcatcttgaagagaagtagtagcgcgctgaccggccacctgagagagctgctgaccactctcgagacactctatggcagcttcagcgtggaggacctgttcggcgcaaacctgaaccgctacgcctggcacagaggcggcggaagcggtagcgctggcagcgccagcggaggcgcttcatccggatccggcggcgcaagcagcctttctgaggtgaagctccacttggacatcgaaggccacgccagccattacaccataccatggaccgagctgatggcaaaggtgcccggccttagcccagaagccctgtggcgagaggccaacgttaccgaagacttggctagtatgttaaacaggtacaaacttatctacaagactagcggcaccctgggcatcgcattagccgagcccgtggacatccccgccgttagcgagggcagcatgcaggtggacgccagcaaggttcaccctggtgtaattagtggcctcaacagcccagcctgtatgttgagcgccccactggagaagcagctgttttactatatcgggaccatgttgcccaatacccggccacacagttacgtgttctaccagctgcggtgtcacctgagctacgtggccctgtctatcaacggcgacaagttccagtacaccggcgccatgaccagcaagttcctgatgggcacctacaagcgggtcaccgagaagggcgacgagcacgtgctgtcacttgtgttcggtaagaccaaggacctgcccgaccttcggggccccttttcataccctagtttgaccagcgcccaaagtggcgactacagccttgtgatagtgaccaccttcgtgcattacgccaacttccacaactacttcgtgcccaacctgaaggacatgttcagccgggcggtaaccatgacggccgcttcttatgcccggtatgtgctgcagaagctggtcctgctggagatgaagggcgggtgccgggagcccgagttggacaccgaaaccctgaccaccatgttcgaggttagcgtggccttcttcaaggtgggtcacgcggtgggcgaaaccggcaatggatgcgtggacttacgctggctggccaagagtttctttgagcttaccgtgctgaaggatatcatcggcatctgctacggcgccaccgtaaagggaatgcagagctatgggctggagagacttgccgccatgctcatggctaccgtgaagatggaggagctgggacacctgacaacagagaagcaggagtacgccctgagactggccaccgtgggctaccctaaggccggcgtgtacagtggactcataggcggagctaccagcgtgctcctatccgcttataacagacaccccttatttcagcccctgcacacagtgatgcgggagactttattcatcggatctcacgtcgtactgcgggagttgcggctgaacgtaaccacccagggccctaatttagcactttatcagctgctcagtactgccctgtgcagcgcacttgagatcggcgaggtactcagaggcctggccctcggtacggagagtggcctgttttctccatgctaccttagcctgcggtttgatctgaccagagacaagttgctgagcatggctcctcaagaggcgacccttgaccaggcagccgtgtcaaacgccgtggacggattcctgggcagactgtctctggaacgggaagaccgggatgcttggcaccttcctgcctataagtgtgtggatcggctggataaggtcctgatgataatccctctgattaacgttactttcatcatctctagcgatcgggaggtccgagggtccgccctctacgaggccagcaccacctatctgagcagcagcctcttcttgtctcctgtgattatgaacaagtgcagccagggtgcagtagccggcgagccccggcagatcccgaagatccagaacttcactcggacccagaagtcttgtatcttctgcggcttcgcccttctgagttacgacgagaaggagggcctggagactacaacttacatcacaagccaggaggtgcagaacagcatcctgtcatctaattactttgacttcgacaacctgcacgttcattatctactgctaacaactaacggtaccgtcatggaaatcgcaggactgtatgaggagcgggccggcagcgggagcgcgggctctgccagcggtggtgccagcagcggcagtggaggtgcctccatgcagatttacgaaggaaagctgaccgccgaaggcctgaggttcggcatcgtggcttccagattcaaccacgcactggtggacaggctggtggagggcgccatcgacgcaatcgtgcgccacggcggcagggaggaggacatcaccctcgtaagagtccctggtagctgggaaattcccgtggccgcgggcgagctggctaggaaggaggatatcgacgccgtcatcgccatcggcgttctcatccggggtgccacaccacatttcgactacatcgcaagcgaggtttcaaagggactcgccgacttaagtctggagctgaggaagcccatcacctttggcgtgatcaccgccgacaccctggagcaggccatcgagcgcgccggcaccaagcacgggaacaagggctgggaggcagccttatcagccatcgaaatggcgaatctgttcaagagcctgcggtgataataggctggagcctcggtggccatgcttcttgccccttgggcctccccccagcccctcctccccttcctgcacccgtacccccgtggtctttgaataaagtctgagtgggcggc

SEQ ID NO: 195 - RNA, 2950 nt, synthetic construct
  source 1..2950: mol_type = other RNA
gggaaataagagagaaaagaagagtaagaagaaatataagagccaccatggactctaagggcagcagccagaagggcagtaggctcctacttctcctcgtggtgagcaacttattgcttccccagggagtggtgcagactccggaacagcccgctccgcccgctaccaccgtccagcctaccgccacgcgccagcagacttccttcccgtttcgcgtctgcgagctctcctctcacggcgatctcttccgcttctcctccgatattcagtgcccctccttcggcacccgcgagaaccataccgagggcctcctcatggtctttaaggacaacatcatcccttactccttcaaggtacgctcctacacaaagatagtcaccaacatcctcatctataatggatggtacgctgactctgttaccaatcgccacgaggagaagttctccgttgattcctacgagactgaccagatggataccatctaccagtgctacaatgccgtcaagatgaccaaggacggcctgacccgcgtctatgtcgaccgcgacggcgttaacataactgtcaacctcaagcctacaggcggcctggccaatggcgtcagacgctacgcctcccagaccgagctctacgacgctcctggctggctgatctggacctaccggacccgcaccactgtcaattgcctcattaccgacatgatggccaagtccaatagcccattcgatttcttcgtaaccaccaccggccagaccgtcgagatgtcgccattctatgacggcaagaataaggaaactttccacgagagagccgatagcttccacgtccgcacgaactacaagattgtcgactacgacaatcgcggcaccaatccacagggcgagcgccgcgccttcctggacaagggcacctacaccctctcctggaaactcgagaacagaaccgcctactgtcccctccagcactggcagacttttgattccaccatcgccactgagactggcaagagtatccatttcgtcaccgacgaaggaacatcttcattcgtcacaaataccactgtgggcattgagctccccgacgccttcaagtgcatcgaggaacaggtcaacaagacaatgcacgagaagtacgaggccgtgcaggaccggtataccaagggccaggaggccattacatacttcatcaccagcggtggattgcttctcgcctggcttccactcacacctcgcagtctggccacagttaagaacctcactgagttgaccactccaacatcgtcaccaccttcttcacctagccctcctgctccttccgccgcccgcggctccactcctgccgctgtcttaaggggctcgggcagcgccggcagcgcatcgggaggggcgagttcagggtcaggcggtgcctccgcaggcaatgctaccacaccagtgccaccaaccgcccctggcaagtccttaggtaccctgaacaatccagcgacagttcagatccagttcgcttacgattcactcaggcgccagatcaaccgcatgcttggtgaccttgcccgggcctggtgcctcgaacagaagcgccagaacatggtacttagggaacttaccaagattaatcctactacagtcatgtcctctatctacggcaaggccgttgctgccaagcgcctgggcgacgtcatcagcgtgtcgcagtgcgtcccagtcaaccaggctaccgttaccctcaggaagtccatgagagtgccaggctccgaaacaatgtgctattcccgccctctggtttcattttccttcatcaacgacacaaagacctacgagggccagttaggaactgacaatgagatattcttgactaagaagatgactgaggtatgccaggcaacttctcaatactacttccagtctggaaatgaaatccacgtatataacgactatcatcactttaagactatcgaactcgacggaattgctacactccaaacattcatctcactgaatacctcccttatcgagaacatcgacttcgcatccctcgagctgtatagcagagacgagcagagagccagcaacgtgttcgacctggagggtatctttcgcgagtacaacttccaggcacagaatatagccggcctccgtaaggacctggataacgccgtgtccaatggccgcaaccagttcgtcgacggtcttggcgaactcatggactctctgggatccgtggggcaatccatcacaaacttagtctctactgtcggtggactattcagcagcctggttagcggatttatctctttctttaagaacccaggcagtggttctgccggctcagccagcggcggcgctagtagcggcagcggaggcgcctcaatgcagatctatgagggtaagttgacagccgaaggcttgaggtttggcatcgtagccagccggttcaaccacgccctggtggacaggctggttgagggtgccattgacgccattgtgaggcacggcggcagggaggaggacattaccctggtcagagtccccggatcatgggagatccccgtggccgccggggagctggccaggaaggaagacatcgacgctgtgatcgccatcggcgtgctgatccgaggagccacaccccatttcgactacatcgccagtgaggttagcaaaggcctagcggacctgagcctggagctgcgcaagcccatcaccttcggcgtcattactgccgacaccttggaacaggccatcgaaagggccggtacaaagcacggcaacaagggctgggaggcagcccttagcgccatcgagatggcaaacctgtttaagtccttgaggtgataataggctggagcctcggtggccatgcttcttgccccttgggcctccccccagcccctcctccccttcctgcacccgtacccccgtggtctttgaataaagtctgagtgggcggc

SEQ ID NO: 196 - RNA, 2761 nt, synthetic construct; identical to SEQ ID NO: 162
  source 1..2761: mol_type = other RNA

SEQ ID NO: 197 - RNA, 2761 nt, synthetic construct; identical to SEQ ID NO: 167
  source 1..2761: mol_type = other RNA

SEQ ID NO: 198 - RNA, 2761 nt, synthetic construct; identical to SEQ ID NO: 172
  source 1..2761: mol_type = other RNA

SEQ ID NO: 199 - RNA, 2761 nt, synthetic construct; identical to SEQ ID NO: 174
  source 1..2761: mol_type = other RNA

SEQ ID NO: 200 - RNA, 2761 nt, synthetic construct; identical to SEQ ID NO: 175
  source 1..2761: mol_type = other RNA

SEQ ID NO: 201 - RNA, 2294 nt, synthetic construct
  source 1..2294: mol_type = other RNA
gggaaataagagagaaaagaagagtaagaagaaatataagaccccggcgccgccaccatgcagttgttgtgcgtgttctgcctcgtgttactctgggaggtgggcgccgccagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctgtgataataggctggagcctcggtggcctagcttcttgccccttgggcctccccccagcccctcctccccttcctgcacccgtacccccgtggtctttgaataaagtctgagtgggcggc

SEQ ID NO: 202 - RNA, 587 nt, synthetic construct
  source 1..587: mol_type = other RNA
gggaaataagagagaaaagaagagtaagaagaaatataagaccccggcgccgccaccatgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggctgataataggctggagcctcggtggcctagcttcttgccccttgggcctccccccagcccctcctccccttcctgcacccgtacccccgtggtctttgaataaagtctgagtgggcggc

SEQ ID NO: 203 - RNA, 845 nt, synthetic construct
  source 1..845: mol_type = other RNA
gggaaataagagagaaaagaagagtaagaagaaatataagaccccggcgccgccaccatggtgagcttcaagcaggtgagagtgcccctgttcaccgccatcgccctggtgatcgtgctgctgctggcctacttcctgccacccagagtgagaggcggcggcagagtggccgccgccgccatcacctgggtgcccaagcccaacgtggaggtgtggcccgtggacccgccacctcccgtgaacttcaacaagaccgccgagcaggagtacggcgacaaggaggtgaagctgccccactggacgccgaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccaccccagacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagctgataataggctggagcctcggtggcctagcttcttgccccttgggcctccccccagcccctcctccccttcctgcacccgtacccccgtggtctttgaataaagtctgagtgggcggc

SEQ ID NO: 204 - RNA, 2771 nt, synthetic construct
  source 1..2771: mol_type = other RNA
gggaaataagagagaaaagaagagtaagaagaaatataagaccccggcgccgccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggcctagcttcttgccccttgggcctccccccagcccctcctccccttcctgcacccgtacccccgtggtctttgaataaagtctgagtgggcggc

SEQ ID NO: 205 - RNA, 2057 nt, synthetic construct
  source 1..2057: mol_type = other RNA
gggaaataagagagaaaagaagagtaagaagaaatataagaccccggcgccgccaccatggacagcaagggcagcagccagaagggatcgaggctattattgttacttgtggtgagcaacttgctcctgcctcagggcgtggtgggcgaggccgccttgttggtgtgccagtacaccatccagagcctcatccacctcaccggcgaggaccccggcttcttcaacgtggagatccccgagttccccttctaccccacctgcaacgtgtgcaccgccgacgtgaacgtgaccatcaacttcgacgtgggcggcaagaagcaccagctggacctggacttcggccagctgaccccgcacaccaaggccgtgtaccagcccaggggcgccttcggcggcagcgagaacgccaccaacctgttcctgctggagctgctgggcgccggcgagctggccctgaccatgaggtccaagaagctgcccatcaatgtgaccacaggagaggagcagcaggtgagcctggagagcgtggacgtgtacttccaggacgtgttcggcaccatgtggtgccaccacgccgagatgcagaacccagtttacttgattcctgagacagtgccctacatcaagtgggacaactgcaacagcaccaacatcaccgccgtggtgagggcccagggcctggacgtgaccctgcccctgagcctgcccaccagcgcccaggatagcaacttcagcgtgaagaccgaaatgctgggcaacgagatcgacatcgagtgcatcatggaggacggcgagatcagccaggtgctgcccggcgacaacaagttcaacattacctgcagcggctacgagagccacgtgcccagcggcggcatcctgacctcaacttcacccgtggccactccgatccccggcaccggctacgcctacagcctgcgtctgacacctaggcccgtgagcaggttcctgggcaataacagcatcctgtacgtgttctacagcggcaacggccccaaggcttccggcggcgactactgtattcaatcaaacatcgtgttcagcgacgagataccagccagccaggacatgcctactaacaccaccgacatcacctatgtgggagacaatgctacttacagcgtgcccatggtgaccagcgaggacgccaacagcccaaatgtcaccgtgaccgccttctgggcctggcccaacaacactgagacagacttcaagtgcaagtggaccctcacatccggcacccctagcggatgcgagaacatcagcggagcgttcgccagcaacaggaccttcgatataacagtgagcggcctgggcaccgctcctaagaccctgatcatcaccaggacggccacaaatgcaactactactacacacaaggtgatcttcagcaaggctccggaatcacaagtgaggcagcagttctcaaaggatatcgagaagcttctgaacgagcaagttaacaaggaaatgcagagcagtaatctctacatgagcatgagcagctggtgctacacccactccctggacggagcaggcctcttcctgttcgaccacgcagccgaggagtacgagcacgctaagaagttgatcattttcttgaacgagaacaacgtgcccgtgcagctaacgtcaatcagcgcacctgagcacaagttcgagggcctgacccagatcttccagaaggcctacgaacacgaacagcacatctccgagagcatcaacaatattgtggatcacgctatcaagtccaaggaccacgctaccttcaacttcctgcagtggtacgtggccgagcaacatgaggaggaggtgctgttcaaggacatcctggacaagatcgagctgatcggtaatgagaatcacggcctgtacctggccgaccagtacgtgaagggcatcgccaagagccggaagtcatgataataggctggagcctcggtggcctagcttcttgccccttgggcctccccccagcccctcctccccttcctgcacccgtacccccgtggtctttgaataaagtctgagtgggcggc

SEQ ID NO: 206 - RNA, 2060 nt, synthetic construct
  source 1..2060: mol_type = other RNA
gggaaataagagagaaaagaagagtaagaagaaatataagaccccggcgccgccaccatggacagcaagggcagcagccagaagggctcccggttgttgctcttgctcgtggtgagcaacttgttgttgcctcagggcgtggtgggcgaggccgccttgttggtgtgccagtacaccatccagagcctcatccacctcaccggcgaggaccctggcttcttcaacgtggagatccctgagttccctttctaccctacctgcaacgtgtgcaccgccgacgtgaacgtgaccatcaacttcgacgtgggcggcaagaagcaccagctggacctggacttcggccagctgacccctcacaccaaggccgtgtaccagcctagaggcgccttcggcggcagcgagaacgccaccaacctgttcctgctggagctgctgggcgccggcgagctggccctgaccatgagaagtaagaagctgcctatcaacgtcactactggcgaggagcagcaggtgagcctggagagcgtggacgtgtacttccaggacgtgttcggcaccatgtggtgccaccacgccgagatgcagaaccctgtgtacctgatcccagaaaccgtgccttacatcaagtgggacaactgcaacagcaccaacatcaccgctgtggtgagagcccagggcctggacgtgaccctgcctctgagcctgcctaccagcgcccaagacagtaacttcagcgtgaagacggagatgctgggcaacgagatcgacatcgagtgcatcatggaggacggcgagatcagccaggtgctgcctggcgacaacaagttcaacataacatgcagcggctacgagagccacgtgcctagcggcggcatcctgacctccaccagccctgtggccacccctatccctggcaccggctacgcctacagcctgagactcacgccaagacctgtgagcagattcttaggaaacaacagcatcctgtacgtgttctacagcggcaacggccctaaggcctccggcggtgactactgtatccagtctaacatcgtgttcagcgacgaaatcccagccagccaggacatgcctacaaacaccaccgacatcacttatgtcggcgacaatgcgacatacagcgtgcctatggtgaccagcgaggacgccaacagccctaacgtaaccgtgaccgccttctgggcctggcctaacaacaccgaaaccgacttcaagtgcaagtggactttaactagtggcaccccttctggctgcgagaacatcagcggcgcattcgccagcaacagaaccttcgatatcaccgtgagcggcctgggcaccgcccctaagaccctgatcatcaccagaaccgcaactaatgcaactacaacaacccacaaggtgatcttcagcaaggcccctggctccggctctgccggcagtggcagcgcaggcagtggatccgccggttcaggttccgccatgcagatctacgagggcaagctgaccgccgagggcctgagattcggcatcgtggccagcaggttcaaccacgccctggtggacagactggtggagggcgccatcgacgccatcgtgagacacggaggaagagaggaggatataacactggtcagagtgcctggatcatgggaaatccctgtcgccgctggtgagctggcaagaaaggaggacatcgatgcggtgatcgccatcggcgtgctgatccgcggtgcaactccacacttcgactacatcgcctctgaagtgtcaaagggccttgcggacctgtccctcgaactcagaaagcctatcacattcggagtgattactgcagataccctggagcaggccatcgagagagccggcaccaagcacggcaacaagggctgggaagccgcgctgtctgccatagaaatggcaaaccttttcaagtcccttagatgataataggctggagcctcggtggcctagcttcttgccccttgggcctccccccagcccctcctccccttcctgcacccgtacccccgtggtctttgaataaagtctgagtgggcggc

SEQ ID NO: 207 - RNA, 2761 nt, synthetic construct
  source 1..2761: mol_type = other RNA
gggaaataagagagaaaagaagagtaagaagaaatataagagccaccatggaagccgccctattagtctgccagtacaccattcaaagcctcatccacttgactggggaggatcctgggttctttaacgtggaaatcccggaattccctttctacccaacttgcaacgtgtgcacggctgacgtcaacgtgactatcaacttcgatgtgggagggaagaaacatcaacttgatctggactttggccagcttactccccacaccaaggccgtgtaccagccccgcggcgcattcggtgggagcgagaacgcgacaaatctattcttgctggaactgctgggcgcaggagagctcgccctgacgatgcgatctaagaagcttcccatcaacgttaccactggcgaggagcagcaggtgtccctcgaaagcgtcgacgtctatttccaagacgtcttcggaacaatgtggtgccaccacgctgagatgcagaaccccgtatatcttattcccgaaaccgtgccatacatcaagtgggacaactgcaactcaactaacatcactgcagtagtgcgcgcccagggactggacgtcactttgccactgtcactgcccacatcagcccaagacagcaacttcagcgtcaagacagaaatgctgggcaacgaaatcgacatcgaatgcataatggaggatggggaaatttcccaggtcctccccggtgataacaagtttaatattacttgttctggatatgaatcccatgtgccgtctggtggtatactgacgagtacatcgccagtggctaccccaatccccgggacaggctatgcctacagcctgagactgacgccaagaccagtttccaggtttctggggaataactctatcctgtatgtgttttattctggtaacggacccaaggcatccggcggtgattattgtatccagagcaatatagtgttctctgatgagattccggcctcccaggatatgccaaccaacacgacagatatcacctatgtcggggacaacgccacatatagcgtgccaatggtgaccagcgaagatgccaattctcccaacgtcacagtgacagccttttgggcgtggcctaataacacagaaactgactttaaatgcaaatggactctaacctcaggcaccccatctggctgcgagaacataagcggcgcgtttgcatcgaacagaacttttgatattacagtatctgggttgggcactgcgcccaagactctaataattacacggacggccactaacgccaccacgacgactcacaaggtgatcttcagcaaagctcccgaatctactactacctcgccgacactgaatacgacaggattcgccgacccaaatactacgaccggccttcccagctcgacacatgtgcctacaaacttaacggcgcccgccagtaccggccccaccgtgagcaccgctgacgtcacctctcctacccctgccgggactacaagtggtgcttccccagttactcccagccctagcccttgggacaacggcactgagagcaaagctccggacatgaccagcagtaccagtccagtgacaactccgacacccaatgcgacgtccccaactcccgccgtgaccacgccaacaccaaatgccacgtcacctactccagcggtcaccaccccaacgccaaacgctactagtcccactctaggcaagacatctcccacctccgctgtcaccacccctacacccaatgctaccggtcccacggtgggtgagactagcccacaggccaacgcaactaatcatacattgggtggcacaagcccaacgcccgtcgtcactagccagcctaagaacgccacaagcgctgtaactaccggccagcacaatataacaagttccagtactagcagtatgtctctgagacccagctcaaatcctgaaaccctctcgccctctacgtcggataactccacgtcccacatgcctctactcacaagcgcgcaccctaccggaggagagaatatcactcaggttaccccagcttctatctcgactcaccatgtctctacttcttccccagcgccacggcccggcactaccagccaggctagcgggccaggtaattcctccactagcactaaacctggcgaagtcaacgttaccaagggaacacctccccagaatgcaacctcgccccaagctccctctggacagaagacagccgtccctaccgtcacaagcaccggtgggaaggcgaattctacgacaggcgggaagcacactaccggacacggagcaaggacatccactgagcccacgaccgactacggtggcgactccaccacgcccagacccaggtacaatgcgactacctacctgccgccttccacttccagcaaactgaggcctcgttggactttcacttctccaccagtcaccaccgctcaggctaccgtgcctgtcccgccgacctctcagccccggttttcaaatttgagtatgctcgtgctgcagtgggccagcctggcagtgcttaccctcctgctcttgctggttatggctgactgcgccttccgacgtaacttatccaccagtcatacttacactacacctccttatgacgacgcggagacctacgtgtgataataggctggagcctcggtggccatgcttcttgccccttgggcctccccccagcccctcctccccttcctgcacccgtacccccgtggtctttgaataaagtctgagtgggcggc

SEQ ID NO: 208 - RNA, 2761 nt, synthetic construct
  source 1..2761: mol_type = other RNA
gggaaataagagagaaaagaagagtaagaagaaatataagagccaccatggaagccgccttactggtctgccagtacactattcagtccctcatccatctcacaggcgaggacccggggttcttcaacgtcgaaatccccgaattcccattctatcccacctgtaacgtctgcacagccgacgttaacgttacaatcaatttcgacgtgggcgggaagaaacaccagctggacctcgactttgggcagctgacgccccacactaaagccgtttaccagcccaggggtgcattcggtggatctgagaatgctacaaacctctttctgcttgagctgcttggtgctggagagctggccctgacaatgcgcagcaagaagttgccaatcaatgtgaccacaggtgaagaacagcaggtgtccctagaatccgtggatgtgtacttccaggacgttttcggcacaatgtggtgccatcacgcagagatgcagaatccggtgtaccttatccccgaaacagtgccgtacattaaatgggataattgtaactccacaaacatcacggcagtggtgcgcgctcaggggctggacgtgacactcccgctttccttaccaacaagtgctcaagattccaacttttctgtgaagaccgagatgctgggcaacgagattgatattgaatgcatcatggaggatggcgagattagccaggtgttgcccggcgacaataaatttaatattacctgttccggctatgagtcacacgtgccaagtggaggcattctcacgagcacctctcccgttgcaacacccattcctgggacagggtacgcatatagtctcaggctgacaccgagaccggtatcgcgcttcttaggtaacaattccatcttgtatgtgttttacagcggaaacgggccaaaggcctcaggaggagactactgcatccagagcaatatagtctttagcgacgagatccctgcctcacaagacatgcccaccaacaccaccgacatcacgtacgtcggtgataatgccacttacagcgtgcctatggtgacctctgaagacgcaaattcacccaatgtcacggttaccgcattctgggcttggccaaataacactgaaaccgacttcaagtgtaaatggaccctaaccagcggcaccccatcagggtgcgagaatatcagcggggccttcgccagcaaccggacattcgacatcaccgtgtcaggactagggactgctccaaagacattaattattacacggacagcaactaacgctacgactacgactcacaaagtgatatttagcaaagcgcccgaatcgactactacgagcccgaccctaaacaccaccggtttcgccgatcccaacacaactactggtctcccatcttcgacccatgttccaacgaatctgacagctccagcaagtacggggcccactgtgagtacagccgacgttactagcccaactcccgcgggtacgactagtggcgcgtctccagtgaccccttcgccttctccgtgggataatggaaccgagtcaaaggctcctgatatgaccagctccaccagtcccgttacaacgccaacgcccaatgcaacgagtccaactccggcggttaccactccgacaccaaacgccacctcgcccacgcctgcagttactacgcccacccctaatgctacgtcacccactctgggaaagacctcaccaacctctgccgtgactactcctacacctaatgccacaggacccacagttggggaaaccagcccacaggctaacgcaactaatcatacgcttggcgggacctctcccacgcccgtggtcacttcacagcctaagaatgccacaagcgctgtgactacaggccagcataatataaccagcagttcaaccagtagcatgtccctgcgtccttcatccaatcctgagactctgagccccagcactagtgacaactcaacttctcacatgccgttgctgacctccgcgcatcccacgggcggtgagaatatcacacaggtgacacccgcgtctattagcacacatcacgtgagtacgtcctcgcccgcaccccgcccaggcacaacctcacaagcatcaggtccggggaatagcagcacaagtaccaagcctggcgaagtgaatgtaaccaagggcacgccaccgcagaacgccacatcccctcaagccccatccggccagaagaccgctgtacccaccgtgacatcaactggggggaaagcgaactcaactaccggtgggaagcatacaacagggcacggtgccagaacttctacagagccaaccacggactacggaggggacagcacaacgcccagaccgcggtacaatgcgaccacatacctgcctccctcaacttcgagtaagctgcgacccagatggacctttacttctcctcctgtcaccaccgctcaagccaccgtcccggttccacctacttcccagcctagattctccaatctgagcatgcttgtcctacagtgggcatcactcgcggtactaaccttgctgctactgttggtgatggcggactgcgcgtttcgccgcaacttatccacctcccacacgtacacaacacctccctatgatgacgctgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 209 - RNA, 2737 nt, synthetic construct
  source 1..2737: mol_type = other RNA
gggaaataagagagaaaagaagagtaagaagaaatataagagccaccatgacccggcggcgggtgttaagcgtggtggtgctcttagccgccttagcctgccggcttggcgcccagactccggagcagcccgctcctcccgccaccaccgtccagcccaccgccacccgccagcagacctccttccccttccgcgtctgcgagctctcctcccacggcgacctcttccgcttctcctccgacatccagtgcccctccttcggcacccgcgagaaccacaccgagggcctcctcatggtcttcaaggacaacatcatcccctactccttcaaggtccgctcctacaccaagatcgtcaccaacatcctcatctacaacggctggtacgccgactctgtgactaatcgccacgaggagaagttctccgtcgactcctacgagactgaccagatggacaccatctaccagtgctacaacgccgtcaagatgaccaaggacggcctcacccgcgtctacgtcgaccgcgacggcgtcaacatcaccgtcaacctcaagcctacaggcggcctcgccaacggcgtccgccgctacgcctcccagaccgagctctacgacgcccctggctggctcatctggacctaccgcaccaggaccactgtcaattgcctcatcaccgacatgatggccaagtccaactcccctttcgacttcttcgtgaccaccacaggccagaccgtcgagatgtcgccattctatgacggcaagaacaaggagactttccacgagagagcggactctttccatgtccgcaccaactacaagattgtcgactacgacaaccgcggcaccaatccacagggcgagcgccgcgccttcctcgacaagggcacctacaccctctcctggaagctcgagaaccgcaccgcctactgtcctctccagcactggcagaccttcgattccaccatcgccaccgagactggcaagtccatccatttcgtcaccgacgaaggaacatcttcattcgtcacaaatacaaccgtgggcatcgagctccccgacgccttcaagtgcatcgaggagcaggtcaacaagaccatgcacgagaagtacgaggccgtccaggaccggtatacaaagggccaggaggccatcacctacttcatcaccagcggaggtctgcttctcgcctggcttccactcacacctcgctccctggccacagtgaagaacctcactgagttgaccaccccaacaagttccccacctagtagtcctagccctcctgctccttccgccgcccgcggctccacgcctgccgccgtcctgcggagacggagaagggacgccggcaacgctaccactcctgtgccgccgacagcccctggaaagtccctaggtaccctcaacaacccagcgacagttcagatccagttcgcctacgactctctcaggcgccagatcaaccgcatgcttggtgaccttgcccgcgcctggtgcctcgagcagaagcgccagaacatggtgcttcgtgaactaacaaagattaatcctactacagtgatgtcctccatctacggcaaggccgtcgccgccaagcgcctcggcgacgtcatctccgtctcccagtgcgtcccagtgaaccaggctaccgtgaccctcaggaagtccatgagagtgccaggctccgagacaatgtgctactcccgcccactcgtctccttctccttcatcaacgacacaaagacctacgagggccagttaggcactgacaacgagatcttcttgactaagaagatgactgaggtatgccaggcaacttctcaatactacttccagtctggaaatgagatccacgtatataacgactaccaccacttcaagactattgaactcgacggaattgccaccctccaaacattcatctcactgaatacctccctcatcgagaacatcgacttcgcctccctcgagctgtatagcagagacgagcagcgcgcctccaacgtcttcgacctcgagggcatcttccgcgagtacaacttccaggcacagaacatagccggcctccgtaaggatctggacaatgccgtgtccaacggccgcaaccagttcgtcgacggtttgggtgaactcatggactctctgggctccgtcggccagtccataactaacttagtctctacggtgggaggcctattcagcagcctggtgagcggcttcatctctttcttcaagaaccccttcggcggcatgctcatcctcgtcctcgtcgccggcgtcgtcatactggtgatctcactcacaaggaggacgcgccaaatgtcccagcagccagtgcagatgctctacccaggcatagacgagctcgctcagcagcacgcgtcgggtgagggaccaggcatcaatcctatctccaagactgagctgcaagccattatgctcgccctccacgagcagaaccaggaacagaagcgggccgcccagcgagctgccggcccctccgtcgccagtagggcactacaagccgcccgggaccgcttcccaggcctgagaaggaggagataccacgacccagagacagccgctgccctccttggcgaagcagaaaccgagttctgataataggctggagcctcggtggccatgcttcttgccccttgggcctccccccagcccctcctccccttcctgcacccgtacccccgtggtctttgaataaagtctgagtgggcggc

SEQ ID NO: 210 - RNA, 2761 nt, synthetic construct
  source 1..2761: mol_type = other RNA
gggaaataagagagaaaagaagagtaagaagaaatataagagccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattacggctgtggtgagggcccagggcttggacgtgactctgcccttatctctacctacttctgcccaagactccaatttcgcggtcaagaccgagatgctcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttgcggtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccgcgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggccatgcttcttgccccttgggcctccccccagcccctcctccccttcctgcacccgtacccccgtggtctttgaataaagtctgagtgggcggc

SEQ ID NO: 211 - RNA, 2595 nt, synthetic construct
  source 1..2595: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctgcgaatctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcagcgaatattacggctgtggtgagggcccagggcttggacgtgactctgcccttatctctacctacttctgcccaagactccaatttcgcggtcaagaccgagatgctcgggaatgagatcgatatcgagtgcatcatggaggacggtgagataagccaggttctgcccggcgacaacaagttcaatatcgcgtgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttgcggtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccgcgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccaaggctcctgatatgacttcctctaccagccccgtgactacacccactcccaacgcaactagcccaaccccagctgtgacgacgcccaccccgaacgcgacatctcccacacctgctgtgacaaccccaacccctaacgccactagccctaccctaggtaagaccagtccgactagcgccgttacaacccctacccctaacgcaaccggcccgaccgtgggcgagacttccccgcaagccaatgcgacaaatcacacattgggcgggacctctcctacaccagtcgttacatctcagcctaagaacgctacctccgctgtcactaccggacagcacaacatcaccagctcaagcacgagttccatgagcttgcggccgagctcaaatcccgagaccctatcaccatccacatcagacaacagtacttcacacatgccactcttgacgagcgctcaccccaccggcggcgagaacatcacccaggtgactccggcgtctatttccacccaccacgtcagcacgtcgtctcccgcaccgagaccagggacgacttctcaggccagtgggcctggcaactcctctacaagcacaaagccaggcgaagttaacgtgacaaagggaacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 212 - RNA, 3389 nt, synthetic construct
  source 1..3389: mol_type = other RNA
gggaaataagagagaaaagaagagtaagaagaaatataagaccccggcgccgccaccatggtgagcttcaagcaggtgagagtgcccctgttcaccgccatcgccctggtgatcgtgctgctgctggcctacttcctgccacccagagtgagaggcggcggcagagtggccgccgccgccatcacctgggtgcccaagcccaacgtggaggtgtggcccgtggacccgccacctcccgtgaacttcaacaagaccgccgagcaggagtacggcgacaaggaggtgaagctgccccactggacgccgaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccaccccagacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagcggcactagcggctctagcggatcaggcagcggagggagtggaggctcaggagggaccagcggcagcagtggctccggaagcggaggcagcccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggcggaggaggtggctccggtggaggtggcagcggaggaggaggcagtggtggcggaggcagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctgtgataataggctggagcctcggtggcctagcttcttgccccttgggcctccccccagcccctcctccccttcctgcacccgtacccccgtggtctttgaataaagtctgagtgggcggc

SEQ ID NO: 213 - RNA, 3213 nt, synthetic construct
  source 1..3213: mol_type = other RNA
atggtgagcttcaagcaggtgagagtgcccctgttcaccgccatcgccctggtgatcgtgctgctgctggcctacttcctgccacccagagtgagaggcggcggcagagtggccgccgccgccatcacctgggtgcccaagcccaacgtggaggtgtggcccgtggacccgccacctcccgtgaacttcaacaagaccgccgagcaggagtacggcgacaaggaggtgaagctgccccactggacgccgaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccaccccagacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagcggcactagcggctctagcggatcaggcagcggagggagtggaggctcaggagggaccagcggcagcagtggctccggaagcggaggcagcccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggcggaggaggtggctccggtggaggtggcagcggaggaggaggcagtggtggcggaggcagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctg

SEQ ID NO: 214 - protein, 1071 aa, synthetic construct
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNSGTSGSSGSGSGGSGGSGGTSGSSGSGSGGSPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGGGGGGSGGGGSGGGGSGGGGSLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 215 - RNA, 3389 nt, synthetic construct
  source 1..3389: mol_type = other RNA
gggaaataagagagaaaagaagagtaagaagaaatataagaccccggcgccgccaccatggtgagcttcaagcaggtgagagtgcccctgttcaccgccatcgccctggtgatcgtgctgctgctggcctacttcctgccacccagagtgagaggcggcggcagagtggccgccgccgccatcacctgggtgcccaagcccaacgtggaggtgtggcccgtggacccgccacctcccgtgaacttcaacaagaccgccgagcaggagtacggcgacaaggaggtgaagctgccccactggacgccgaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccaccccagacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagcggaggaggaggatctggaggcggcggaagtggtggaggaggaagcggaggaggtggcagcggaggtggtggaagtggcggcggaggaagcccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggcggaggaggtggctccggtggaggtggcagcggaggaggaggcagtggtggcggaggcagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctgtgataataggctggagcctcggtggcctagcttcttgccccttgggcctccccccagcccctcctccccttcctgcacccgtacccccgtggtctttgaataaagtctgagtgggcggc

SEQ ID NO: 216 - RNA, 3213 nt, synthetic construct
  source 1..3213: mol_type = other RNA
atggtgagcttcaagcaggtgagagtgcccctgttcaccgccatcgccctggtgatcgtgctgctgctggcctacttcctgccacccagagtgagaggcggcggcagagtggccgccgccgccatcacctgggtgcccaagcccaacgtggaggtgtggcccgtggacccgccacctcccgtgaacttcaacaagaccgccgagcaggagtacggcgacaaggaggtgaagctgccccactggacgccgaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccaccccagacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagcggaggaggaggatctggaggcggcggaagtggtggaggaggaagcggaggaggtggcagcggaggtggtggaagtggcggcggaggaagcccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggcggaggaggtggctccggtggaggtggcagcggaggaggaggcagtggtggcggaggcagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctg

SEQ ID NO: 217 - protein, 1071 aa, synthetic construct
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNSGGGGSGGGGSGGGGSGGGGSGGGGSGGGGSPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGGGGGGSGGGGSGGGGSGGGGSLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 218 - RNA, 2693 nt, synthetic construct
  source 1..2693: mol_type = other RNA
gggaaataagagagaaaagaagagtaagaagaaatataagaccccggcgccgccaccatgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggcggaggaggtggcagcggaggaggagggtctggaggtggcggcagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctgtgataataggctggagcctcggtggcctagcttcttgccccttgggcctccccccagcccctcctccccttcctgcacccgtacccccgtggtctttgaataaagtctgagtgggcggc

SEQ ID NO: 219 - RNA, 2517 nt, synthetic construct
  source 1..2517: mol_type = other RNA
atgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggcggaggaggtggcagcggaggaggagggtctggaggtggcggcagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctg

SEQ ID NO: 220 - protein, 839 aa, synthetic construct
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGGGGGGSGGGGSGGGGSLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 221 - RNA, 2708 nt, synthetic construct
  source 1..2708: mol_type = other RNA
gggaaataagagagaaaagaagagtaagaagaaatataagaccccggcgccgccaccatgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggcggaggaggtggctccggtggaggtggcagcggaggaggaggcagtggtggcggaggcagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctgtgataataggctggagcctcggtggcctagcttcttgccccttgggcctccccccagcccctcctccccttcctgcacccgtacccccgtggtctttgaataaagtctgagtgggcggc

SEQ ID NO: 222 - RNA, 2532 nt, synthetic construct
  source 1..2532: mol_type = other RNA
atgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggcggaggaggtggctccggtggaggtggcagcggaggaggaggcagtggtggcggaggcagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctg

SEQ ID NO: 223 - protein, 844 aa, synthetic construct
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGGGGGGSGGGGSGGGGSGGGGSLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 224 - protein, 15 aa, synthetic construct
GGGGSGGGGSGGGGS

SEQ ID NO: 225 - protein, 20 aa, synthetic construct
GGGGSGGGGSGGGGSGGGGS

SEQ ID NO: 226 - protein, 4 aa, synthetic construct
GGGS

SEQ ID NO: 227 - protein, 5 aa, synthetic construct
GGGGS

SEQ ID NO: 228 - RNA, 2294 nt, synthetic construct
  source 1..2294: mol_type = other RNA
gggaaataagagagaaaagaagagtaagaagaaatataagaccccggcgccgccaccatgcagttgttgtgcgtgttctgcctcgtgttactctgggaggtgggcgccgccagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctgtgataataggctggagcctcggtggccatgcttcttgccccttgggcctccccccagcccctcctccccttcctgcacccgtacccccgtggtctttgaataaagtctgagtgggcggc

SEQ ID NO: 229 - RNA, 587 nt, synthetic construct
  source 1..587: mol_type = other RNA
gggaaataagagagaaaagaagagtaagaagaaatataagaccccggcgccgccaccatgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggctgataataggctggagcctcggtggccatgcttcttgccccttgggcctccccccagcccctcctccccttcctgcacccgtacccccgtggtctttgaataaagtctgagtgggcggc

(19) United States
(12) Patent Application Publication
(10) Pub. No.: US 2025/0312442 A1
(43) Pub. Date: Oct. 9, 2025

(54) EPSTEIN-BARR VIRUS VACCINES

(71) Applicant: ModernaTX, Inc., Cambridge, MA (US)

(72) Inventors: Giuseppe Ciaramella, Sudbury, MA (US); Shinu John, Somerville, MA (US); Elisabeth Narayanan, Cambridge, MA (US); Brooke Bollman, Brookline, MA (US)

(21) Appl. No.: 19/084,092

(22) Filed: Mar. 19, 2025

Related U.S. Application Data
(63) Continuation of application No. 16/765,285, filed on May 19, 2020, now abandoned.
(60) Provisional application No. 62/589,170, filed on Nov. 21, 2017.

(51) Int. Cl. A61K 39/245; A61K 9/00; A61K 9/51; A61K 39/00; C12N 7/00

(52) U.S. Cl. CPC A61K 39/245; A61K 9/0019; A61K 9/5123; C12N 7/00; A61K 2039/53; A61K 2039/545; A61K 2039/57; C12N 2710/16234

(57) ABSTRACT

The disclosure relates to EBV ribonucleic acid vaccines as well as methods of using the vaccines and compositions comprising the vaccines.

### BACKGROUND

Epstein-Barr virus (EBV), also referred to as human herpesvirus 4, is one of the most common human viruses worldwide. Ninety five percent of adults are infected with this virus. EBV spreads most commonly through bodily fluids, primarily saliva, and is the primary cause of infectious mononucleosis (“mono”) and other illnesses. Seventy five percent of college students (18-22 years) with primary EBV infection will develop mono. Symptoms of EBV can include fatigue, fever, inflamed throat, swollen lymph nodes in the neck, enlarged spleen, swollen liver, and rash. While many people are infected with EBV in childhood, childhood symptoms are not distinguished from other mild, brief childhood illnesses. Typically, only teenagers and adults exhibit symptoms characteristic of EBV infection, and although recover is about two to four weeks, some people may feel fatigued for several weeks or even months. Following an EBV infection, the virus becomes latent and, in some cases, may be reactivated. Those with weakened immune systems are more likely to develop symptoms if EBV is reactivated. Currently, there is no vaccine to prevent primary infection or disease.

### SUMMARY

Provided herein, in some embodiments, are Epstein-Barr virus (EBV) ribonucleic acid (RNA) (e.g., mRNA) vaccines (e.g., combination vaccines) that elicit potent neutralizing antibodies and robust T cell responses, inhibit the production of viral immunomodulatory factors, and/or prevent viral latency. In some aspects, the EBV vaccines include a RNA having an open reading frame (ORF) encoding an EBV antigen, wherein intramuscular (IM) administration of a therapeutically effective amount of the vaccine to a subject induces in the subject a neutralizing antibody titer and/or a T cell immune response (e.g., a CD4+ and/or a CD8+ T cell immune response).

In some embodiments, the neutralizing antibody titer is at least 100 (e.g., at least 500, or at least 1000) NT₅₀ following, for example, a single dose (e.g., a single 10 μg-200 μg dose) of an EBV RNA vaccine. In some embodiments, the neutralizing antibody titer is at least 100 (e.g., at least 500, or at least 1000) NT₅₀ following a booster (second) dose of an EBV RNA vaccine.

In some embodiments, the neutralizing antibody titer is sufficient to reduce EBV infection of B cells by at least 50% (e.g., by at least 60%, 70%, 80% or 90%), or relative to a neutralizing antibody titer of an unvaccinated control subject or relative to a neutralizing antibody titer of a subject vaccinated with a live attenuated EBV vaccine, an inactivated EBV vaccine, or a protein subunit EBV vaccine.

In some embodiments, the neutralizing antibody titer is induced in the subject following fewer than three (one or two) doses of the vaccine.

In some embodiments, a single dose of an EBV RNA vaccine is of 10 μg-100 μg.

In some embodiments, the neutralizing antibody titer and/or a T cell immune response is sufficient to reduce the rate of symptomatic infectious mononucleosis relative to the neutralizing antibody titer of unvaccinated control subjects.

In some embodiments, the neutralizing antibody titer and/or a T cell immune response is sufficient to reduce the rate of asymptomatic EBV infection relative to the neutralizing antibody titer of unvaccinated control subjects.

In some embodiments, the neutralizing antibody titer and/or a T cell immune response is sufficient to prevent EBV latency the subject.

In some embodiments, the neutralizing antibody titer and/or a T cell immune response is sufficient to reduce chronic fatigue in the subject.

In some embodiments, the neutralizing antibody titer is sufficient to block fusion of EBV with epithelial cells and/or B cells of the subject.

In some embodiments, the neutralizing antibody titer is induced within 20 days following a single 10-100 μg of the vaccine. In some embodiments, the neutralizing antibody titer is induced within 40 days following a second 10-100 μg dose of the vaccine.

In some embodiments, the ability of a vaccine of the present disclosure to induce a neutralizing antibody response can be demonstrated by injecting animals, e.g., mice or non-human primates, with the vaccine and testing the ability of serum from the animal to neutralize the ability of the virus to infect human B cells.

In some embodiments, the T cell immune response comprises a CD4⁺ T cell immune response. In some embodiments, the T cell immune response comprises a CD8⁺ T cell immune response. In some embodiments, the T cell immune response comprises both a CD4⁺ T cell immune response and CD8⁺ T cell immune response.

In some embodiments, after vaccination, the EBV antigen is expressed on the surface of cells of the subject. In some embodiments, the ability of the vaccine to be expressed can be tested in a model system, e.g., a mouse or non-human primate model. In some embodiments, the ability of the vaccine to be expressed can be tested in vitro, e.g., using human cells.

In some embodiments, a single 2 μg dose of the vaccine induces in mice NT₅₀ neutralizing antibody titers of about 100. In some embodiments, a 2 μg booster dose of the vaccine induces in mice NT₅₀ neutralizing antibody titers of about 1000.

In some embodiments, the EBV vaccine comprises a RNA having an ORF encoding two EBV antigens, or two RNAs, each having an ORF encoding an EBV antigen.

In some embodiments, the vaccine comprises a RNA having an ORF encoding two (at least two) EBV antigens formulated in a lipid nanoparticle. In some embodiments, the vaccine comprises two (at least two) RNAs, each having an ORF encoding an EBV antigen, wherein the two RNAs are formulated in a single lipid nanoparticle. In some embodiments, the vaccine comprises two RNAs, each having an ORF encoding an EBV antigen, wherein the each RNAs is formulated in a separate lipid nanoparticle.

In some embodiments, the EBV vaccines further include at least one (e.g., 2, 3, 4, 5 or more) additional RNA having an ORF encoding at least one (e.g., 2, 3, 4, 5 or more) additional EBV antigen.

In some embodiments, the lipid nanoparticle comprises a molar ratio of 20-60% ionizable cationic lipid, 5-25% non-cationic lipid, 25-55% sterol, and 0.5-15% PEG-modified lipid

In some embodiments, the EBV antigens are selected from the group consisting of: gp350, gH, gL, gB, gp42, LMP1, LMP2, EBNA1, and EBNA3.

In some embodiments, the EBV antigen is a gH-gL fusion, whereby gH is linked to gL through a linker, such as a GGGGS linker. In some embodiments, the GGGGS linker comprises three GGGGS motifs (SEQ ID NO: 224). In some embodiments, the GGGGS linker comprises four GGGGS motifs (SEQ ID NO: 225)). In some embodiments, the EBV RNA comprises the nucleotide sequence of SEQ ID NO: 218. In some embodiments, the EBV RNA comprises the nucleotide sequence of SEQ ID NO: 221.

In some embodiments, the EBV antigens include EBV gp350 antigen, EBV gH antigen, and EBV gL antigen. In some embodiments, the EBV antigens further include EBV gp42 antigen and/or gB antigen.

In some embodiments, the EBV gp350 antigen is a wild-type EBV gp350 antigen, a mutated EBV gp350 antigen, or a truncated EBV gp350 antigen.

In some embodiments, the EBV antigens are selected from the EBV antigens listed in the Sequence Listing.

In some embodiments, the EBV antigens (one or more EBV antigens) are fused to a scaffold moiety. In some embodiments, the scaffold moiety is selected from the group consisting of: ferritin, encapsulin, lumazine synthase, hepatitis B surface antigen, and hepatitis B core antigen.

In some embodiments, the RNA comprises messenger RNA (mRNA).

In some embodiments, the RNA further comprises a 5′ UTR. In some embodiments, the 5′ UTR comprises a sequence identified by SEQ ID NO: 1 or SEQ ID NO: 104.

In some embodiments, the RNA further comprises a 3′ UTR. In some embodiments, the 3′ UTR comprises a sequence identified by SEQ ID NO: 3 or SEQ ID NO: 106.

In some embodiments, the EBV antigen is fused to a signal peptide. In some embodiments, the signal peptide is a bovine prolactin signal peptide, optionally comprising SEQ ID NO: 115.

In some embodiments, the RNA is unmodified.

In some embodiments, the RNA comprise at least one modified nucleotide. In some embodiments, at least 80% (e.g., 90% or 100%) of the uracil in the ORF comprise 1-methyl-pseudouridine modification.

Also provided herein, in some aspects, are methods that include administering to a subject an EBV vaccine of the present disclosure in a therapeutically effective amount to induce in the subject a neutralizing antibody titer and/or a T cell immune response.

In some embodiments, efficacy of the EBV vaccine is at least 80% (e.g., 85%, 90%, 95%, 98% or 100%) relative to unvaccinated control subjects.

In some embodiments, detectable levels of EBV antigen are produced in the serum of the subject at 1-72 hours post administration of the vaccine.

In some embodiments, a neutralizing antibody titer of at least 100 (e.g., at least 500 or at least 1000) NU/ml is produced in the serum of the subject at 1-72 hours post administration of the vaccine.

In some embodiments, the therapeutically effective amount is a total dose of 20 μg-200 μg (e.g., 50 μg-100 μg).

### BRIEF DESCRIPTION OF THE DRAWINGS

FIG. 1A shows data from a flow cytometry analysis of indel-free codon-optimized glycoprotein 350 (gp350) variant surface expression in HeLa cells using 72A1 antibody. FIGS. 1B and 1C are bar graphs showing percent gp350 variant expression (percent 72A1 positive) on the surface of HeLa cells transfected with 1 μg (FIG. 1B) or 0.5 μg (FIG. 1C) of mRNA.

FIG. 2A shows data from a flow cytometry analysis of expression of gp350 mRNA having one of two different 5′ untranslated region (UTR) sequences (compare UTR A and UTR B). FIG. 2B is a bar graph showing percent gp350 expression on the surface of HeLa cells transfected with 0.5 μg of a gp350 mRNA having one of the two different 5′ UTR sequences.

FIG. 3 is a graph showing the geometric mean (with 95% confidence interval) of neutralizing antibody titers produced in Balb/c mice following intramuscular (IM) vaccination with mRNA encoding EBV gp350 variants formulated in a lipid nanoparticle. A 2 μg dose was administered on Day 1 and then again on Day 21. Mice were bled on Day 21 and Day 43. NT₅₀ titers represent the reciprocal serum dilutions to block 50% viral entry. All gp350 variants exhibited comparable neutralizing titers.

FIG. 4A shows data from a flow cytometry analysis of surface expression of the indicated EBV antigens (EBV gp42) and EBV antigen complexes (EBV gH/gL/gp42 with indicated 5′ UTR, or EBV gH/gp42) in HeLa cells using 2D4 antibody. FIG. 4B is a bar graph showing percent antigen expression (percent 2D4 positive) on the surface of HeLa cells transfected with 0.25 μg of the mRNA 24 hours post transfection.

FIG. 5 is a graph showing the geometric mean (with 95% confidence interval) of neutralizing antibody titers produced in Balb/c mice following IM vaccination with mRNA encoding indicated EBV antigens (EBV gp350) and EBV antigen complexes (EBV gH/gL/gp42 or gH/gL/gp42/gp350) formulated in a lipid nanoparticle. Various indicated doses were administered on Day 1 and then again on Day 29. Mice were bled on Day 28 and Day 57. NT₅₀ titers represent the reciprocal serum dilutions to block 50% viral entry. These data show that gp350 and gH/gL/gp42 elicit comparable B cell neutralizing titers. There is no interference observed by combining all of the mRNAs in one LNP vaccine.

FIG. 6A shows data from a flow cytometry analysis of surface expression of EBV gH antigen and EBV gH/gL antigen complex in HeLa cells using 2A8 antibody, or surface expression of EBV gH/gL antigen complex in HeLa cells using 2D4 antibody. FIG. 6B is a bar graph showing percent gH/gL expression on the surface of HeLa cells transfected with 0.25 μg of a gH mRNA and a gL mRNA, having one of the two different 5′ UTR sequences, 24 hours post transfection.

FIG. 7 is a graph showing gH/gL-specific binding antibody titers (log₁₀) produced in Balb/c mice following IM vaccination with mRNA encoding indicated EBV antigen complexes (EBV gH/gL, EBV gH/gL/gB, EBV gH/gL/gp350, or EBV gH/gL/gB/gp350) formulated in a lipid nanoparticle. Various indicated doses were administered.

FIG. 8 is a graph showing gB-specific binding antibody titers (log₁₀) produced in Balb/c mice following IM vaccination with mRNA encoding indicated EBV antigen (EBV gB) or EBV antigen complexes (EBV gH/gL/gB, EBV gB/gp350, or EBV gH/gL/gB/gp350) formulated in a lipid nanoparticle. Various indicated doses were administered.

FIG. 9 is a graph showing gp350-specific binding antibody titers (log₁₀) produced in Balb/c mice following IM vaccination with mRNA encoding indicated EBV antigen (EBV gp350) or EBV antigen complexes (EBV gH/gL/gp350, EBV gB/gp350, or EBV gH/gL/gB/gp350) formulated in a lipid nanoparticle. Various indicated doses were administered.

FIGS. 10A-10D show antigen-specific CD8 T cell responses to various EBV latent genes in Balb/c mice following IM vaccination with mRNA encoding either LMP1, LMP2, EBNA1 (EBAN1d1-400), EBNA3A alone or a combination (combo) of LMP1, LMP2, EBNA1, EBNA3A and gp350.

FIGS. 11A-11D show antigen-specific CD4 T cell responses to various EBV latent genes in Balb/c mice following IM vaccination with mRNA encoding either LMP1, LMP2, EBNA1 (EBAN1d1-400), EBNA3A alone or a combination (combo) of LMP1, LMP2, EBNA1, EBNA3A and gp350.

FIG. 12 shows schematics of EBV gp350 variants of the present disclosure.

FIGS. 13A-13B show data from a flow cytometry analysis for surface expression in HeLa cells transfected with mRNA encoding either a linked glycoprotein L (gL) and glycoprotein H (gH) construct (gL-gH Linker A or gL-gH Linker B) or individual gH and gL that are co-transfected using 2A8 antibody. FIG. 13B is a bar graph showing percent gL-gH expression (percent 2A8 positive and percent CL40 positive) on the surface of the HeLa cells transfected with the indicated mRNA. Mean fluorescence intensity (MFI) is also shown.

FIG. 14 shows EBNA1-specific polyclonal CD4 and CD8 T cell responses (e.g., IFNγ, TNFα, and IL-2 secretion) in Balb/c mice following IM vaccination with various mRNAs encoding EBV antigens (LMP2, EBNA1, gH, gL, and gp350, with UTR1 or UTR2) or mRNA encoding EBNA1 antigen alone.

FIG. 15 shows LMP2-specific polyclonal CD4 and CD8 T cell responses (e.g., IFNγ, TNFα, and IL-2 secretion) in Balb/c mice following IM vaccination with mRNAs encoding various EBV antigens (LMP2, EBNA1, gH, gL, and gp350, with UTR1 or UTR2) or mRNA encoding LMP2 antigen alone.

FIG. 16 shows a schematic of the experimental protocol (top) and graphs of the resulting gp350-specific IgG titers (bottom left) and gH/gL-specific IgG titers (bottom right) in immune sera of non human primates (NHPs) vaccinated with a combination EBV mRNA vaccine (gp350, gH, gL, LMP2, and EBNA1) or control.

FIG. 17 is a graph showing the neutralizing titers against EBV infection of Raji B cells in immune sera of NHPs vaccinated with the indicated doses and constructs or the neutralizing titers present in EBV+ human sera.

FIG. 18 is a graph showing the gp350-specific IgG titers following cell transfection with EBV vaccine constructs generated using different downstream purification processes.

FIG. 19 is a graph showing the gH/gL-specific IgG titers following cell transfection with EBV vaccine constructs generated using different downstream purification processes.

### DETAILED DESCRIPTION

The Epstein-Barr virus (EBV) is a double-stranded DNA γ-herpesvirus that infects B cells and epithelial cells, causing infectious mononucleosis, and that has been linked to malignancies, such as Burkitt's lymphoma, Hodgkin's lymphoma, and nasopharyngeal carcinoma, in both cell types in vivo. Nearly 95% of the population is infected by EBV by adulthood and carries EBV DNA throughout life. EBV is maintained in a latent state in infected B lymphocytes, with periodic reactivation of lytic replication.

EBV, widespread in all human populations, can be isolated in vitro via its ability to transform resting human B cells into permanent lymphoblastoid cell lines (LCLs) expressing the virus-coded antigens EBNA1, 2, 3A, 3B, 3C, and LP and the latent membrane proteins (LMPs) 1, 2A, and 2B. EBV isolates can be categorized as type 1 or type 2 on the basis of marked allelic polymorphisms within the EBNA2, 3A, 3B, and 3C genes and into distinct strains on the basis of more-subtle sequence variations within the EBNA1, EBNA2, and LMP1 genes and certain lytic cycle genes.

EBV has three glycoproteins, glycoprotein B (gB), gH, and gL, that form the core membrane fusion machinery mediating viral entry into a cell. The gH and gL proteins associate to form a heterodimeric complex, which is necessary for efficient membrane fusion and is also implicated in direct binding to epithelial cell receptors required for viral entry. EBV uses different pathways for the infection of epithelial cells and B lymphocytes. For both cell types, the minimal viral glycoprotein components that mediate membrane fusion have been identified. As with other herpesviruses, EBV uses the core viral entry glycoproteins, glycoprotein B (gB) and the gH/gL complex. For the infection of B lymphocytes, EBV requires an additional protein, gp42, which binds to host HLA class II molecules, triggering the membrane fusion step. gp42 has multiple functional sites for interaction with gH/gL, HLA class II, and potentially, another unknown binding ligand that could be engaged through a large surface-exposed hydrophobic pocket. The gp42 protein binds to the gH/gL complex with nanomolar affinity through its N-terminal region, and this interaction can be recapitulated with a synthetic peptide of ˜35 aa residues. EBV glycoprotein-mediated membrane fusion with epithelial cells does not require gp42 but only gB and gH/gL. Recent observations indicate that EBV gH/gL engages integrins αvβ6 and/or αvβ8 on epithelial cells to trigger membrane fusion and entry.

The EBV gp350 glycoprotein encoded by BLLF1 is important for efficient EBV infection of resting B cells. Gp350 is the most abundant viral protein in the viral envelope. This large protein is heavily glycosylated and localizes to various subcellular compartments (cytoplasm, endoplasmic reticulum, Golgi, and plasma membrane) of replicating cells. EBV binds to primary B cells through its interaction with CD21, the complement receptor 2 (CR2) via gp350. Several gp350 domains appear to be involved in the formation of a stable complex with CD21, one of which has been identified as the receptor-binding site (amino acids [aa] 142 to 161). This glycan-free domain is also recognized by the neutralizing gp350-specific antibody 72A.

The present disclosure is not limited by a particular strain of EBV. The strain of EBV used in a vaccine may be any strain of EBV.

The present disclosure provides RNA (e.g., mRNA) vaccines against EBV infection—vaccines that elicit potent neutralizing antibodies and robust T cell responses against EBV antigens, inhibit the production of viral immunomodulatory factors, and/or prevent viral latency.

In some embodiments, vaccines disclosed herein are used therapeutically, i.e., following infection with EBV (to treat the infection). In some embodiments, the vaccines of the present disclosure can be used to prevent or reduce the frequency of Hodgkin's lymphoma, Burkitt's lymphoma, gastric carcinoma, nasopharyngeal carcinoma, post-transplant lymphoproliferative disease, diffuse B cell lymphoma, and/or NK/T cell lymphoma.

The EBV RNA vaccines described herein are superior to current vaccines in several ways. For example, the lipid nanoparticle (LNP) delivery system used herein increases the efficacy of RNA vaccines in comparison to other formulations, including a protamine-based approach described in the literature. The use of this LNP delivery system enables the effective delivery of chemically-modified RNA vaccines or unmodified RNA vaccines, without requiring additional adjuvant to produce a therapeutic result (e.g., production neutralizing antibody titer and/or a T cell response). In some embodiments, the EBV RNA vaccines disclosed herein are superior to conventional vaccines by a factor of at least 10 fold, 20, fold, 40, fold, 50 fold, 100 fold, 500 fold, or 1,000 fold when administered intramuscularly (IM) or intradermally (ID). These results can be achieved even when significantly lower doses of the RNA (e.g., mRNA) are administered in comparison with RNA doses used in other classes of lipid based formulations.

The LNP used in the studies described herein has been used previously to deliver siRNA in various animal models as well as in humans. In view of the observations made in association with the siRNA delivery of LNP formulations, the fact that LNP is useful in vaccines is quite surprising, particularly when immunity to an antigen has been hard to generate, as in the case of EBV. It has been observed that therapeutic delivery of siRNA formulated in LNP causes an undesirable inflammatory response associated with a transient IgM response, typically leading to a reduction in antigen production and a compromised immune response. In contrast to the findings observed with siRNA, the LNP-mRNA formulations of the present disclosure are demonstrated herein to generate enhanced IgG levels, sufficient for prophylactic and therapeutic methods rather than transient IgM responses.

### Exemplary Epstein-Barr Virus (EBV) Antigens

Antigens are proteins capable of inducing an immune response (e.g., causing an immune system to produce antibodies against the antigens). Herein, use of the term antigen encompasses immunogenic proteins and immunogenic fragments (an immunogenic fragment that induces (or is capable of inducing) an immune response to EBV), unless otherwise stated. It should be understood that the term “protein” encompasses peptides and the term “antigen” encompasses antigenic fragments.

A number of different antigens are associated with EBV. EBV vaccines, as provided herein, comprise at least one (one or more) ribonucleic acid (RNA, e.g., mRNA) having an open reading frame encoding at least one EBV antigen. Non-limiting examples of EBV antigens are provided below.

Exemplary EBV antigens are provided in the Sequence Listing elsewhere herein. For example, the antigens may be encoded by (thus the RNA may comprise or consist of) any one of sequences set forth in SEQ ID NO: 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, and/or 210. In some embodiments, the aforementioned sequences may further comprise a 5′ cap (e.g., 7mG(5′)ppp(5′)NlmpNp), a polyA tail, or a 5′ cap and a polyA tail.

It should be understood that the EBV vaccines of the present disclosure may comprise any of the RNA open reading frames (ORFs), or encode any of the protein ORFs, described herein, with or without a signal sequence. It should also be understood that the EBV vaccines of the present disclosure may include any 5′ untranslated region (UTR) and/or any 3′ UTR. Exemplary UTR sequences are provided in the Sequence Listing (e.g., SEQ ID NOs: 1, 3, 104 and 106; however, other UTR sequences (e.g., of the prior art) may be used or exchanged for any of the UTR sequences described herein. UTRs may also be omitted from the vaccine constructs provided herein.

EBV entry into B cells is initiated by attachment of glycoprotein gp350 to the complement receptor type 2 (CR2). A complex of three glycoproteins, gH, gL, and gp42, is subsequently required for penetration. gp42 binds to HLA class II, which functions as an entry mediator or co-receptor and, by analogy with other herpesviruses, gH is then thought to be involved virus-cell fusion. Entry of virus into epithelial cells is different. It can be initiated by attachment by an unknown glycoprotein in the absence of CR2. There is no interaction between gp42 and HLA class II and instead a distinct complex of only the two glycoproteins gH and gL interacts with a novel entry mediator.

EBV gH-gL complex includes of three glycoproteins, gp85, the gH homolog, which is the product of the BXLF2 open reading frame (ORF); gp25, the gL homolog, which is the product of the BKRF2 ORF; and gp42, which is the product of the BZLF2 ORF. The complex behaves in many respects like its counterparts in other herpesviruses. Glycoprotein gH is dependent on gL for authentic processing and transport, and the complex as a whole has been implicated as important to the ability of virus to fuse with the cell membrane and penetrate into the cytoplasm

The gp350 glycoprotein encoded by BLLF1 is important for efficient Epstein-Barr virus (EBV) infection of resting B cells.

The major EBV glycoprotein gp350 mediates docking of EBV on B cells by binding receptor type 2 (CR2) (Nemerow et al., J of Virol. (61):1416-1420 (1987); Szakonyi et al., Nat Struct Mol Biol. (13): 996-1001 (2006)). Due to alternative splicing, BLLF1 encodes gp350 and gp220, which are glycosylated and are approximately 350 and 220 kilodaltons in molecular weight, respectively (Beisel et al., J Virol. (54):665-674 (1985); Hummel et al., J Virol. (49):413-417 (1984)). In some embodiments, the EBV gp350 antigen comprises the sequence identified by SEQ ID NO: 81, 204, 185, 182, 207, or 208.

After EBV docking, EBV fuses with the plasma membrane of the host cell using a complex of glycoproteins. The core EBV membrane fusion machinery for entry into B cells and epithelial cells includes glycoprotein B (gB), glycoprotein H (gH) and glycoprotein L (gL) (Hutt-Fletcher et al., J Virol. (81): 7825-7832 (2007)).

gB is a single pass type 1 membrane protein also referred to as gp110 and is encoded by the BALF4 open reading frame (ORF) (Herrold et al., J of Virol. (70):2049-2054 (1996); Haan et al., Virology (290): 106-114 (2001)); McShane et al., Proc Nat Acad Sci USA. (101): 17474-17479 (2004)). In some embodiments, the EBV gB antigen comprises the sequence identified by SEQ ID NO: 209.

gH (also referred to as gp85) is a type 1 transmembrane protein encoded by the open reading frame (ORF) of the BXLF2 gene (Heineman et al., J Virol. (62):1101-1107 (1988)); Oba et al., J Virol. (62):1108-1114 (1988)). In some embodiments, the EBV gH antigen comprises the sequence identified by SEQ ID NO: 187.

gL (also referred to as gp25) and encoded by BKRF2 ORF is required for proper folding and localization of gH (Li et al., J Virol. (69): 3987-3994 (1995); Yaswen et al., Virology. (195): 387-396 (1993)). Therefore, gH and gL often functions as a complex to mediate viral fusion and this complex has been crystallized (Matsurra et al., Proc Nat Acad Sci USA. (107):22641-2264 (2010)). In some embodiments, the EBV gL antigen comprises the sequence identified by SEQ ID NO: 188.

In addition to the core membrane fusion machinery, EBV entry into B cells requires gp42, which is encoded by a BZLF2 ORF (Kirschner et al., J. Virol. (80):9444-54 (2006); Wang et al., J. Virol., (72):5552-5558 (1998); Silva et al., J. Virol. (78): 5946-5956 (2004); Li et al. J.

Virol., (69):3987-3994 (1995). EBV gp42 mediates viral fusion with B cells by binding MHC class II molecules (Mullen et al., Molecular Cell. (9):375-385 (2002); Haan et al. J Virol. (74): 2451-4 (2000)). In some embodiments, the EBV gp42 antigen comprises the sequence identified by SEQ ID NO: 189.

Latent membrane protein 1 (LMP1) is a six transmembrane domain protein that promotes immortalization of resting B cells and helps protect EBV-infected B cells from apoptosis (Hennessy et al., Proc Natl. Acad. Sci USA. (81): 7207-11 (1984); Kaye et al., Proc Nat Acad Sci USA. (90): 9150-9154 (1993); Henderson et al., Cell (65): 1107-1115 (1991)). A number of signaling pathways may be activated by LMP1, including tumor necrosis factor receptor family signaling and DNA synthesis (Peng et al. Oncogene. (7): 1775-1782; Masialos et al., Cell. (80):389-399 (1995); Li et al., J Biomed Sci. (10):490-504 (2003)). Furthermore, LMP1 signaling can upregulate expression of the anti-apoptosis Bcl-2 oncogene in B cells (Rowe et al., J Virol. (68):5602-12 (1994)). In some embodiments, the EBV LMP1 antigen comprises the sequence identified by SEQ ID NO: 179.

Similar to LMP1, Latent membrane protein 2 (LMP2) is an EBV-encoded transmembrane protein that is often expressed in latently infected cells. There are two isoforms of LMP2 (LMP2A and LMP2B) (Laux et al., EMBO J. (7):769-74 (1988); Longnecker et al., J Virol. (64):2319-26 (1990)). LMP2A is implicated in maintaining EBV latency. For example, LMP2A can exclude B-cell receptor (BCR) from lipid rafts to prevent lytic induction (Dykstra et al., Immunity. (14):57-67 (2001)). LMP2A can also activate the phosphatidylinositol 3-kinase (PI3-K)/Akt pathway to promote cell survival (Scholle et al., J Virol. (74): 10681-10689 (2000); Swart et al. J Virol. (74): 10838-10845 (2000); Fukuda et al., J. Virol. (78): 1697-16705 (2004)). LMP2B protein generally lacks 119 amino-terminal amino acids compared to LMP2A and is implicated in epithelial cell spreading and motility (Allen et al., J Virol. (79):1789-1802 (2005)). In some embodiments, the EBV LMP2 antigen comprises the sequence identified by SEQ ID NO: 181.

Epstein-Barr nuclear antigens (ENBAs) that help establish latent infection include EBNA1, EBNA2, EBNA3A and EBNA3C. EBNA1 encoded by BKRF1 promotes viral DNA replication, episomal maintenance and episomal partitioning (Rawlins et al., Cell (42): 859-68 (1985); (Hung et al., Proc Natl Acad Sciences USA (98): 1865-1870 (2001)). In particular, EBNA1 can bind family of repeats and dyad symmetry elements of the latent origin oriP. In some embodiments, the EBV EBNA1 antigen comprises the sequence identified by SEQ ID NO: 178.

There are three members of the EBNA3 family: EBNA3A, EBNA3B and EBNA3C. EBNA3s regulate transcription by binding RBPJ, which is a transcriptional regulator in the Notch signaling pathway (Zhao et al., J Virol. (70):4228-4236 (1996); Robertson et al., J Virol. (69):3108-3116 (1995); Robertson et al. J Virol. (70):3068-3074 (1996)). In particular, EBNA3A and EBNA3C have been shown to be required for EBV-mediated transformation of B cells (Tomkinson et al. J Virol. (67):2014-25 (1993)). In some embodiments, the EBV EBNA3A antigen comprises the sequence identified by SEQ ID NO: 177.

### Nucleic Acids

The EBV vaccines of the present disclosure comprise at least one (one or more) ribonucleic acid (RNA) having an open reading frame encoding at least one EBV antigen. In some embodiments, the RNA is a messenger RNA (mRNA) having an open reading frame encoding at least one EBV antigen. In some embodiments, the RNA (e.g., mRNA) further comprises a (at least one) 5′UTR, 3′UTR, a polyA tail and/or a 5′cap.

Nucleic acids comprise a polymer of nucleotides (nucleotide monomers), also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any ribonucleic acid that encodes a (at least one) protein (a naturally-occurring, non-naturally-occurring, or modified polymer of amino acids) and can be translated to produce the encoded protein in vitro, in vivo, in situ or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in a vaccine of the present disclosure.

### Variants

In some embodiments, an RNA of the present disclosure encodes an EBV antigen variant. Antigen or other polypeptide variants refers to molecules that differ in their amino acid sequence from a wild-type, native or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, an EBV vaccine comprises an mRNA ORF having a nucleotide sequence identified by any one of the sequences provided herein (see e.g., Sequence Listing), or having a nucleotide sequence at least 80%, at least 85%, at least 90%, at least 95%, at least 96%, at least 97%, at least 98%, or at least 99% identical to a nucleotide sequence identified by any one of the sequence provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of EBV antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the EBV pathogen. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a vaccine includes at least one RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, EBV RNA vaccines may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, EBV RNA vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, EBV RNA vaccines do not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

In some embodiments, EBV RNA vaccines may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, EBV RNA vaccines may have one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

### Signal Peptides

In some embodiments, an EBV vaccine comprises a RNA having an ORF that encodes a signal peptide fused to the EBV antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than EBV antigens in nature) are known in the art and can be tested for desired properties and then incorporated into a nucleic acid of the disclosure. In some embodiments, the signal peptide is a bovine prolactin signal peptide. For example, the bovine prolactin signal peptide may comprise sequence MDSKGSSQKGSRLLLLLVVSNLLLPQGVVG (SEQ ID NO: 115). Other signal peptide sequences may also be used. For example, the signal peptide may comprise one of the following sequences: MDWTWILFLVAAATRVHS (SEQ ID NO: 116); METPAQLLFLLLLWLPDTTG (SEQ ID NO: 117); MLGSNSGQRVVFTILLLLVAPAYS (SEQ ID NO: 118); MKCLLYLAFLFIGVNCA (SEQ ID NO: 119); MWLVSLAIVTACAGA (SEQ ID NO: 120).

### Fusion Proteins

In some embodiments, an EBV RNA vaccine of the present disclosure includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the EBV antigen. Antigenic fusion proteins, in some embodiments, retain the functional property from each original protein.

### Scaffold Moieties

The RNA (e.g., mRNA) vaccines as provided herein, in some embodiments, encode fusion proteins which comprise EBV antigens linked to scaffold moieties. In some embodiments, such scaffold moieties impart desired properties to an antigen encoded by a nucleic acid of the disclosure. For example scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments an EBV antigen is fused to HBsAG or HBcAG to facilitate self-assembly of nanoparticles displaying the EBV antigen.

In another embodiment, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS linker or a GGGGS linker, for example, including one or more (e.g., 1, 2, 3, 4, or more) repeat GGGS (SEQ ID NO: 226) or GGGGS (SEQ ID NO: 227) sequences (e.g., GGGGS GGGGS GGGGS (SEQ ID NO: 224) and/or GGGGS GGGGS GGGGS GGGGS (SEQ ID NO: 225)). In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017/127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding an EBV antigen). In some embodiments, a codon optimized sequence shares less than 90% sequence identity to a naturally-occurring or wild-type sequence (e.g., a naturally-occurring or wild-type mRNA sequence encoding an EBV antigen). In some embodiments, a codon optimized sequence shares less than 85% sequence identity to a naturally-occurring or wild-type sequence (e.g., a naturally-occurring or wild-type mRNA sequence encoding an EBV antigen). In some embodiments, a codon optimized sequence shares less than 80% sequence identity to a naturally-occurring or wild-type sequence (e.g., a naturally-occurring or wild-type mRNA sequence encoding an EBV antigen). In some embodiments, a codon optimized sequence shares less than 75% sequence identity to a naturally-occurring or wild-type sequence (e.g., a naturally-occurring or wild-type mRNA sequence encoding an EBV antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding an EBV antigen). In some embodiments, a codon optimized sequence shares between 65% and 75% or about 80% sequence identity to a naturally-occurring or wild-type sequence (e.g., a naturally-occurring or wild-type mRNA sequence encoding an EBV antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than an EBV antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, at least one RNA (e.g., mRNA) of an EBV vaccines of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

EBV RNA vaccines of the present disclosure comprise, in some embodiments, at least one nucleic acid (e.g., RNA) having an open reading frame encoding at least one EBV antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/TB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The nucleic acids of the present disclosure may comprise one or more regions or parts which act or function as an untranslated region. Where nucleic acids are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 121), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (TE1) gene (US20140206753, WO2013/185069), the sequence GGGAUCCUACC (SEQ ID NO: 122) (WO2014/144196) may also be used. In another embodiment, 5′ UTR of a TOP gene is a 5′ UTR of a TOP gene lacking the 5′ TOP motif (the oligopyrimidine tract) (e.g., WO2015/101414, WO2015/101415, WO2015/062738, WO2015/024667, WO2015/024668; 5′ UTR element derived from ribosomal protein Large 32 (L32) gene (WO2015/101414, WO2015/101415, WO2015/062738), 5′ UTR element derived from the 5′UTR of an hydroxysteroid (17-β) dehydrogenase 4 gene (HSD17B4) (WO2015/024667), or a 5′ UTR element derived from the 5′ UTR of ATP5A1 (WO2015/024667) can be used. In some embodiments, an internal ribosome entry site (IRES) is used instead of a 5′ UTR.

In some embodiments, a 5′ UTR of the present disclosure comprises a sequence selected from SEQ ID NO: 1 and SEQ ID NO: 104.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) (SEQ ID NO: 123) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US20110086907). A modified β-globin construct with enhanced stability in some cell types by cloning two sequential human β-globin 3′UTRs head to tail has been developed and is well known in the art (US2012/0195936, WO2014/071963). In addition a2-globin, a1-globin, UTRs and mutants thereof are also known in the art (WO2015/101415, WO2015/024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014/152774), rabbit β globin and hepatitis B virus (HBV), α-globin 3′ UTR and Viral VEEV 3′ UTR sequences are also known in the art. In some embodiments, the sequence UUUGAAUU (WO2014/144196) is used. In some embodiments, 3′ UTRs of human and mouse ribosomal protein are used. Other examples include rps9 3′UTR (WO2015/101414), FIG. 4 (WO2015/101415), and human albumin 7 (WO2015/101415).

In some embodiments, a 3′ UTR of the present disclosure comprises a sequence selected from SEQ ID NO: 3 and SEQ ID NO: 106.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### In Vitro Transcription of RNA

cDNA encoding the polynucleotides described herein may be transcribed using an in vitro transcription (IVT) system. In vitro transcription of RNA is known in the art and is described in International Publication WO/2014/152027, which is incorporated by reference herein in its entirety.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to EBV RNA, e.g. EBV mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedback, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present invention may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Pharmaceutical Formulations

Provided herein are compositions (e.g., pharmaceutical compositions), methods, kits and reagents for prevention or treatment of EBV in humans and other mammals, for example. EBV RNA (e.g., mRNA) vaccines can be used as therapeutic or prophylactic agents. They may be used in medicine to prevent and/or treat infectious disease.

In some embodiments, an EBV vaccine containing RNA polynucleotides as described herein can be administered to a subject (e.g., a mammalian subject, such as a human subject), and the RNA polynucleotides are translated in vivo to produce an antigenic polypeptide (antigen).

An “effective amount” of an EBV vaccine is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of an EBV vaccine provides an induced or boosted immune response as a function of antigen production in the cells of the subject. In some embodiments, an effective amount of the EBV RNA vaccine containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, RNA vaccines (including polynucleotides and their encoded polypeptides) in accordance with the present disclosure may be used for treatment or prevention of EBV. EBV RNA vaccines may be administered prophylactically or therapeutically as part of an active immunization scheme to healthy individuals or early in infection during the incubation phase or during active infection after onset of symptoms. In some embodiments, the amount of RNA vaccines of the present disclosure provided to a cell, a tissue or a subject may be an amount effective for immune prophylaxis.

EBV RNA (e.g., mRNA) vaccines may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, EBV RNA vaccines may be administered intramuscularly, intranasally or intradermally, similarly to the administration of inactivated vaccines known in the art.

The EBV RNA vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including EBV RNA vaccines and RNA vaccine compositions and/or complexes optionally in combination with one or more pharmaceutically acceptable excipients.

EBV RNA (e.g., mRNA) vaccines may be formulated or administered alone or in conjunction with one or more other components. For instance, EBV RNA vaccines (vaccine compositions) may comprise other components including, but not limited to, adjuvants.

In some embodiments, EBV RNA vaccines do not include an adjuvant (they are adjuvant free).

EBV RNA (e.g., mRNA) vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, EBV RNA vaccines are administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, EBV RNA vaccines are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with EBV RNA vaccines (e.g., for transplantation into a subject), hyaluronidase, nanoparticle mimics and combinations thereof.

### Lipid Nanoparticles (LNPs)

In some embodiments, EBV RNA (e.g., mRNA) vaccines of the disclosure are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, an ionizable cationic lipid of the disclosure comprises a compound of Formula (I):

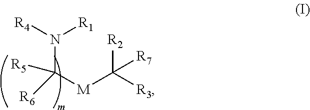

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (T) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and -CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

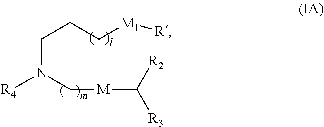

or a salt or isomer thereof, wherein 1 is selected from 1, 2, 3, 4, and 5; m is selected from 5, 6, 7, 8, and 9; Mi is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

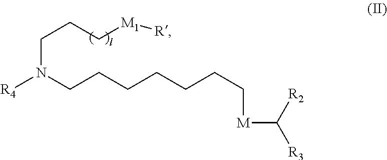

or a salt or isomer thereof, wherein 1 is selected from 1, 2, 3, 4, and 5; Mi is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

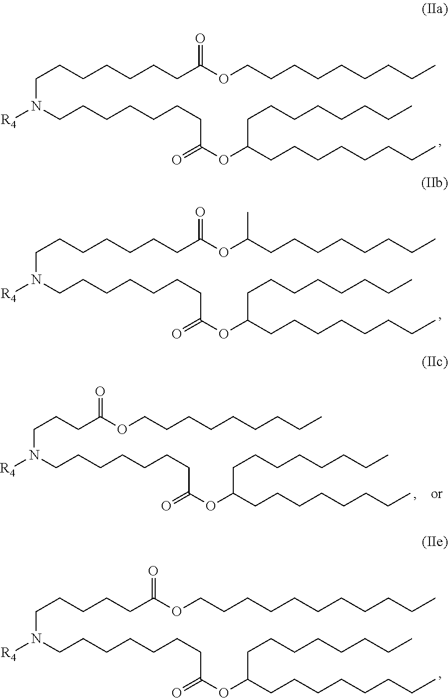

or a salt or isomer thereof, wherein R₄ is as described herein.

In some embodiments, a subset of compounds of Formula (I) includes those of Formula (IId):

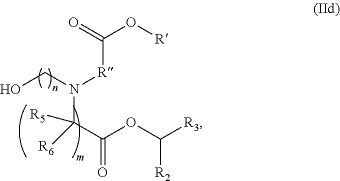

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

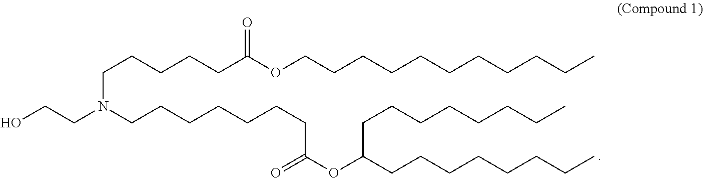 [SMILES: CCCCCCCCCCCOC(=O)CCCCCN(CCO)CCCCCCCC(=O)OC(CCCCCCCC)CCCCCCCC]

In some embodiments, an ionizable cationic lipid of the disclosure comprises a compound having structure:

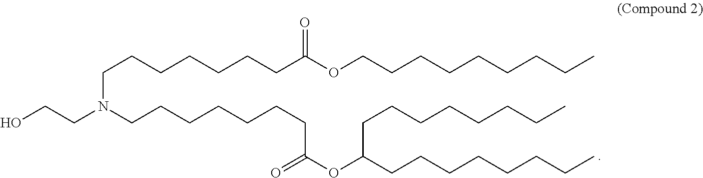 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-diolcoyl-sn-glycero-3-phosphocthanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphocthanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Multivalent Vaccines

The EBV vaccines, as provided herein, may include an RNA (e.g. mRNA) or multiple RNAs encoding two or more antigens of the same or different EBV species. In some embodiments, an EBV vaccine includes an RNA or multiple RNAs encoding two or more antigens selected from gp350, gH, gL, gB, gp42, LMP1, LMP2, EBNA1 and EBNA3 antigens. In some embodiments, the RNA (at least one RNA) of an EBV vaccine may encode 1, 2, 3, 4, 5, 6, 7, 8, 9, 10, 11, 12, or more antigens.

In some embodiments, an EBV vaccine comprises at least one RNA encoding a gp350 antigen and a gH antigen. In some embodiments, an EBV vaccine comprises at least one RNA encoding a gp350 antigen and a gL antigen. In some embodiments, an EBV vaccine comprises at least one RNA encoding a gp350 antigen and a gB antigen. In some embodiments, an EBV vaccine comprises at least one RNA encoding a gp350 antigen and a gp42 antigen. In some embodiments, an EBV vaccine comprises at least one RNA encoding a gp350 antigen and a LMP (e.g., LMP1 and/or LMP2) antigen. In some embodiments, an EBV vaccine comprises at least one RNA encoding a gp350 antigen and an EBNA (e.g., EBNA1 and/or EBNA3) antigen.

In some embodiments, an EBV vaccine comprises at least one RNA encoding a gp350 antigen, gH antigen and a gL antigen. In some embodiments, an EBV vaccine comprises at least one RNA encoding a gp350 antigen, gH antigen and a gB antigen. In some embodiments, an EBV vaccine comprises at least one RNA encoding a gp350 antigen, gH antigen and a gp42 antigen. In some embodiments, an EBV vaccine comprises at least one RNA encoding a gp350 antigen, gH antigen and a LMP (e.g., LMP1 and/or LMP2) antigen. In some embodiments, an EBV vaccine comprises at least one RNA encoding a gp350 antigen, gH antigen and an EBNA (e.g., EBNA1 and/or EBNA3) antigen.

In some embodiments, an EBV vaccine comprises at least one RNA encoding a gp350 antigen, gL antigen and a gB antigen. In some embodiments, an EBV vaccine comprises at least one RNA encoding a gp350 antigen, gL antigen and a gp42 antigen. In some embodiments, an EBV vaccine comprises at least one RNA encoding a gp350 antigen, gL antigen and a LMP (e.g., LMP1 and/or LMP2) antigen. In some embodiments, an EBV vaccine comprises at least one RNA encoding a gp350 antigen, gL antigen and an EBNA (e.g., EBNA1 and/or EBNA3) antigen.

In some embodiments, an EBV vaccine comprises at least one RNA encoding a gp350 antigen, gB antigen and a gp42 antigen. In some embodiments, an EBV vaccine comprises at least one RNA encoding a gp350 antigen, gB antigen and a LMP (e.g., LMP1 and/or LMP2) antigen. In some embodiments, an EBV vaccine comprises at least one RNA encoding a gp350 antigen, gB antigen and an EBNA (e.g., EBNA1 and/or EBNA3) antigen.

In some embodiments, an EBV vaccine comprises at least one RNA encoding a gp350 antigen, gp42 antigen and a LMP (e.g., LMP1 and/or LMP2) antigen. In some embodiments, an EBV vaccine comprises at least one RNA encoding a gp350 antigen, gp42 antigen and an EBNA (e.g., EBNA1 and/or EBNA3) antigen.

In some embodiments, an EBV vaccine comprises at least one RNA encoding a gp350 antigen, a LMP (e.g., LMP1 and/or LMP2) antigen, and an EBNA (e.g., EBNA1 and/or EBNA3) antigen.

In some embodiments, an EBV vaccine comprises at least one RNA encoding a gH antigen and a gL antigen. In some embodiments, an EBV vaccine comprises at least one RNA encoding a gH antigen and a gB antigen. In some embodiments, an EBV vaccine comprises at least one RNA encoding a gH antigen and a gp42 antigen. In some embodiments, an EBV vaccine comprises at least one RNA encoding a gH antigen and a LMP (e.g., LMP1 and/or LMP2) antigen. In some embodiments, an EBV vaccine comprises at least one RNA encoding a gH antigen and an EBNA (e.g., EBNA1 and/or EBNA3) antigen.

In some embodiments, an EBV vaccine comprises at least one RNA encoding a gL antigen and a gB antigen. In some embodiments, an EBV vaccine comprises at least one RNA encoding a gL antigen and a gp42 antigen. In some embodiments, an EBV vaccine comprises at least one RNA encoding a gL antigen and a LMP (e.g., LMP1 and/or LMP2) antigen. In some embodiments, an EBV vaccine comprises at least one RNA encoding a gL antigen and an EBNA (e.g., EBNA1 and/or EBNA3) antigen.

In some embodiments, an EBV vaccine comprises at least one RNA encoding a gB antigen and a gp42 antigen. In some embodiments, an EBV vaccine comprises at least one RNA encoding a gB antigen and a LMP (e.g., LMP1 and/or LMP2) antigen. In some embodiments, an EBV vaccine comprises at least one RNA encoding a gB antigen and an EBNA (e.g., EBNA1 and/or EBNA3) antigen.

In some embodiments, an EBV vaccine comprises at least one RNA encoding a gp42 antigen and a LMP (e.g., LMP1 and/or LMP2) antigen. In some embodiments, an EBV vaccine comprises at least one RNA encoding a gp42 antigen and an EBNA (e.g., EBNA1 and/or EBNA3) antigen.

In some embodiments, an EBV vaccine comprises at least one RNA encoding a LMP (e.g., LMP1 and/or LMP2) antigen and an EBNA (e.g., EBNA1 and/or EBNA3) antigen.

In some embodiments, two or more different RNA (e.g., mRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The EBV vaccines, as provided herein, may include an RNA or multiple RNAs encoding two or more antigens of the same or different EBV strains. Also provided herein are combination vaccines that include RNA encoding one or more EBV antigen(s) and one or more antigen(s) of a different organisms (e.g., bacterial and/or viral organism). Thus, the vaccines of the present disclosure may be combination vaccines that target one or more antigens of the same strain/species, or one or more antigens of different strains/species, e.g., antigens which induce immunity to organisms which are found in the same geographic areas where the risk of EBV infection is high or organisms to which an individual is likely to be exposed to when exposed to EBV.

### Dosing/Administration

Provided herein are compositions (e.g., pharmaceutical compositions), methods, kits and reagents for prevention and/or treatment of EBV in humans and other mammals. EBV RNA vaccines can be used as therapeutic or prophylactic agents. In some aspects, the RNA vaccines of the disclosure are used to provide prophylactic protection from EBV. In some aspects, the RNA vaccines of the disclosure are used to treat an EBV infection. In some embodiments, the EBV vaccines of the present disclosure are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, the EBV vaccines are administered to a subject (e.g., a mammalian subject, such as a human subject) in an effective amount to induce an antigen-specific immune response. The RNA encoding the EBV antigen is expressed and translated in vivo to produce the antigen, which then stimulates an immune response in the subject.

Prophylactic protection from EBV can be achieved following administration of an EBV RNA vaccine of the present disclosure. Vaccines can be administered once, twice, three times, four times or more but it is likely sufficient to administer the vaccine once (optionally followed by a single booster). It is possible, although less desirable, to administer the vaccine to an infected individual to achieve a therapeutic response. Dosing may need to be adjusted accordingly.

A method of eliciting an immune response in a subject against EBV is provided in aspects of the present disclosure. The method involves administering to the subject an EBV RNA vaccine comprising at least one RNA (e.g., mRNA) having an open reading frame encoding at least one EBV antigen, thereby inducing in the subject an immune response specific to EBV antigen, wherein anti-antigen antibody titer in the subject is increased following vaccination relative to anti-antigen antibody titer in a subject vaccinated with a prophylactically effective dose of a traditional vaccine against the EBV. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the EBV or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the EBV or an unvaccinated subject.

A method of eliciting an immune response in a subject against an EBV is provided in other aspects of the disclosure. The method involves administering to the subject an EBV RNA vaccine comprising at least one RNA polynucleotide having an open reading frame encoding at least one EBV antigen, thereby inducing in the subject an immune response specific to EBV antigen, wherein the immune response in the subject is equivalent to an immune response in a subject vaccinated with a traditional vaccine against the EBV at 2 times to 100 times the dosage level relative to the RNA vaccine.

In some embodiments, the immune response in the subject is equivalent to an immune response in a subject vaccinated with a traditional vaccine at twice the dosage level relative to the EBV RNA vaccine. In some embodiments, the immune response in the subject is equivalent to an immune response in a subject vaccinated with a traditional vaccine at three times the dosage level relative to the EBV RNA vaccine. In some embodiments, the immune response in the subject is equivalent to an immune response in a subject vaccinated with a traditional vaccine at 4 times, 5 times, 10 times, 50 times, or 100 times the dosage level relative to the EBV RNA vaccine. In some embodiments, the immune response in the subject is equivalent to an immune response in a subject vaccinated with a traditional vaccine at 10 times to 1000 times the dosage level relative to the EBV RNA vaccine. In some embodiments, the immune response in the subject is equivalent to an immune response in a subject vaccinated with a traditional vaccine at 100 times to 1000 times the dosage level relative to the EBV RNA vaccine.

In other embodiments, the immune response is assessed by determining [protein] antibody titer in the subject. In other embodiments, the ability of serum or antibody from an immunized subject is tested for its ability to neutralize viral uptake or reduce EBV transformation of human B lymphocytes. In other embodiments, the ability to promote a robust T cell response(s) is measured using art recognized techniques.

Other aspects the disclosure provide methods of eliciting an immune response in a subject against an EBV by administering to the subject an EBV RNA vaccine comprising at least one RNA polynucleotide having an open reading frame encoding at least one EBV antigen, thereby inducing in the subject an immune response specific to EBV antigen, wherein the immune response in the subject is induced 2 days to 10 weeks earlier relative to an immune response induced in a subject vaccinated with a prophylactically effective dose of a traditional vaccine against the EBV. In some embodiments, the immune response in the subject is induced in a subject vaccinated with a prophylactically effective dose of a traditional vaccine at 2 times to 100 times the dosage level relative to the RNA vaccine.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against an EBV by administering to the subject an EBV RNA vaccine having an open reading frame encoding a first antigen, wherein the RNA polynucleotide does not include a stabilization element, and wherein an adjuvant is not co-formulated or co-administered with the vaccine.

EBV RNA (e.g., mRNA) vaccines may be administered by any route which results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. EBV RNA (e.g., mRNA) vaccines compositions are typically formulated in dosage unit form for ease of administration and uniformity of dosage. It will be understood, however, that the total daily usage of EBV RNA (e.g., mRNA)vaccines compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of an EBV vaccine, as provided herein, may be as low as 20 μg, administered for example as a single dose or as two 10 μg doses. In some embodiments, the effective amount is a total dose of 20 μg-200 μg. For example, the effective amount may be a total dose of 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg or 200 μg. In some embodiments, the effective amount is a total dose of 25 μg-200 μg. In some embodiments, the effective amount is a total dose of 50 μg-200 μg.

In some embodiments, EBV RNA (e.g., mRNA) vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, EBV RNA (e.g., mRNA) vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, EBV RNA (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, EBV RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, an EBV RNA (e.g., mRNA) vaccine composition may be administered three or four times.

In some embodiments, EBV RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the EBV RNA (e.g., mRNA) vaccine for use in a method of vaccinating a subject is administered the subject a single dosage of between 10 μg/kg and 400 g/kg of the nucleic acid vaccine in an effective amount to vaccinate the subject. In some embodiments, the RNA vaccine for use in a method of vaccinating a subject is administered the subject a single dosage of between 10 μg and 400 μg of the nucleic acid vaccine in an effective amount to vaccinate the subject. In some embodiments, an EBV RNA (e.g., mRNA) vaccine for use in a method of vaccinating a subject is administered to the subject as a single dosage of 25-1000 μg (e.g., a single dosage of mRNA encoding an EBV antigen). In some embodiments, an EBV RNA vaccine is administered to the subject as a single dosage of 25, 50, 100, 150, 200, 250, 300, 350, 400, 450, 500, 550, 600, 650, 700, 750, 800, 850, 900, 950 or 1000 μg. For example, an EBV RNA vaccine may be administered to a subject as a single dose of 25-100, 25-500, 50-100, 50-500, 50-1000, 100-500, 100-1000, 250-500, 250-1000, or 500-1000 μg. In some embodiments, an EBV RNA (e.g., mRNA) vaccine for use in a method of vaccinating a subject is administered to the subject as two dosages, the combination of which equals 25-1000 μg of the EBV RNA (e.g., mRNA) vaccine.

AN EBV RNA (e.g., mRNA) vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the EBV RNA (e.g., mRNA) vaccine, wherein the EBV RNA vaccine is formulated in an effective amount to produce an antigen specific immune response in a subject (e.g., production of antibodies specific to an anti-EBV antigen). “An effective amount” is a dose of an EBV RNA (e.g., mRNA) vaccine effective to produce an antigen-specific immune response. Also provided herein are methods of inducing an antigen-specific immune response in a subject.

As used herein, an immune response to a vaccine or LNP of the present invention is the development in a subject of a humoral and/or a cellular immune response to a (one or more) EBV protein(s) present in the vaccine. For purposes of the present invention, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function, and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-EBV antigen antibody titer produced in a subject administered an EBV RNA (e.g., mRNA) vaccine as provided herein. An antibody titer is a measurement of the amount of antibodies within a subject, for example, antibodies that are specific to a particular antigen (e.g., an anti-EBV antigen) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the EBV RNA (e.g., mRNA) vaccine.

In some embodiments, an anti-EBV antigen antibody titer produced in a subject is increased by at least 1 log relative to a control. For example, anti-EBV antigen antibody titer produced in a subject may be increased by at least 1.5, at least 2, at least 2.5, or at least 3 log relative to a control. In some embodiments, the anti-EBV antigen antibody titer produced in the subject is increased by 1, 1.5, 2, 2.5 or 3 log relative to a control. In some embodiments, the anti-EBV antigen antibody titer produced in the subject is increased by 1-3 log relative to a control. For example, the anti-EBV antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-EBV antigen antibody titer produced in a subject is increased at least 2 times relative to a control. For example, the anti-EBV antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-EBV antigen antibody titer produced in the subject is increased 2, 3, 4, 5, 6, 7, 8, 9, or 10 times relative to a control. In some embodiments, the anti-EBV antigen antibody titer produced in a subject is increased 2-10 times relative to a control. For example, the anti-EBV antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the anti-EBV antigen antibody titer produced in a subject who has not been administered an EBV RNA (e.g., mRNA) vaccine. In some embodiments, a control is an anti-EBV antigen antibody titer produced in a subject administered a recombinant or purified EBV protein vaccine. Recombinant protein vaccines typically include protein antigens that either have been produced in a heterologous expression system (e.g., bacteria or yeast) or purified from large amounts of the pathogenic organism.

In some embodiments, the ability of an EBV vaccine to be effective is measured in a murine model. For example, the EBV vaccines may be administered to a murine model and the murine model assayed for induction of neutralizing antibody titers. Viral challenge studies may also be used to assess the efficacy of a vaccine of the present disclosure. For example, the EBV vaccines may be administered to a murine model, the murine model challenged with EBV, and the murine model assayed for survival and/or immune response (e.g., neutralizing antibody response, T cell response (e.g., cytokine response)).

In some embodiments, an effective amount of an EBV RNA (e.g., mRNA) vaccine is a dose that is reduced compared to the standard of care dose of a recombinant EBV protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified EBV protein vaccine, or a live attenuated or inactivated EBV vaccine, or an EBV VLP vaccine, that a physician/clinician or other medical professional would administer to a subject to treat or prevent EBV, or an EBV-related condition, while following the standard of care guideline for treating or preventing EBV, or an EBV-related condition.

In some embodiments, the anti-EBV antigen antibody titer produced in a subject administered an effective amount of an EBV RNA vaccine is equivalent to an anti-EBV antigen antibody titer produced in a control subject administered a standard of care dose of a recombinant or purified EBV protein vaccine, or a live attenuated or inactivated EBV vaccine, or an EBV VLP vaccine.

In some embodiments, an effective amount of an EBV RNA (e.g., mRNA) vaccine is a dose equivalent to an at least 2-fold reduction in a standard of care dose of a recombinant or purified EBV protein vaccine. For example, an effective amount of an EBV RNA vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified EBV protein vaccine. In some embodiments, an effective amount of an EBV RNA vaccine is a dose equivalent to an at least at least 100-fold, at least 500-fold, or at least 1000-fold reduction in a standard of care dose of a recombinant or purified EBV protein vaccine. In some embodiments, an effective amount of an EBV RNA vaccine is a dose equivalent to a 2-, 3-, 4-, 5-, 6-, 7-, 8-, 9-, 10-, 20-, 50-, 100-, 250-, 500-, or 1000-fold reduction in a standard of care dose of a recombinant or purified EBV protein vaccine. In some embodiments, the anti-EBV antigen antibody titer produced in a subject administered an effective amount of an EBV RNA vaccine is equivalent to an anti-EBV antigen antibody titer produced in a control subject administered the standard of care dose of a recombinant or protein EBV protein vaccine, or a live attenuated or inactivated EBV vaccine, or an EBV VLP vaccine. In some embodiments, an effective amount of an EBV RNA (e.g., mRNA) vaccine is a dose equivalent to a 2-fold to 1000-fold (e.g., 2-fold to 100-fold, 10-fold to 1000-fold) reduction in the standard of care dose of a recombinant or purified EBV protein vaccine, wherein the anti-EBV antigen antibody titer produced in the subject is equivalent to an anti-EBV antigen antibody titer produced in a control subject administered the standard of care dose of a recombinant or purified EBV protein vaccine, or a live attenuated or inactivated EBV vaccine, or an EBV VLP vaccine.

In some embodiments, the effective amount of an EBV RNA (e.g., mRNA) vaccine is a dose equivalent to a 2 to 1000-, 2 to 900-, 2 to 800-, 2 to 700-, 2 to 600-, 2 to 500-, 2 to 400-, 2 to 300-, 2 to 200-, 2 to 100-, 2 to 90-, 2 to 80-, 2 to 70-, 2 to 60-, 2 to 50-, 2 to 40-, 2 to 30-, 2 to 20-, 2 to 10-, 2 to 9-, 2 to 8-, 2 to 7-, 2 to 6-, 2 to 5-, 2 to 4-, 2 to 3-, 3 to 1000-, 3 to 900-, 3 to 800-, 3 to 700-, 3 to 600-, 3 to 500-, 3 to 400-, 3 to 3 to 00-, 3 to 200-, 3 to 100-, 3 to 90-, 3 to 80-, 3 to 70-, 3 to 60-, 3 to 50-, 3 to 40-, 3 to 30-, 3 to 20-, 3 to 10-, 3 to 9-, 3 to 8-, 3 to 7-, 3 to 6-, 3 to 5-, 3 to 4-, 4 to 1000-, 4 to 900-, 4 to 800-, 4 to 700-, 4 to 600-, 4 to 500-, 4 to 400-, 4 to 300-, 4 to 200-, 4 to 100-, 4 to 90-, 4 to 80-, 4 to 70-, 4 to 60-, 4 to 50-, 4 to 40-, 4 to 30-, 4 to 20-, 4 to 10-, 4 to 9-, 4 to 8-, 4 to 7-, 4 to 6-, 4 to 5-, 4 to 4-, 5 to 1000-, 5 to 900-, 5 to 800-, 5 to 700-, 5 to 600-, 5 to 500-, 5 to 400-, 5 to 300-, 5 to 200-, 5 to 100-, 5 to 90-, 5 to 80-, 5 to 70-, 5 to 60-, 5 to 50-, 5 to 40-, 5 to 30-, 5 to 20-, 5 to 10-, 5 to 9-, 5 to 8, 5 to 7-, 5 to 6-, 6 to 1000-, 6 to 900-, 6 to 800-, 6 to 700-, 6 to 600-, 6 to 500-, 6 to 400-, 6 to 300-, 6 to 200-, 6 to 100-, 6 to 90-, 6 to 80-, 6 to 70-, 6 to 60-, 6 to 50-, 6 to 40-, 6 to 30-, 6 to 20-, 6 to 10-, 6 to 9-, 6 to 8-, 6 to 7-, 7 to 1000-, 7 to 900-, 7 to 800-, 7 to 700-, 7 to 600-, 7 to 500-, 7 to 400-, 7 to 300-, 7 to 200-, 7 to 100-, 7 to 90-, 7 to 80-, 7 to 70-, 7 to 60-, 7 to 50-, 7 to 40-, 7 to 30-, 7 to 20-, 7 to 10-, 7 to 9-, 7 to 8-, 8 to 1000-, 8 to 900-, 8 to 800-, 8 to 700-, 8 to 600-, 8 to 500-, 8 to 400-, 8 to 300-, 8 to 200-, 8 to 100-, 8 to 90-, 8 to 80-, 8 to 70-, 8 to 60-, 8 to 50-, 8 to 40-, 8 to 30-, 8 to 20-, 8 to 10-, 8 to 9-, 9 to 1000-, 9 to 900-, 9 to 800-, 9 to 700-, 9 to 600-, 9 to 500-, 9 to 400-, 9 to 300-, 9 to 200-, 9 to 100-, 9 to 90-, 9 to 80-, 9 to 70-, 9 to 60-, 9 to 50-, 9 to 40-, 9 to 30-, 9 to 20-, 9 to 10-, 10 to 1000-, 10 to 900-, 10 to 800-, 10 to 700-, 10 to 600-, 10 to 500-, 10 to 400-, 10 to 300-, 10 to 200-, 10 to 100-, 10 to 90-, 10 to 80-, 10 to 70-, 10 to 60-, 10 to 50-, 10 to 40-, 10 to 30-, 10 to 20-, 20 to 1000-, 20 to 900-, 20 to 800-, 20 to 700-, 20 to 600-, 20 to 500-, 20 to 400-, 20 to 300-, 20 to 200-, 20 to 100-, 20 to 90-, 20 to 80-, 20 to 70-, 20 to 60-, 20 to 50-, 20 to 40-, 20 to 30-, 30 to 1000-, 30 to 900-, 30 to 800-, 30 to 700-, 30 to 600-, 30 to 500-, 30 to 400-, 30 to 300-, 30 to 200-, 30 to 100-, 30 to 90-, 30 to 80-, 30 to 70-, 30 to 60-, 30 to 50-, 30 to 40-, 40 to 1000-, 40 to 900-, 40 to 800-, 40 to 700-, 40 to 600-, 40 to 500-, 40 to 400-, 40 to 300-, 40 to 200-, 40 to 100-, 40 to 90-, 40 to 80-, 40 to 70-, 40 to 60-, 40 to 50-, 50 to 1000-, 50 to 900-, 50 to 800-, 50 to 700-, 50 to 600-, 50 to 500-, 50 to 400-, 50 to 300-, 50 to 200-, 50 to 100-, 50 to 90-, 50 to 80-, 50 to 70-, 50 to 60-, 60 to 1000-, 60 to 900-, 60 to 800-, 60 to 700-, 60 to 600-, 60 to 500-, 60 to 400-, 60 to 300-, 60 to 200-, 60 to 100-, 60 to 90-, 60 to 80-, 60 to 70-, 70 to 1000-, 70 to 900-, 70 to 800-, 70 to 700-, 70 to 600-, 70 to 500-, 70 to 400-, 70 to 300-, 70 to 200-, 70 to 100-, 70 to 90-, 70 to 80-, 80 to 1000-, 80 to 900-, 80 to 800-, 80 to 700-, 80 to 600-, 80 to 500-, 80 to 400-, 80 to 300-, 80 to 200-, 80 to 100-, 80 to 90-, 90 to 1000-, 90 to 900-, 90 to 800-, 90 to 700-, 90 to 600-, 90 to 500-, 90 to 400-, 90 to 300-, 90 to 200-, 90 to 100-, 100 to 1000-, 100 to 900-, 100 to 800-, 100 to 700-, 100 to 600-, 100 to 500-, 100 to 400-, 100 to 300-, 100 to 200-, 200 to 1000-, 200 to 900-, 200 to 800-, 200 to 700-, 200 to 600-, 200 to 500-, 200 to 400-, 200 to 300-, 300 to 1000-, 300 to 900-, 300 to 800-, 300 to 700-, 300 to 600-, 300 to 500-, 300 to 400-, 400 to 1000-, 400 to 900-, 400 to 800-, 400 to 700-, 400 to 600-, 400 to 500-, 500 to 1000-, 500 to 900-, 500 to 800-, 500 to 700-, 500 to 600-, 600 to 1000-, 600 to 900-, 600 to 800-, 600 to 700-, 700 to 1000-, 700 to 900-, 700 to 800-, 800 to 1000-, 800 to 900-, or 900 to 1000-fold reduction in the standard of care dose of a recombinant EBV protein vaccine. In some embodiments, such as the foregoing, the anti-EBV antigen antibody titer produced in the subject is equivalent to an anti-EBV antigen antibody titer produced in a control subject administered the standard of care dose of a recombinant or purified EBV protein vaccine, or a live attenuated or inactivated EBV vaccine, or an EBV VLP vaccine. In some embodiments, the effective amount is a dose equivalent to (or equivalent to an at least) 2-, 3-, 4-, 5-, 6-, 7-, 8-, 9-, 10-, 20-, 30-, 40-, 50-, 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 460-, 470-, 480-, 490-, 500-, 510-, 520-, 530-, 540-, 550-, 560-, 570-, 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820-, 830-, 840-, 850-, 860-, 870-, 880-, 890-, 900-, 910-, 920-, 930-, 940-, 950-, 960-, 970-, 980-, 990-, or 1000-fold reduction in the standard of care dose of a recombinant EBV protein vaccine. In some embodiments, such as the foregoing, an anti-EBV antigen antibody titer produced in the subject is equivalent to an anti-EBV antigen antibody titer produced in a control subject administered the standard of care dose of a recombinant or purified EBV protein vaccine, or a live attenuated or inactivated EBV vaccine, or an EBV VLP vaccine.

In some embodiments, the effective amount of an EBV RNA (e.g., mRNA) vaccine is a total dose of 50-1000 μg. In some embodiments, the effective amount of an EBV RNA (e.g., mRNA) vaccine is a total dose of 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-900, 600-700, 700-1000, 700-900, 700-800, 800-1000, 800-900, or 900-1000 μg. In some embodiments, the effective amount of an EBV RNA (e.g., mRNA) vaccine is a total dose of 50, 100, 150, 200, 250, 300, 350, 400, 450, 500, 550, 600, 650, 700, 750, 800, 850, 900, 950 or 1000 μg. In some embodiments, the effective amount is a dose of 25-500 μg administered to the subject a total of two times. In some embodiments, the effective amount of an EBV RNA (e.g., mRNA) vaccine is a dose of 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-200, 200-500, 200-400, 200-300, 250-500, 250-400, 250-300, 300-500, 300-400, 350-500, 350-400, 400-500 or 450-500 μg administered to the subject a total of two times. In some embodiments, the effective amount of an EBV RNA (e.g., mRNA) vaccine is a total dose of 25, 50, 100, 150, 200, 250, 300, 350, 400, 450, or 500 μg administered to the subject a total of two times.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of the EBV vaccine is at least 60% relative to unvaccinated control subjects. For example, efficacy of the EBV vaccine may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of an EBV vaccine of the present disclosure is sufficient to provide sterilizing immunity in the subject for at least 1 year. For example, the effective amount of an EBV vaccine of the present disclosure is sufficient to provide sterilizing immunity in the subject for at least 2 years, at least 3 years, at least 4 years, or at least 5 years. In some embodiments, the effective amount of an EBV vaccine of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of an EBV vaccine of the present disclosure is sufficient to produce detectable levels of EBV antigen as measured in serum of the subject at 1-72 hours post administration.

Titer. An antibody titer is a measurement of the amount of antibodies within a subject, for example, antibodies that are specific to a particular antigen (e.g., an anti-EBV antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of an EBV vaccine of the present disclosure is sufficient to produce a 1,000-10,000 neutralizing antibody titer produced by neutralizing antibody against the EBV antigen as measured in serum of the subject at 1-72 hours post administration. In some embodiments, the effective amount is sufficient to produce a 1,000-5,000 neutralizing antibody titer produced by neutralizing antibody against the EBV antigen as measured in serum of the subject at 1-72 hours post administration. In some embodiments, the effective amount is sufficient to produce a 5,000-10,000 neutralizing antibody titer produced by neutralizing antibody against the EBV antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-EBV antigen antibody titer produced in the subject is increased by at least 1 log relative to a control. For example, an anti-EBV antigen antibody titer produced in the subject may be increased by at least 2, 3, 4, 5, 6, 7, 8, 9 or 10 log relative to a control.

In some embodiments, an anti-EBV antigen antibody titer produced in the subject is increased at least 2 times relative to a control. For example, an anti-EBV antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated EBV vaccine, an inactivated EBV vaccine, or a protein subunit EBV vaccine.

### EXAMPLES

### Example 1

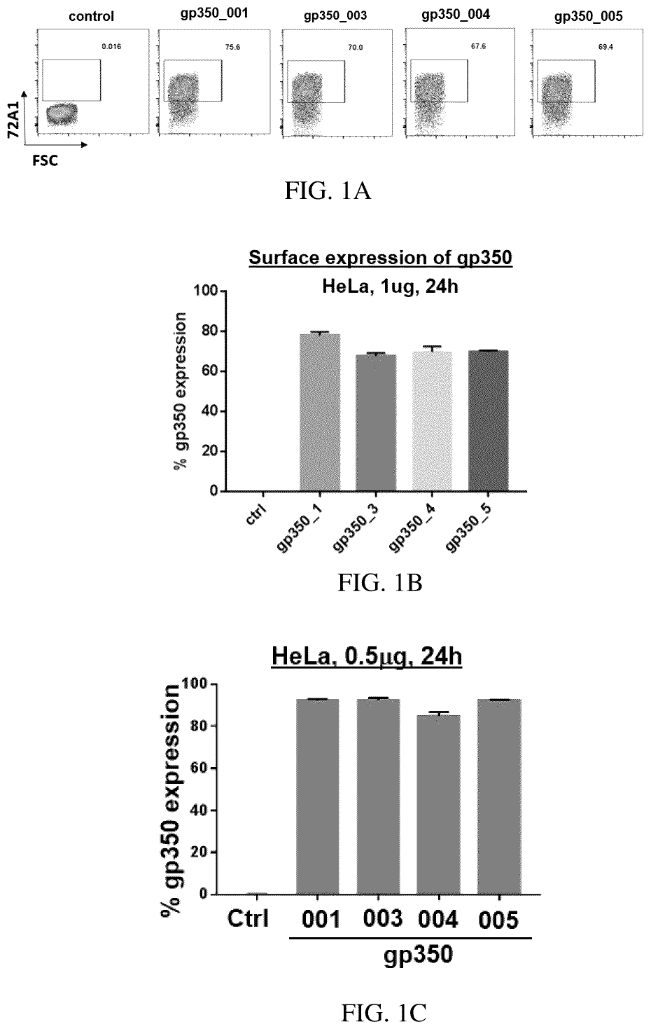

EBV glycoprotein 350 (gp350) variants were produced and their expression tested in HeLa cells. HeLa cells were transiently transfected for 24 hours with mRNA encoding each of EBV glycoprotein 350 (gp350) variants (SEQ ID NOs: 185, 182, 207, and 208). Flow cytometry analyses (FIG. 1A, 1 μg dose mRNA) and immunoassays (FIG. 1B, 1 μg dose mRNA; FIG. 1C, 0.5 μg dose mRNA) using an EBV neutralizing antibody that binds conformational epitopes in gp350 (“72A1”) demonstrate that all EBV gp350 variants tested show equivalent expression at the surface of the transfected HeLa cells. Unless otherwise stated, all mRNA vaccines may be formulated in lipid nanoparticles comprising Compound 1 lipids, e.g., 20-60% ionizable cationic lipid, 5-25% non-cationic lipid, 25-55% sterol, and 0.5-15% PEG-modified lipid.

### Example 2

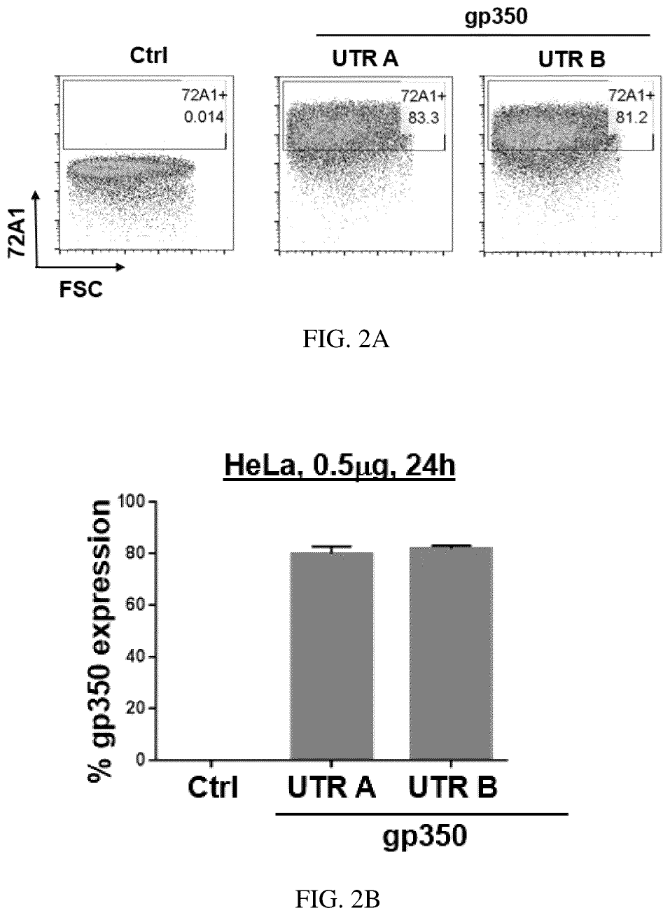

EBV gp350 mRNA sequences with two different 5′ UTR sequences (SEQ ID NOs: 81 and 204) were produced and their expression tested in HeLa cells. HeLa cells were transiently transfected for 24 hours with a 0.5 μg dose of mRNA encoding the EBV gp350 antigens. Flow cytometry analyses (FIG. 2A) and immunoassays (FIG. 2B) using the anti-72A1 antibody demonstrate that both EBV gp350 mRNA constructs tested show equivalent expression at the surface of the transfected HeLa cells.

### Example 3

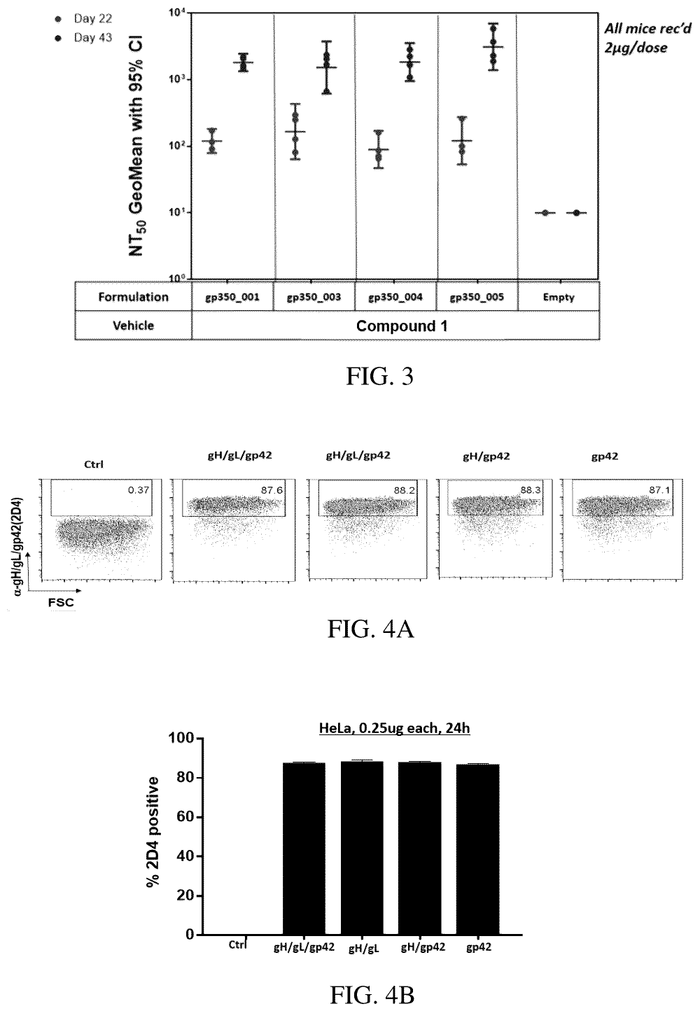

Balb/c mice were vaccinated intramuscularly with EBV vaccines comprising mRNA encoding EBV gp350 variants (SEQ ID NOs: 185, 182, 207, and 208) formulated in a lipid nanoparticle. A 2 μg dose was administered on Day 1 and then again on Day 22. Mice were bled on Day 21 and Day 43. Results demonstrate that all the EBV gp350 vaccines tested induced serum gp350-specific IgG antibody titers at day 21 (3 weeks post prime) and day 43 (3 weeks post boost) following vaccination (FIG. 3).

### Example 4

Additional EBV antigens and antigen complexes were produced and their expression tested in HeLa cells. HeLa cells were transiently transfected for 24 hours with 0.25 μg of (1) mRNA encoding EBV gH (SEQ ID NO: 187), mRNA encoding EBV gL (SEQ ID NO: 188), and mRNA encoding EBV gp42 (SEQ ID NO: 189) (EBV gH/gL/gp42 UTR A); (2) mRNA encoding EBV gH (SEQ ID NO: 201), mRNA encoding EBV gL (SEQ ID NO: 202), and mRNA encoding EBV gp42 (SEQ ID NO: 203) (EBV gH/gL/gp42 UTR B); (3) mRNA encoding EBV gH (SEQ ID NO: 187) and mRNA encoding gp42 (SEQ ID NO:189); or (4) mRNA encoding EBV gp42 (SEQ ID NO:189). Flow cytometry analyses (FIG. 4A) and immunoassays (FIG. 4B) using an anti-gH/gL/gp42 (2D4) antibody demonstrate that all EBV mRNA constructs tested show equivalent expression at the surface of the transfected HeLa cells.

### Example 5

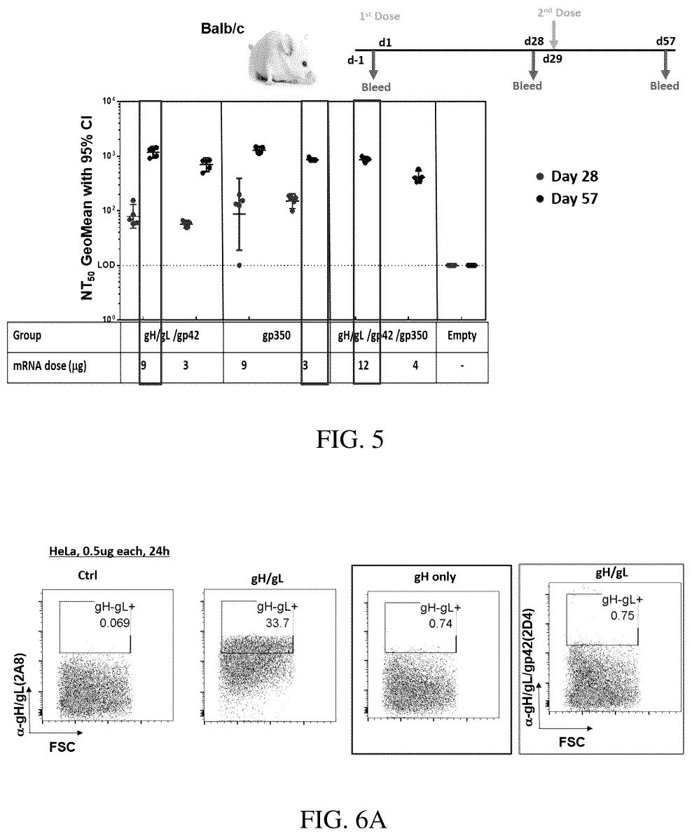

Balb/c mice were vaccinated intramuscularly with EBV vaccines comprising (1) mRNA encoding EBV gH (SEQ ID NO: 187), mRNA encoding EBV gL (SEQ ID NO: 188), and mRNA encoding EBV gp42 (SEQ ID NO: 189); (2) mRNA encoding EBV gp350 (SEQ ID NO: 185); or (3) mRNA encoding EBV gH (SEQ ID NO: 187), mRNA encoding EBV gL (SEQ ID NO: 188), mRNA encoding EBV gp42 (SEQ ID NO: 189), and mRNA encoding EBV gp350 (SEQ ID NO: 185). A 2 μg dose was administered on Day 1 and then again on Day 29. Mice were bled on Day 28 and Day 57. Results demonstrate that all the EBV vaccines tested induced neutralizing antibody titers at day 28 (4 weeks post prime) and day 57 (4 weeks post boost) following vaccination (FIG. 5).

### Example 6

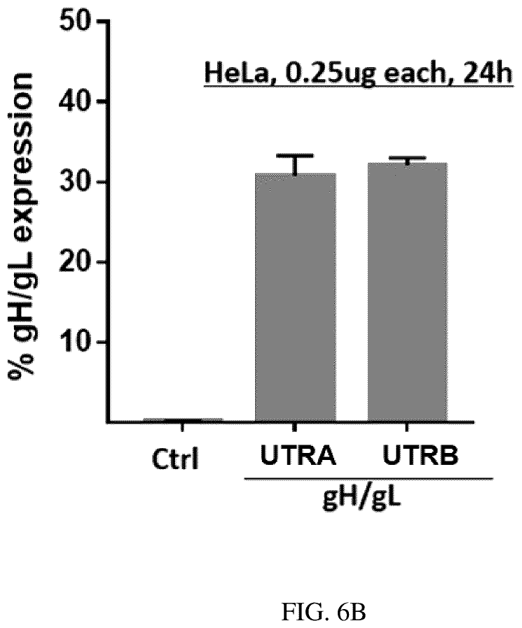

HeLa cells were transiently transfected for 24 hours with 0.5 μg of (1) mRNA encoding EBV gH (SEQ ID NO: 201) and mRNA encoding EBV gL (SEQ ID NO: 202); or (2) mRNA encoding EBV gH only (SEQ ID NO: 201). Flow cytometry analyses (FIG. 6A) using an anti-gH/gL (2A8) antibody or an anti-gH/gL/gp42 (2D4) antibody demonstrate that the 2A8 antibody binds specifically to EBV gL expressed on the surface of the HeLa cells (compare to data presented in Example 4). Immunoassays following 24 hour transfection with 0.25 μg of either (1) mRNA encoding EBV mRNA encoding EBV gH (SEQ ID NO: 187) and mRNA encoding EBV gL (SEQ ID NO: 188); or (2) mRNA encoding EBV mRNA encoding EBV gH (SEQ ID NO: 201) and mRNA encoding EBV gL (SEQ ID NO: 202) show comparable expression of both EBV antigen complexes at the surface of HeLa cells.

### Example 7

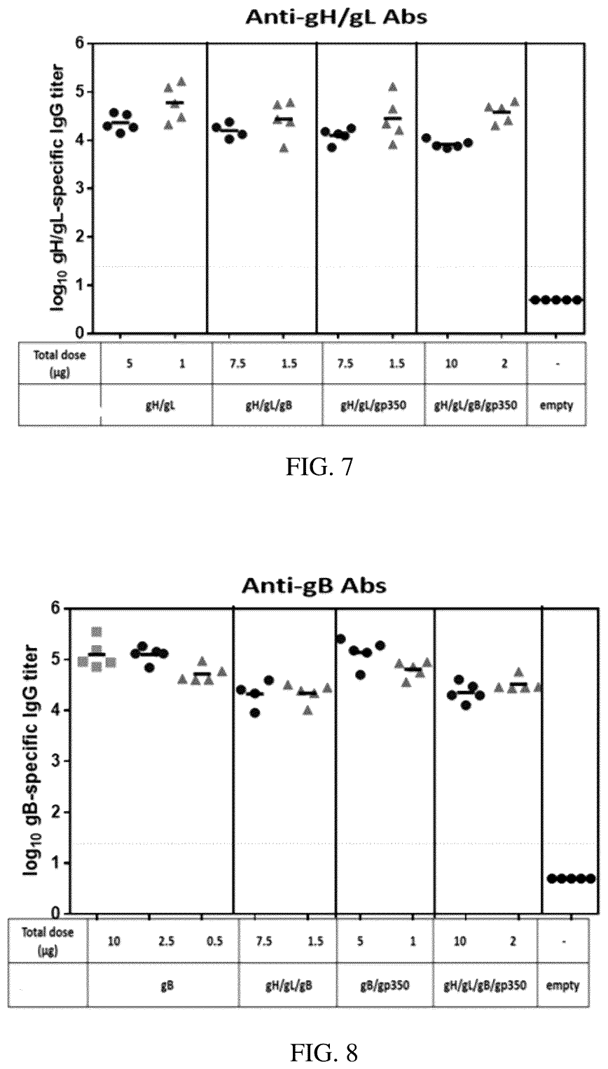

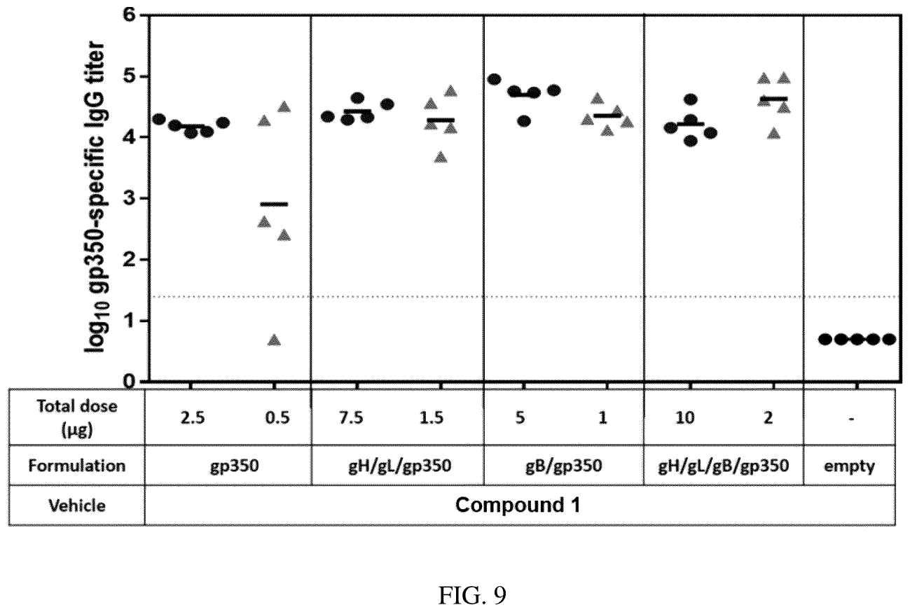

Three additional immunogenicity studies were performed using various mRNA vaccines of the present disclosure (formulated in lipid nanoparticles) and various antigen-specific antibodies. Balb/c mice were vaccinated intramuscularly with (1) a 5 μg dose or a 1 μg dose of mRNA encoding EBV gH (SEQ ID NO: 187) and mRNA encoding EBV gL (SEQ ID NO: 188); (2) a 7.5 μg dose or a 1.5 μg dose of mRNA encoding EBV gH (SEQ ID NO: 187), mRNA encoding EBV gL (SEQ ID NO: 188), and mRNA encoding EBV gB (SEQ ID NO: 209); (3) a 7.5 μg dose or a 1.5 μg dose of mRNA encoding EBV gH (SEQ ID NO: 187), mRNA encoding EBV gL (SEQ ID NO: 188), and mRNA encoding EBV gp350 (SEQ ID NO: 185); or (4) a 10 μg dose or a 2 μg dose of mRNA encoding EBV gH (SEQ ID NO: 187), mRNA encoding EBV gL (SEQ ID NO: 188), mRNA encoding EBV gB (SEQ ID NO: 209), and mRNA encoding EBV gp350 (SEQ ID NO: 185). Mice were bled on Day 57 Results following detection with anti-gH/gL antibody (FIG. 7), anti-gB antibody (FIG. 8), or anti-gp350 antibody (FIG. 9) demonstrate induction of EBV antigen-specific neutralizing antibodies.

### Example 8

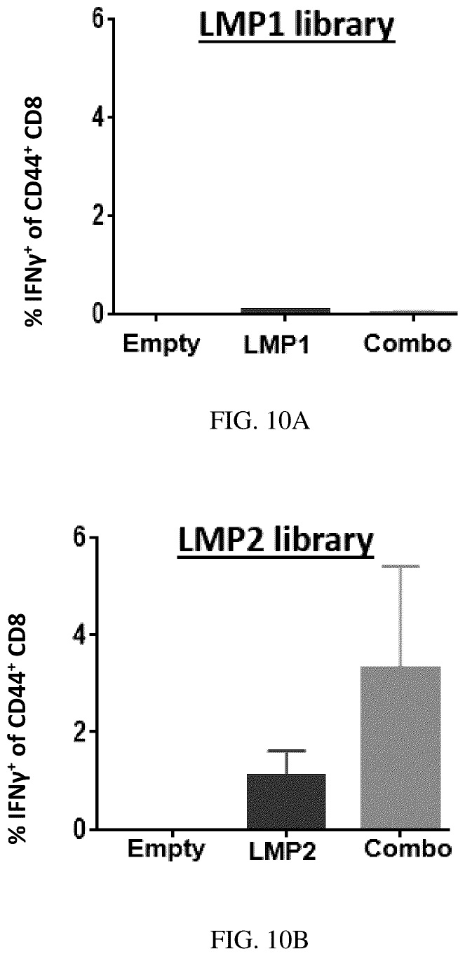

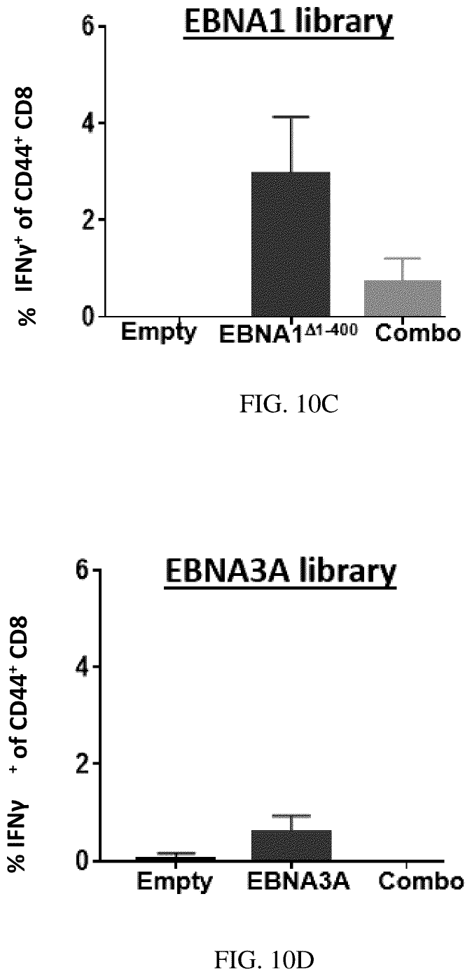

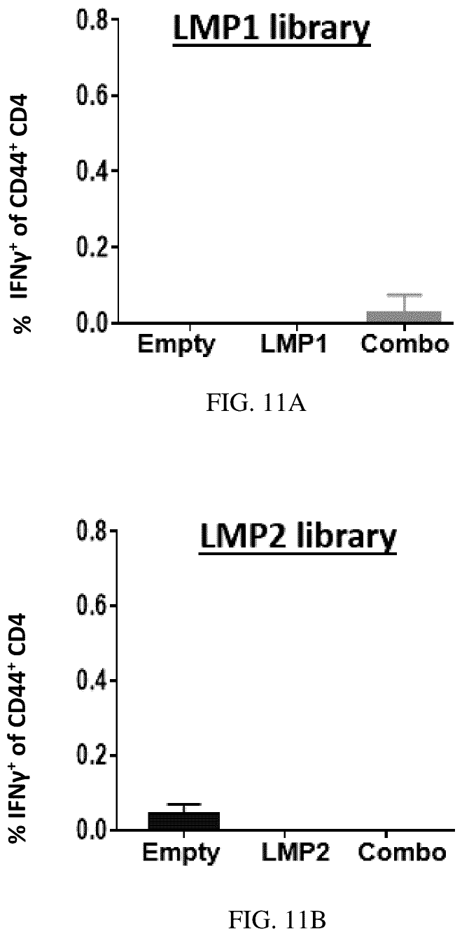

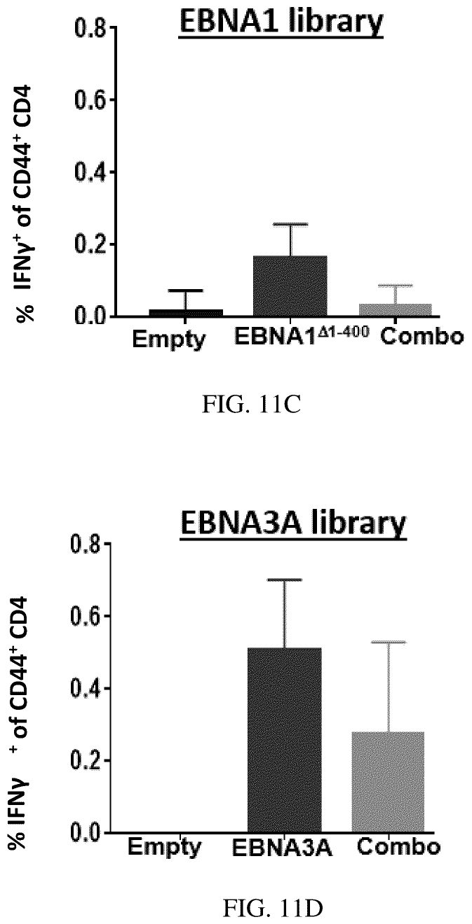

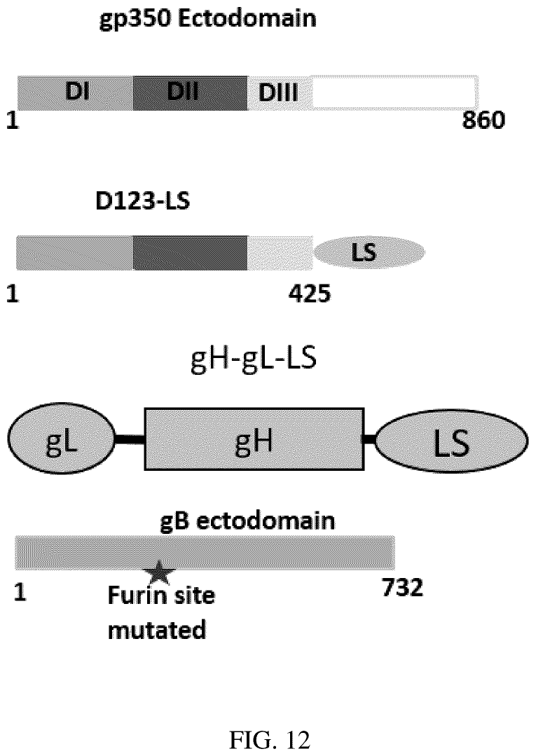

Mice were vaccinated with a 2 μg dose of mRNA encoding one of four EBV latent genes (LMP1 (SEQ ID NO: 179), LMP2 (SEQ ID NO: 181), EBNA1^{Δ1-400} (SEQ ID NO: 178) or EBNA3A (SEQ ID NO: 177)) or a combination of all four mRNA vaccines (LMP1 (SEQ ID NO: 179), LMP2 (SEQ ID NO: 181), EBNA1^{Δ1-400} (SEQ ID NO: 178) and EBNA3 Å (SEQ ID NO: 177). Cells were harvested from vaccinated mice and stimulated with LMP1, LMP2, EBNA1 or EBNA3A peptides. All peptide libraries comprise 15mer peptides overlapping by 11 amino acids. CD8 T cell responses are shown in FIGS. 10A-10D, and CD4 T cell responses are shown in FIGS. 11A-11D.

### Example 9

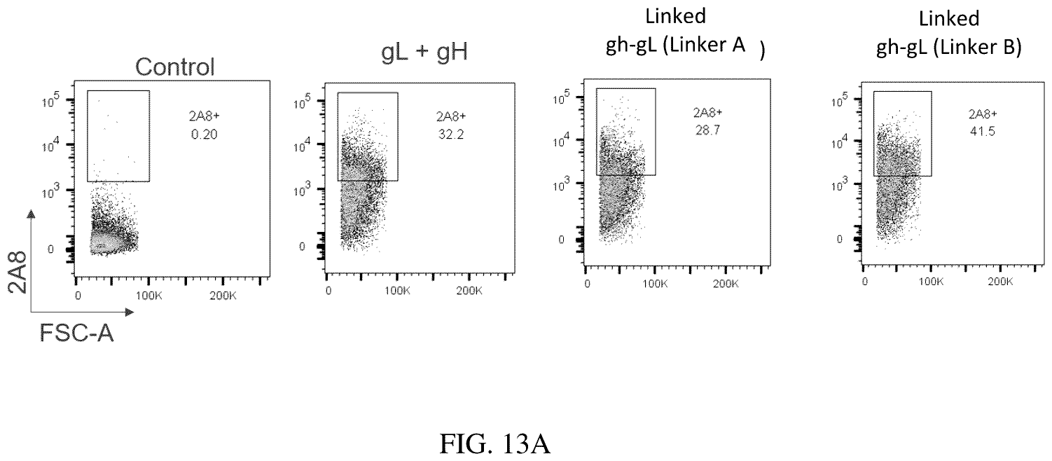

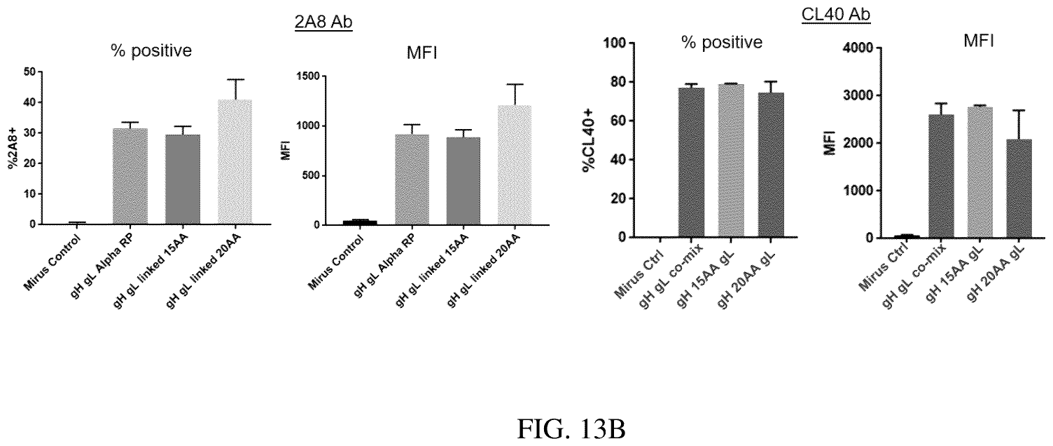

mRNA encoding EBV glycoprotein H-glycoprotein L (gH-gL) linked constructs (SEQ ID NO: 218 or 221) were produced and their expression tested in HeLa cells. HeLa cells were transiently transfected for 24 hours with mRNA encoding two EBV gH-gL variants with different linkers (SEQ ID NO: 218 or SEQ ID NO: 221), or with mRNA encoding EBV gH (EBV gH mRNA; SEQ ID NO: 228) and mRNA encoding EBV gL (EBV gL mRNA; SEQ ID NO: 229). Flow cytometry analyses (FIG. 13A, 0.5 μg dose of EBV gH-gL linked mRNA or 0.25 μg dose of each of EBV gH mRNA and EBV gL mRNA) and immunoassays (FIG. 13B; 0.5 μg dose of gH-gL linked construct or 0.25 μg dose of each of gH and gL) using EBV neutralizing antibodies 2A8 and CL40, which binds conformational epitopes in gH, demonstrate that both EBV gH-gL constructs (SEQ ID NO: 218 and 221) tested show expression at the surface of the transfected HeLa cells.

### Example 10

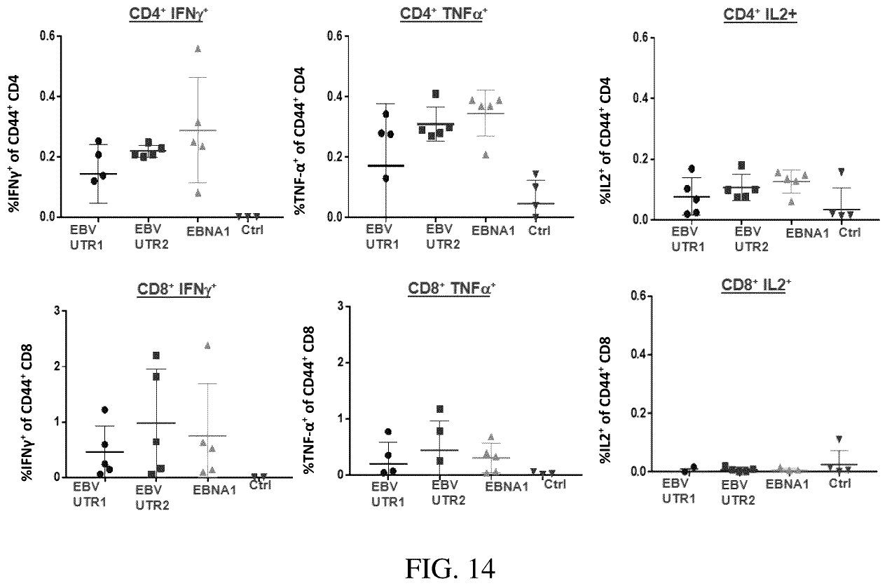

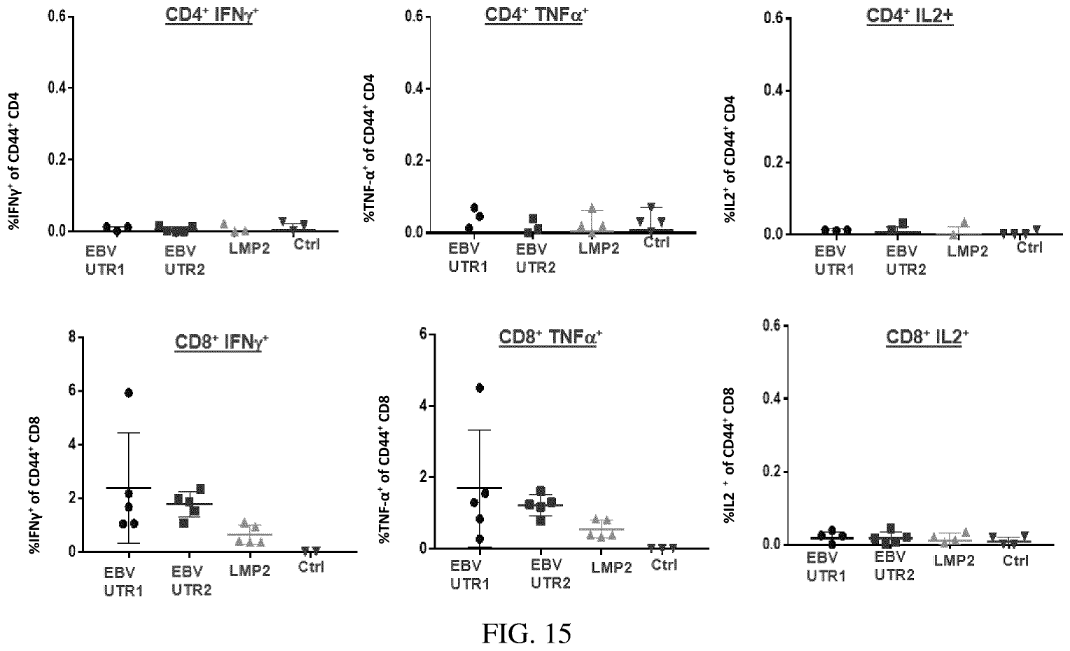

In a further immunogenicity study to test the effect of different untranslated regions (UTRs), mice were vaccinated intramuscularly with lipid nanoparticles (comprising Compound 1 lipids, e.g., 20-60% ionizable cationic lipid, 5-25% non-cationic lipid, 25-55% sterol, and 0.5-15% PEG-modified lipid) comprising (1) 10 μg mRNA encoding gp350 (SEQ ID NO: 185), mRNA encoding gH (SEQ ID NO: 187), mRNA encoding gL (SEQ ID NO: 188), mRNA encoding LMP2 antigen (SEQ ID NO: 181), and mRNA encoding EBNA1 antigen (SEQ ID NO: 178), each of the transcripts comprising UTRA; (2) 10 μg mRNA encoding gp350 (SEQ ID NO: 185), mRNA encoding gH (SEQ ID NO: 187), mRNA encoding gL (SEQ ID NO: 188), mRNA encoding LMP2 antigen (SEQ ID NO: 181), and mRNA encoding EBNA1 antigen (SEQ ID NO: 178), each of the transcripts comprising UTRB; (3) 2 μg mRNA encoding EBNA1 antigen (SEQ ID NO: 178) with UTRB; (4) 2 μg mRNA encoding LMP2 antigen with UTRB; or (5) empty lipid nanoparticles. The mice received one dose on Day 1 and a second dose on Day 29. Blood samples were taken just prior to dosing, and on Day 57. Spleens were collected from a subset of animals on Day 36. Cells were harvested from vaccinated mice and stimulated with peptides from an EBNA1 peptide library (FIG. 14) or an LMP2 library (FIG. 15). The peptide library comprised 15mer peptides overlapping by 11 amino acids. CD4 T cell cytokine responses for the EBV formulations (groups 1 and 2), the EBNA1 formulation (group 3), and the control (empty nanoparticles; group 5) are shown in the top row of FIG. 14; the bottom row of FIG. 14 shows CD8 T cell responses. FIG. 15 shows the CD4 T cell responses (top row) and CD8 T cell cytokine responses (bottom row) for EBV formulations (groups 1 and 2), the LMP2 formulation (group 4), and the control (empty nanoparticles; group 5). Similar T cell responses were found between groups regardless of the UTR used.

### Example 11

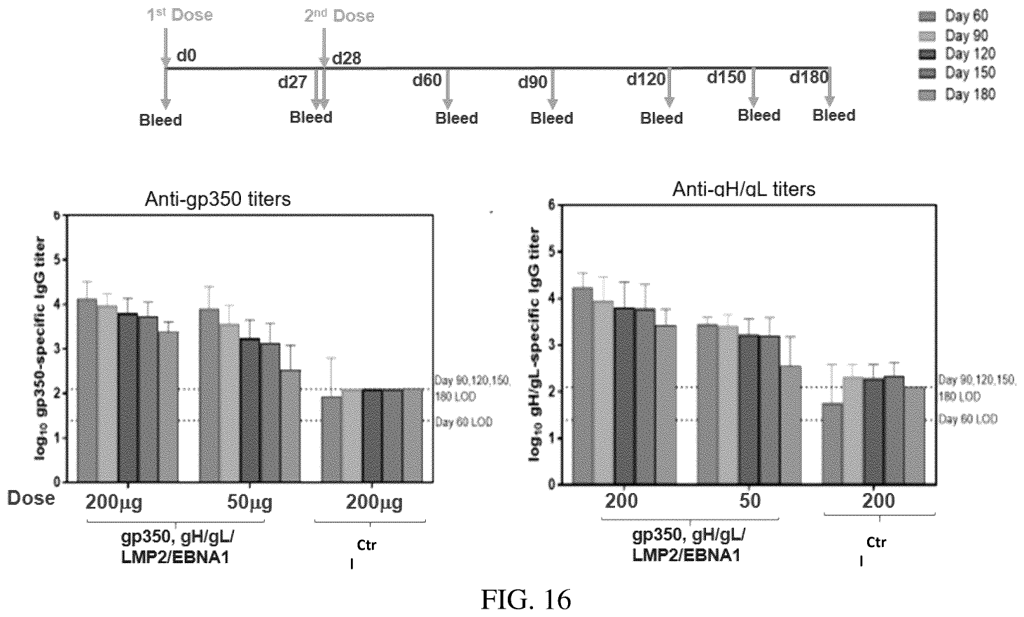

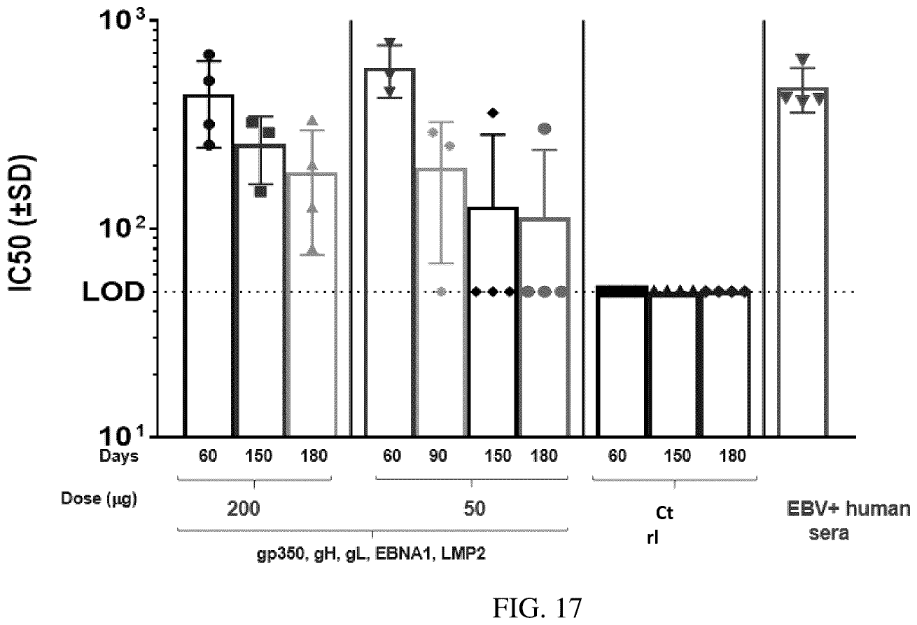

The constructs were tested in a non-human primate (Rhesus macaque) model. The subjects were vaccinated intramuscularly with lipid nanoparticles comprising (1) 200 μg of mRNA encoding gp350 (SEQ ID NO: 185), mRNA encoding gH (SEQ ID NO: 187), mRNA encoding gL (SEQ ID NO: 188), mRNA encoding LMP2 antigen (SEQ ID NO: 181), and mRNA encoding EBNA1 antigen (SEQ ID NO: 178); (2) 50 μg of mRNA encoding gp350 (SEQ ID NO: 185), mRNA encoding gH (SEQ ID NO: 187), mRNA encoding gL (SEQ ID NO: 188), mRNA encoding LMP2 antigen (SEQ ID NO: 181), and mRNA encoding EBNA1 antigen (SEQ ID NO: 178); or (3) 200 μg of mRNA encoding a control. As shown in FIG. 16, the subjects received one dose on day 0 and a second dose on day 28. Blood samples were drawn on days 0, 27, 28, 60, 90, 120, 150, and 180. Results following detection with anti-gp350 titer and anti-gH/gL antibody demonstrate that vaccination with the selected formulations results in increased and durable anti-gp350 and anti-gH/gL antibody titers (FIG. 16). While neutralizing antibody titers were found to be durable at high doses, a significant drop in neutralizing antibody titers at low doses of the EBV vaccine was observed (FIG. 17).

### Example 12

The effect of various downstream processes on EBV vaccine immunogenicity was examined. EBV vaccines comprising gp350 (SEQ ID NO: 185), gH (SEQ ID NO: 187), and gL (SEQ ID NO: 188) were synthesized using different downstream purification processes.

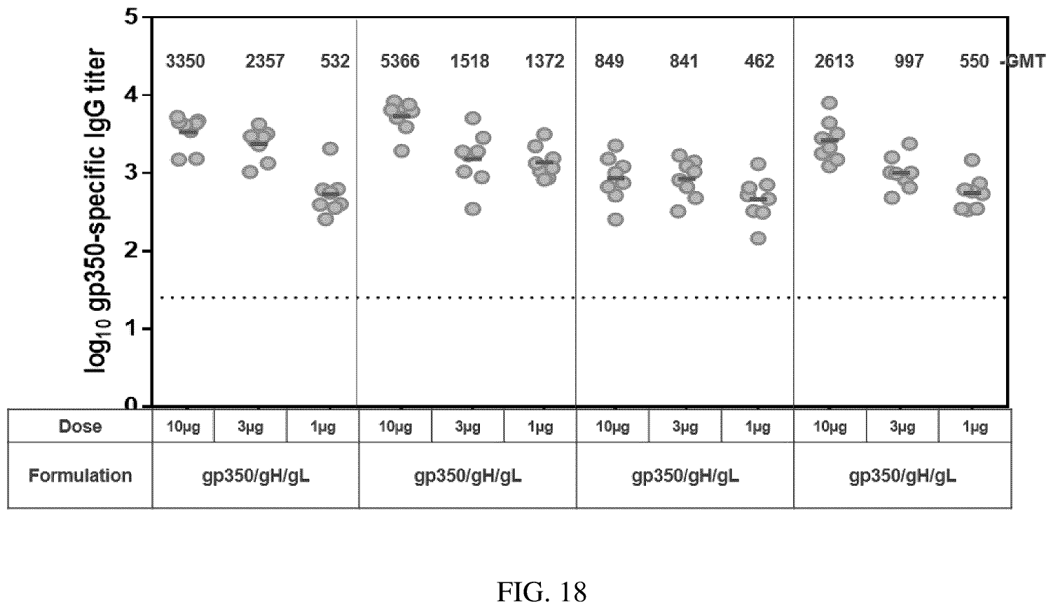

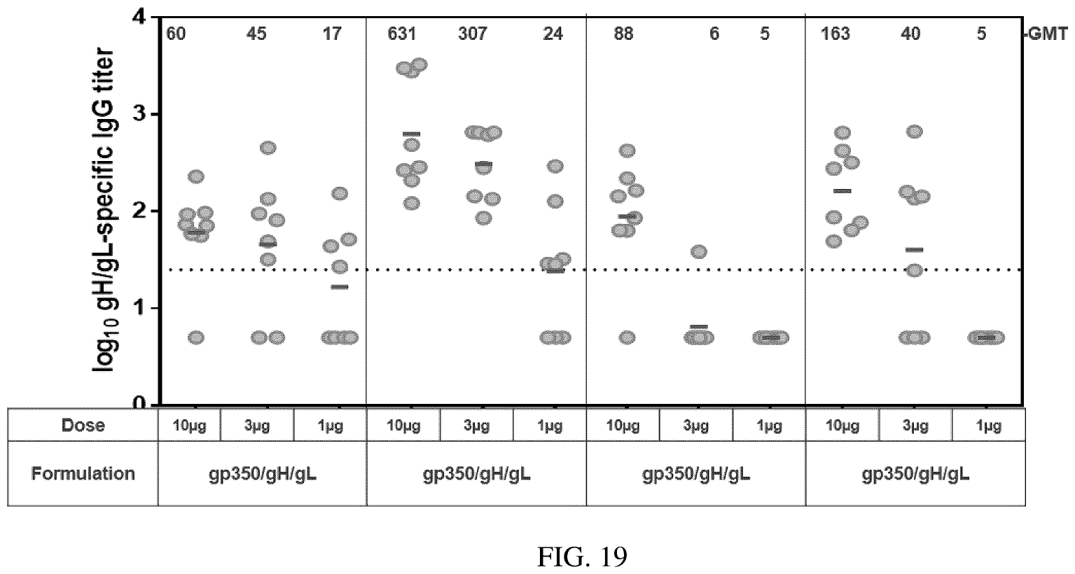

The resulting mRNA was used to form doses (10 μg, 3 μg, and 1 μg doses for each of the four groups; PBS was used as a control group; n=8/group) that were administered to mice on Days 1 and 22. Blood samples were collected on Days 1, 21, 22, 36, 82, and 142. Antibody titers from gp250 (FIG. 18) and for gH/gL (FIG. 19) were measured.

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

The entire contents of International Application Nos. PCT/US2015/02740, PCT/US2016/043348, PCT/US2016/043332, PCT/US2016/058327, PCT/US2016/058324, PCT/US2016/058314, PCT/US2016/058310, PCT/US2016/058321, PCT/US2016/058297, PCT/US2016/058319, and PCT/US2016/058314 are incorporated herein by reference.

### SEQUENCE LISTING

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a polyA tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

(SEQ ID NO: 1)
5′ UTR: GGGAAAUAAGAGAGAAAAGAAGAGUAAGAAGAAAUAUA
AGAGCCACC
(SEQ ID NO: 104)
5′ UTR: GGGAAAUAAGAGAGAAAAGAAGAGUAAGAAGAAAUAUA
AGACCCCGGCGCCGCCACC
(SEQ ID NO: 3)
3′ UTR: UGAUAAUAGGCUGGAGCCUCGGUGGCCAUGCUUCUUGC
CCCUUGGGCCUCCCCCCAGCCCCUCCUCCCCUUCCUGCACCCGUAC
CCCCGUGGUCUUUGAAUAAAGUCUGAGUGGGCGGC
(SEQ ID NO: 106)
3′ UTR: UGAUAAUAGGCUGGAGCCUCGGUGGCCUAGCUUCUUGC
CCCUUGGGCCUCCCCCCAGCCCCUCCUCCCCUUCCUGCACCCGUAC
CCCCGUGGUCUUUGAAUAAAGUCUGAGUGGGCGGC

SEQ
ID
NO:

EBNA MO1

| SEQ ID NO: 130 consists of from 5′ end to 3′ end, | | 130 |
|---|---|---|
| 5′ UTR SEQ ID NO: 1, mRNA ORF SEQ ID NO: 2, and | | |
| 3′ UTR SEQ ID NO: 3. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGAGCGACGAGGGUCCGGGCACAGGACCCGGUAACGGG | 2 |
| Construct | UUGGGCGAAAAGGGUGACACUAGCGGGCCCGAAGGUUCC | |
| (excluding | GGCGGAUCUGGCCCGCAACGCAGAGGUGGUGAUAACCAC | |
| the stop | GGUCGCGGUAGAGGCAGAGGCCGUGGCCGGGGUGGCGGA | |
| codon) | AGGCCCGGUGCCCCUGGAGGGUCAGGGUCUGGCCCAAGG | |
| | CACCGUGAUGGGGUGCGUCGCCCCCAGAAACGCCCGUCCU | |
| | GUAUCGGCUGCAAGGGCACGCAUGGCGGAACGGGAGGUG | |
| | GCGCAGGAGCCGGAGGUGCAAGAGGUCGCGGGGGGUCAG | |
| | GGGGGAGAGGUAGAGGGGGAUCAGGGGGGCGGAGAGGGC | |
| | GGGGUCGAGAAAGAGCAAGAGGUGGCUCCAGAGAGCGCG | |
| | CUCGGGGUCGCGGGCGAGGCCGGGGUGAAAAACGGCCCC | |
| | GGAGCCCAUCCUCGCAAUCAUCAAGCUCGGGAUCGCCACC | |
| | UCGGCGGCCGCCCCCCGGCAGGCGUCCCUUUUUCCACCCA | |
| | GUGGGCGAAGCCGACUACUUCGAGUACCACCAGGAGGGA | |
| | GGACCGGAUGGUGAGCCGGAUGUUCCUCCCGGCGCCAUC | |
| | GAACAAGGUCCUGCCGAUGAUCCAGGCGAGGGGCCCUCA | |
| | ACAGGCCCCCGAGGGCAGGGGGACGGGGGACGUCGGAAG | |
| | AAAGGAGGCUGGUUUGGCAAGCACCGAGGACAGGGGGGU | |
| | AGCAACCCGAAAUUUGAGAACAUCGCCGAGGGGCUCAGG | |
| | GCUCUCCUAGCCCGUAGCCACGUCGAAAGAACUACCGAU | |
| | GAGGGCACCUGGGUUGCCGGCGUGUUCGUGUACGGGGGU | |
| | AGCAAGACCUCUCUAUACAACCUUCGGAGGGGGACUGCA | |
| | CUUGCUAUCCCCCAAUGCCGCCUCACACCACUGUCUCGCC | |
| | UGCCAUUCGGCAUGGCCCCCGGCCCGGGGCCACAGCCUGG | |
| | GCCCCUACGGGAAUCUAUUGUUUGCUAUUUUAUGGUGUU | |
| | CCUGCAGACCCAUAUCUUCGCAGAAGUCCUGAAAGACGC | |
| | CAUCAAAGACUUAGUUAUGACUAAACCCGCACCCACCUG | |
| | CAACAUUCGCGUGACCGUAUGCAGCUUUGAUGAUGGCGU | |
| | CGAUCUGCCCCCAUGGUUCCCCCCUAUGGUCGAGGGCGCC | |
| | GCCGCCGAAGGGGACGACGGGGAUGACGGGGAUGAAGGC | |
| | GGGGACGGCGAUGAGGGCGAGGAGGGCCAGGAA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MSDEGPGTGPGNGLGEKGDTSGPEGSGGSGPQRRGGDNHGR | 4 |
| amino acid | GRGRGRGRGGGRPGAPGGSGSGPRHRDGVRRPQKRPSCIGCK | |
| sequence | GTHGGTGGGAGAGGARGRGGSGGRGRGGSGGRRGRGRERA | |
| | RGGSRERARGRGRGRGEKRPRSPSSQSSSSGSPPRRPPPGRRPF | |
| | FHPVGEADYFEYHQEGGPDGEPDVPPGAIEQGPADDPGEGPST | |
| | GPRGQGDGGRRKKGGWFGKHRGQGGSNPKFENIAEGLRALL | |
| | ARSHVERTTDEGTWVAGVFVYGGSKTSLYNLRRGTALAIPQC | |
| | RLTPLSRLPFGMAPGPGPQPGPLRESIVCYFMVFLQTHIFAEVL | |
| | KDAIKDLVMTKPAPTCNIRVTVCSFDDGVDLPPWFPPMVEGA | |
| | AAEGDDGDDGDEGGDGDEGEEGQE | |
| PolyA tail | 100 nt | |

EBNA1_mod_002

| SEQ ID NO: 131 consists of from 5′ end to 3′ end: | | 131 |
|---|---|---|
| 5′ UTR SEQ ID NO: 1, mRNA ORF SEQ ID NO: 5, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGAGUGACGAGGGACCUGGGACUGGCCCCGGGAACGGU | 5 |
| Construct | CUGGGUGAGAAAGGUGACACGUCUGGUCCUGAAGGAUCU | |
| (excluding | GGCGGUUCAGGGCCGCAGCGCAGAGGAGGAGACAACCAC | |
| the stop | GGUAGAGGACGUGGACGCGGGAGAGGCCGAGGGGGCGGA | |
| codon) | AGACCUGGUGCACCGGGCGGUUCCGGCUCGGGGCCCCGCC | |
| | ACAGGGAUGGGGUGCGGAGACCACAGAAGCGCCCCUCUU | |
| | GCAUCGGCUGCAAGGGAACCCAUGGGGGUACCGGAGGGG | |
| | GAGCUGGUGCAGGUGGCGCCCGUGGUAGGGGUGGCAGCG | |
| | GGGGGCGUGGAAGAGGUGGUUCAGGCGGACGGAGGGGAC | |
| | GCGGACGUGAACGUGCCCGGGGAGGAUCCCGGGAGAGGG | |
| | CACGGGGCCGCGGCAGAGGCAGAGGGGAGAAGAGACCUA | |
| | GAUCCCCUAGCUCUCAAUCCAGCAGCAGUGGGUCCCCCCC | |
| | GAGGCGACCGCCUCCAGGUAGGCGCCCUUUCUUCCACCCC | |
| | GUUGGCGAGGCAGAUUACUUCGAAUAUCAUCAAGAGGGG | |
| | GGGCCCGAUGGCGAGCCGGACGUACCACCCGGUGCUAUC | |
| | GAGCAAGGUCCGGCUGACGAUCCUGGUGAAGGGCCAAGU | |
| | ACCGGACCUAGGGGGCAGGGCGACGGUGGUAGGCGUAAA | |
| | AAAGGAGGGUGGUUCGGCAAACAUCGCGGCCAGGGAGGG | |
| | AGCAACCCCAAGUUUGAAAAUAUCGCCGAAGGGCUACGA | |
| | GCUUUGCUCGCGAGGUCCCAUGUGGAACGAACUACAGAC | |
| | GAGGGCACCUGGGUGGCUGGUGUCUUCGUUUAUGGCGGG | |
| | AGCAAGACCUCCCUGUAUAAUCUAAGGCGGGGCACAGCC | |
| | CUGGCCAUACCGCAGUGUCGCCUGACACCCCUGUCUCGCC | |
| | UGCCUUUUGGCAUGGCGCCAGGGCCAGGACCACAACCUG | |
| | GGCCCCUGCGCGAAAGUAUUGUGUGUUACUUCAUGGUCU | |
| | UCCUGCAGACACAUAUUUUCGCAGAAGUCCUAAAGGAUG | |
| | CAAUAAAGGAUCUCGUAAUGACGAAGCCCGCCCCAACGU | |
| | GUAAUAUUAGGGUUACGGUCUGUAGUUUCGAUGACGGGG | |
| | UAGAUCUCCCCCCCUGGUUCCCACCUAUGGUCGAGGGUGC | |
| | CGCUGCGGAGGGGGACGAUGGCGACGACGGCGAUGAAGG | |
| | CGGGGAUGGCGACGAGGGGGAGGAGGGACAGGAA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MSDEGPGTGPGNGLGEKGDTSGPEGSGGSGPQRRGGDNHGR | 4 |
| amino acid | GRGRGRGRGGGRPGAPGGSGSGPRHRDGVRRPQKRPSCIGCK | |
| sequence | GTHGGTGGGAGAGGARGRGGSGGRGRGGSGGRRGRGRERA | |
| | RGGSRERARGRGRGRGEKRPRSPSSQSSSSGSPPRRPPPGRRPF | |
| | FHPVGEADYFEYHQEGGPDGEPDVPPGAIEQGPADDPGEGPST | |
| | GPRGQGDGGRRKKGGWFGKHRGQGGSNPKFENIAEGLRALL | |
| | ARSHVERTTDEGTWVAGVFVYGGSKTSLYNLRRGTALAIPQC | |
| | RLTPLSRLPFGMAPGPGPQPGPLRESIVCYFMVFLQTHIFAEVL | |
| | KDAIKDLVMTKPAPTCNIRVTVCSFDDGVDLPPWFPPMVEGA | |
| | AAEGDDGDDGDEGGDGDEGEEGQE | |
| PolyA tail | 100 nt | |

EBNA1_mod_003

| SEQ ID NO: 132 consists of from 5′ end to 3′ end: | | 132 |
|---|---|---|
| 5′ UTR SEQ ID NO: 1, mRNA ORF SEQ ID NO: 6, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGUCAGACGAGGGCCCAGGCACGGGCCCUGGAAACGGA | 6 |
| Construct | CUGGGAGAGAAGGGGGAUACCUCAGGGCCAGAGGGUAGU | |
| (excluding | GGGGGUAGCGGGCCUCAGAGAAGGGGGGGCGACAAUCAC | |
| the stop | GGUCGGGGAAGAGGGAGAGGUAGGGGUAGAGGAGGAGGC | |
| codon) | CGUCCCGGGGCUCCUGGCGGUUCCGGGUCCGGCCCUCGCC | |
| | ACAGAGAUGGCGUGAGGAGACCUCAGAAGCGUCCGUCCU | |
| | GUAUUGGGUGCAAGGGGACCCACGGUGGCACCGGUGGCG | |
| | GAGCUGGGGCAGGAGGUGCUAGAGGAAGAGGCGGUUCAG | |
| | GUGGAAGGGGUCGCGGUGGUUCAGGAGGACGGCGCGGCC | |
| | GGGGGAGAGAGCGGGCAAGAGGCGGCAGCAGGGAGCGGG | |
| | CAAGAGGACGAGGGAGGGGCCGAGGCGAGAAGCGACCUA | |
| | GGUCGCCUAGUUCACAGAGCUCCAGUUCUGGGUCGCCCCC | |
| | CCGGCGGCCGCCUCCUGGGCGAAGACCCUUUUUCCACCCC | |
| | GUGGGGGAGGCUGACUACUUCGAGUACCACCAGGAGGGC | |
| | GGCCCAGACGGAGAGCCCGAUGUUCCGCCUGGGGCCAUU | |
| | GAACAAGGCCCUGCAGAUGACCCAGGGGAAGGACCAUCA | |
| | ACCGGCCCUAGGGGACAGGGCGAUGGGGGGAGACGGAAA | |
| | AAGGGAGGGUGGUUCGGGAAGCACCGCGGCCAGGGGGGG | |
| | UCUAAUCCGAAAUUCGAAAACAUUGCCGAGGGCCUCCGU | |
| | GCUUUGUUGGCUAGAUCACACGUGGAAAGAACCACGGAU | |
| | GAAGGCACGUGGGUGGCGGGCGUCUUCGUGUAUGGUGGG | |
| | AGUAAGACCUCCCUGUAUAACCUCAGGCGUGGCACAGCC | |
| | CUUGCCAUCCCACAGUGCAGGCUCACACCCCUUAGCAGGC | |
| | UUCCCUUUGGCAUGGCUCCUGGUCCCGGCCCCCAGCCCGG | |
| | UCCACUGCGAGAAUCCAUUGUAUGUUACUUCAUGGUUUU | |
| | CUUGCAGACACAUAUCUUCGCCGAGGUGCUGAAAGACGC | |
| | CAUUAAAGACCUGGUGAUGACAAAGCCUGCCCCCACGUG | |
| | CAAUAUCCGUGUGACCGUCUGCUCCUUUGAUGACGGAGU | |
| | CGACCUGCCUCCCUGGUUUCCCCCAAUGGUCGAGGGCGCU | |
| | GCGGCGGAAGGGGACGACGGCGAUGAUGGUGACGAGGGC | |
| | GGUGAUGGCGACGAAGGUGAAGAGGGUCAGGAG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MSDEGPGTGPGNGLGEKGDTSGPEGSGGSGPQRRGGDNHGR | 4 |
| amino acid | GRGRGRGRGGGRPGAPGGSGSGPRHRDGVRRPQKRPSCIGCK | |
| sequence | GTHGGTGGGAGAGGARGRGGSGGRGRGGSGGRRGRGRERA | |
| | RGGSRERARGRGRGRGEKRPRSPSSQSSSSGSPPRRPPPGRRPF | |
| | FHPVGEADYFEYHQEGGPDGEPDVPPGAIEQGPADDPGEGPST | |
| | GPRGQGDGGRRKKGGWFGKHRGQGGSNPKFENIAEGLRALL | |
| | ARSHVERTTDEGTWVAGVFVYGGSKTSLYNLRRGTALAIPQC | |
| | RLTPLSRLPFGMAPGPGPQPGPLRESIVCYFMVFLQTHIFAEVL | |
| | KDAIKDLVMTKPAPTCNIRVTVCSFDDGVDLPPWFPPMVEGA | |
| | AAEGDDGDDGDEGGDGDEGEEGQE | |
| PolyA tail | 100 nt | |

EBNA1_mod_004

| SEQ ID NO: 133 consists of from 5′ end to 3′ end: | | 133 |
|---|---|---|
| 5′ UTR SEQ ID NO: 1, mRNA ORF SEQ ID NO: 7, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGAGCGACGAGGGUCCAGGCACAGGCCCGGGGAACGGG | 7 |
| Construct | UUGGGAGAAAAAGGGGACACUUCCGGUCCAGAAGGCUCC | |
| (excluding | GGGGGGAGCGGACCUCAGAGAAGAGGUGGUGAUAAUCAC | |
| the stop | GGGAGAGGGAGAGGGCGGGGCAGGGGACGUGGGGGCGGU | |
| codon) | AGACCUGGGGCGCCGGGGGGUUCCGGCUCCGGACCACGG | |
| | CAUAGGGAUGGUGUGCGAAGGCCGCAGAAGCGGCCGUCU | |
| | UGUAUAGGCUGCAAAGGCACUCACGGUGGUACCGGGGGG | |
| | GGAGCAGGAGCAGGAGGCGCUCGAGGUCGGGGCGGGUCC | |
| | GGCGGCAGAGGCCGCGGUGGUUCUGGGGGAAGGAGGGGA | |
| | AGAGGGAGAGAACGCGCCCGCGGAGGCUCUCGCGAACGG | |
| | GCCAGAGGGCGGGGGAGGGGGCGCGGGGAGAAAAGGCCC | |
| | AGGUCGCCAAGUAGUCAGUCCUCCAGUAGCGGCAGCCCA | |
| | CCCCGCAGACCUCCUCCAGGACGACGACCUUUCUUUCAUC | |
| | CCGUCGGCGAGGCCGAUUACUUUGAGUACCAUCAGGAAG | |
| | GGGGGCCUGAUGGAGAACCGGAUGUUCCUCCUGGUGCUA | |
| | UCGAACAGGGCCCCGCCGACGAUCCAGGCGAGGGCCCCAG | |
| | UACGGGCCCUAGAGGCCAGGGAGAUGGAGGUAGAAGGAA | |
| | GAAGGGGGGAUGGUUUGGCAAGCACAGAGGGCAGGGAGG | |
| | UAGUAAUCCCAAAUUUGAGAAUAUUGCCGAGGGUCUGCG | |
| | GGCACUUCUGGCCCGGUCUCAUGUUGAGAGAACGACUGA | |
| | UGAGGGAACAUGGGUGGCUGGCGUCUUUGUGUAUGGCGG | |
| | UAGUAAAACCAGCCUCUACAAUUUACGGCGUGGGACAGC | |
| | GCUAGCUAUUCCGCAAUGCCGGCUGACCCCUUUGUCUAG | |
| | GCUGCCCUUUGGUAUGGCCCCCGGCCCCGGCCCACAGCCU | |
| | GGGCCUUUGCGGGAAAGCAUCGUCUGCUACUUUAUGGUG | |
| | UUCCUCCAGACCCACAUCUUUGCAGAGGUGCUUAAGGAC | |
| | GCCAUAAAGGACCUGGUUAUGACCAAACCCGCCCCUACCU | |
| | GCAAUAUCCGCGUCACGGUGUGCUCCUUCGACGACGGGG | |
| | UUGACUUGCCUCCAUGGUUCCCACCCAUGGUAGAGGGCG | |
| | CUGCUGCCGAGGGGGAUGAUGGAGACGACGGAGACGAGG | |
| | GCGGCGAUGGCGAUGAAGGCGAAGAAGGCCAGGAA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| Corresponding | MSDEGPGTGPGNGLGEKGDTSGPEGSGGSGPQRRGGDNHGR | 4 |
| amino acid | GRGRGRGRGGGRPGAPGGSGSGPRHRDGVRRPQKRPSCIGCK | |
| sequence | GTHGGTGGGAGAGGARGRGGSGGRGRGGSGGRRGRGRERA | |
| | RGGSRERARGRGRGRGEKRPRSPSSQSSSSGSPPRRPPPGRRPF | |
| | FHPVGEADYFEYHQEGGPDGEPDVPPGAIEQGPADDPGEGPST | |
| | GPRGQGDGGRRKKGGWFGKHRGQGGSNPKFENIAEGLRALL | |
| | ARSHVERTTDEGTWVAGVFVYGGSKTSLYNLRRGTALAIPQC | |
| | RLTPLSRLPFGMAPGPGPQPGPLRESIVCYFMVFLQTHIFAEVL | |
| | KDAIKDLVMTKPAPTCNIRVTVCSFDDGVDLPPWFPPMVEGA | |
| | AAEGDDGDDGDEGGDGDEGEEGQE | |
| PolyA tail | 100 nt | |

EBNA1_mod_005

| SEQ ID NO: 134 consists of from 5′ end to 3′ end: | | 134 |
|---|---|---|
| 5′ UTR SEQ ID NO: 1, mRNA ORF SEQ ID NO: 8, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGUCUGACGAAGGCCCGGGCACAGGCCCAGGCAACGGA | 8 |
| Construct | UUGGGCGAAAAAGGUGAUACAAGCGGGCCAGAAGGGAGC | |
| (excluding | GGGGGCUCUGGGCCCCAGAGACGGGGGGGUGACAAUCAC | |
| the stop | GGCCGAGGAAGGGGAAGGGGCAGGGGAAGAGGCGGAGGA | |
| codon) | CGGCCUGGCGCUCCUGGAGGCUCUGGCUCAGGUCCAAGA | |
| | CAUAGGGAUGGGGUCAGAAGACCACAGAAGAGACCUAGC | |
| | UGUAUUGGAUGCAAGGGUACACACGGUGGCACGGGCGGC | |
| | GGUGCUGGCGCUGGGGGCGCGAGAGGACGUGGAGGUUCA | |
| | GGCGGGAGAGGUCGAGGAGGCUCUGGAGGCCGCAGGGGG | |
| | AGAGGACGAGAGAGGGCCCGGGGGGGGUCCCGGGAAAGA | |
| | GCUAGAGGGAGGGGACGCGGGCGGGGGGAAAAGCGGCCU | |
| | CGCUCUCCUUCUUCUCAAUCUUCCUCCUCUGGUUCUCCGC | |
| | CCCGACGCCCCCCACCGGGCAGGAGGCCAUUUUUUCACCC | |
| | UGUCGGCGAGGCAGACUACUUCGAGUACCACCAAGAAGG | |
| | AGGCCCAGACGGCGAGCCUGAUGUGCCGCCAGGUGCCAU | |
| | CGAGCAGGGGCCAGCUGAUGACCCGGGGGAGGGACCUAG | |
| | CACGGGGCCCCGCGGUCAGGGCGACGGAGGGAGGAGAAA | |
| | AAAAGGUGGGUGGUUUGGCAAGCAUAGAGGGCAGGGUGG | |
| | CUCAAACCCAAAGUUUGAAAACAUCGCAGAAGGUCUUCG | |
| | GGCUCUGCUGGCCCGCUCUCAUGUAGAGCGCACUACUGA | |
| | CGAAGGCACCUGGGUGGCCGGGGUGUUCGUGUACGGAGG | |
| | AUCUAAGACCAGUCUGUACAACCUCCGGAGGGGGACAGC | |
| | UCUCGCUAUCCCCCAGUGUAGGCUCACCCCUUUGAGUCGC | |
| | CUUCCGUUCGGAAUGGCUCCUGGCCCGGGCCCCCAGCCCG | |
| | GGCCACUAAGAGAGAGCAUCGUAUGCUACUUUAUGGUAU | |
| | UUUUGCAAACCCACAUCUUCGCUGAAGUGCUUAAGGACG | |
| | CCAUCAAAGACCUGGUGAUGACAAAACCAGCUCCAACAU | |
| | GCAAUAUUAGGGUAACCGUAUGCUCCUUCGAUGAUGGCG | |
| | UCGACCUUCCACCUUGGUUCCCCCCAAUGGUGGAGGGAG | |
| | CCGCAGCAGAAGGAGACGAUGGUGAUGAUGGAGACGAAG | |
| | GCGGGGAUGGCGACGAAGGAGAGGAGGGACAGGAA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MSDEGPGTGPGNGLGEKGDTSGPEGSGGSGPQRRGGDNHGR | 4 |
| amino acid | GRGRGRGRGGGRPGAPGGSGSGPRHRDGVRRPQKRPSCIGCK | |
| sequence | GTHGGTGGGAGAGGARGRGGSGGRGRGGSGGRRGRGRERA | |
| | RGGSRERARGRGRGRGEKRPRSPSSQSSSSGSPPRRPPPGRRPF | |
| | FHPVGEADYFEYHQEGGPDGEPDVPPGAIEQGPADDPGEGPST | |
| | GPRGQGDGGRRKKGGWFGKHRGQGGSNPKFENIAEGLRALL | |
| | ARSHVERTTDEGTWVAGVFVYGGSKTSLYNLRRGTALAIPQC | |
| | RLTPLSRLPFGMAPGPGPQPGPLRESIVCYFMVFLQTHIFAEVL | |
| | KDAIKDLVMTKPAPTCNIRVTVCSFDDGVDLPPWFPPMVEGA | |
| | AAEGDDGDDGDEGGDGDEGEEGQE | |
| PolyA tail | 100 nt | |

EBNA1_trunc_001

| SEQ ID NO: 135 consists of from 5′ end to 3′ end: | | 135 |
|---|---|---|
| 5′ UTR SEQ ID NO: 1, mRNA ORF SEQ ID NO: 9, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGCAGAAGACCCUUCUUUCACCCCGUGGGAGAAGCC | 9 |
| Construct | GACUAUUUCGAGUACCACCAGGAGGGCGGUCCUGACGGC | |
| (excluding | GAGCCCGACGUGCCUCCAGGCGCCAUCGAACAGGGCCCCG | |
| the stop | CUGACGAUCCUGGCGAGGGGCCUUCCACAGGCCCUCGCGG | |
| codon) | CCAGGGAGAUGGGGGAAGGCGGAAGAAGGGGGGCUGGUU | |
| | UGGCAAGCAUAGGGGCCAGGGCGGCUCCAAUCCCAAGUU | |
| | CGAGAAUAUCGCCGAGGGGCUCCGGGCUCUGCUGGCCCG | |
| | GUCGCACGUCGAACGUACCACUGACGAGGGGACGUGGGU | |
| | GGCCGGCGUCUUUGUGUACGGAGGAUCCAAGACCUCCCU | |
| | GUACAACCUGAGAAGGGGGACCGCCCUUGCCAUCCCUCA | |
| | GUGCAGACUCACCCCCCUGAGCAGGCUCCCCUUUGGGAUG | |
| | GCUCCUGGGCCAGGCCCCCAGCCCGGGCCCCUCAGGGAAA | |
| | GCAUCGUGUGUUACUUUAUGGUGUUUCUGCAGACACACA | |
| | UUUUUGCAGAAGUUCUGAAAGAUGCAAUCAAGGAUCUGG | |
| | UGAUGACCAAGCCUGCACCUACGUGCAAUAUCAGGGUGA | |
| | CAGUAUGCUCCUUCGACGACGGUGUGGACCUCCCCCCCUG | |
| | GUUCCCACCCAUGGUCGAGGGGGCCGCCGCCGAGGGAGA | |
| | CGAUGGCGACGACGGUGAUGAGGGGGGAGACGGAGAUGA | |
| | AGGCGAGGAGGGGCAGGAG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGRRPFFHPVGEADYFEYHQEGGPDGEPDVPPGAIEQGPADD | 10 |
| amino acid | PGEGPSTGPRGQGDGGRRKKGGWFGKHRGQGGSNPKFENIA | |
| sequence | EGLRALLARSHVERTTDEGTWVAGVFVYGGSKTSLYNLRRG | |
| | TALAIPQCRLTPLSRLPFGMAPGPGPQPGPLRESIVCYFMVFLQ | |
| | THIFAEVLKDAIKDLVMTKPAPTCNIRVTVCSFDDGVDLPPWF | |
| | PPMVEGAAAEGDDGDDGDEGGDGDEGEEGQE | |
| PolyA tail | 100 nt | |

EBNA1_trunc_002

| SEQ ID NO: 136 consists of from 5′ end to 3′ end: | | 136 |
|---|---|---|
| 5′ UTR SEQ ID NO: 1, mRNA ORF SEQ ID NO: 11, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGACGGAGACCAUUCUUCCACCCCGUGGGAGAAGCC | 11 |
| Construct | GACUACUUCGAGUACCACCAGGAGGGCGGUCCAGACGGC | |
| (excluding | GAACCCGACGUCCCUCCUGGAGCCAUCGAGCAGGGCCCCG | |
| the stop | CCGACGACCCUGGCGAGGGCCCUAGCACCGGACCUAGAGG | |
| codon) | ACAAGGCGACGGCGGGAGGAGAAAGAAGGGGGGCUGGUU | |
| | CGGGAAGCACCGCGGGCAAGGCGGCAGCAACCCAAAGUU | |
| | CGAGAACAUCGCCGAGGGGUUGCGGGCCCUGCUCGCCCG | |
| | UUCCCACGUGGAGCGGACAACCGACGAGGGAACCUGGGU | |
| | UGCCGGGGUAUUUGUGUACGGCGGCAGUAAGACCAGCCU | |
| | GUAUAACCUCAGAAGAGGAACAGCCCUGGCCAUCCCACA | |
| | GUGCAGGCUGACACCCCUCUCCCGGCUGCCAUUUGGAAU | |
| | GGCACCCGGGCCUGGACCCCAGCCUGGCCCACUGCGCGAG | |
| | UCUAUCGUGUGUUACUUUAUGGUCUUCCUGCAAACCCAC | |
| | AUCUUCGCCGAGGUUUUAAAGGAUGCCAUCAAAGACCUC | |
| | GUGAUGACAAAGCCCGCUCCCACCUGCAAUAUCAGAGUG | |
| | ACCGUAUGCAGCUUUGACGACGGCGUGGACCUGCCUCCC | |
| | UGGUUCCCUCCUAUGGUGGAGGGCGCCGCCGCAGAAGGG | |
| | GACGACGGUGACGAUGGCGAUGAGGGAGGGGAUGGAGAC | |
| | GAAGGCGAGGAGGGCCAGGAG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGRRPFFHPVGEADYFEYHQEGGPDGEPDVPPGAIEQGPADD | 10 |
| amino acid | PGEGPSTGPRGQGDGGRRKKGGWFGKHRGQGGSNPKFENIA | |
| sequence | EGLRALLARSHVERTTDEGTWVAGVFVYGGSKTSLYNLRRG | |
| | TALAIPQCRLTPLSRLPFGMAPGPGPQPGPLRESIVCYFMVFLQ | |
| | THIFAEVLKDAIKDLVMTKPAPTCNIRVTVCSFDDGVDLPPWF | |
| | PPMVEGAAAEGDDGDDGDEGGDGDEGEEGQE | |
| PolyA tail | 100 nt | |

EBNA1_trunc_003

| SEQ ID NO: 137 consists of from 5′ end to 3′ end: | | 137 |
|---|---|---|
| 5′ UTR SEQ ID NO: 1, mRNA ORF SEQ ID NO: 12, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGCCGCAGACCCUUCUUUCACCCCGUGGGAGAGGCC | 12 |
| Construct | GACUAUUUUGAGUACCACCAGGAGGGCGGGCCCGACGGC | |
| (excluding | GAACCUGACGUGCCACCUGGCGCCAUUGAACAAGGCCCA | |
| the stop | GCCGACGACCCUGGGGAGGGCCCUAGCACAGGGCCCAGG | |
| codon) | GGCCAGGGAGACGGGGGUAGGCGUAAGAAGGGAGGCUGG | |
| | UUCGGUAAGCACCGCGGCCAGGGUGGCAGCAACCCUAAA | |
| | UUCGAGAAUAUUGCCGAAGGUCUGCGAGCCCUCUUGGCC | |
| | AGAAGCCACGUGGAACGCACUACUGAUGAGGGCACCUGG | |
| | GUCGCCGGAGUGUUCGUGUACGGCGGAAGCAAAACCAGU | |
| | CUGUACAACCUGCGCCGGGGAACCGCUCUUGCCAUCCCCC | |
| | AGUGUCGCCUGACCCCAUUGUCCAGACUGCCAUUUGGCA | |
| | UGGCCCCCGGCCCCGGCCCCCAGCCCGGCCCCCUGAGGGA | |
| | GAGCAUCGUGUGCUACUUCAUGGUGUUCCUGCAGACCCA | |
| | CAUAUUCGCUGAGGUGCUGAAGGACGCCAUUAAGGACCU | |
| | GGUGAUGACCAAGCCCGCCCCUACCUGCAACAUCCGCGUC | |
| | ACCGUCUGUAGUUUCGAUGACGGCGUGGAUCUCCCACCA | |
| | UGGUUUCCUCCAAUGGUGGAGGGCGCUGCUGCCGAGGGG | |
| | GACGACGGAGACGACGGAGACGAGGGCGGGGACGGGGAC | |
| | GAAGGCGAAGAGGGGCAAGAG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGRRPFFHPVGEADYFEYHQEGGPDGEPDVPPGAIEQGPADD | 10 |
| amino acid | PGEGPSTGPRGQGDGGRRKKGGWFGKHRGQGGSNPKFENIA | |
| sequence | EGLRALLARSHVERTTDEGTWVAGVFVYGGSKTSLYNLRRG | |
| | TALAIPQCRLTPLSRLPFGMAPGPGPQPGPLRESIVCYFMVFLQ | |
| | THIFAEVLKDAIKDLVMTKPAPTCNIRVTVCSFDDGVDLPPWF | |
| | PPMVEGAAAEGDDGDDGDEGGDGDEGEEGQE | |
| PolyA tail | 100 nt | |

EBNA1_trunc_004

| SEQ ID NO: 138 consists of from 5′ end to 3′ end: | | 138 |
|---|---|---|
| 5′ UTR SEQ ID NO: 1, mRNA ORF SEQ ID NO: 13, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGACGGAGACCUUUCUUUCACCCCGUAGGUGAAGCC | 13 |
| Construct | GACUACUUUGAAUACCACCAGGAGGGCGGCCCAGACGGC | |
| (excluding | GAGCCCGACGUUCCCCCCGGGGCUAUCGAGCAGGGGCCUG | |
| the stop | CGGACGACCCAGGAGAGGGACCUAGCACUGGACCCCGAG | |
| codon) | GUCAGGGCGAUGGGGGGCGAAGAAAGAAGGGCGGCUGGU | |
| | UCGGCAAACAUAGGGGCCAGGGCGGGUCUAACCCAAAGU | |
| | UCGAGAACAUAGCCGAGGGCCUGAGAGCCCUGCUGGCAA | |
| | GAAGCCACGUGGAGCGAACCACAGAUGAAGGCACCUGGG | |
| | UGGCCGGCGUGUUCGUGUACGGGGGCAGCAAGACCUCCC | |
| | UGUAUAACCUCAGAAGAGGGACCGCCCUGGCCAUCCCCCA | |
| | GUGCCGACUGACCCCCCUGAGCAGGCUGCCCUUCGGAAUG | |
| | GCCCCUGGUCCCGGUCCCCAACCGGGCCCGCUUCGGGAGU | |
| | CCAUAGUGUGCUACUUCAUGGUGUUCCUGCAGACACACA | |
| | UCUUCGCCGAGGUGCUGAAGGAUGCAAUCAAGGACCUGG | |
| | UGAUGACCAAGCCAGCUCCCACCUGCAACAUCCGGGUGAC | |
| | CGUGUGCAGCUUUGAUGACGGCGUGGACCUGCCACCCUG | |
| | GUUCCCGCCUAUGGUGGAGGGCGCUGCAGCCGAGGGCGA | |
| | CGACGGGGACGACGGCGACGAGGGCGGAGAUGGCGACGA | |
| | GGGCGAGGAGGGCCAGGAG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGRRPFFHPVGEADYFEYHQEGGPDGEPDVPPGAIEQGPADD | 10 |
| amino acid | PGEGPSTGPRGQGDGGRRKKGGWFGKHRGQGGSNPKFENIA | |
| sequence | EGLRALLARSHVERTTDEGTWVAGVFVYGGSKTSLYNLRRG | |
| | TALAIPQCRLTPLSRLPFGMAPGPGPQPGPLRESIVCYFMVFLQ | |
| | THIFAEVLKDAIKDLVMTKPAPTCNIRVTVCSFDDGVDLPPWF | |
| | PPMVEGAAAEGDDGDDGDEGGDGDEGEEGQE | |
| PolyA tail | 100 nt | |

EBNA1_trunc_005

| SEQ ID NO: 139 consists of from 5′ end to 3′ end: | | 139 |
|---|---|---|
| 5′ UTR SEQ ID NO: 1, mRNA ORF SEQ ID NO: 14, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGCAGACGGCCCUUCUUCCACCCCGUGGGCGAAGCA | 14 |
| Construct | GACUAUUUUGAGUAUCACCAGGAGGGGGGCCCCGACGGC | |
| (excluding | GAGCCAGACGUCCCACCUGGUGCAAUCGAGCAAGGUCCC | |
| the stop | GCCGACGACCCUGGGGAGGGCCCCAGCACCGGCCCCAGAG | |
| codon) | GCCAGGGGGACGGGGGCAGGAGAAAAAAGGGUGGCUGGU | |
| | UCGGCAAGCACAGAGGGCAGGGGGGGAGCAACCCAAAGU | |
| | UCGAGAACAUCGCCGAGGGCUUGAGAGCACUGCUGGCCA | |
| | GAAGCCACGUGGAGAGAACCACCGAUGAGGGAACCUGGG | |
| | UUGCUGGCGUGUUCGUCUACGGCGGCAGCAAGACCAGCC | |
| | UGUACAACCUUAGAAGAGGCACCGCCCUGGCCAUCCCCCA | |
| | GUGCCGCCUGACACCGCUGAGCAGACUCCCCUUCGGCAUG | |
| | GCUCCUGGCCCCGGCCCCCAGCCCGGGCCCCUGAGAGAGA | |
| | GCAUCGUGUGCUACUUCAUGGUGUUUCUUCAGACGCACA | |
| | UCUUCGCUGAGGUCCUGAAAGACGCCAUUAAGGACCUGG | |
| | UGAUGACAAAACCAGCUCCGACCUGCAACAUCAGGGUCA | |
| | CUGUCUGUAGCUUCGACGAUGGCGUGGAUCUCCCUCCCU | |
| | GGUUCCCCCCAAUGGUGGAGGGCGCUGCCGCUGAGGGGG | |
| | ACGACGGCGACGACGGGGAUGAAGGCGGCGACGGCGACG | |
| | AGGGGGAAGAGGGCCAGGAG | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGRRPFFHPVGEADYFEYHQEGGPDGEPDVPPGAIEQGPADD | 10 |
| amino acid | PGEGPSTGPRGQGDGGRRKKGGWFGKHRGQGGSNPKFENIA | |
| sequence | EGLRALLARSHVERTTDEGTWVAGVFVYGGSKTSLYNLRRG | |
| | TALAIPQCRLTPLSRLPFGMAPGPGPQPGPLRESIVCYFMVFLQ | |
| | THIFAEVLKDAIKDLVMTKPAPTCNIRVTVCSFDDGVDLPPWF | |
| | PPMVEGAAAEGDDGDDGDEGGDGDEGEEGQE | |
| PolyA tail | 100 nt | |

LMP1_001

| SEQ ID NO: 140 consists of from 5′ end to 3′ end: | | 140 |
|---|---|---|
| 5′ UTR SEQ ID NO: 1, mRNA ORF SEQ ID NO: 15, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGCACGACCUGGAGAGGGGGCCACCAGGCCCCAGG | 15 |
| Construct | CGACCCCCAAGAGGACCACCACUGAGCUCCAGCCUGGGAC | |
| (excluding | UGGCCCUCCUGCUGUUGCUCCUCGCUUUACUGUUUUGGC | |
| the stop | UGUAUAUAGUCAUGUCUGACUGGACAGGCGGGGCAUUAU | |
| codon) | UGGUGCUCUACAGCUUUGCACUGAUGCUCAUUAUCAUUA | |
| | UUCUGAUUAUCUUUAUUUUUAGACGGGACUUGCUCUGUC | |
| | CAUUAGGCGCGCUGUGUAUUUUGCUGCUGAUGAUCACAC | |
| | UGCUGCUGAUUGCGCUGUGGAACCUCCACGGUCAGGCCC | |
| | UUUUUCUCGGCAUCGUGCUGUUUAUCUUUGGGUGCCUCU | |
| | UGGUGCUUGGGAUUUGGAUCUACCUUCUCGAAAUGUUAU | |
| | GGCGGCUCGGCGCUACAAUCUGGCAGCUCCUGGCCUUCU | |
| | UCUUAGCAUUUUUUCUGGAUCUGAUCCUGCUGAUAAUAG | |
| | CCCUGUACCUACAACAGAACUGGUGGACUCUGCUGGUUG | |
| | ACCUGUUAUGGCUGCUCCUGUUCCUCGCCAUUCUCAUUU | |
| | GGAUGUACUACCACGGGCAGCGUCAUUCAGACGAACACC | |
| | ACCACGAUGAUAGCCUGCCGCACCCUCAACAAGCUACGGA | |
| | UGACUCCGGACACGAGUCCGAUAGUAACUCCAACGAGGG | |
| | ACGCCAUCACUUGCUGGUCUCUGGGGCUGGCGACGGGCC | |
| | UCCACUGUGUAGCCAAAAUCUGGGAGCCCCCGGCGGGGG | |
| | CCCUGAUAAUGGGCCUCAGGAUCCUGAUAACACGGAUGA | |
| | UAACGGCCCACAGGAUCCAGAUAAUACCGACGAUAAUGG | |
| | ACCUCAUGAUCCUCUUCCACAGGAUCCCGACAAUACAGA | |
| | UGACAAUGGGCCGCAGGAUCCAGACAAUACUGACGAUAA | |
| | UGGCCCACAUGAUCCCCUGCCACACAGCCCCUCAGACUCA | |
| | GCGGGAAAUGAUGGCGGGCCCCCACAGUUAACCGAGGAA | |
| | GUGGAAAACAAAGGGGGAGAUCAGGGUCCUCCCCUCAUG | |
| | ACAGAUGGCGGGGGCGGCCACAGUCAUGAUAGUGGGCAC | |
| | GGGGGUGGCGAUCCACAUCUCCCCACACUGCUGCUAGGC | |
| | AGCUCCGGUAGCGGGGGAGAUGAUGACGAUCCCCACGGC | |
| | CCUGUGCAGUUGAGCUAUUACGAC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEHDLERGPPGPRRPPRGPPLSSSLGLALLLLLLALLFWLYIV | 16 |
| amino acid | MSDWTGGALLVLYSFALMLIIIILIIFIFRRDLLCPLGALCILLLM | |
| sequence | ITLLLIALWNLHGQALFLGIVLFIFGCLLVLGIWIYLLEMLWRL | |
| | GATIWQLLAFFLAFFLDLILLIIALYLQQNWWTLLVDLLWLLL | |
| | FLAILIWMYYHGQRHSDEHHHDDSLPHPQQATDDSGHESDSN | |
| | SNEGRHHLLVSGAGDGPPLCSQNLGAPGGGPDNGPQDPDNTD | |
| | DNGPQDPDNTDDNGPHDPLPQDPDNTDDNGPQDPDNTDDNG | |
| | PHDPLPHSPSDSAGNDGGPPQLTEEVENKGGDQGPPLMTDGG | |
| | GGHSHDSGHGGGDPHLPTLLLGSSGSGGDDDDPHGPVQLSYY | |
| | D | |
| PolyA tail | 100 nt | |

LMP1_002

| SEQ ID NO: 141 consists of from 5′ end to 3′ end: | | 141 |
|---|---|---|
| 5′ UTR SEQ ID NO: 1, mRNA ORF SEQ ID NO: 17, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGCACGACCUGGAGCGCGGGCCUCCAGGACCCCGAC | 17 |
| Construct | GGCCGCCCAGGGGGCCACCGCUGUCAUCCAGUCUGGGCCU | |
| (excluding | UGCUCUGUUGCUGUUACUCCUUGCCCUGCUAUUCUGGUU | |
| the stop | GUACAUCGUCAUGAGCGAUUGGACAGGGGGUGCUCUGCU | |
| codon) | GGUGUUGUACAGCUUUGCGCUGAUGUUGAUUAUUAUCAU | |
| | CCUGAUUAUCUUUAUAUUUCGAAGGGACCUCCUUUGUCC | |
| | CCUGGGAGCGCUGUGCAUACUGCUCUUAAUGAUCACGUU | |
| | GCUCCUCAUUGCUUUAUGGAAUCUGCACGGACAGGCUCU | |
| | AUUCCUCGGCAUCGUUUUAUUCAUUUUCGGGUGCCUCCU | |
| | UGUGCUGGGAAUCUGGAUCUAUCUGCUAGAGAUGCUGUG | |
| | GCGGCUCGGCGCCACCAUUUGGCAGCUUCUGGCAUUUUU | |
| | CCUAGCGUUUUUUCUGGAUCUCAUCCUACUUAUUAUCGC | |
| | CUUGUAUUUGCAGCAGAACUGGUGGACUCUCCUGGUGGA | |
| | UCUCCUGUGGCUCCUCCUGUUCCUCGCCAUAUUGAUAUG | |
| | GAUGUACUACCACGGACAGCGCCACAGCGACGAGCACCAC | |
| | CACGAUGAUAGUCUGCCUCACCCUCAACAGGCUACAGAU | |
| | GACUCAGGCCACGAAAGCGAUUCCAACUCAAAUGAAGGC | |
| | CGGCACCACUUGUUGGUGUCCGGAGCCGGCGACGGGCCA | |
| | CCACUCUGCAGCCAGAAUCUGGGCGCUCCCGGCGGAGGUC | |
| | CAGACAAUGGGCCACAGGACCCUGAUAACACGGAUGAUA | |
| | ACGGACCGCAGGAUCCUGACAAUACAGAUGACAACGGCC | |
| | CGCAUGAUCCACUGCCUCAGGAUCCUGAUAAUACUGACG | |
| | AUAACGGACCCCAGGAUCCUGAUAACACCGACGACAACG | |
| | GGCCUCACGAUCCCCUACCCCAUUCCCCAUCUGACUCUGC | |
| | UGGCAACGAUGGCGGCCCACCGCAGCUGACCGAAGAGGU | |
| | AGAAAACAAGGGCGGGGAUCAGGGCCCUCCACUGAUGAC | |
| | AGACGGCGGCGGGGGACACUCCCAUGAUUCGGGCCAUGG | |
| | GGGAGGGGAUCCUCACCUUCCCACACUCUUGCUGGGAAG | |
| | UAGCGGGUCCGGCGGGGACGAUGAUGAUCCCCAUGGUCC | |
| | AGUCCAGUUGUCAUAUUACGAU | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEHDLERGPPGPRRPPRGPPLSSSLGLALLLLLLALLFWLYIV | 16 |
| amino acid | MSDWTGGALLVLYSFALMLIIIILIIFIFRRDLLCPLGALCILLLM | |
| sequence | ITLLLIALWNLHGQALFLGIVLFIFGCLLVLGIWIYLLEMLWRL | |
| | GATIWQLLAFFLAFFLDLILLIIALYLQQNWWTLLVDLLWLLL | |
| | FLAILIWMYYHGQRHSDEHHHDDSLPHPQQATDDSGHESDSN | |
| | SNEGRHHLLVSGAGDGPPLCSQNLGAPGGGPDNGPQDPDNTD | |
| | DNGPQDPDNTDDNGPHDPLPQDPDNTDDNGPQDPDNTDDNG | |
| | PHDPLPHSPSDSAGNDGGPPQLTEEVENKGGDQGPPLMTDGG | |
| | GGHSHDSGHGGGDPHLPTLLLGSSGSGGDDDDPHGPVQLSYY | |
| | D | |
| PolyA tail | 100 nt | |

LMP1_003

| SEQ ID NO: 142 consists of from 5′ end to 3′ end: | | 142 |
|---|---|---|
| 5′ UTR SEQ ID NO: 1, mRNA ORF SEQ ID NO: 18, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGCACGACCUUGAGCGGGGUCCACCAGGCCCACGG | 18 |
| Construct | CGCCCUCCGCGCGGGCCUCCUUUGUCAUCCUCCCUGGGAC | |
| (excluding | UGGCACUGCUGCUGUUGCUUCUGGCCCUGCUGUUCUGGC | |
| the stop | UGUACAUCGUUAUGUCUGAUUGGACAGGAGGAGCUCUGC | |
| codon) | UGGUUCUUUACUCUUUCGCGCUGAUGCUGAUCAUCAUCA | |
| | UCUUGAUCAUUUUCAUUUUCAGACGAGACCUCCUUUGUC | |
| | CUCUCGGAGCCCUGUGUAUCCUCCUGCUGAUGAUAACCC | |
| | UUCUGCUGAUUGCUUUGUGGAAUCUGCAUGGGCAAGCUC | |
| | UGUUUCUUGGAAUUGUCUUAUUUAUUUUUGGGUGUCUGC | |
| | UGGUGUUGGGAAUCUGGAUCUAUCUUCUUGAAAUGCUGU | |
| | GGCGGCUGGGCGCCACAAUCUGGCAGCUACUCGCCUUUU | |
| | UCCUAGCUUUCUUUUUGGAUCUGAUUCUGCUGAUUAUUG | |
| | CUCUCUACCUGCAGCAGAAUUGGUGGACCCUCUUAGUCG | |
| | AUCUGCUGUGGCUGUUGCUCUUUCUCGCCAUCCUGAUUU | |
| | GGAUGUACUAUCAUGGGCAGCGCCAUAGUGAUGAACAUC | |
| | AUCACGAUGAUUCCCUGCCACAUCCUCAGCAGGCCACAGA | |
| | UGAUUCAGGCCACGAAUCAGACAGCAAUAGCAAUGAGGG | |
| | CCGCCACCACCUGCUGGUUUCAGGGGCCGGUGACGGCCCU | |
| | CCAUUGUGCAGUCAGAACCUGGGAGCCCCGGGGGGUGGA | |
| | CCUGAUAACGGGCCGCAGGAUCCUGACAAUACUGAUGAC | |
| | AACGGACCCCAGGAUCCAGAUAACACUGACGACAAUGGC | |
| | CCCCAUGACCCCCUACCCCAGGAUCCCGACAAUACAGACG | |
| | ACAACGGACCGCAGGACCCCGACAACACUGAUGAUAAUG | |
| | GGCCACACGAUCCUCUGCCUCACUCUCCAUCAGACUCUGC | |
| | CGGAAAUGACGGUGGACCGCCACAGUUAACUGAAGAAGU | |
| | CGAAAACAAGGGCGGCGAUCAGGGGCCGCCGCUCAUGAC | |
| | AGAUGGCGGGGGAGGUCACAGCCACGAUAGCGGACACGG | |
| | AGGAGGUGACCCUCAUCUGCCUACACUUCUGUUGGGUAG | |
| | CUCUGGGAGUGGGGGGGAUGACGACGACCCUCACGGACC | |
| | UGUUCAGCUCUCAUACUACGAC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEHDLERGPPGPRRPPRGPPLSSSLGLALLLLLLALLFWLYIV | 16 |
| amino acid | MSDWTGGALLVLYSFALMLIIIILIIFIFRRDLLCPLGALCILLLM | |
| sequence | ITLLLIALWNLHGQALFLGIVLFIFGCLLVLGIWIYLLEMLWRL | |
| | GATIWQLLAFFLAFFLDLILLIIALYLQQNWWTLLVDLLWLLL | |
| | FLAILIWMYYHGQRHSDEHHHDDSLPHPQQATDDSGHESDSN | |
| | SNEGRHHLLVSGAGDGPPLCSQNLGAPGGGPDNGPQDPDNTD | |
| | DNGPQDPDNTDDNGPHDPLPQDPDNTDDNGPQDPDNTDDNG | |
| | PHDPLPHSPSDSAGNDGGPPQLTEEVENKGGDQGPPLMTDGG | |
| | GGHSHDSGHGGGDPHLPTLLLGSSGSGGDDDDPHGPVQLSYY | |
| | D | |
| PolyA tail | 100 nt | |

LMP1_004

| SEQ ID NO: 143 consists of from 5′ end to 3′ end: | | 143 |
|---|---|---|
| 5′ UTR SEQ ID NO: 1, mRNA ORF SEQ ID NO: 19, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAACACGAUCUUGAACGGGGGCCGCCGGGACCGAGG | 19 |
| Construct | AGGCCGCCACGGGGGCCCCCCCUGAGCAGUAGCUUGGGGC | |
| (excluding | UUGCGCUGCUUCUCCUGCUGUUGGCCCUCCUGUUUUGGC | |
| the stop | UGUAUAUUGUGAUGUCCGACUGGACCGGCGGCGCCCUGC | |
| codon) | UCGUCCUGUAUAGCUUUGCUUUGAUGCUGAUAAUUAUUA | |
| | UAUUGAUCAUCUUUAUUUUCCGGCGUGAUUUGCUCUGUC | |
| | CUCUUGGUGCCCUUUGUAUCCUGCUGUUAAUGAUCACUC | |
| | UGCUCCUCAUUGCCCUAUGGAACCUCCACGGACAAGCCCU | |
| | GUUUCUUGGCAUCGUUCUCUUUAUAUUCGGAUGCCUCUU | |
| | AGUGCUUGGGAUCUGGAUAUACCUGUUGGAAAUGCUGUG | |
| | GAGGCUUGGAGCCACGAUAUGGCAAUUAUUGGCAUUCUU | |
| | UCUGGCCUUCUUCCUGGAUCUGAUCCUGUUGAUAAUUGC | |
| | UCUGUACCUGCAGCAGAACUGGUGGACACUACUUGUUGA | |
| | CCUGCUCUGGCUACUCCUGUUCCUUGCGAUCCUGAUUUG | |
| | GAUGUACUAUCACGGACAGAGACACUCCGACGAGCAUCA | |
| | UCACGACGAUUCCUUACCACACCCUCAGCAGGCGACAGAU | |
| | GAUUCUGGCCAUGAGAGCGAUUCCAACAGCAACGAGGGC | |
| | CGGCACCAUUUGCUGGUGUCGGGGGCUGGAGACGGGCCU | |
| | CCGUUAUGUAGCCAAAACUUGGGCGCGCCCGGCGGCGGG | |
| | CCCGAUAACGGCCCGCAGGACCCGGACAAUACCGACGAUA | |
| | AUGGGCCUCAAGAUCCCGACAAUACUGAUGACAAUGGAC | |
| | CACACGAUCCUCUGCCACAGGAUCCCGACAACACAGAUGA | |
| | CAAUGGGCCGCAAGACCCCGACAAUACAGACGAUAAUGG | |
| | CCCCCACGACCCACUACCACAUUCUCCCUCUGACAGUGCG | |
| | GGGAAUGAUGGUGGUCCCCCACAACUGACCGAGGAGGUG | |
| | GAGAAUAAGGGGGGGGACCAAGGUCCACCCCUCAUGACU | |
| | GAUGGAGGAGGAGGACACUCGCACGAUUCAGGUCACGGC | |
| | GGGGGGGACCCUCACUUGCCCACUCUGCUGCUGGGCAGC | |
| | AGCGGUUCUGGGGGCGACGAUGAUGAUCCCCACGGCCCG | |
| | GUGCAGCUAAGUUAUUACGAC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEHDLERGPPGPRRPPRGPPLSSSLGLALLLLLLALLFWLYIV | 16 |
| amino acid | MSDWTGGALLVLYSFALMLIIIILIIFIFRRDLLCPLGALCILLLM | |
| sequence | ITLLLIALWNLHGQALFLGIVLFIFGCLLVLGIWIYLLEMLWRL | |
| | GATIWQLLAFFLAFFLDLILLIIALYLQQNWWTLLVDLLWLLL | |
| | FLAILIWMYYHGQRHSDEHHHDDSLPHPQQATDDSGHESDSN | |
| | SNEGRHHLLVSGAGDGPPLCSQNLGAPGGGPDNGPQDPDNTD | |
| | DNGPQDPDNTDDNGPHDPLPQDPDNTDDNGPQDPDNTDDNG | |
| | PHDPLPHSPSDSAGNDGGPPQLTEEVENKGGDQGPPLMTDGG | |
| | GGHSHDSGHGGGDPHLPTLLLGSSGSGGDDDDPHGPVQLSYY | |
| | D | |
| PolyA tail | 100 nt | |

LMP1_005

| SEQ ID NO: 144 consists of from 5′ end to 3′ end: | | 144 |
|---|---|---|
| 5′ UTR SEQ ID NO: 1, mRNA ORF SEQ ID NO: 20, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGCACGAUUUAGAGAGGGGCCCACCUGGCCCCAGG | 20 |
| Construct | CGCCCACCUAGAGGCCCACCCUUGUCGUCGUCAUUAGGGC | |
| (excluding | UGGCAUUGCUCCUGCUCCUCCUGGCCUUGCUGUUCUGGC | |
| the stop | UUUACAUCGUGAUGAGCGACUGGACUGGCGGGGCCCUGC | |
| codon) | UGGUCCUCUAUAGCUUUGCACUGAUGCUGAUUAUAAUCA | |
| | UAUUGAUCAUCUUCAUCUUCCGACGGGAUUUACUUUGCC | |
| | CCCUGGGGGCAUUAUGUAUUCUAUUGCUGAUGAUAACAC | |
| | UUCUGCUUAUUGCUUUAUGGAAUCUACACGGUCAGGCUC | |
| | UUUUUCUGGGAAUCGUGCUCUUUAUCUUUGGAUGCCUUC | |
| | UGGUACUAGGAAUUUGGAUAUACCUCUUAGAAAUGCUGU | |
| | GGCGGCUCGGCGCAACCAUCUGGCAACUCCUUGCGUUUU | |
| | UUCUCGCGUUUUUCCUCGACCUGAUUCUGCUAAUAAUCG | |
| | CCUUAUACCUUCAGCAGAAUUGGUGGACACUCCUGGUGG | |
| | ACCUGCUGUGGCUCCUGCUAUUCCUUGCAAUCCUCAUUU | |
| | GGAUGUAUUACCAUGGCCAGAGGCACUCUGAUGAGCACC | |
| | ACCACGAUGAUAGUCUACCUCAUCCACAGCAGGCUACGG | |
| | ACGAUUCAGGGCAUGAGUCAGAUUCUAAUUCCAACGAAG | |
| | GAAGACACCAUCUGUUGGUGUCAGGGGCCGGAGACGGCC | |
| | CCCCUCUGUGUAGCCAGAACCUCGGGGCACCAGGGGGAG | |
| | GUCCUGACAAUGGGCCGCAAGACCCUGACAAUACUGAUG | |
| | AUAAUGGCCCCCAAGAUCCUGACAAUACCGAUGACAACG | |
| | GCCCGCACGACCCCCUGCCGCAGGAUCCGGAUAACACCGA | |
| | CGACAAUGGACCCCAGGACCCCGAUAACACGGACGAUAA | |
| | CGGCCCUCACGAUCCCUUACCCCAUUCUCCGUCCGACUCU | |
| | GCAGGGAACGACGGGGGCCCUCCUCAGUUAACCGAAGAG | |
| | GUCGAAAAUAAGGGGGGAGACCAGGGCCCCCCAUUAAUG | |
| | ACAGACGGGGGCGGCGGUCACAGUCACGACUCAGGUCAC | |
| | GGCGGCGGGGACCCCCAUUUGCCUACACUGCUGUUAGGG | |
| | UCUUCUGGCUCAGGCGGGGACGAUGAUGAUCCACACGGU | |
| | CCGGUGCAGCUGUCAUAUUAUGAU | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEHDLERGPPGPRRPPRGPPLSSSLGLALLLLLLALLFWLYIV | 16 |
| amino acid | MSDWTGGALLVLYSFALMLIIIILIIFIFRRDLLCPLGALCILLLM | |
| sequence | ITLLLIALWNLHGQALFLGIVLFIFGCLLVLGIWIYLLEMLWRL | |
| | GATIWQLLAFFLAFFLDLILLIIALYLQQNWWTLLVDLLWLLL | |
| | FLAILIWMYYHGQRHSDEHHHDDSLPHPQQATDDSGHESDSN | |
| | SNEGRHHLLVSGAGDGPPLCSQNLGAPGGGPDNGPQDPDNTD | |
| | DNGPQDPDNTDDNGPHDPLPQDPDNTDDNGPQDPDNTDDNG | |
| | PHDPLPHSPSDSAGNDGGPPQLTEEVENKGGDQGPPLMTDGG | |
| | GGHSHDSGHGGGDPHLPTLLLGSSGSGGDDDDPHGPVQLSYY | |
| | D | |
| PolyA tail | 100 nt | |

LMP2_001

| SEQ ID NO: 145 consists of from 5′ end to 3′ end: | | 145 |
|---|---|---|
| 5′ UTR SEQ ID NO: 1, mRNA ORF SEQ ID NO: 21, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGUAGUCUUGAGAUGGUACCAAUGGGCGCAGGUCCA | 21 |
| Construct | CCGAGCCCUGGAGGCGAUCCUGACGGGUACGACGGGGGA | |
| (excluding | AAUAACUCGCAAUAUCCCUCAGCCUCAGGCUCAUCGGGU | |
| the stop | AAUACUCCGACCCCUCCAAACGACGAGGAAAGGGAAUCC | |
| codon) | AAUGAGGAACCGCCUCCACCAUAUGAGGAUCCGUACUGG | |
| | GGGAACGGUGAUAGGCACAGUGACUACCAGCCACUUGGG | |
| | ACACAAGACCAAAGCCUGUACCUCGGACUUCAGCACGAU | |
| | GGUAACGACGGUUUGCCGCCUCCCCCUUAUAGCCCUAGA | |
| | GAUGAUAGCUCCCAGCAUAUAUACGAGGAGGCUGGUAGG | |
| | GGCUCCAUGAAUCCAGUAUGCUUGCCCGUUAUCGUAGCC | |
| | CCCUACCUCUUCUGGCUUGCGGCGAUCGCAGCUAGCUGU | |
| | UUUACAGCUUCCGUGAGCACAGUCGUAACCGCGACAGGA | |
| | CUGGCACUGUCUCUACUCCUCUUGGCAGCGGUUGCCAGC | |
| | AGCUACGCCGCCGCUCAGCGGAAACUGCUCACCCCUGUUA | |
| | CAGUCUUAACUGCAGUUGUGACCUUCUUCGCCAUUUGUU | |
| | UGACAUGGAGGAUCGAAGAUCCUCCAUUUAAUAGUCUCC | |
| | UGUUUGCACUGCUGGCCGCUGCGGGCGGGCUACAAGGGA | |
| | UAUAUGUGCUAGUAAUGCUCGUCCUACUUAUUCUGGCCU | |
| | AUAGGCGGAGGUGGCGGCGCCUGACAGUGUGCGGAGGGA | |
| | UAAUGUUCCUAGCCUGUGUGCUCGUGCUCAUUGUGGACG | |
| | CUGUUCUACAGUUGUCUCCUCUGCUGGGUGCAGUGACGG | |
| | UGGUCUCAAUGACCCUGCUGUUACUUGCCUUCGUGCUUU | |
| | GGUUGUCAUCUCCAGGGGGACUGGGCACACUGGGCGCCG | |
| | CCCUGUUAACCCUGGCGGCCGCUCUGGCACUCCUGGCCUC | |
| | ACUGAUUCUGGGAACUCUGAAUCUCACCACUAUGUUUCU | |
| | UCUGAUGUUAUUGUGGACUUUGGUGGUGUUGCUGAUUUG | |
| | CAGCUCCUGCAGUUCUUGCCCCCUAUCCAAAAUAUUACU | |
| | GGCGCGGCUGUUCCUUUAUGCCCUCGCCUUACUGCUGCUC | |
| | GCUAGCGCCUUAAUUGCUGGGGGCUCCAUUCUUCAGACC | |
| | AACUUUAAAUCCCUGUCCAGCACUGAAUUCAUCCCCAACC | |
| | UUUUUUGCAUGCUGCUCCUAAUCGUGGCUGGGAUUCUGU | |
| | UUAUUCUGGCCAUUUUGACCGAGUGGGGCAGUGGCAACA | |
| | GGACCUACGGCCCUGUGUUCAUGUGUCUGGGGGGGCUCU | |
| | UAACGAUGGUCGCUGGCGCUGUCUGGCUCACGGUGAUGA | |
| | GUAACACUUUGCUCAGCGCGUGGAUCCUGACAGCCGGCU | |
| | UUCUCAUUUUUCUGAUUGGGUUCGCUUUGUUUGGAGUCA | |
| | UUCGGUGUUGCCGCUAUUGCUGUUACUAUUGUUUGACAU | |
| | UGGAGUCCGAGGAGAGGCCACCCACCCCCUACCGAAAUAC | |
| | GGUA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGSLEMVPMGAGPPSPGGDPDGYDGGNNSQYPSASGSSGNT | 22 |
| amino acid | PTPPNDEERESNEEPPPPYEDPYWGNGDRHSDYQPLGTQDQSL | |
| sequence | YLGLQHDGNDGLPPPPYSPRDDSSQHIYEEAGRGSMNPVCLP | |
| | VIVAPYLFWLAAIAASCFTASVSTVVTATGLALSLLLLAAVAS | |
| | SYAAAQRKLLTPVTVLTAVVTFFAICLTWRIEDPPFNSLLFALL | |
| | AAAGGLQGIYVLVMLVLLILAYRRRWRRLTVCGGIMFLACVL | |
| | VLIVDAVLQLSPLLGAVTVVSMTLLLLAFVLWLSSPGGLGTL | |
| | GAALLTLAAALALLASLILGTLNLTTMFLLMLLWTLVVLLICS | |
| | SCSSCPLSKILLARLFLYALALLLLASALIAGGSILQTNFKSLSS | |
| | TEFIPNLFCMLLLIVAGILFILAILTEWGSGNRTYGPVFMCLGG | |
| | LLTMVAGAVWLTVMSNTLLSAWILTAGFLIFLIGFALFGVIRC | |
| | CRYCCYYCLTLESEERPPTPYRNTV | |
| PolyA tail | 100 nt | |

LMP2_002

| SEQ ID NO: 146 consists of from 5′ end to 3′ end: | | 146 |
|---|---|---|
| 5′ UTR SEQ ID NO: 1, mRNA ORF SEQ ID NO: 23, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGAUCCUUGGAAAUGGUGCCCAUGGGAGCCGGACCC | 23 |
| Construct | CCCUCACCAGGGGGAGACCCAGACGGAUACGACGGGGGA | |
| (excluding | AACAAUUCUCAGUAUCCGAGCGCGUCCGGCAGCUCAGGA | |
| the stop | AACACACCCACCCCCCCCAACGACGAGGAGAGAGAAUCCA | |
| codon) | ACGAGGAGCCUCCUCCCCCAUACGAAGACCCUUACUGGGG | |
| | GAAUGGAGACCGUCAUUCUGACUAUCAACCUCUAGGCAC | |
| | UCAGGACCAGUCUCUUUACCUAGGGUUACAACACGAUGG | |
| | UAAUGAUGGGUUGCCCCCGCCGCCCUAUAGUCCCCGGGAC | |
| | GACAGUAGUCAGCAUAUAUAUGAGGAAGCCGGCAGAGGU | |
| | UCUAUGAAUCCCGUUUGUCUACCGGUCAUUGUCGCCCCC | |
| | UAUUUAUUCUGGCUUGCAGCCAUCGCCGCUUCGUGCUUC | |
| | ACCGCCUCCGUGUCCACGGUGGUGACAGCUACAGGGCUG | |
| | GCACUCUCUCUGCUACUCCUGGCUGCUGUGGCCAGCUCCU | |
| | ACGCAGCGGCUCAGCGGAAGCUUCUCACUCCUGUAACUG | |
| | UCCUCACCGCGGUAGUGACCUUUUUUGCCAUAUGCCUGA | |
| | CAUGGAGGAUUGAGGACCCACCCUUUAAUUCUCUGCUGU | |
| | UUGCGCUUUUGGCCGCUGCCGGAGGACUGCAAGGUAUUU | |
| | AUGUGCUGGUAAUGUUGGUUCUACUUAUUCUCGCAUACC | |
| | GGCGACGGUGGCGGAGGCUGACUGUAUGCGGGGGGAUUA | |
| | UGUUCCUGGCGUGUGUUCUGGUUUUGAUCGUCGAUGCCG | |
| | UGCUGCAGUUGUCACCGCUGCUCGGGGCGGUCACCGUUG | |
| | UGAGUAUGACACUGCUUCUGCUGGCUUUCGUGCUCUGGC | |
| | UAUCUUCCCCCGGGGGGCUGGGGACCUUGGGCGCUGCGC | |
| | UUCUGACUCUGGCAGCUGCUCUGGCCCUGCUGGCCUCUCU | |
| | UAUUCUUGGCACACUGAAUUUGACUACCAUGUUCCUCUU | |
| | GAUGUUGCUGUGGACCUUAGUUGUGCUGCUGAUCUGUUC | |
| | UUCCUGUAGCAGCUGUCCACUGUCCAAGAUUCUCCUGGC | |
| | CAGGUUGUUUCUGUAUGCCCUAGCGCUGCUGCUGUUAGC | |
| | UUCUGCUCUGAUAGCCGGCGGCAGUAUCCUGCAAACCAA | |
| | CUUCAAGAGCCUCUCCAGCACAGAAUUCAUCCCUAAUCUC | |
| | UUUUGCAUGCUACUGCUCAUCGUGGCAGGCAUACUGUUC | |
| | AUUCUUGCUAUCCUGACUGAAUGGGGCUCUGGCAAUAGA | |
| | ACAUACGGGCCAGUGUUCAUGUGCUUGGGCGGCCUGUUG | |
| | ACAAUGGUUGCUGGAGCCGUGUGGCUGACGGUAAUGUCC | |
| | AACACUCUGCUGAGCGCAUGGAUCUUAACAGCCGGAUUC | |
| | CUGAUCUUUUUAAUCGGAUUCGCACUCUUCGGAGUGAUC | |
| | CGGUGCUGUAGGUAUUGUUGUUAUUACUGCCUGACACUC | |
| | GAGUCGGAAGAGAGGCCCCCUACACCUUACCGGAAUACA | |
| | GUU | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGSLEMVPMGAGPPSPGGDPDGYDGGNNSQYPSASGSSGNT | 22 |
| amino acid | PTPPNDEERESNEEPPPPYEDPYWGNGDRHSDYQPLGTQDQSL | |
| sequence | YLGLQHDGNDGLPPPPYSPRDDSSQHIYEEAGRGSMNPVCLP | |
| | VIVAPYLFWLAAIAASCFTASVSTVVTATGLALSLLLLAAVAS | |
| | SYAAAQRKLLTPVTVLTAVVTFFAICLTWRIEDPPFNSLLFALL | |
| | AAAGGLQGIYVLVMLVLLILAYRRRWRRLTVCGGIMFLACVL | |
| | VLIVDAVLQLSPLLGAVTVVSMTLLLLAFVLWLSSPGGLGTL | |
| | GAALLTLAAALALLASLILGTLNLTTMFLLMLLWTLVVLLICS | |
| | SCSSCPLSKILLARLFLYALALLLLASALIAGGSILQTNFKSLSS | |
| | TEFIPNLFCMLLLIVAGILFILAILTEWGSGNRTYGPVFMCLGG | |
| | LLTMVAGAVWLTVMSNTLLSAWILTAGFLIFLIGFALFGVIRC | |
| | CRYCCYYCLTLESEERPPTPYRNTV | |
| PolyA tail | 100 nt | |

LMP2_003

| SEQ ID NO: 147 consists of from 5′ end to 3′ end: | | 147 |
|---|---|---|
| 5′ UTR SEQ ID NO: 1, mRNA ORF SEQ ID NO: 24, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGGUCGUUGGAGAUGGUCCCCAUGGGCGCUGGUCCU | 24 |
| Construct | CCAAGCCCCGGCGGGGACCCGGACGGAUACGACGGGGGC | |
| (excluding | AAUAAUAGUCAGUAUCCGAGUGCAAGCGGAAGCAGUGGG | |
| the stop | AAUACGCCUACACCACCAAACGACGAGGAGCGGGAGUCU | |
| codon) | AACGAGGAGCCUCCACCCCCUUACGAGGAUCCUUACUGG | |
| | GGGAACGGAGAUCGACAUUCUGACUACCAGCCUUUAGGG | |
| | ACACAGGAUCAGUCUCUCUAUCUCGGACUGCAGCAUGAU | |
| | GGCAACGACGGACUGCCCCCACCACCUUAUUCUCCACGCG | |
| | ACGACAGCUCACAGCACAUAUACGAAGAGGCCGGGCGCG | |
| | GGUCUAUGAACCCGGUUUGCCUGCCAGUCAUCGUCGCAC | |
| | CCUACCUCUUUUGGCUGGCGGCCAUUGCCGCGUCCUGUU | |
| | UCACAGCCUCAGUGUCGACUGUGGUAACCGCAACAGGGC | |
| | UCGCACUCAGCCUACUGCUGCUCGCUGCCGUCGCGUCCUC | |
| | UUAUGCUGCUGCCCAGCGCAAACUGCUCACCCCAGUUACG | |
| | GUGCUCACUGCUGUUGUUACCUUUUUCGCUAUUUGUCUC | |
| | ACCUGGAGAAUCGAAGACCCGCCGUUCAACAGUCUGUUA | |
| | UUUGCCCUCUUGGCUGCAGCAGGCGGCCUUCAGGGCAUC | |
| | UAUGUCCUGGUGAUGCUGGUCCUGCUCAUCCUGGCUUAC | |
| | AGGAGAAGAUGGCGUCGGCUGACCGUGUGUGGUGGCAUC | |
| | AUGUUUCUCGCGUGUGUUCUGGUGCUCAUCGUCGAUGCC | |
| | GUGCUACAGCUGAGCCCACUGCUUGGAGCCGUGACAGUC | |
| | GUUAGCAUGACACUCCUGCUAUUGGCUUUCGUGCUCUGG | |
| | CUGAGUAGCCCAGGUGGGCUCGGCACACUUGGUGCCGCG | |
| | CUUUUAACACUGGCCGCGGCCCUCGCACUGCUGGCCUCUC | |
| | UAAUUCUCGGAACAUUGAACCUGACAACCAUGUUCCUCC | |
| | UGAUGCUCCUCUGGACACUGGUGGUACUGUUAAUCUGUA | |
| | GCUCUUGCUCAUCAUGCCCACUGAGCAAGAUACUCCUGG | |
| | CGAGACUUUUCCUAUACGCCCUGGCCCUGCUGCUGUUGG | |
| | CAUCGGCUCUGAUCGCUGGGGGAAGCAUCCUUCAAACAA | |
| | AUUUCAAGAGCCUUAGCAGUACUGAGUUUAUACCUAACC | |
| | UGUUCUGUAUGCUUCUGCUGAUAGUCGCUGGGAUACUUU | |
| | UUAUUCUGGCAAUCCUAACAGAGUGGGGGUCAGGUAACA | |
| | GGACCUAUGGGCCUGUUUUUAUGUGCCUCGGAGGCCUCC | |
| | UCACCAUGGUUGCCGGCGCAGUUUGGUUGACAGUGAUGU | |
| | CCAACACCCUACUGAGUGCUUGGAUACUGACGGCCGGGU | |
| | UCCUGAUUUUCCUAAUAGGAUUUGCCCUGUUUGGGGUUA | |
| | UCCGGUGCUGCCGUUACUGCUGCUACUAUUGUUUGACCC | |
| | UUGAGUCUGAAGAAAGGCCGCCCACCCCCUACAGAAACA | |
| | CUGUA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGSLEMVPMGAGPPSPGGDPDGYDGGNNSQYPSASGSSGNT | 22 |
| amino acid | PTPPNDEERESNEEPPPPYEDPYWGNGDRHSDYQPLGTQDQSL | |
| sequence | YLGLQHDGNDGLPPPPYSPRDDSSQHIYEEAGRGSMNPVCLP | |
| | VIVAPYLFWLAAIAASCFTASVSTVVTATGLALSLLLLAAVAS | |
| | SYAAAQRKLLTPVTVLTAVVTFFAICLTWRIEDPPFNSLLFALL | |
| | AAAGGLQGIYVLVMLVLLILAYRRRWRRLTVCGGIMFLACVL | |
| | VLIVDAVLQLSPLLGAVTVVSMTLLLLAFVLWLSSPGGLGTL | |
| | GAALLTLAAALALLASLILGTLNLTTMFLLMLLWTLVVLLICS | |
| | SCSSCPLSKILLARLFLYALALLLLASALIAGGSILQTNFKSLSS | |
| | TEFIPNLFCMLLLIVAGILFILAILTEWGSGNRTYGPVFMCLGG | |
| | LLTMVAGAVWLTVMSNTLLSAWILTAGFLIFLIGFALFGVIRC | |
| | CRYCCYYCLTLESEERPPTPYRNTV | |
| PolyA tail | 100 nt | |

LMP2_004

| SEQ ID NO: 148 consists of from 5′ end to 3′ end: | | 148 |
|---|---|---|
| 5′ UTR SEQ ID NO: 1, mRNA ORF SEQ ID NO: 25, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGGUCAUUGGAGAUGGUGCCUAUGGGGGCCGGCCCA | 25 |
| Construct | CCCAGCCCAGGGGGCGAUCCUGACGGAUACGACGGGGGC | |
| (excluding | AAUAAUUCACAAUAUCCUAGCGCCAGCGGAUCUUCUGGG | |
| the stop | AAUACCCCCACACCACCUAACGACGAGGAACGGGAGUCA | |
| codon) | AACGAGGAACCCCCACCGCCGUACGAGGACCCAUACUGGG | |
| | GGAACGGGGAUCGACACUCAGACUACCAGCCUCUCGGAA | |
| | CGCAGGAUCAAAGCCUGUACCUGGGACUGCAGCACGAUG | |
| | GUAACGAUGGACUGCCACCACCUCCUUACAGUCCAAGGG | |
| | AUGACUCCAGCCAGCACAUUUAUGAAGAAGCCGGCCGAG | |
| | GUAGUAUGAAUCCAGUGUGUCUGCCCGUGAUUGUCGCCC | |
| | CUUAUCUCUUUUGGCUGGCCGCCAUUGCCGCCAGCUGUU | |
| | UCACCGCGUCGGUUUCAACCGUAGUGACCGCCACAGGCCU | |
| | GGCUCUCAGCUUGUUACUGUUGGCCGCUGUGGCGAGCUC | |
| | CUACGCCGCCGCCCAGCGCAAGCUACUUACCCCUGUGACA | |
| | GUGCUGACGGCGGUGGUUACAUUCUUCGCCAUUUGCCUG | |
| | ACCUGGCGAAUUGAGGAUCCACCAUUCAAUUCCUUAUUG | |
| | UUUGCCCUCCUCGCCGCUGCUGGAGGGCUCCAGGGUAUC | |
| | UACGUGCUGGUGAUGCUCGUUUUGCUGAUAUUGGCUUAC | |
| | CGGCGGCGCUGGCGCCGCCUUACCGUCUGCGGAGGAAUC | |
| | AUGUUUUUGGCAUGCGUGCUCGUUCUCAUUGUGGAUGCU | |
| | GUUUUGCAGCUGUCACCCCUGUUGGGUGCCGUGACAGUU | |
| | GUAAGUAUGACCCUGCUCCUGCUCGCAUUCGUGCUCUGG | |
| | CUGAGCUCUCCCGGGGGGCUGGGCACUCUCGGAGCAGCU | |
| | CUGCUGACACUAGCCGCAGCACUAGCCCUUCUGGCCAGUC | |
| | UUAUCCUCGGAACGCUGAACCUGACGACCAUGUUCCUCC | |
| | UGAUGUUGCUCUGGACCCUCGUAGUGCUGUUAAUAUGCU | |
| | CUAGCUGUAGCUCCUGCCCUCUGAGCAAGAUUCUCCUUG | |
| | CUAGGCUGUUCCUAUACGCCCUCGCUCUCCUAUUGCUGGC | |
| | GAGCGCUCUCAUCGCCGGUGGGAGCAUCCUGCAGACUAA | |
| | CUUCAAGUCCCUGAGUUCAACAGAAUUCAUUCCCAACCU | |
| | CUUUUGUAUGCUGCUGCUUAUCGUGGCCGGCAUCCUCUU | |
| | UAUCCUCGCCAUCUUGACUGAGUGGGGGUCCGGAAAUCG | |
| | CACAUAUGGCCCAGUUUUCAUGUGUCUGGGGGGCCUGCU | |
| | CACGAUGGUGGCCGGCGCAGUGUGGCUGACGGUAAUGUC | |
| | AAACACUUUGCUCUCUGCGUGGAUCUUGACCGCAGGCUU | |
| | CCUGAUCUUUUUGAUCGGCUUCGCCCUGUUCGGUGUCAU | |
| | CCGGUGCUGCAGAUACUGCUGCUACUAUUGCCUCACACU | |
| | CGAAUCCGAAGAGAGGCCUCCCACACCUUACCGAAAUAC | |
| | AGUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGSLEMVPMGAGPPSPGGDPDGYDGGNNSQYPSASGSSGNT | 22 |
| amino acid | PTPPNDEERESNEEPPPPYEDPYWGNGDRHSDYQPLGTQDQSL | |
| sequence | YLGLQHDGNDGLPPPPYSPRDDSSQHIYEEAGRGSMNPVCLP | |
| | VIVAPYLFWLAAIAASCFTASVSTVVTATGLALSLLLLAAVAS | |
| | SYAAAQRKLLTPVTVLTAVVTFFAICLTWRIEDPPFNSLLFALL | |
| | AAAGGLQGIYVLVMLVLLILAYRRRWRRLTVCGGIMFLACVL | |
| | VLIVDAVLQLSPLLGAVTVVSMTLLLLAFVLWLSSPGGLGTL | |
| | GAALLTLAAALALLASLILGTLNLTTMFLLMLLWTLVVLLICS | |
| | SCSSCPLSKILLARLFLYALALLLLASALIAGGSILQTNFKSLSS | |
| | TEFIPNLFCMLLLIVAGILFILAILTEWGSGNRTYGPVFMCLGG | |
| | LLTMVAGAVWLTVMSNTLLSAWILTAGFLIFLIGFALFGVIRC | |
| | CRYCCYYCLTLESEERPPTPYRNTV | |
| PolyA tail | 100 nt | |

LMP2_005

| SEQ ID NO: 149 consists of from 5′ end to 3′ end: | | 149 |
|---|---|---|
| 5′ UTR SEQ ID NO: 1, mRNA ORF SEQ ID NO: 26, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGGUCUUUAGAAAUGGUCCCCAUGGGUGCCGGCCCA | 26 |
| Construct | CCCUCGCCAGGAGGCGAUCCAGACGGCUACGACGGGGGA | |
| (excluding | AACAACAGCCAGUACCCCAGCGCCAGCGGUAGCAGCGGCA | |
| the stop | ACACCCCGACCCCACCAAACGACGAAGAGCGGGAGAGCAA | |
| codon) | UGAGGAGCCCCCACCCCCCUAUGAGGACCCUUACUGGGGA | |
| | AAUGGCGAUCGACACAGCGAUUACCAGCCCCUGGGCACA | |
| | CAGGAUCAGAGCCUGUACCUGGGCUUGCAGCAUGAUGGU | |
| | AACGACGGGCUUCCGCCCCCACCAUACUCACCCAGAGACG | |
| | AUUCAAGUCAGCACAUCUACGAGGAGGCUGGUAGGGGUA | |
| | GUAUGAAUCCUGUGUGUCUCCCUGUGAUCGUCGCCCCCU | |
| | ACCUAUUUUGGUUAGCUGCGAUCGCUGCCUCCUGCUUCA | |
| | CCGCUAGCGUGUCAACAGUGGUUACGGCAACCGGGCUGG | |
| | CGUUAAGUCUGCUUCUGCUUGCGGCAGUGGCAUCUUCUU | |
| | AUGCAGCGGCCCAGCGGAAGCUUUUGACCCCUGUGACAG | |
| | UACUGACCGCAGUGGUUACGUUUUUUGCGAUUUGCUUGA | |
| | CGUGGAGAAUAGAGGAUCCCCCUUUUAACUCACUGCUCU | |
| | UUGCACUGCUCGCGGCUGCUGGGGGCCUGCAGGGGAUUU | |
| | ACGUCCUCGUUAUGCUGGUGCUCCUGAUCUUAGCCUACA | |
| | GAAGGAGAUGGCGCCGACUGACAGUAUGCGGGGGGAUUA | |
| | UGUUUUUGGCAUGUGUACUCGUAUUGAUCGUGGAUGCCG | |
| | UCUUGCAGCUUUCCCCCCUGCUGGGCGCAGUCACCGUCGU | |
| | GAGUAUGACUCUGCUGUUACUGGCCUUCGUACUGUGGCU | |
| | UAGCAGCCCUGGAGGACUAGGCACGCUGGGCGCCGCUUU | |
| | AUUGACCCUGGCAGCCGCCCUGGCUCUGCUGGCCUCCCUG | |
| | AUACUGGGGACAUUAAAUUUAACAACUAUGUUUCUGCUG | |
| | AUGCUGCUGUGGACUCUAGUUGUGCUCUUGAUAUGUUCC | |
| | UCCUGUUCCAGCUGCCCUCUGAGCAAGAUCCUGUUGGCU | |
| | CGGCUAUUCCUGUACGCGCUGGCUCUCCUCCUGCUAGCUU | |
| | CAGCGCUAAUUGCAGGCGGGUCUAUUCUCCAGACGAAUU | |
| | UUAAGAGCCUCAGUAGCACUGAGUUUAUCCCGAAUCUGU | |
| | UCUGCAUGUUGCUAUUGAUCGUCGCAGGCAUUCUCUUCA | |
| | UUCUGGCCAUACUUACAGAGUGGGGUAGCGGCAACCGGA | |
| | CUUAUGGCCCUGUGUUCAUGUGCUUGGGGGGCCUUCUCA | |
| | CAAUGGUGGCAGGGGCGGUAUGGCUGACCGUGAUGUCUA | |
| | ACACCUUGUUAUCAGCCUGGAUCCUUACCGCCGGCUUCCU | |
| | AAUUUUUCUGAUCGGCUUCGCCCUCUUCGGAGUAAUACG | |
| | AUGCUGUCGGUACUGCUGCUACUAUUGCCUUACUUUGGA | |
| | GAGCGAAGAGCGGCCUCCCACUCCCUAUAGAAACACCGU | |
| | U | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGSLEMVPMGAGPPSPGGDPDGYDGGNNSQYPSASGSSGNT | 22 |
| amino acid | PTPPNDEERESNEEPPPPYEDPYWGNGDRHSDYQPLGTQDQSL | |
| sequence | YLGLQHDGNDGLPPPPYSPRDDSSQHIYEEAGRGSMNPVCLP | |
| | VIVAPYLFWLAAIAASCFTASVSTVVTATGLALSLLLLAAVAS | |
| | SYAAAQRKLLTPVTVLTAVVTFFAICLTWRIEDPPFNSLLFALL | |
| | AAAGGLQGIYVLVMLVLLILAYRRRWRRLTVCGGIMFLACVL | |
| | VLIVDAVLQLSPLLGAVTVVSMTLLLLAFVLWLSSPGGLGTL | |
| | GAALLTLAAALALLASLILGTLNLTTMFLLMLLWTLVVLLICS | |
| | SCSSCPLSKILLARLFLYALALLLLASALIAGGSILQTNFKSLSS | |
| | TEFIPNLFCMLLLIVAGILFILAILTEWGSGNRTYGPVFMCLGG | |
| | LLTMVAGAVWLTVMSNTLLSAWILTAGFLIFLIGFALFGVIRC | |
| | CRYCCYYCLTLESEERPPTPYRNTV | |
| PolyA tail | 100 nt | |

gp350_mod_001

| SEQ ID NO: 150 consists of from 5′ end to 3′ end: | | 150 |
|---|---|---|
| 5′ UTR SEQ ID NO: 1, mRNA ORF SEQ ID NO: 27, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 27 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UUUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUU | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACCCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUUUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAAACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACCCCCGCAGGUACAACCUCAGGCG | |
| | CCAGCCCAGUGACCCCUUCCCCAAGCCCCUGGGAUAAUGG | |
| | AACGGAGUCCAAGGCUCCUGAUAUGACUUCCUCUACCAG | |
| | CCCCGUGACUACACCCACUCCCAACGCAACUAGCCCAACC | |
| | CCAGCUGUGACGACCCCCACCCCGAACGCGACAUCUCCCA | |
| | CACCUGCUGUGACAACCCCAACCCCUAACGCCACUAGCCC | |
| | UACCCUAGGUAAGACCAGUCCGACUAGCGCCGUUACAAC | |
| | CCCUACCCCUAACGCAACCGGCCCGACCGUGGGCGAGACU | |
| | UCCCCGCAAGCCAAUGCGACAAAUCACACAUUGGGCGGG | |
| | ACCUCUCCUACACCAGUCGUUACAUCUCAGCCUAAGAACG | |
| | CUACCUCCGCUGUCACUACCGGACAGCACAACAUCACCAG | |
| | CUCAAGCACGAGUUCCAUGAGCUUGCGGCCGAGCUCAAA | |
| | UCCCGAGACCCUAUCACCAUCCACAUCAGACAACAGUACU | |
| | UCACACAUGCCACUCUUGACGAGCGCUCACCCCACCGGCG | |
| | GCGAGAACAUCACCCAGGUGACUCCGGCGUCUAUUUCCA | |
| | CCCACCACGUCAGCACGUCGUCUCCCGCACCGAGACCAGG | |
| | GACGACUUCUCAGGCCAGUGGGCCUGGCAACUCCUCUAC | |
| | AAGCACAAAACCAGGCGAAGUUAACGUGACAAAAGGAAC | |
| | GCCCCCCCAGAACGCAACCAGUCCUCAGGCCCCCAGCGGG | |
| | CAGAAAACUGCGGUGCCAACUGUGACCAGCACCGGUGGC | |
| | AAGGCCAACUCAACAACUGGAGGCAAGCAUACGACGGGG | |
| | CACGGCGCCCGGACCUCCACUGAACCCACGACCGAUUACG | |
| | GAGGUGACAGCACAACACCGCGGCCACGAUAUAAUGCCA | |
| | CCACUUAUCUGCCACCAUCCACAAGCUCCAAGCUGCGGCC | |
| | ACGGUGGACCUUCACAAGCCCACCCGUGACGACUGCCCAA | |
| | GCGACGGUGCCAGUGCCCCCUACAAGCCAGCCACGCUUCU | |
| | CCAACCUUAGUAUGCUCGUUCUCCAGUGGGCCAGCCUUG | |
| | CUGUUCUGACCCUCCUCCUGCUGCUCGUGAUGGCCGAUU | |
| | GCGCCUUUAGGAGAAACCUCAGCACUAGCCACACGUACA | |
| | CCACGCCCCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 28 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

gp350_mod_002

| SEQ ID NO: 151 consists of from 5′ end to 3′ end: | | 151 |
|---|---|---|
| 5′ UTR SEQ ID NO: 1, mRNA ORF SEQ ID NO: 29, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCUGCUCUAUUGGUGUGCCAAUACACCAUUCAG | 29 |
| Construct | UCGCUGAUCCACUUGACUGGGGAGGAUCCCGGAUUUUUC | |
| (excluding | AACGUGGAGAUUCCGGAAUUCCCUUUUUACCCCACCUGC | |
| the stop | AACGUGUGCACUGCCGACGUAAACGUGACCAUAAACUUU | |
| codon) | GACGUCGGUGGGAAGAAGCACCAGCUGGACCUGGACUUC | |
| | GGGCAGUUAACACCCCACACAAAGGCAGUUUACCAGCCC | |
| | AGAGGAGCCUUCGGCGGAUCGGAGAAUGCAACAAAUCUU | |
| | UUCCUGCUAGAACUGCUCGGCGCCGGCGAGUUGGCCCUG | |
| | ACAAUGAGAAGCAAGAAACUUCCUAUCAACGUAACCACA | |
| | GGGGAGGAGCAGCAGGUCAGCCUCGAAAGUGUUGACGUC | |
| | UAUUUCCAGGAUGUCUUUGGGACUAUGUGGUGCCAUCAC | |
| | GCCGAAAUGCAGAAUCCCGUGUACCUCAUUCCCGAGACA | |
| | GUACCCUAUAUCAAGUGGGACAACUGCAACUCUACUAAU | |
| | AUUACAGCCGUGGUGCGCGCUCAGGGACUUGAUGUAACA | |
| | UUGCCUUUGUCUUUGCCCACAAGCGCUCAGGAUUCUAAU | |
| | UUCUCUGUAAAGACGGAGAUGCUGGGGAAUGAAAUCGAC | |
| | AUAGAAUGUAUUAUGGAAGACGGUGAAAUCAGUCAGGUG | |
| | CUACCGGGAGACAACAAAUUCAAUAUUACAUGUUCUGGC | |
| | UAUGAGAGCCACGUGCCGAGCGGGGGCAUCCUGACUUCG | |
| | ACUUCUCCCGUCGCCACACCCAUACCAGGCACAGGCUACG | |
| | CUUAUAGUCUGAGGCUGACUCCCAGGCCAGUAUCCCGAU | |
| | UUCUGGGUAAUAAUUCCAUCCUUUAUGUGUUUUACUCCG | |
| | GUAACGGCCCCAAAGCUUCGGGGGGCGACUAUUGUAUCC | |
| | AGUCUAAUAUAGUGUUCUCCGAUGAGAUUCCCGCGAGUC | |
| | AGGAUAUGCCCACCAACACCACCGACAUCACGUAUGUUG | |
| | GGGAUAACGCUACCUAUAGUGUGCCGAUGGUGACAUCCG | |
| | AGGACGCAAACUCUCCUAACGUAACCGUGACCGCCUUUU | |
| | GGGCAUGGCCAAAUAAUACCGAAACCGAUUUUAAAUGCA | |
| | AGUGGACCCUGACAUCCGGGACUCCCUCCGGAUGCGAAA | |
| | AUAUUUCUGGAGCUUUUGCCAGCAAUCGGACCUUUGAUA | |
| | UCACUGUGAGUGGUUUAGGCACCGCCCCAAAAACACUGA | |
| | UCAUAACCAGGACAGCCACAAAUGCGACCACAACUACAC | |
| | ACAAAGUUAUAUUUAGUAAAGCACCUGAGUCCACCACAA | |
| | CUUCUCCGACCCUCAACACUACCGGCUUCGCUGAUCCGAA | |
| | UACCACCACAGGUCUUCCAUCUUCCACCCACGUUCCAACU | |
| | AAUCUUACGGCGCCUGCUAGCACCGGUCCAACCGUGUCCA | |
| | CCGCCGAUGUGACUAGCCCCACGCCGGCUGGAACAACAUC | |
| | AGGGGCCAGCCCUGUAACCCCAAGCCCUUCACCCUGGGAC | |
| | AAUGGUACCGAGAGUAAGGCGCCCGACAUGACCUCAUCA | |
| | ACUUCUCCUGUUACAACUCCAACACCAAAUGCCACUAGUC | |
| | CUACCCCCGCAGUCACUACCCCAACGCCUAAUGCAACGUC | |
| | ACCAACUCCUGCAGUGACCACACCAACACCCAAUGCUACU | |
| | UCGCCGACCCUGGGCAAGACCAGCCCAACCAGUGCCGUGA | |
| | CGACACCGACUCCAAAUGCGACCGGACCUACUGUCGGUG | |
| | AGACAAGCCCCCAGGCAAACGCGACCAACCACACCCUCGG | |
| | CGGGACUUCUCCAACACCCGUCGUAACGUCCCAACCAAAA | |
| | AAUGCUACAUCGGCCGUGACUACCGGACAGCAUAAUAUC | |
| | ACCUCAAGCUCCACGUCGUCAAUGAGCCUGCGGCCGUCAA | |
| | GUAAUCCAGAGACUCUGUCGCCAUCUACCUCAGAUAACU | |
| | CAACUAGCCACAUGCCUCUACUCACUAGUGCCCACCCCAC | |
| | CGGCGGGGAGAACAUCACGCAGGUAACACCAGCGUCUAU | |
| | CAGUACUCACCAUGUGAGUACCAGCUCCCCCGCUCCUCGA | |
| | CCGGGAACCACGAGCCAGGCCAGCGGACCGGGUAAUAGU | |
| | AGCACUUCUACUAAACCGGGAGAGGUCAAUGUGACUAAG | |
| | GGGACUCCCCCCCAAAACGCUACCAGCCCACAGGCUCCCU | |
| | CAGGGCAGAAGACCGCUGUUCCAACAGUUACAUCUACUG | |
| | GGGGCAAGGCAAAUAGCACAACCGGUGGCAAGCAUACUA | |
| | CUGGGCACGGUGCCCGCACAAGCACCGAGCCUACAACGGA | |
| | CUAUGGGGGGGAUUCUACCACACCACGUCCCCGGUAUAA | |
| | CGCCACCACGUAUCUUCCGCCUAGCACAUCAAGCAAACUC | |
| | AGACCUCGCUGGACAUUUACAUCACCCCCAGUUACCACUG | |
| | CUCAGGCAACCGUCCCGGUGCCUCCUACUUCACAACCACG | |
| | UUUCAGCAACUUGAGCAUGCUGGUCCUGCAGUGGGCUUC | |
| | CCUCGCCGUGCUGACAUUACUGUUAUUGCUGGUGAUGGC | |
| | CGAUUGCGCGUUUCGCAGGAACCUGUCUACCAGCCACAC | |
| | AUACACAACGCCCCCGUAUGACGACGCAGAGACUUAUGU | |
| | G | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 28 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

gp350_mod_003

| SEQ ID NO: 152 consists of from 5′ end to 3′ end: | | 152 |
|---|---|---|
| 5′ UTR SEQ ID NO: 1, mRNA ORF SEQ ID NO: 30, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAAGCCGCUCUUCUGGUCUGCCAGUAUACAAUCCAG | 30 |
| Construct | UCGCUGAUUCAUCUCACCGGCGAGGAUCCAGGUUUCUUU | |
| (excluding | AACGUGGAGAUUCCAGAAUUCCCUUUCUAUCCAACUUGU | |
| the stop | AACGUGUGUACUGCCGACGUUAACGUUACUAUUAACUUU | |
| codon) | GACGUGGGCGGAAAGAAGCACCAGCUGGAUCUUGAUUUC | |
| | GGCCAGCUAACACCUCAUACCAAGGCCGUCUACCAACCAC | |
| | GCGGGGCAUUUGGCGGUUCUGAGAAUGCUACAAACCUGU | |
| | UCCUUUUGGAACUCCUCGGAGCAGGGGAACUGGCACUUA | |
| | CAAUGAGGAGUAAGAAGCUGCCCAUCAACGUCACCACGG | |
| | GGGAAGAACAGCAGGUGUCCCUGGAGUCCGUUGAUGUGU | |
| | AUUUCCAGGACGUUUUCGGGACAAUGUGGUGUCAUCAUG | |
| | CCGAAAUGCAGAAUCCGGUGUAUCUCAUUCCUGAGACCG | |
| | UGCCCUAUAUUAAAUGGGAUAACUGUAAUUCGACAAACA | |
| | UAACUGCCGUAGUGCGUGCCCAGGGUCUGGAUGUAACUU | |
| | UGCCUCUCUCCCUUCCCACUUCUGCCCAGGACUCCAAUUU | |
| | UUCAGUGAAGACCGAAAUGCUUGGGAACGAGAUCGACAU | |
| | CGAGUGUAUCAUGGAGGAUGGGGAGAUCAGUCAGGUGUU | |
| | GCCUGGUGACAAUAAGUUUAAUAUAACAUGUUCCGGCUA | |
| | UGAAUCACAUGUUCCAAGCGGAGGUAUUCUCACAAGUAC | |
| | AUCUCCCGUAGCCACGCCGAUUCCGGGAACCGGAUACGCC | |
| | UAUUCACUUCGGCUGACUCCUCGGCCAGUGUCACGAUUC | |
| | CUGGGCAAUAACUCAAUUCUGUACGUCUUUUACAGCGGC | |
| | AACGGCCCAAAGGCCAGUGGAGGCGAUUACUGUAUCCAG | |
| | UCUAACAUCGUAUUUUCAGAUGAGAUUCCAGCAAGCCAA | |
| | GACAUGCCAACUAAUACUACAGACAUUACAUACGUCGGU | |
| | GAUAACGCCACUUAUUCAGUCCCUAUGGUCACAUCCGAG | |
| | GACGCGAACUCACCCAAUGUUACCGUUACCGCAUUCUGG | |
| | GCAUGGCCCAAUAAUACGGAAACAGACUUCAAAUGUAAG | |
| | UGGACCCUGACCAGUGGCACCCCUUCUGGCUGCGAAAAU | |
| | AUAAGCGGGGCAUUUGCUAGCAACAGAACAUUCGAUAUU | |
| | ACUGUGUCUGGCCUGGGGACCGCCCCCAAAACACUCAUU | |
| | AUAACGCGAACAGCUACGAACGCUACCACAACCACACAU | |
| | AAAGUGAUUUUCAGCAAGGCUCCAGAAUCAACUACAACC | |
| | AGUCCGACCCUUAAUACCACAGGCUUCGCAGACCCGAACA | |
| | CAACGACGGGACUGCCCUCCUCAACGCAUGUGCCGACUAA | |
| | UCUGACUGCUCCCGCAAGUACCGGCCCAACAGUCUCCACA | |
| | GCAGACGUCACCUCUCCGACUCCCGCCGGCACUACAUCUG | |
| | GAGCCUCACCGGUGACUCCUAGUCCUUCCCCCUGGGACAA | |
| | UGGGACCGAGUCUAAAGCCCCGGACAUGACGAGUUCUAC | |
| | CUCACCAGUUACAACCCCAACACCUAAUGCCACAAGCCCU | |
| | ACGCCAGCCGUGACCACCCCUACCCCCAAUGCCACCAGCC | |
| | CAACACCCGCUGUUACAACGCCAACCCCCAACGCUACUUC | |
| | UCCAACGCUAGGAAAAACAUCACCCACCUCAGCUGUAACC | |
| | ACCCCCACACCAAACGCUACCGGGCCAACCGUUGGCGAAA | |
| | CCUCCCCCCAGGCCAAUGCGACAAACCACACCCUUGGGGG | |
| | GACUUCUCCCACUCCUGUGGUGACUUCACAACCGAAGAA | |
| | CGCGACGUCGGCCGUUACGACCGGGCAGCACAAUAUCACC | |
| | UCGUCCUCCACGUCGUCCAUGUCCUUGCGGCCUUCCAGUA | |
| | ACCCAGAGACACUGUCUCCUAGUACCAGUGACAACAGCA | |
| | CCUCUCACAUGCCAUUACUGACUUCCGCCCACCCAACCGG | |
| | CGGAGAGAACAUCACUCAGGUGACCCCCGCAUCUAUUUC | |
| | UACCCACCACGUGAGCACUAGCAGCCCGGCACCAAGACCA | |
| | GGGACCACCUCCCAGGCUAGCGGCCCGGGCAACUCCUCCA | |
| | CUAGCACGAAACCAGGAGAGGUGAACGUGACCAAGGGCA | |
| | CACCGCCACAGAAUGCAACUUCCCCACAGGCCCCCUCCGG | |
| | CCAGAAAACUGCUGUACCUACGGUCACUAGCACUGGCGG | |
| | AAAAGCGAAUUCAACCACCGGUGGUAAGCAUACCACCGG | |
| | ACACGGCGCUCGGACUAGCACAGAGCCAACCACUGAUUA | |
| | CGGGGGUGACUCAACGACCCCGAGGCCCAGGUACAACGC | |
| | AACCACCUAUCUCCCACCAUCCACAUCUUCCAAGCUCAGG | |
| | CCUCGUUGGACUUUCACCUCCCCGCCUGUGACUACAGCGC | |
| | AGGCCACAGUGCCAGUGCCUCCAACGUCACAGCCGCGGUU | |
| | UAGCAAUCUCUCCAUGCUGGUCCUGCAGUGGGCAUCUCU | |
| | GGCAGUCCUGACACUGCUUCUUUUGCUCGUGAUGGCCGA | |
| | UUGUGCGUUUAGACGCAACCUUUCAACAUCCCACACAUA | |
| | UACAACUCCACCCUAUGAUGACGCAGAGACUUAUGUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 28 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

gp350_mod_004

| SEQ ID NO: 153 consists of from 5′ end to 3′ end: | | 153 |
|---|---|---|
| 5′ UTR SEQ ID NO: 1, mRNA ORF SEQ ID NO: 31, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAAGCCGCCCUAUUAGUCUGCCAGUACACCAUUCAA | 31 |
| Construct | AGCCUCAUCCACUUGACUGGGGAGGAUCCUGGGUUCUUU | |
| (excluding | AACGUGGAAAUCCCGGAAUUCCCUUUCUACCCAACUUGC | |
| the stop | AACGUGUGCACGGCUGACGUCAACGUGACUAUCAACUUC | |
| codon) | GAUGUGGGGGGGAAGAAACAUCAACUUGAUCUGGACUUU | |
| | GGCCAGCUUACUCCCCACACCAAGGCCGUGUACCAGCCCC | |
| | GCGGCGCAUUCGGUGGGAGCGAGAACGCGACAAAUCUAU | |
| | UCUUGCUGGAACUGCUGGGCGCAGGAGAGCUCGCCCUGA | |
| | CGAUGCGAUCUAAAAAGCUUCCCAUCAACGUUACCACUG | |
| | GCGAGGAGCAGCAGGUGUCCCUCGAAAGCGUCGACGUCU | |
| | AUUUCCAAGACGUCUUCGGAACAAUGUGGUGCCACCACG | |
| | CUGAGAUGCAGAACCCCGUAUAUCUUAUUCCCGAAACCG | |
| | UGCCAUACAUCAAGUGGGACAACUGCAACUCAACUAACA | |
| | UCACUGCAGUAGUGCGCGCCCAGGGACUGGACGUCACUU | |
| | UGCCACUGUCACUGCCCACAUCAGCCCAAGACAGCAACUU | |
| | CAGCGUCAAGACAGAAAUGCUGGGCAACGAAAUCGACAU | |
| | CGAAUGCAUAAUGGAGGAUGGGGAAAUUUCCCAGGUCCU | |
| | CCCCGGUGAUAACAAGUUUAAUAUUACUUGUUCUGGAUA | |
| | UGAAUCCCAUGUGCCGUCUGGUGGUAUACUGACGAGUAC | |
| | AUCGCCAGUGGCUACCCCAAUCCCCGGGACAGGCUAUGCC | |
| | UACAGCCUGAGACUGACGCCAAGACCAGUUUCCAGGUUU | |
| | CUGGGGAAUAACUCUAUCCUGUAUGUGUUUUAUUCUGGU | |
| | AACGGACCCAAGGCAUCCGGCGGUGAUUAUUGUAUCCAG | |
| | AGCAAUAUAGUGUUCUCUGAUGAGAUUCCGGCCUCCCAG | |
| | GAUAUGCCAACCAACACGACAGAUAUCACCUAUGUCGGG | |
| | GACAACGCCACAUAUAGCGUGCCAAUGGUGACCAGCGAA | |
| | GAUGCCAAUUCUCCCAACGUCACAGUGACAGCCUUUUGG | |
| | GCGUGGCCUAAUAACACAGAAACUGACUUUAAAUGCAAA | |
| | UGGACUCUAACCUCAGGCACCCCAUCUGGCUGCGAGAAC | |
| | AUAAGCGGCGCGUUUGCAUCGAACAGAACUUUUGAUAUU | |
| | ACAGUAUCUGGGUUGGGCACUGCCCCCAAAACUCUAAUA | |
| | AUUACACGGACGGCCACUAACGCCACCACGACGACUCACA | |
| | AGGUGAUCUUCAGCAAAGCUCCCGAAUCUACUACUACCU | |
| | CGCCGACACUGAAUACGACAGGAUUCGCCGACCCAAAUA | |
| | CUACGACCGGCCUUCCCAGCUCGACACAUGUGCCUACAAA | |
| | CUUAACGGCCCCCGCCAGUACCGGCCCCACCGUGAGCACC | |
| | GCUGACGUCACCUCUCCUACCCCUGCCGGGACUACAAGUG | |
| | GUGCUUCCCCAGUUACUCCCAGCCCUAGCCCUUGGGACAA | |
| | CGGCACUGAGAGCAAAGCUCCGGACAUGACCAGCAGUAC | |
| | CAGUCCAGUGACAACUCCGACACCCAAUGCGACGUCCCCA | |
| | ACUCCCGCCGUGACCACGCCAACACCAAAUGCCACGUCAC | |
| | CUACUCCAGCGGUCACCACCCCAACGCCAAACGCUACUAG | |
| | UCCCACUCUAGGCAAGACAUCUCCCACCUCCGCUGUCACC | |
| | ACCCCUACACCCAAUGCUACCGGUCCCACGGUGGGUGAGA | |
| | CUAGCCCACAGGCCAACGCAACUAAUCAUACAUUGGGGG | |
| | GCACAAGCCCAACCCCCGUCGUCACUAGCCAGCCUAAAAA | |
| | CGCCACAAGCGCUGUAACUACCGGCCAGCACAAUAUAAC | |
| | AAGUUCCAGUACUAGCAGUAUGUCUCUGAGACCCAGCUC | |
| | AAAUCCUGAAACCCUCUCCCCCUCUACGUCGGAUAACUCC | |
| | ACGUCCCACAUGCCUCUACUCACAAGCGCGCACCCUACCG | |
| | GGGGAGAAAAUAUCACUCAGGUUACCCCAGCUUCUAUCU | |
| | CGACUCACCAUGUCUCUACUUCUUCCCCAGCGCCACGGCC | |
| | CGGCACUACCAGCCAGGCUAGCGGGCCAGGUAAUUCCUCC | |
| | ACUAGCACUAAACCUGGCGAAGUCAACGUUACCAAGGGA | |
| | ACACCCCCCCAGAAUGCAACCUCCCCCCAAGCUCCCUCUG | |
| | GACAGAAAACAGCCGUCCCUACCGUCACAAGCACCGGUG | |
| | GGAAGGCGAAUUCUACGACAGGCGGGAAGCACACUACCG | |
| | GACACGGAGCAAGGACAUCCACUGAGCCCACGACCGACU | |
| | ACGGUGGCGACUCCACCACCCCCAGACCCAGGUACAAUGC | |
| | GACUACCUACCUGCCCCCUUCCACUUCCAGCAAACUGAGG | |
| | CCUCGUUGGACUUUCACUUCUCCCCCAGUCACCACCGCUC | |
| | AGGCUACCGUGCCUGUCCCGCCGACCUCUCAGCCCCGGUU | |
| | UUCAAAUUUGAGUAUGCUCGUGCUGCAGUGGGCCAGCCU | |
| | GGCAGUGCUUACCCUCCUGCUCUUGCUGGUUAUGGCUGA | |
| | CUGCGCCUUCCGACGUAACUUAUCCACCAGUCAUACUUAC | |
| | ACUACACCCCCUUAUGACGACGCGGAGACCUACGUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 28 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

gp350_mod_005

| SEQ ID NO: 154 consists of from 5′ end to 3′ end: | | 154 |
|---|---|---|
| 5′ UTR SEQ ID NO: 1, mRNA ORF SEQ ID NO: 32, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAAGCCGCCUUACUGGUCUGCCAGUACACUAUUCAG | 32 |
| Construct | UCCCUCAUCCAUCUCACAGGCGAGGACCCGGGGUUCUUCA | |
| (excluding | ACGUCGAAAUCCCCGAAUUCCCAUUCUAUCCCACCUGUAA | |
| the stop | CGUCUGCACAGCCGACGUUAACGUUACAAUCAAUUUCGA | |
| codon) | CGUGGGCGGGAAAAAACACCAGCUGGACCUCGACUUUGG | |
| | GCAGCUGACGCCCCACACUAAAGCCGUUUACCAGCCCAGG | |
| | GGUGCAUUCGGUGGAUCUGAAAAUGCUACAAACCUCUUU | |
| | CUGCUUGAGCUGCUUGGUGCUGGAGAGCUGGCCCUGACA | |
| | AUGCGCAGCAAAAAGUUGCCAAUCAAUGUGACCACAGGU | |
| | GAAGAACAGCAGGUGUCCCUAGAAUCCGUGGAUGUGUAC | |
| | UUCCAGGACGUUUUCGGCACAAUGUGGUGCCAUCACGCA | |
| | GAGAUGCAAAAUCCGGUGUACCUUAUCCCCGAAACAGUG | |
| | CCGUACAUUAAAUGGGAUAAUUGUAACUCCACAAACAUC | |
| | ACGGCAGUGGUGCGCGCUCAGGGGCUGGACGUGACACUC | |
| | CCCCUUUCCUUACCAACAAGUGCUCAAGAUUCCAACUUU | |
| | UCUGUGAAAACCGAGAUGCUGGGCAACGAGAUUGAUAUU | |
| | GAAUGCAUCAUGGAGGAUGGCGAGAUUAGCCAGGUGUUG | |
| | CCCGGCGACAAUAAAUUUAAUAUUACCUGUUCCGGCUAU | |
| | GAGUCACACGUGCCAAGUGGAGGCAUUCUCACGAGCACC | |
| | UCUCCCGUUGCAACACCCAUUCCUGGGACAGGGUACGCA | |
| | UAUAGUCUCAGGCUGACACCGAGACCGGUAUCGCGCUUC | |
| | UUAGGUAACAAUUCCAUCUUGUAUGUGUUUUACAGCGGA | |
| | AACGGGCCAAAGGCCUCAGGAGGAGACUACUGCAUCCAG | |
| | AGCAAUAUAGUUUUUAGCGACGAGAUCCCUGCCUCACAA | |
| | GACAUGCCCACCAACACCACCGACAUCACGUACGUCGGUG | |
| | AUAAUGCCACUUACAGCGUGCCUAUGGUGACCUCUGAAG | |
| | ACGCAAAUUCACCCAAUGUCACGGUUACCGCAUUCUGGG | |
| | CUUGGCCAAAUAACACUGAAACCGACUUCAAGUGUAAAU | |
| | GGACCCUAACCAGCGGCACCCCAUCAGGGUGCGAGAAUA | |
| | UCAGCGGGGCCUUCGCCAGCAACCGGACAUUCGACAUCAC | |
| | CGUGUCAGGACUAGGGACUGCUCCAAAAACAUUAAUUAU | |
| | UACACGGACAGCAACUAACGCUACGACUACGACUCACAA | |
| | AGUGAUAUUUAGCAAAGCCCCCGAAUCGACUACUACGAG | |
| | CCCGACCCUAAACACCACCGGUUUCGCCGAUCCCAACACA | |
| | ACUACUGGUCUCCCAUCUUCGACCCAUGUUCCAACGAAUC | |
| | UGACAGCUCCAGCAAGUACGGGGCCCACUGUGAGUACAG | |
| | CCGACGUUACUAGCCCAACUCCCGCGGGUACGACUAGUG | |
| | GCGCGUCUCCAGUGACCCCUUCGCCUUCUCCGUGGGAUAA | |
| | UGGAACCGAGUCAAAGGCUCCUGAUAUGACCAGCUCCAC | |
| | CAGUCCCGUUACAACGCCAACCCCCAAUGCAACGAGUCCA | |
| | ACUCCGGCGGUUACCACUCCGACACCAAACGCCACCUCCC | |
| | CCACGCCUGCAGUUACUACGCCCACCCCUAAUGCUACGUC | |
| | ACCCACUCUGGGAAAGACCUCACCAACCUCUGCCGUGACU | |
| | ACUCCUACACCUAAUGCCACAGGACCCACAGUUGGGGAA | |
| | ACCAGCCCACAGGCUAACGCAACUAAUCAUACGCUUGGG | |
| | GGGACCUCUCCCACGCCCGUGGUCACUUCACAGCCUAAGA | |
| | AUGCCACAAGCGCUGUGACUACAGGCCAGCAUAAUAUAA | |
| | CCAGCAGUUCAACCAGUAGCAUGUCCCUGCGUCCUUCAUC | |
| | CAAUCCUGAGACUCUGAGCCCCAGCACUAGUGACAACUC | |
| | AACUUCUCACAUGCCGUUGCUGACCUCCGCGCAUCCCACG | |
| | GGCGGUGAGAAUAUCACACAGGUGACACCCGCGUCUAUU | |
| | AGCACACAUCACGUGAGUACGUCCUCCCCCGCACCCCGCC | |
| | CAGGCACAACCUCACAAGCAUCAGGUCCGGGGAACAGCA | |
| | GCACAAGUACCAAGCCUGGCGAAGUGAAUGUAACCAAGG | |
| | GCACGCCCCCGCAGAACGCCACAUCCCCUCAAGCCCCAUC | |
| | CGGCCAGAAGACCGCUGUACCCACCGUGACAUCAACUGG | |
| | GGGGAAAGCGAACUCAACUACCGGGGGGAAGCAUACAAC | |
| | AGGGCACGGUGCCAGAACUUCUACAGAGCCAACCACGGA | |
| | CUACGGAGGGGACAGCACAACGCCCAGACCGCGGUACAA | |
| | UGCGACCACAUACCUGCCUCCCUCAACUUCGAGUAAGCUG | |
| | CGACCCAGAUGGACCUUUACUUCUCCCCCUGUCACCACCG | |
| | CUCAAGCCACCGUCCCGGUUCCACCUACUUCCCAGCCUAG | |
| | AUUCUCCAAUCUGAGCAUGCUUGUCCUACAGUGGGCAUC | |
| | ACUCGCGGUACUAACCUUGCUGCUACUGUUGGUGAUGGC | |
| | GGACUGCGCGUUUCGCCGCAACUUAUCCACCUCCCACACG | |
| | UACACAACACCUCCCUAUGAUGACGCUGAGACUUACGUC | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 28 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gp42 (B95-8)_DX

| SEQ ID NO: 155 consists of from 5′ end to 3′ end: | | 155 |
|---|---|---|
| 5′ UTR SEQ ID NO: 1, mRNA ORF SEQ ID NO: 33, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGUGAGCUUCAAGCAGGUGAGAGUGCCCCUGUUCACC | 33 |
| Construct | GCCAUCGCCCUGGUGAUCGUGCUGCUGCUGGCCUACUUCC | |
| (excluding | UGCCCCCCAGAGUGAGAGGCGGCGGCAGAGUGGCCGCCG | |
| the stop | CCGCCAUCACCUGGGUGCCCAAGCCCAACGUGGAGGUGU | |
| codon) | GGCCCGUGGACCCCCCACCCCCCGUGAACUUCAACAAGAC | |
| | CGCCGAGCAGGAGUACGGCGACAAGGAGGUGAAGCUGCC | |
| | CCACUGGACCCCCACCCUGCACACCUUCCAGGUGCCCCAG | |
| | AACUACACCAAGGCCAACUGCACCUACUGCAACACCAGAG | |
| | AGUACACCUUCAGCUACAAGGGCUGCUGCUUCUACUUCA | |
| | CCAAGAAGAAGCACACCUGGAACGGCUGCUUCCAGGCCU | |
| | GCGCCGAGCUGUACCCCUGCACCUACUUCUACGGCCCCAC | |
| | CCCCGACAUCCUGCCCGUGGUGACCAGAAACCUGAACGCC | |
| | AUCGAGAGCCUGUGGGUGGGCGUGUACAGAGUGGGCGAG | |
| | GGCAACUGGACCAGCCUGGACGGCGGCACCUUCAAGGUG | |
| | UACCAGAUCUUCGGCAGCCACUGCACCUACGUGAGCAAG | |
| | UUCAGCACCGUGCCCGUGAGCCACCACGAGUGCAGCUUCC | |
| | UGAAGCCCUGCCUGUGCGUGAGCCAGAGAAGCAACAGC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MVSFKQVRVPLFTAIALVIVLLLAYFLPPRVRGGGRVAAAAIT | 34 |
| amino acid | WVPKPNVEVWPVDPPPPVNFNKTAEQEYGDKEVKLPHWTPT | |
| sequence | LHTFQVPQNYTKANCTYCNTREYTFSYKGCCFYFTKKKHTW | |
| | NGCFQACAELYPCTYFYGPTPDILPVVTRNLNAIESLWVGVYR | |
| | VGEGNWTSLDGGTFKVYQIFGSHCTYVSKFSTVPVSHHECSFL | |
| | KPCLCVSQRSNS | |
| PolyA tail | 100 nt | |

EBV gL (B95-8)

| SEQ ID NO: 156 consists of from 5′ end to 3′ end: | | 156 |
|---|---|---|
| 5′ UTR SEQ ID NO: 1, mRNA ORF SEQ ID NO: 35, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGAGAGCCGUGGGCGUGUUCCUGGCCAUCUGCCUGGUG | 35 |
| Construct | ACCAUCUUCGUGCUGCCCACCUGGGGCAACUGGGCCUACC | |
| (excluding | CCUGCUGCCACGUGACCCAGCUGAGAGCCCAGCACCUGCU | |
| the stop | GGCCCUGGAGAACAUCAGCGACAUCUACCUGGUGAGCAA | |
| codon) | CCAGACCUGCGACGGCUUCAGCCUGGCCAGCCUGAACAGC | |
| | CCCAAGAACGGCAGCAACCAGCUGGUGAUCAGCAGAUGC | |
| | GCCAACGGCCUGAACGUGGUGAGCUUCUUCAUCAGCAUC | |
| | CUGAAGAGAAGCAGCAGCGCCCUGACCGGCCACCUGAGA | |
| | GAGCUGCUGACCACCCUGGAGACCCUGUACGGCAGCUUC | |
| | AGCGUGGAGGACCUGUUCGGCGCCAACCUGAACAGAUAC | |
| | GCCUGGCACAGAGGCGGC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MRAVGVFLAICLVTIFVLPTWGNWAYPCCHVTQLRAQHLLA | 36 |
| amino acid | LENISDIYLVSNQTCDGFSLASLNSPKNGSNQLVISRCANGLNV | |
| sequence | VSFFISILKRSSSALTGHLRELLTTLETLYGSFSVEDLFGANLNR | |
| | YAWHRGG | |
| PolyA tail | 100 nt | |

EBV gH (BXLF2)_RX

| SEQ ID NO: 157 consists of from 5′ end to 3′ end: | | 157 |
|---|---|---|
| 5′ UTR SEQ ID NO: 1, mRNA ORF SEQ ID NO: 37, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGCAGUUGUUGUGCGUGUUCUGCCUCGUGUUACUCUGG | 37 |
| Construct | GAGGUGGGCGCCGCCAGCCUUAGCGAGGUGAAGCUCCAC | |
| (excluding | UUGGACAUCGAGGGCCACGCCAGCCACUACACCAUCCCCU | |
| the stop | GGACCGAGCUCAUGGCCAAGGUGCCCGGCCUUAGCCCCGA | |
| codon) | GGCCCUGUGGCGGGAGGCCAACGUGACCGAGGACCUGGC | |
| | CAGCAUGCUGAACCGGUACAAGCUGAUCUACAAGACCAG | |
| | CGGCACCCUGGGCAUCGCCCUGGCCGAGCCCGUGGACAUC | |
| | CCCGCCGUUAGCGAAGGCAGCAUGCAGGUGGACGCCAGC | |
| | AAGGUGCACCCCGGCGUGAUCAGCGGCCUGAACAGCCCCG | |
| | CCUGUAUGUUGAGCGCCCCACUGGAGAAGCAGCUGUUCU | |
| | ACUACAUCGGCACCAUGCUGCCCAACACCCGGCCCCACAG | |
| | CUACGUGUUCUACCAGCUGCGGUGCCACCUGAGCUACGU | |
| | UGCCCUGAGCAUCAACGGCGACAAGUUCCAGUACACCGG | |
| | CGCCAUGACCAGCAAGUUCCUGAUGGGCACCUACAAGCG | |
| | GGUCACCGAGAAGGGCGACGAGCACGUGCUGUCACUGGU | |
| | GUUCGGCAAGACCAAGGACCUGCCCGACCUGCGGGGCCCC | |
| | UUCAGCUACCCUAGUUUGACCAGCGCCCAGAGCGGCGAC | |
| | UACAGCUUGGUGAUCGUGACCACCUUCGUGCACUACGCC | |
| | AACUUCCACAACUACUUCGUGCCCAACCUGAAGGACAUG | |
| | UUCAGCCGGGCCGUGACCAUGACUGCCGCUUCUUACGCCC | |
| | GGUACGUGCUGCAGAAGCUGGUCCUGCUGGAGAUGAAGG | |
| | GCGGCUGCCGGGAGCCCGAGCUGGACACCGAAACACUGA | |
| | CCACCAUGUUCGAGGUGAGCGUGGCCUUCUUCAAGGUGG | |
| | GUCACGCGGUGGGCGAAACCGGCAACGGCUGCGUGGACU | |
| | UACGCUGGCUGGCCAAGAGCUUCUUCGAGCUGACCGUGC | |
| | UGAAGGAUAUCAUCGGCAUCUGCUACGGCGCCACCGUGA | |
| | AGGGCAUGCAGAGCUACGGCCUGGAGCGGCUGGCCGCCA | |
| | UGCUUAUGGCAACAGUGAAGAUGGAGGAGCUGGGACACC | |
| | UGACAACAGAGAAGCAGGAGUACGCCCUGAGACUGGCCA | |
| | CAGUGGGCUACCCAAAGGCCGGCGUGUACAGUGGACUGA | |
| | UCGGCGGCGCAACCAGCGUGCUGCUAUCCGCUUACAACCG | |
| | GCACCCGCUGUUCCAGCCCCUGCACACCGUGAUGCGGGAA | |
| | ACCCUGUUCAUCGGAAGCCACGUCGUGCUGCGGGAGCUG | |
| | AGGCUGAACGUAACCACCCAGGGCCCUAAUCUGGCCCUG | |
| | UAUCAGCUCCUCAGUACCGCCCUGUGCAGCGCCCUUGAGA | |
| | UCGGCGAGGUGCUCAGAGGCCUGGCCCUCGGUACCGAGA | |
| | GCGGCCUCUUCAGCCCAUGCUACUUAAGCCUGCGGUUCG | |
| | ACCUGACCCGGGACAAGUUGCUGAGCAUGGCCCCGCAGG | |
| | AGGCCACACUGGACCAGGCAGCUGUAUCCAACGCCGUGG | |
| | ACGGCUUCCUGGGCAGACUGUCCCUGGAACGGGAGGACC | |
| | GGGACGCCUGGCACCUGCCUGCCUACAAGUGUGUGGAUC | |
| | GGCUGGACAAGGUGCUGAUGAUCAUCCCUCUGAUUAAUG | |
| | UCACCUUCAUCAUCAGCAGCGACCGGGAGGUGCGGGGAU | |
| | CCGCCCUCUACGAGGCCAGCACCACCUAUCUGAGCAGCAG | |
| | CCUGUUCCUGUCUCCUGUGAUCAUGAACAAGUGCAGCCA | |
| | GGGCGCCGUGGCCGGCGAGCCCCGGCAGAUCCCCAAGAUC | |
| | CAGAACUUCACCCGGACCCAGAAGUCUUGCAUCUUCUGC | |
| | GGCUUCGCCCUUUUGUCCUACGACGAGAAGGAGGGCUUG | |
| | GAGACUACAACCUACAUCACCAGCCAGGAGGUGCAGAAC | |
| | AGCAUCCUGUCAUCUAAUUACUUCGACUUCGACAACCUG | |
| | CACGUUCAUUACCUGCUCCUCACCACCAACGGUACCGUCA | |
| | UGGAAAUCGCCGGACUGUACGAGGAGCGGGCCCAUGUUG | |
| | UGCUGGCCAUCAUCCUGUACUUCAUCGCUUUCGCACUUG | |
| | GCAUCUUCCUGGUGCACAAGAUCGUGAUGUUCUUCCUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MQLLCVFCLVLLWEVGAASLSEVKLHLDIEGHASHYTIPWTE | 38 |
| amino acid | LMAKVPGLSPEALWREANVTEDLASMLNRYKLIYKTSGTLGI | |
| sequence | ALAEPVDIPAVSEGSMQVDASKVHPGVISGLNSPACMLSAPLE | |
| | KQLFYYIGTMLPNTRPHSYVFYQLRCHLSYVALSINGDKFQYT | |
| | GAMTSKFLMGTYKRVTEKGDEHVLSLVFGKTKDLPDLRGPFS | |
| | YPSLTSAQSGDYSLVIVTTFVHYANFHNYFVPNLKDMFSRAV | |
| | TMTAASYARYVLQKLVLLEMKGGCREPELDTETLTTMFEVS | |
| | VAFFKVGHAVGETGNGCVDLRWLAKSFFELTVLKDIIGICYG | |
| | ATVKGMQSYGLERLAAMLMATVKMEELGHLTTEKQEYALR | |
| | LATVGYPKAGVYSGLIGGATSVLLSAYNRHPLFQPLHTVMRE | |
| | TLFIGSHVVLRELRLNVTTQGPNLALYQLLSTALCSALEIGEVL | |
| | RGLALGTESGLFSPCYLSLRFDLTRDKLLSMAPQEATLDQAA | |
| | VSNAVDGFLGRLSLEREDRDAWHLPAYKCVDRLDKVLMIIPL | |
| | INVTFIISSDREVRGSALYEASTTYLSSSLFLSPVIMNKCSQGAV | |
| | AGEPRQIPKIQNFTRTQKSCIFCGFALLSYDEKEGLETTTYITSQ | |
| | EVQNSILSSNYFDFDNLHVHYLLLTTNGTVMEIAGLYEERAH | |
| | VVLAIILYFIAFALGIFLVHKIVMFFL | |
| PolyA tail | 100 nt | |

EBV LMP1_DX (modified)

| SEQ ID NO: 158 consists of from 5′ end to 3′ end: | | 158 |
|---|---|---|
| 5′ UTR SEQ ID NO: 1, mRNA ORF SEQ ID NO: 39, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAACACGACCUUGAGAGGGGCCCACCGGGCCCGCGA | 39 |
| Construct | CGGCCACCUCGAGGACCUCCCCUCUCCUCUUCCCUAGGCC | |
| (excluding | UUGCUCUCCUUCUCCUCCUCUUGGCGCUACUGUUUUGGC | |
| the stop | UGUACAUCGUUAUGAGUGACUGGACUGGAGGAGCCCUCC | |
| codon) | UUGUCCUCUAUUCCUUUGCUCUCAUGCUUAUAAUUAUAA | |
| | UUUUGAUCAUCUUUAUCUUCAGAAGAGACCUUCUCUGUC | |
| | CACUUGGAGCCCUUUGUAUACUCCUACUGAUGAUCACCC | |
| | UCCUGCUCAUCGCUCUCUGGAAUUUGCACGGACAGGCAU | |
| | UGUUCCUUGGAAUUGUGCUGUUCAUCUUCGGGUGCUUAC | |
| | UUGUCUUAGGUAUCUGGAUCUACUUAUUGGAGAUGCUCU | |
| | GGCGACUUGGUGCCACCAUCUGGCAGCUUUUGGCCUUCU | |
| | UCCUAGCCUUCUUCCUAGACCUCAUCCUGCUCAUUAUUGC | |
| | UCUCUAUCUACAACAGAACUGGUGGACUCUAUUGGUUGA | |
| | UCUCCUUUGGCUCCUCCUGUUUCUGGCGAUUUUAAUCUG | |
| | GAUGUAUUACCAUGGACAACGACACAGUGAUGAACACCA | |
| | CCACGAUGACUCCCUCCCGCACCCUCAACAAGCUACCGAU | |
| | GAUUCUGGCCAUGAAUCUGACUCUAACUCCAACGAGGGC | |
| | AGACACCACCUGCUCGUGAGUGGAGCCGGCGACGGACCA | |
| | CCACUCUGCUCUCAGAACCUAGGCGCACCUGGAGGUGGU | |
| | CCUGACAAUGGCCCACAGGACCCUGACAACACGGAUGAU | |
| | AAUGGCCCACAGGACCCUGAUAACACUGAUGACAAUGGC | |
| | CCACAUGACCCGCUGCCUCAAGACCCAGACAAUACUGAUG | |
| | ACAAUGGCCCACAGGACCCUGACAACACUGAUGACAAUG | |
| | GCCCGCACGACCCGUUACCUCAUAGCCCUAGCGACUCUGC | |
| | UGGAAAUGAUGGAGGCCCUCCACAAUUGACGGAAGAGGU | |
| | UGAGAACAAAGGAGGUGACCAGGGCCCGCCUUUGAUGAC | |
| | AGACGGAGGCGGCGGUCAUAGUCAUGAUUCCGGCCAUGG | |
| | CGGCGGUGAUCCACACCUUCCUACGCUGCUGUUGGGUUC | |
| | UUCUGGUUCCGGUGGAGAUGAUGACGACCCGCACGGCCC | |
| | AGUUCAGCUAAGCUACUAUGAC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEHDLERGPPGPRRPPRGPPLSSSLGLALLLLLLALLFWLYIV | 16 |
| amino acid | MSDWTGGALLVLYSFALMLIIIILIIFIFRRDLLCPLGALCILLLM | |
| sequence | ITLLLIALWNLHGQALFLGIVLFIFGCLLVLGIWIYLLEMLWRL | |
| | GATIWQLLAFFLAFFLDLILLIIALYLQQNWWTLLVDLLWLLL | |
| | FLAILIWMYYHGQRHSDEHHHDDSLPHPQQATDDSGHESDSN | |
| | SNEGRHHLLVSGAGDGPPLCSQNLGAPGGGPDNGPQDPDNTD | |
| | DNGPQDPDNTDDNGPHDPLPQDPDNTDDNGPQDPDNTDDNG | |
| | PHDPLPHSPSDSAGNDGGPPQLTEEVENKGGDQGPPLMTDGG | |
| | GGHSHDSGHGGGDPHLPTLLLGSSGSGGDDDDPHGPVQLSYY | |
| | D | |
| PolyA tail | 100 nt | |

EBV LMP2

| SEQ ID NO: 159 consists of from 5′ end to 3′ end: | | 159 |
|---|---|---|
| 5′ UTR SEQ ID NO: 1, mRNA ORF SEQ ID NO: 40, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGGUCCCUAGAAAUGGUGCCAAUGGGCGCGGGUCCU | 40 |
| Construct | CCUAGCCCCGGCGGGGAUCCGGAUGGGUACGAUGGCGGA | |
| (excluding | AACAACUCCCAAUAUCCAUCUGCUUCUGGCUCUUCUGGG | |
| the stop | AACACGCCCACCCCACCGAACGAUGAGGAACGUGAAUCU | |
| codon) | AAUGAAGAGCCACCACCGCCUUAUGAGGACCCAUAUUGG | |
| | GGCAAUGGCGACCGUCACUCGGACUAUCAACCACUAGGA | |
| | ACCCAAGAUCAAAGUCUGUACUUGGGAUUGCAACACGAC | |
| | GGGAAUGACGGGCUCCCUCCACCUCCCUACUCUCCACGGG | |
| | AUGACUCAUCUCAACACAUAUACGAAGAAGCGGGCAGAG | |
| | GAAGUAUGAAUCCAGUAUGCCUGCCUGUAAUUGUUGCGC | |
| | CCUACCUCUUUUGGCUGGCGGCUAUUGCCGCCUCGUGUU | |
| | UCACGGCCUCAGUUAGUACCGUUGUGACCGCCACCGGCU | |
| | UGGCCCUCUCACUUCUACUCUUGGCAGCAGUGGCCAGCUC | |
| | AUAUGCCGCUGCACAAAGGAAACUGCUGACACCGGUGAC | |
| | AGUGCUUACUGCGGUUGUCACUUUCUUUGCAAUUUGCCU | |
| | AACAUGGAGGAUUGAGGACCCACCUUUUAAUUCUCUUCU | |
| | GUUUGCAUUGCUGGCCGCAGCUGGCGGACUACAAGGCAU | |
| | UUACGUUCUGGUGAUGCUUGUGCUCCUGAUACUAGCGUA | |
| | CAGAAGGAGAUGGCGCCGUUUGACUGUUUGUGGCGGCAU | |
| | CAUGUUCUUGGCAUGUGUACUUGUCCUCAUCGUCGACGC | |
| | UGUUUUGCAGCUGAGUCCCCUCCUUGGAGCUGUAACUGU | |
| | GGUUUCCAUGACGCUGCUGCUACUGGCUUUCGUCCUCUG | |
| | GCUCUCUUCGCCAGGUGGCCUAGGUACUCUUGGUGCAGC | |
| | CCUUUUAACAUUGGCAGCAGCUCUGGCACUGCUAGCGUC | |
| | ACUGAUUUUGGGCACACUUAACUUGACUACAAUGUUCCU | |
| | UCUCAUGCUCCUAUGGACACUUGUGGUUCUCCUGAUUUG | |
| | CUCUUCGUGCUCUUCAUGUCCACUGAGCAAGAUCCUUCU | |
| | GGCACGACUGUUCCUAUAUGCUCUCGCACUCUUGUUGCU | |
| | AGCCUCCGCGCUAAUCGCUGGUGGCAGUAUUUUGCAAAC | |
| | AAACUUCAAGAGUUUAAGCAGCACUGAAUUUAUACCCAA | |
| | UUUGUUCUGCAUGUUAUUACUGAUUGUCGCUGGCAUACU | |
| | CUUCAUUCUUGCUAUCCUGACCGAAUGGGGCAGUGGAAA | |
| | UAGAACAUACGGUCCAGUCUUUAUGUGCCUCGGUGGCCU | |
| | GCUCACCAUGGUAGCCGGCGCUGUGUGGCUGACGGUGAU | |
| | GUCUAACACGCUUUUGUCUGCCUGGAUUCUUACAGCAGG | |
| | AUUCCUGAUUUUCCUCAUUGGCUUUGCCCUCUUUGGGGU | |
| | CAUUAGAUGCUGCCGCUACUGCUGCUACUACUGCCUUAC | |
| | ACUGGAAAGUGAGGAGCGCCCACCGACCCCAUAUCGCAA | |
| | CACUGUA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGSLEMVPMGAGPPSPGGDPDGYDGGNNSQYPSASGSSGNT | 22 |
| amino acid | PTPPNDEERESNEEPPPPYEDPYWGNGDRHSDYQPLGTQDQSL | |
| sequence | YLGLQHDGNDGLPPPPYSPRDDSSQHIYEEAGRGSMNPVCLP | |
| | VIVAPYLFWLAAIAASCFTASVSTVVTATGLALSLLLLAAVAS | |
| | SYAAAQRKLLTPVTVLTAVVTFFAICLTWRIEDPPFNSLLFALL | |
| | AAAGGLQGIYVLVMLVLLILAYRRRWRRLTVCGGIMFLACVL | |
| | VLIVDAVLQLSPLLGAVTVVSMTLLLLAFVLWLSSPGGLGTL | |
| | GAALLTLAAALALLASLILGTLNLTTMFLLMLLWTLVVLLICS | |
| | SCSSCPLSKILLARLFLYALALLLLASALIAGGSILQTNFKSLSS | |
| | TEFIPNLFCMLLLIVAGILFILAILTEWGSGNRTYGPVFMCLGG | |
| | LLTMVAGAVWLTVMSNTLLSAWILTAGFLIFLIGFALFGVIRC | |
| | CRYCCYYCLTLESEERPPTPYRNTV | |
| PolyA tail | 100 nt | |

EBNA1_trunc_001 (modified)

| SEQ ID NO: 160 consists of from 5′ end to 3′ end: | | 160 |
|---|---|---|
| 5′ UTR SEQ ID NO: 1, mRNA ORF SEQ ID NO: 41, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGCAGAAGACCCUUCUUUCACCCCGUGGGAGAAGCC | 41 |
| Construct | GACUAUUUCGAGUACCACCAGGAGGGCGGUCCUGACGGC | |
| (excluding | GAGCCCGACGUGCCUCCAGGCGCCAUCGAACAGGGCCCCG | |
| the stop | CUGACGAUCCUGGCGAGGGGCCUUCCACAGGCCCUCGCGG | |
| codon) | CCAGGGAGAUGGCGGAAGGCGGAAGAAGGGCGGCUGGUU | |
| | UGGCAAGCAUAGGGGCCAGGGCGGCUCCAAUCCCAAGUU | |
| | CGAGAAUAUCGCCGAGGGGCUCCGGGCUCUGCUGGCCCG | |
| | GUCGCACGUCGAACGUACCACUGACGAGGGGACGUGGGU | |
| | GGCCGGCGUCUUUGUGUACGGAGGAUCCAAGACCUCCCU | |
| | GUACAACCUGAGAAGGGGCACCGCCCUUGCCAUCCCUCAG | |
| | UGCAGACUCACGCCCCUGAGCAGGCUCCCCUUUGGGAUG | |
| | GCUCCUGGGCCAGGCCCGCAGCCCGGGCCCCUCAGGGAAA | |
| | GCAUCGUGUGUUACUUUAUGGUGUUUCUGCAGACACACA | |
| | UCUUUGCAGAAGUUCUGAAAGAUGCAAUCAAGGAUCUGG | |
| | UGAUGACCAAGCCUGCACCUACGUGCAAUAUCAGGGUGA | |
| | CAGUAUGCUCCUUCGACGACGGUGUGGACCUCCCGCCCUG | |
| | GUUCCCACCCAUGGUCGAGGGUGCCGCCGCCGAGGGAGA | |
| | CGAUGGCGACGACGGUGAUGAGGGUGGAGACGGAGAUGA | |
| | AGGCGAGGAGGGGCAGGAG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGRRPFFHPVGEADYFEYHQEGGPDGEPDVPPGAIEQGPADD | 10 |
| amino acid | PGEGPSTGPRGQGDGGRRKKGGWFGKHRGQGGSNPKFENIA | |
| sequence | EGLRALLARSHVERTTDEGTWVAGVFVYGGSKTSLYNLRRG | |
| | TALAIPQCRLTPLSRLPFGMAPGPGPQPGPLRESIVCYFMVFLQ | |
| | THIFAEVLKDAIKDLVMTKPAPTCNIRVTVCSFDDGVDLPPWF | |
| | PPMVEGAAAEGDDGDDGDEGGDGDEGEEGQE | |
| PolyA tail | 100 nt | |

EBV EBNA3 (B95-8)_RX

| SEQ ID NO: 161 consists of from 5′ end to 3′ end: | | 161 |
|---|---|---|
| 5′ UTR SEQ ID NO: 1, mRNA ORF SEQ ID NO: 42, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGACAAGGACAGGCCCGGCCCUCCCGCCUUGGACGACA | 42 |
| Construct | ACAUGGAGGAGGAGGUGCCCAGCACCAGCGUGGUGCAGG | |
| (excluding | AGCAGGUGAGCGCCGGCGACUGGGAGAACGUGUUAAUCG | |
| the stop | AGUUGAGCGACAGCAGCAGCGAGAAGGAGGCCGAGGACG | |
| codon) | CCCACCUGGAGCCCGCCCAGAAGGGCACCAAGAGGAAGA | |
| | GGGUGGACCACGACGCUGGCGGAUCUGCCCCUGCCAGGCC | |
| | CAUGCUGCCACCCCAGCCCGACCUGCCCGGCAGGGAGGCC | |
| | AUCCUGAGGAGGUUCCCACUGGACCUGAGGACCCUGCUG | |
| | CAGGCCAUCGGCGCCGCCGCCACCAGGAUCGACACCAGGG | |
| | CCAUCGACCAGUUCUUCGGCAGCCAGAUCAGCAACACCGA | |
| | GAUGUACAUCAUGUACGCCAUGGCCAUCAGACAAGCAAU | |
| | UAGGGAUCGACGCAGAAAUCCUGCCAGCAGGAGGGACCA | |
| | GGCCAAGUGGAGGCUGCAGACCCUGGCCGCCGGCUGGCCC | |
| | AUGGGCUACCAGGCCUACAGCAGCUGGAUGUACAGCUAC | |
| | ACCGACCACCAGACCACUCCGACCUUCGUGCACCUCCAGG | |
| | CUACCCUGGGCUGCACUGGCGGUCGCAGGUGCCACGUGA | |
| | CCUUCUCAGCGGGCACCUUCAAGCUGCCCAGGUGCACACC | |
| | UGGCGACAGGCAGUGGCUGUACGUGCAGAGCAGCGUGGG | |
| | CAACAUUGUUCAGAGUUGCAACCCCAGGUACAGCAUCUU | |
| | CUUCGACUACAUGGCGAUUCACAGGAGCCUGACCAAGAU | |
| | CUGGGAGGAAGUCCUGACCCCUGACCAGAGGGUGAGCUU | |
| | CAUGGAGUUCCUGGGCUUCCUGCAGAGGACCGACCUGAG | |
| | CUACAUCAAGAGCUUCGUGAGCGACGCCCUGGGCACCACC | |
| | AGCAUCCAGACUCCUUGGAUCGACGAUAACCCUAGCACC | |
| | GAAACCGCCCAGGCCUGGAACGCCGGUUUCCUGAGGGGC | |
| | AGGGCCUACGGCAUCGACCUGCUCCGGACCGAGGGCGAG | |
| | CACGUGGAGGGCGCCACCGGCGAGACUAGGGAGGAGAGC | |
| | GAGGACACUGAGAGCGACGGCGACGACGAGGAUCUGCCG | |
| | UGCAUCGUGAGCAGGGGCGGCCCCAAGGUGAAGAGGCCU | |
| | CCCAUCUUCAUCCGGAGGCUUCACAGGCUGCUGCUGAUG | |
| | AGGGCCGGCAAGCGUACUGAACAGGGCAAGGAGGUACUG | |
| | GAGAAGGCCAGGGGCAGCACAUAUGGCACCCCUAGACCG | |
| | CCUGUGCCCAAGCCGCGUCCUGAAGUCCCGCAGAGUGAU | |
| | GAGACAGCGACUAGCCAUGGCAGUGCUCAGGUGCCCGAG | |
| | CCACCCACCAUCCAUCUCGCCGCACAGGGCAUGGCCUACC | |
| | CACUGCACGAGCAGCACGGCAUGGCGCCCUGCCCCGUGGC | |
| | CCAGGCUCCUCCAACCCCGCUUCCUCCAGUAUCCCCUGGU | |
| | GACCAGUUGCCAGGCGUGUUCAGCGACGGUAGGGUGGCC | |
| | UGCGCGCCAGUUCCUGCACCAGCAGGCCCAAUUGUGAGG | |
| | CCCUGGGAGCCCUCCCUAACCCAGGCGGCAGGUCAGGCCU | |
| | UCGCUCCAGUUAGACCACAGCACAUGCCCGUGGAACCGG | |
| | UGCCAGUGCCUACGGUGGCGCUGGAGAGGCCCGUGUACC | |
| | CAAAGCCUGUGCGACCUGCGCCUCCUAAGAUCGCCAUGCA | |
| | AGGCCCGGGCGAGACUAGCGGUAUCAGGCGGGCCAGGGA | |
| | GAGGUGGCGCCCGGCACCGUGGACUCCCAACCCACCAAGG | |
| | UCACCUAGUCAGAUGAGCGUGCGUGAUAGGCUGGCACGG | |
| | CUUCGGGCAGAGGCUCAGGUAAAGCAGGCCAGUGUGGAG | |
| | GUGCAGCCACCACAGCUGACUCAGGUGUCCCCGCAGCAGC | |
| | CCAUGGAGGGCCCACUGGUGCCGGAACAGCAGAUGUUCC | |
| | CCGGCGCCCCGUUCAGCCAGGUGGCCGACGUGGUUAGGG | |
| | CUCCUGGCGUGCCUGCUAUGCAGCCUCAGUACUUCGACCU | |
| | ACCGCUGAUCCAGCCCAUCAGUCAAGGUGCCCCAGUGGCC | |
| | CCACUGAGGGCUAGCAUGGGCCCGGUCCCUCCUGUUCCAG | |
| | CCACCCAGCCUCAAUAUUUCGACAUUCCUCUGACCGAGCC | |
| | UAUAAACCAGGGCGCGUCUGCGGCCCACUUCCUGCCUCAA | |
| | CAGCCAAUGGAAGGCCCGCUGGUCCCAGAACAGUGGAUG | |
| | UUCCCUGGAGCUGCCCUGAGCCAAUCAGUGAGGCCGGGA | |
| | GUUGCCCAGAGCCAGUAUUUCGAUCUCCCUUUGACACAG | |
| | CCUAUCAAUCACGGAGCACCUGCCGCUCACUUCCUUCACC | |
| | AGCCUCCGAUGGAAGGUCCUUGGGUUCCAGAGCAAUGGA | |
| | UGUUCCAAGGUGCGCCACCAUCUCAGGGCACAGACGUGG | |
| | UCCAGCAUCAGCUGGACGCGCUUGGCUACACUCUACACG | |
| | GCCUGAACCACCCGGGCGUUCCAGUGAGUCCAGCCGUGA | |
| | ACCAGUACCAUCUCUCUCAGGCUGCCUUCGGCCUGCCCAU | |
| | UGACGAGGACGAGAGCGGCGAGGGCAGCGAUACCAGCGA | |
| | GCCCUGCGAAGCCCUGGAUCUUUCCAUCCACGGCCGACCU | |
| | UGCCCACAGGCUCCAGAGUGGCCCGUACAGGAAGAAGGC | |
| | GGCCAGGACGCCACCGAGGUCCUGGACUUGUCCAUACAU | |
| | GGCAGACCGCGCCCUAGGACCCCGGAGUGGCCAGUUCAG | |
| | GGUGAAGGUGGUCAGAACGUGACCGGCCCGGAAACCCGA | |
| | AGGGUGGUCGUGUCCGCUGUUGUCCACAUGUGUCAGGAU | |
| | GACGAAUUCCCAGAUCUGCAAGAUCCACCCGACGAGGCC | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDKDRPGPPALDDNMEEEVPSTSVVQEQVSAGDWENVLIELS | 43 |
| amino acid | DSSSEKEAEDAHLEPAQKGTKRKRVDHDAGGSAPARPMLPPQ | |
| sequence | PDLPGREAILRRFPLDLRTLLQAIGAAATRIDTRAIDQFFGSQIS | |
| | NTEMYIMYAMAIRQAIRDRRRNPASRRDQAKWRLQTLAAGW | |
| | PMGYQAYSSWMYSYTDHQTTPTFVHLQATLGCTGGRRCHVT | |
| | FSAGTFKLPRCTPGDRQWLYVQSSVGNIVQSCNPRYSIFFDYM | |
| | AIHRSLTKIWEEVLTPDQRVSFMEFLGFLQRTDLSYIKSFVSDA | |
| | LGTTSIQTPWIDDNPSTETAQAWNAGFLRGRAYGIDLLRTEGE | |
| | HVEGATGETREESEDTESDGDDEDLPCIVSRGGPKVKRPPIFIR | |
| | RLHRLLLMRAGKRTEQGKEVLEKARGSTYGTPRPPVPKPRPE | |
| | VPQSDETATSHGSAQVPEPPTIHLAAQGMAYPLHEQHGMAPC | |
| | PVAQAPPTPLPPVSPGDQLPGVFSDGRVACAPVPAPAGPIVRP | |
| | WEPSLTQAAGQAFAPVRPQHMPVEPVPVPTVALERPVYPKPV | |
| | RPAPPKIAMQGPGETSGIRRARERWRPAPWTPNPPRSPSQMSV | |
| | RDRLARLRAEAQVKQASVEVQPPQLTQVSPQQPMEGPLVPEQ | |
| | QMFPGAPFSQVADVVRAPGVPAMQPQYFDLPLIQPISQGAPV | |
| | APLRASMGPVPPVPATQPQYFDIPLTEPINQGASAAHFLPQQP | |
| | MEGPLVPEQWMFPGAALSQSVRPGVAQSQYFDLPLTQPINHG | |
| | APAAHFLHQPPMEGPWVPEQWMFQGAPPSQGTDVVQHQLD | |
| | ALGYTLHGLNHPGVPVSPAVNQYHLSQAAFGLPIDEDESGEG | |
| | SDTSEPCEALDLSIHGRPCPQAPEWPVQEEGGQDATEVLDLSI | |
| | HGRPRPRTPEWPVQGEGGQNVTGPETRRVVVSAVVHMCQDD | |
| | EFPDLQDPPDEA | |
| PolyA tail | 100 nt | |

EBV gp350 A44Y V10 Stab

| SEQ ID NO: 162 consists of from 5′ end to 3′ end: | | 162 |
|---|---|---|
| 5′ UTR SEQ ID NO: 1, mRNA ORF SEQ ID NO: 44, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 44 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUUACGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTY | 45 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gp350 1150F V11 Stab

| SEQ ID NO: 163 consists of from 5′ end to 3′ end: | | 163 |
|---|---|---|
| 5′ UTR SEQ ID NO: 1, mRNA ORF SEQ ID NO: 46, and | | |
| 3′ UTR SEQ ID NO: 3 | | |
| Chemistry | 1-methylpseudouridine | |
| Cap | 7mG(5′)ppp(5′)NlmpNp | |
| 5′UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 46 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAUUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 47 |
| amino acid | DVNVTFNFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gp350 M2021 V12 Stab

| SEQ ID NO: 164 consists of from 5′ end to 3′ end: | | 164 |
|---|---|---|
| 5′ UTR SEQ ID NO: 1, mRNA ORF SEQ ID NO: 48, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 48 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUACUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 49 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEILGNEIDIECIME | |
| | DGEISQVLPGDNKFNITCSGYESHVPSGGILTSTSPVATPIPGTG | |
| | YAYSLRLTPRPVSRFLGNNSILYVFYSGNGPKASGGDYCIQSNI | |
| | VFSDEIPASQDMPTNTTDITYVGDNATYSVPMVTSEDANSPNV | |
| | TVTAFWAWPNNTETDFKCKWTLTSGTPSGCENISGAFASNRT | |
| | FDITVSGLGTAPKTLIITRTATNATTTTHKVIFSKAPESTTTSPT | |
| | LNTTGFADPNTTTGLPSSTHVPTNLTAPASTGPTVSTADVTSPT | |
| | PAGTTSGASPVTPSPSPWDNGTESKAPDMTSSTSPVTTPTPNA | |
| | TSPTPAVTTPTPNATSPTPAVTTPTPNATSPTLGKTSPTSAVTTP | |
| | TPNATGPTVGETSPQANATNHTLGGTSPTPVVTSQPKNATSAV | |
| | TTGQHNITSSSTSSMSLRPSSNPETLSPSTSDNSTSHMPLLTSAH | |
| | PTGGENITQVTPASISTHHVSTSSPAPRPGTTSQASGPGNSSTST | |
| | KPGEVNVTKGTPPQNATSPQAPSGQKTAVPTVTSTGGKANST | |
| | TGGKHTTGHGARTSTEPTTDYGGDSTTPRPRYNATTYLPPSTS | |
| | SKLRPRWTFTSPPVTTAQATVPVPPTSQPRFSNLSMLVLQWAS | |
| | LAVLTLLLLLVMADCAFRRNLSTSHTYTTPPYDDAETYV | |
| PolyA tail | 100 nt | |

EBV gp350 11417W V13 Stab

| SEQ ID NO: 165 consists of from 5′ end to 3′ end: | | 165 |
|---|---|---|
| 5′ UTR SEQ ID NO: 1, mRNA ORF SEQ ID NO: 50, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 50 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCUGGAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 51 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTWKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gp350 V14 Stab4 mut

| SEQ ID NO: 166 consists of from 5′ end to 3′ end: | | 166 |
|---|---|---|
| 5′ UTR SEQ ID NO: 1, mRNA ORF SEQ ID NO: 52, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 52 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUUACGACGUGAACGUGACAUUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUACUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCUGGAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTY | 53 |
| amino acid | DVNVTFNFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEILGNEIDIECIME | |
| | DGEISQVLPGDNKFNITCSGYESHVPSGGILTSTSPVATPIPGTG | |
| | YAYSLRLTPRPVSRFLGNNSILYVFYSGNGPKASGGDYCIQSNI | |
| | VFSDEIPASQDMPTNTTDITYVGDNATYSVPMVTSEDANSPNV | |
| | TVTAFWAWPNNTETDFKCKWTLTSGTPSGCENISGAFASNRT | |
| | FDITVSGLGTAPKTLIITRTATNATTTTWKVIFSKAPESTTTSPT | |
| | LNTTGFADPNTTTGLPSSTHVPTNLTAPASTGPTVSTADVTSPT | |
| | PAGTTSGASPVTPSPSPWDNGTESKAPDMTSSTSPVTTPTPNA | |
| | TSPTPAVTTPTPNATSPTPAVTTPTPNATSPTLGKTSPTSAVTTP | |
| | TPNATGPTVGETSPQANATNHTLGGTSPTPVVTSQPKNATSAV | |
| | TTGQHNITSSSTSSMSLRPSSNPETLSPSTSDNSTSHMPLLTSAH | |
| | PTGGENITQVTPASISTHHVSTSSPAPRPGTTSQASGPGNSSTST | |
| | KPGEVNVTKGTPPQNATSPQAPSGQKTAVPTVTSTGGKANST | |
| | TGGKHTTGHGARTSTEPTTDYGGDSTTPRPRYNATTYLPPSTS | |
| | SKLRPRWTFTSPPVTTAQATVPVPPTSQPRFSNLSMLVLQWAS | |
| | LAVLTLLLLLVMADCAFRRNLSTSHTYTTPPYDDAETYV | |
| PolyA tail | 100 nt | |

EBV gp350 V15 Stab4_NGM6

| SEQ ID NO: 167 consists of from 5′ end to 3′ end: | | 167 |
|---|---|---|
| 5′ UTR SEQ ID NO: 1, mRNA ORF SEQ ID NO: 54, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 54 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUUACGACGUGAACGUGACAUUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUGCGAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAGCGAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | GCGGUCAAGACCGAGAUACUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCGCGUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUGCGGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCGCGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCUGG | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTY | 55 |
| amino acid | DVNVTFNFDVGGKKHQLDLDFGQLTPHTKAVYQPRGAFGGS | |
| sequence | ENAANLFLLELLGAGELALTMRSKKLPINVTTGEEQQVSLESV | |
| | DVYFQDVFGTMWCHHAEMQNPVYLIPETVPYIKWDNCNSAN | |
| | ITAVVRAQGLDVTLPLSLPTSAQDSNFAVKTEILGNEIDIECIM | |
| | EDGEISQVLPGDNKFNIACSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVAVTAFWAWPNNTETDFKCKWTLTSGTPSGCENISGAFASN | |
| | RTFDITVSGLGTAPKTLIITRTATNAATTTHKVIFSKAPESTTTS | |
| | PTLNTTGFADPNTTTGLPSSTHVPTNLTAPASTGPTVSTWDVT | |
| | SPTPAGTTSGASPVTPSPSPWDNGTESKAPDMTSSTSPVTTPTP | |
| | NATSPTPAVTTPTPNATSPTPAVTTPTPNATSPTLGKTSPTSAV | |
| | TTPTPNATGPTVGETSPQANATNHTLGGTSPTPVVTSQPKNAT | |
| | SAVTTGQHNITSSSTSSMSLRPSSNPETLSPSTSDNSTSHMPLLT | |
| | SAHPTGGENITQVTPASISTHHVSTSSPAPRPGTTSQASGPGNS | |
| | STSTKPGEVNVTKGTPPQNATSPQAPSGQKTAVPTVTSTGGK | |
| | ANSTTGGKHTTGHGARTSTEPTTDYGGDSTTPRPRYNATTYL | |
| | PPSTSSKLRPRWTFTSPPVTTAQATVPVPPTSQPRFSNLSMLVL | |
| | QWASLAVLTLLLLLVMADCAFRRNLSTSHTYTTPPYDDAETY | |
| | V | |
| PolyA tail | 100 nt | |

V1_NGM 36 mut_EBV_gp350_001

| SEQ ID NO: 168 consists of from 5′ end to 3′ end: | | 168 |
|---|---|---|
| 5′ UTR SEQ ID NO: 1, mRNA ORF SEQ ID NO: 56, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 56 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGGCGAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUGCGAAUCUGU | |
| | UCUUACUGGAACUGCUUGGUGCAGGAGAGCUGGCACUAA | |
| | CCAUGCGAAGCAAGAAGCUCCCCAUCAACGUUGCGACCG | |
| | GGGAAGAACAGCAAGUCAGCCUUGAAUCCGUGGAUGUGU | |
| | ACUUUCAGGAUGUUUUCGGCACCAUGUGGUGUCACCACG | |
| | CUGAGAUGCAGAAUCCCGUGUAUCUCAUACCCGAGACUG | |
| | UACCCUACAUCAAGUGGGAUAAUUGCAAUUCAGCGAAUA | |
| | UUGCGGCUGUGGUGAGGGCCCAGGGCUUGGACGUGACUC | |
| | UGCCCUUAUCUCUACCUACUUCUGCCCAAGACUCCAAUUU | |
| | CGCGGUCAAGACCGAGAUGCUCGGGAAUGAGAUCGAUAU | |
| | CGAGUGCAUCAUGGAGGACGGUGAGAUAAGCCAGGUUCU | |
| | GCCCGGCGACAACAAGUUCAAUAUCGCGUGUUCUGGCUA | |
| | CGAGUCCCAUGUGCCUAGUGGUGGCAUACUCACAAGUAC | |
| | UUCUCCCGUAGCCACGCCCAUUCCCGGAACCGGAUACGCC | |
| | UACAGUCUGCGUCUGACCCCACGGCCUGUGUCCAGAUUCC | |
| | UGGGUAACAAUGCGAUCUUAUACGUGUUUUAUAGCGGAA | |
| | ACGGCCCUAAAGCGUCCGGAGGGGACUAUUGUAUUCAGA | |
| | GUAAUAUCGUUUUCUCUGAUGAGAUUCCUGCCAGUCAGG | |
| | ACAUGCCGACAAACACAGCGGAUAUUACCUACGUGGGCG | |
| | ACAAUGCCGCGUAUUCAGUGCCCAUGGUCACGAGCGAGG | |
| | ACGCCAAUUCACCAAAUGUUGCGGUAACAGCUUUCUGGG | |
| | CCUGGCCAAAUAACGCGGAGACUGACUUCAAAUGUAAGU | |
| | GGACUUUGACCUCUGGAACUCCGUCGGGUUGCGAGAAUA | |
| | UCGCGGGGGCCUUUGCUUCCAACAGGGCGUUCGACAUCA | |
| | CUGUCUCAGGGCUGGGGACAGCACCGAAGACAUUAAUCA | |
| | UAACACGGACCGCCACCAACGCCGCGACUACAACCCAUAA | |
| | GGUGAUCUUUUCCAAGGCACCUGAGUCCACCACUACCUCC | |
| | CCGACUCUUAACACUGCGGGCUUCGCUGAUCCCAACACCG | |
| | CGACGGGGUUGCCUAGCUCGACACAUGUGCCGACGAACC | |
| | UGGCGGCCCCUGCAUCGACCGGGCCCACAGUUUCGACCGC | |
| | CGAUGUGACAUCACCAACGCCCGCAGGUACAACCUCAGGC | |
| | GCCAGCCCAGUGACCCCUUCCCCAAGCCCCUGGGAUAAUG | |
| | GAGCGGAGUCCAAGGCUCCUGAUAUGACUUCCUCUACCA | |
| | GCCCCGUGACUACACCCACUCCCAACGCAGCGAGCCCAAC | |
| | CCCAGCUGUGACGACGCCCACCCCGAACGCGGCGUCUCCC | |
| | ACACCUGCUGUGACAACCCCAACCCCUAACGCCGCGAGCC | |
| | CUACCCUAGGUAAGACCAGUCCGACUAGCGCCGUUACAA | |
| | CCCCUACCCCUAACGCAGCGGGCCCGACCGUGGGCGAGAC | |
| | UUCCCCGCAAGCCAAUGCGGCGAAUCACGCGUUGGGCGG | |
| | GACCUCUCCUACACCAGUCGUUACAUCUCAGCCUAAGAAC | |
| | GCUGCGUCCGCUGUCACUACCGGACAGCACAACAUCGCGA | |
| | GCUCAAGCACGAGUUCCAUGAGCUUGCGGCCGAGCUCAA | |
| | AUCCCGAGACCCUAUCACCAUCCACAUCAGACAACAGUGC | |
| | GUCACACAUGCCACUCUUGACGAGCGCUCACCCCACCGGC | |
| | GGCGAGAACAUCGCGCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCGCGA | |
| | CAAGCACAAAGCCAGGCGAAGUUAACGUGGCGAAGGGAA | |
| | CGCCUCCCCAGAACGCAGCGAGUCCUCAGGCGCCCAGCGG | |
| | GCAGAAGACUGCGGUGCCAACUGUGACCAGCACCGGUGG | |
| | CAAGGCCAACUCAGCGACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | GCGACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUGCGAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCGCGACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 57 |
| amino acid | DVNVAINFDVGGKKHQLDLDFGQLTPHTKAVYQPRGAFGGS | |
| sequence | ENAANLFLLELLGAGELALTMRSKKLPINVATGEEQQVSLESV | |
| | DVYFQDVFGTMWCHHAEMQNPVYLIPETVPYIKWDNCNSAN | |
| | IAAVVRAQGLDVTLPLSLPTSAQDSNFAVKTEMLGNEIDIECI | |
| | MEDGEISQVLPGDNKFNIACSGYESHVPSGGILTSTSPVATPIP | |
| | GTGYAYSLRLTPRPVSRFLGNNAILYVFYSGNGPKASGGDYCI | |
| | QSNIVFSDEIPASQDMPTNTADITYVGDNAAYSVPMVTSEDAN | |
| | SPNVAVTAFWAWPNNAETDFKCKWTLTSGTPSGCENIAGAF | |
| | ASNRAFDITVSGLGTAPKTLIITRTATNAATTTHKVIFSKAPEST | |
| | TTSPTLNTAGFADPNTATGLPSSTHVPTNLAAPASTGPTVSTA | |
| | DVTSPTPAGTTSGASPVTPSPSPWDNGAESKAPDMTSSTSPVT | |
| | TPTPNAASPTPAVTTPTPNAASPTPAVTTPTPNAASPTLGKTSP | |
| | TSAVTTPTPNAAGPTVGETSPQANAANHALGGTSPTPVVTSQP | |
| | KNAASAVTTGQHNIASSSTSSMSLRPSSNPETLSPSTSDNSASH | |
| | MPLLTSAHPTGGENIAQVTPASISTHHVSTSSPAPRPGTTSQAS | |
| | GPGNSATSTKPGEVNVAKGTPPQNAASPQAPSGQKTAVPTVT | |
| | STGGKANSATGGKHTTGHGARTSTEPTTDYGGDSTTPRPRYN | |
| | AATYLPPSTSSKLRPRWTFTSPPVTTAQATVPVPPTSQPRFSNL | |
| | AMLVLQWASLAVLTLLLLLVMADCAFRRNLATSHTYTTPPY | |
| | DDAETYV | |
| PolyA tail | 100 nt | |

V2_NGM 18 mut_EBV_gp350_001

| SEQ ID NO: 169 consists of from 5′ end to 3′ end: | | 169 |
|---|---|---|
| 5′ UTR SEQ ID NO: 1, mRNA ORF SEQ ID NO: 58, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 58 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGGCGAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUGCGAAUCUGU | |
| | UCUUACUGGAACUGCUUGGUGCAGGAGAGCUGGCACUAA | |
| | CCAUGCGAAGCAAGAAGCUCCCCAUCAACGUUGCGACCG | |
| | GGGAAGAACAGCAAGUCAGCCUUGAAUCCGUGGAUGUGU | |
| | ACUUUCAGGAUGUUUUCGGCACCAUGUGGUGUCACCACG | |
| | CUGAGAUGCAGAAUCCCGUGUAUCUCAUACCCGAGACUG | |
| | UACCCUACAUCAAGUGGGAUAAUUGCAAUUCAGCGAAUA | |
| | UUGCGGCUGUGGUGAGGGCCCAGGGCUUGGACGUGACUC | |
| | UGCCCUUAUCUCUACCUACUUCUGCCCAAGACUCCAAUUU | |
| | CGCGGUCAAGACCGAGAUGCUCGGGAAUGAGAUCGAUAU | |
| | CGAGUGCAUCAUGGAGGACGGUGAGAUAAGCCAGGUUCU | |
| | GCCCGGCGACAACAAGUUCAAUAUCGCGUGUUCUGGCUA | |
| | CGAGUCCCAUGUGCCUAGUGGUGGCAUACUCACAAGUAC | |
| | UUCUCCCGUAGCCACGCCCAUUCCCGGAACCGGAUACGCC | |
| | UACAGUCUGCGUCUGACCCCACGGCCUGUGUCCAGAUUCC | |
| | UGGGUAACAAUGCGAUCUUAUACGUGUUUUAUAGCGGAA | |
| | ACGGCCCUAAAGCGUCCGGAGGGGACUAUUGUAUUCAGA | |
| | GUAAUAUCGUUUUCUCUGAUGAGAUUCCUGCCAGUCAGG | |
| | ACAUGCCGACAAACACAGCGGAUAUUACCUACGUGGGCG | |
| | ACAAUGCCGCGUAUUCAGUGCCCAUGGUCACGAGCGAGG | |
| | ACGCCAAUUCACCAAAUGUUGCGGUAACAGCUUUCUGGG | |
| | CCUGGCCAAAUAACGCGGAGACUGACUUCAAAUGUAAGU | |
| | GGACUUUGACCUCUGGAACUCCGUCGGGUUGCGAGAAUA | |
| | UCGCGGGGGCCUUUGCUUCCAACAGGGCGUUCGACAUCA | |
| | CUGUCUCAGGGCUGGGGACAGCACCGAAGACAUUAAUCA | |
| | UAACACGGACCGCCACCAACGCCGCGACUACAACCCAUAA | |
| | GGUGAUCUUUUCCAAGGCACCUGAGUCCACCACUACCUCC | |
| | CCGACUCUUAACACUGCGGGCUUCGCUGAUCCCAACACCG | |
| | CGACGGGGUUGCCUAGCUCGACACAUGUGCCGACGAACC | |
| | UGGCGGCCCCUGCAUCGACCGGGCCCACAGUUUCGACCGC | |
| | CGAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 59 |
| amino acid | DVNVAINFDVGGKKHQLDLDFGQLTPHTKAVYQPRGAFGGS | |
| sequence | ENAANLFLLELLGAGELALTMRSKKLPINVATGEEQQVSLESV | |
| | DVYFQDVFGTMWCHHAEMQNPVYLIPETVPYIKWDNCNSAN | |
| | IAAVVRAQGLDVTLPLSLPTSAQDSNFAVKTEMLGNEIDIECI | |
| | MEDGEISQVLPGDNKFNIACSGYESHVPSGGILTSTSPVATPIP | |
| | GTGYAYSLRLTPRPVSRFLGNNAILYVFYSGNGPKASGGDYCI | |
| | QSNIVFSDEIPASQDMPTNTADITYVGDNAAYSVPMVTSEDAN | |
| | SPNVAVTAFWAWPNNAETDFKCKWTLTSGTPSGCENIAGAF | |
| | ASNRAFDITVSGLGTAPKTLIITRTATNAATTTHKVIFSKAPEST | |
| | TTSPTLNTAGFADPNTATGLPSSTHVPTNLAAPASTGPTVSTA | |
| | DVTSPTPAGTTSGASPVTPSPSPWDNGTESKAPDMTSSTSPVT | |
| | TPTPNATSPTPAVTTPTPNATSPTPAVTTPTPNATSPTLGKTSPT | |
| | SAVTTPTPNATGPTVGETSPQANATNHTLGGTSPTPVVTSQPK | |
| | NATSAVTTGQHNITSSSTSSMSLRPSSNPETLSPSTSDNSTSHM | |
| | PLLTSAHPTGGENITQVTPASISTHHVSTSSPAPRPGTTSQASGP | |
| | GNSSTSTKPGEVNVTKGTPPQNATSPQAPSGQKTAVPTVTSTG | |
| | GKANSTTGGKHTTGHGARTSTEPTTDYGGDSTTPRPRYNATT | |
| | YLPPSTSSKLRPRWTFTSPPVTTAQATVPVPPTSQPRFSNLSML | |
| | VLQWASLAVLTLLLLLVMADCAFRRNLSTSHTYTTPPYDDAE | |
| | TYV | |
| PolyA tail | 100 nt | |

V4_NGM N87_EBV_gp350_001

| SEQ ID NO: 170 consists of from 5′ end to 3′ end: | | 170 |
|---|---|---|
| 5′ UTR SEQ ID NO: 1, mRNA ORF SEQ ID NO: 60, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 60 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUGCGAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 61 |
| amino acid | DVNVTINFDVGGKKHQLDLDFGQLTPHTKAVYQPRGAFGGS | |
| sequence | ENAANLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

V5_NGM N166_EBV_gp350_001

| SEQ ID NO: 171 consists of from 5′ end to 3′ end: | | 171 |
|---|---|---|
| 5′ UTR SEQ ID NO: 1, mRNA ORF SEQ ID NO: 62, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 62 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAGCGAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 63 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSAN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

V6_NGM N195_EBV_gp350_001

| SEQ ID NO: 172 consists of from 5′ end to 3′ end: | | 172 |
|---|---|---|
| 5′ UTR SEQ ID NO: 1, mRNA ORF SEQ ID NO: 64, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 64 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | GCGGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 65 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFAVKTEMLGNEIDIECI | |
| | MEDGEISQVLPGDNKFNITCSGYESHVPSGGILTSTSPVATPIPG | |
| | TGYAYSLRLTPRPVSRFLGNNSILYVFYSGNGPKASGGDYCIQ | |
| | SNIVFSDEIPASQDMPTNTTDITYVGDNATYSVPMVTSEDANS | |
| | PNVTVTAFWAWPNNTETDFKCKWTLTSGTPSGCENISGAFAS | |
| | NRTFDITVSGLGTAPKTLIITRTATNATTTTHKVIFSKAPESTTT | |
| | SPTLNTTGFADPNTTTGLPSSTHVPTNLTAPASTGPTVSTADVT | |
| | SPTPAGTTSGASPVTPSPSPWDNGTESKAPDMTSSTSPVTTPTP | |
| | NATSPTPAVTTPTPNATSPTPAVTTPTPNATSPTLGKTSPTSAV | |
| | TTPTPNATGPTVGETSPQANATNHTLGGTSPTPVVTSQPKNAT | |
| | SAVTTGQHNITSSSTSSMSLRPSSNPETLSPSTSDNSTSHMPLLT | |
| | SAHPTGGENITQVTPASISTHHVSTSSPAPRPGTTSQASGPGNS | |
| | STSTKPGEVNVTKGTPPQNATSPQAPSGQKTAVPTVTSTGGK | |
| | ANSTTGGKHTTGHGARTSTEPTTDYGGDSTTPRPRYNATTYL | |
| | PPSTSSKLRPRWTFTSPPVTTAQATVPVPPTSQPRFSNLSMLVL | |
| | QWASLAVLTLLLLLVMADCAFRRNLSTSHTYTTPPYDDAETY | |
| | V | |
| PolyA tail | 100 nt | |

V7_NGM N229_EBV_gp350_001

| SEQ ID NO: 173 consists of from 5′ end to 3′ end: | | 173 |
|---|---|---|
| 5′ UTR SEQ ID NO: 1, mRNA ORF SEQ ID NO: 66, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 66 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCGCGUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 67 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNIACSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

V8_NGM N345_EBV_gp350_001

| SEQ ID NO: 174 consists of from 5′ end to 3′ end: | | 174 |
|---|---|---|
| 5′ UTR SEQ ID NO: 1, mRNA ORF SEQ ID NO: 68, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 68 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUGCGGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 114 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVAVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

V9_NGM N411_EBV_gp350_001

| SEQ ID NO: 175 consists of from 5′ end to 3′ end: | | 175 |
|---|---|---|
| 5′ UTR SEQ ID NO: 1, mRNA ORF SEQ ID NO: 69, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 69 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCGCGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 70 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNAATTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gp42 (B95-8)_DX

| SEQ ID NO: 176 consists of from 5′ end to 3′ end: | | 176 |
|---|---|---|
| 5′ UTR SEQ ID NO: 1, mRNA ORF SEQ ID NO: 70, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGUGAGCUUCAAGCAGGUGAGAGUGCCCCUGUUCACC | 71 |
| Construct | GCCAUCGCCCUGGUGAUCGUGCUGCUGCUGGCCUACUUCC | |
| (excluding | UGCCCCCCAGAGUGAGAGGCGGCGGCAGAGUGGCCGCCG | |
| the stop | CCGCCAUCACCUGGGUGCCCAAGCCCAACGUGGAGGUGU | |
| codon) | GGCCCGUGGACCCCCCACCCCCCGUGAACUUCAACAAGAC | |
| | CGCCGAGCAGGAGUACGGCGACAAGGAGGUGAAGCUGCC | |
| | CCACUGGACCCCCACCCUGCACACCUUCCAGGUGCCCCAG | |
| | AACUACACCAAGGCCAACUGCACCUACUGCAACACCAGAG | |
| | AGUACACCUUCAGCUACAAGGGCUGCUGCUUCUACUUCA | |
| | CCAAGAAGAAGCACACCUGGAACGGCUGCUUCCAGGCCU | |
| | GCGCCGAGCUGUACCCCUGCACCUACUUCUACGGCCCCAC | |
| | CCCCGACAUCCUGCCCGUGGUGACCAGAAACCUGAACGCC | |
| | AUCGAGAGCCUGUGGGUGGGCGUGUACAGAGUGGGCGAG | |
| | GGCAACUGGACCAGCCUGGACGGCGGCACCUUCAAGGUG | |
| | UACCAGAUCUUCGGCAGCCACUGCACCUACGUGAGCAAG | |
| | UUCAGCACCGUGCCCGUGAGCCACCACGAGUGCAGCUUCC | |
| | UGAAGCCCUGCCUGUGCGUGAGCCAGAGAAGCAACAGC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MVSFKQVRVPLFTAIALVIVLLLAYFLPPRVRGGGRVAAAAIT | 34 |
| amino acid | WVPKPNVEVWPVDPPPPVNFNKTAEQEYGDKEVKLPHWTPT | |
| sequence | LHTFQVPQNYTKANCTYCNTREYTFSYKGCCFYFTKKKHTW | |
| | NGCFQACAELYPCTYFYGPTPDILPVVTRNLNAIESLWVGVYR | |
| | VGEGNWTSLDGGTFKVYQIFGSHCTYVSKFSTVPVSHHECSFL | |
| | KPCLCVSQRSNS | |
| PolyA tail | 100 nt | |

EBV EBNA3 (B95-8)_RX

| SEQ ID NO: 177 consists of from 5′ end to 3′ end: | | 177 |
|---|---|---|
| 5′ UTR SEQ ID NO: 1, mRNA ORF SEQ ID NO: 72, and | | |
| 3′ UTR SEQ ID NO: 3 | | |
| Chemistry | 1-methylpseudouridine | |
| Cap | 7mG(5′)ppp(5′)NlmpNp | |
| 5′UTR | GGGAAAUAAGAGAGAAAAGAAGAGUAAGAAGAAAUAUA | 1 |
| | AGAGCCACC | |
| ORF of mRNA | AUGGACAAGGACAGGCCCGGCCCUCCCGCCUUGGACGACA | 72 |
| Construct | ACAUGGAGGAGGAGGUGCCCAGCACCAGCGUGGUGCAGG | |
| (excluding | AGCAGGUGAGCGCCGGCGACUGGGAGAACGUGUUAAUCG | |
| the stop | AGUUGAGCGACAGCAGCAGCGAGAAGGAGGCCGAGGACG | |
| codon) | CCCACCUGGAGCCCGCCCAGAAGGGCACCAAGAGGAAGA | |
| | GGGUGGACCACGACGCUGGCGGAUCUGCCCCUGCCAGGCC | |
| | CAUGCUGCCACCCCAGCCCGACCUGCCCGGCAGGGAGGCC | |
| | AUCCUGAGGAGGUUCCCACUGGACCUGAGGACCCUGCUG | |
| | CAGGCCAUCGGCGCCGCCGCCACCAGGAUCGACACCAGGG | |
| | CCAUCGACCAGUUCUUCGGCAGCCAGAUCAGCAACACCGA | |
| | GAUGUACAUCAUGUACGCCAUGGCCAUCAGACAAGCAAU | |
| | UAGGGAUCGACGCAGAAAUCCUGCCAGCAGGAGGGACCA | |
| | GGCCAAGUGGAGGCUGCAGACCCUGGCCGCCGGCUGGCCC | |
| | AUGGGCUACCAGGCCUACAGCAGCUGGAUGUACAGCUAC | |
| | ACCGACCACCAGACCACUCCGACCUUCGUGCACCUCCAGG | |
| | CUACCCUGGGCUGCACUGGCGGUCGCAGGUGCCACGUGA | |
| | CCUUCUCAGCGGGCACCUUCAAGCUGCCCAGGUGCACACC | |
| | UGGCGACAGGCAGUGGCUGUACGUGCAGAGCAGCGUGGG | |
| | CAACAUUGUUCAGAGUUGCAACCCCAGGUACAGCAUCUU | |
| | CUUCGACUACAUGGCGAUUCACAGGAGCCUGACCAAGAU | |
| | CUGGGAGGAAGUCCUGACCCCUGACCAGAGGGUGAGCUU | |
| | CAUGGAGUUCCUGGGCUUCCUGCAGAGGACCGACCUGAG | |
| | CUACAUCAAGAGCUUCGUGAGCGACGCCCUGGGCACCACC | |
| | AGCAUCCAGACUCCUUGGAUCGACGAUAACCCUAGCACC | |
| | GAAACCGCCCAGGCCUGGAACGCCGGUUUCCUGAGGGGC | |
| | AGGGCCUACGGCAUCGACCUGCUCCGGACCGAGGGCGAG | |
| | CACGUGGAGGGCGCCACCGGCGAGACUAGGGAGGAGAGC | |
| | GAGGACACUGAGAGCGACGGCGACGACGAGGAUCUGCCG | |
| | UGCAUCGUGAGCAGGGGCGGCCCCAAGGUGAAGAGGCCU | |
| | CCCAUCUUCAUCCGGAGGCUUCACAGGCUGCUGCUGAUG | |
| | AGGGCCGGCAAGCGUACUGAACAGGGCAAGGAGGUACUG | |
| | GAGAAGGCCAGGGGCAGCACAUAUGGCACCCCUAGACCG | |
| | CCUGUGCCCAAGCCGCGUCCUGAAGUCCCGCAGAGUGAU | |
| | GAGACAGCGACUAGCCAUGGCAGUGCUCAGGUGCCCGAG | |
| | CCACCCACCAUCCAUCUCGCCGCACAGGGCAUGGCCUACC | |
| | CACUGCACGAGCAGCACGGCAUGGCGCCCUGCCCCGUGGC | |
| | CCAGGCUCCUCCAACCCCGCUUCCUCCAGUAUCCCCUGGU | |
| | GACCAGUUGCCAGGCGUGUUCAGCGACGGUAGGGUGGCC | |
| | UGCGCGCCAGUUCCUGCACCAGCAGGCCCAAUUGUGAGG | |
| | CCCUGGGAGCCCUCCCUAACCCAGGCGGCAGGUCAGGCCU | |
| | UCGCUCCAGUUAGACCACAGCACAUGCCCGUGGAACCGG | |
| | UGCCAGUGCCUACGGUGGCGCUGGAGAGGCCCGUGUACC | |
| | CAAAGCCUGUGCGACCUGCGCCUCCUAAGAUCGCCAUGCA | |
| | AGGCCCGGGCGAGACUAGCGGUAUCAGGCGGGCCAGGGA | |
| | GAGGUGGCGCCCGGCACCGUGGACUCCCAACCCACCAAGG | |
| | UCACCUAGUCAGAUGAGCGUGCGUGAUAGGCUGGCACGG | |
| | CUUCGGGCAGAGGCUCAGGUAAAGCAGGCCAGUGUGGAG | |
| | GUGCAGCCACCACAGCUGACUCAGGUGUCCCCGCAGCAGC | |
| | CCAUGGAGGGCCCACUGGUGCCGGAACAGCAGAUGUUCC | |
| | CCGGCGCCCCGUUCAGCCAGGUGGCCGACGUGGUUAGGG | |
| | CUCCUGGCGUGCCUGCUAUGCAGCCUCAGUACUUCGACCU | |
| | ACCGCUGAUCCAGCCCAUCAGUCAAGGUGCCCCAGUGGCC | |
| | CCACUGAGGGCUAGCAUGGGCCCGGUCCCUCCUGUUCCAG | |
| | CCACCCAGCCUCAAUAUUUCGACAUUCCUCUGACCGAGCC | |
| | UAUAAACCAGGGCGCGUCUGCGGCCCACUUCCUGCCUCAA | |
| | CAGCCAAUGGAAGGCCCGCUGGUCCCAGAACAGUGGAUG | |
| | UUCCCUGGAGCUGCCCUGAGCCAAUCAGUGAGGCCGGGA | |
| | GUUGCCCAGAGCCAGUAUUUCGAUCUCCCUUUGACACAG | |
| | CCUAUCAAUCACGGAGCACCUGCCGCUCACUUCCUUCACC | |
| | AGCCUCCGAUGGAAGGUCCUUGGGUUCCAGAGCAAUGGA | |
| | UGUUCCAAGGUGCGCCACCAUCUCAGGGCACAGACGUGG | |
| | UCCAGCAUCAGCUGGACGCGCUUGGCUACACUCUACACG | |
| | GCCUGAACCACCCGGGCGUUCCAGUGAGUCCAGCCGUGA | |
| | ACCAGUACCAUCUCUCUCAGGCUGCCUUCGGCCUGCCCAU | |
| | UGACGAGGACGAGAGCGGCGAGGGCAGCGAUACCAGCGA | |
| | GCCCUGCGAAGCCCUGGAUCUUUCCAUCCACGGCCGACCU | |
| | UGCCCACAGGCUCCAGAGUGGCCCGUACAGGAAGAAGGC | |
| | GGCCAGGACGCCACCGAGGUCCUGGACUUGUCCAUACAU | |
| | GGCAGACCGCGCCCUAGGACCCCGGAGUGGCCAGUUCAG | |
| | GGUGAAGGUGGUCAGAACGUGACCGGCCCGGAAACCCGA | |
| | AGGGUGGUCGUGUCCGCUGUUGUCCACAUGUGUCAGGAU | |
| | GACGAAUUCCCAGAUCUGCAAGAUCCACCCGACGAGGCC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDKDRPGPPALDDNMEEEVPSTSVVQEQVSAGDWENVLIELS | 43 |
| amino acid | DSSSEKEAEDAHLEPAQKGTKRKRVDHDAGGSAPARPMLPPQ | |
| sequence | PDLPGREAILRRFPLDLRTLLQAIGAAATRIDTRAIDQFFGSQIS | |
| | NTEMYIMYAMAIRQAIRDRRRNPASRRDQAKWRLQTLAAGW | |
| | PMGYQAYSSWMYSYTDHQTTPTFVHLQATLGCTGGRRCHVT | |
| | FSAGTFKLPRCTPGDRQWLYVQSSVGNIVQSCNPRYSIFFDYM | |
| | AIHRSLTKIWEEVLTPDQRVSFMEFLGFLQRTDLSYIKSFVSDA | |
| | LGTTSIQTPWIDDNPSTETAQAWNAGFLRGRAYGIDLLRTEGE | |
| | HVEGATGETREESEDTESDGDDEDLPCIVSRGGPKVKRPPIFIR | |
| | RLHRLLLMRAGKRTEQGKEVLEKARGSTYGTPRPPVPKPRPE | |
| | VPQSDETATSHGSAQVPEPPTIHLAAQGMAYPLHEQHGMAPC | |
| | PVAQAPPTPLPPVSPGDQLPGVFSDGRVACAPVPAPAGPIVRP | |
| | WEPSLTQAAGQAFAPVRPQHMPVEPVPVPTVALERPVYPKPV | |
| | RPAPPKIAMQGPGETSGIRRARERWRPAPWTPNPPRSPSQMSV | |
| | RDRLARLRAEAQVKQASVEVQPPQLTQVSPQQPMEGPLVPEQ | |
| | QMFPGAPFSQVADVVRAPGVPAMQPQYFDLPLIQPISQGAPV | |
| | APLRASMGPVPPVPATQPQYFDIPLTEPINQGASAAHFLPQQP | |
| | MEGPLVPEQWMFPGAALSQSVRPGVAQSQYFDLPLTQPINHG | |
| | APAAHFLHQPPMEGPWVPEQWMFQGAPPSQGTDVVQHQLD | |
| | ALGYTLHGLNHPGVPVSPAVNQYHLSQAAFGLPIDEDESGEG | |
| | SDTSEPCEALDLSIHGRPCPQAPEWPVQEEGGQDATEVLDLSI | |
| | HGRPRPRTPEWPVQGEGGQNVTGPETRRVVVSAVVHMCQDD | |
| | EFPDLQDPPDEA | |
| PolyA tail | 100 nt | |

EBNA1_trunc_001 (modified)

| SEQ ID NO: 178 consists of from 5′ end to 3′ end: | | 178 |
|---|---|---|
| 5′ UTR SEQ ID NO: 1, mRNA ORF SEQ ID NO: 73, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGCAGAAGACCCUUCUUUCACCCCGUGGGAGAAGCC | 73 |
| Construct | GACUAUUUCGAGUACCACCAGGAGGGCGGUCCUGACGGC | |
| (excluding | GAGCCCGACGUGCCUCCAGGCGCCAUCGAACAGGGCCCCG | |
| the stop | CUGACGAUCCUGGCGAGGGGCCUUCCACAGGCCCUCGCGG | |
| codon) | CCAGGGAGAUGGCGGAAGGCGGAAGAAGGGCGGCUGGUU | |
| | UGGCAAGCAUAGGGGCCAGGGCGGCUCCAAUCCCAAGUU | |
| | CGAGAAUAUCGCCGAGGGGCUCCGGGCUCUGCUGGCCCG | |
| | GUCGCACGUCGAACGUACCACUGACGAGGGGACGUGGGU | |
| | GGCCGGCGUCUUUGUGUACGGAGGAUCCAAGACCUCCCU | |
| | GUACAACCUGAGAAGGGGCACCGCCCUUGCCAUCCCUCAG | |
| | UGCAGACUCACGCCCCUGAGCAGGCUCCCCUUUGGGAUG | |
| | GCUCCUGGGCCAGGCCCGCAGCCCGGGCCCCUCAGGGAAA | |
| | GCAUCGUGUGUUACUUUAUGGUGUUUCUGCAGACACACA | |
| | UCUUUGCAGAAGUUCUGAAAGAUGCAAUCAAGGAUCUGG | |
| | UGAUGACCAAGCCUGCACCUACGUGCAAUAUCAGGGUGA | |
| | CAGUAUGCUCCUUCGACGACGGUGUGGACCUCCCGCCCUG | |
| | GUUCCCACCCAUGGUCGAGGGUGCCGCCGCCGAGGGAGA | |
| | CGAUGGCGACGACGGUGAUGAGGGUGGAGACGGAGAUGA | |
| | AGGCGAGGAGGGGCAGGAG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGRRPFFHPVGEADYFEYHQEGGPDGEPDVPPGAIEQGPADD | 10 |
| amino acid | PGEGPSTGPRGQGDGGRRKKGGWFGKHRGQGGSNPKFENIA | |
| sequence | EGLRALLARSHVERTTDEGTWVAGVFVYGGSKTSLYNLRRG | |
| | TALAIPQCRLTPLSRLPFGMAPGPGPQPGPLRESIVCYFMVFLQ | |
| | THIFAEVLKDAIKDLVMTKPAPTCNIRVTVCSFDDGVDLPPWF | |
| | PPMVEGAAAEGDDGDDGDEGGDGDEGEEGQE | |
| PolyA tail | 100 nt | |

EBV LMP1_DX_(modified)_DX

| SEQ ID NO: 179 consists of from 5′ end to 3′ end: | | 179 |
|---|---|---|
| 5′ UTR SEQ ID NO: 1, mRNA ORF SEQ ID NO: 74, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAACACGACCUUGAGAGGGGCCCACCGGGCCCGCGA | 74 |
| Construct | CGGCCACCUCGAGGACCUCCCCUCUCCUCUUCCCUAGGCC | |
| (excluding | UUGCUCUCCUUCUCCUCCUCUUGGCGCUACUGUUUUGGC | |
| the stop | UGUACAUCGUUAUGAGUGACUGGACUGGAGGAGCCCUCC | |
| codon) | UUGUCCUCUAUUCCUUUGCUCUCAUGCUUAUAAUUAUAA | |
| | UUUUGAUCAUCUUUAUCUUCAGAAGAGACCUUCUCUGUC | |
| | CACUUGGAGCCCUUUGUAUACUCCUACUGAUGAUCACCC | |
| | UCCUGCUCAUCGCUCUCUGGAAUUUGCACGGACAGGCAU | |
| | UGUUCCUUGGAAUUGUGCUGUUCAUCUUCGGGUGCUUAC | |
| | UUGUCUUAGGUAUCUGGAUCUACUUAUUGGAGAUGCUCU | |
| | GGCGACUUGGUGCCACCAUCUGGCAGCUUUUGGCCUUCU | |
| | UCCUAGCCUUCUUCCUAGACCUCAUCCUGCUCAUUAUUGC | |
| | UCUCUAUCUACAACAGAACUGGUGGACUCUAUUGGUUGA | |
| | UCUCCUUUGGCUCCUCCUGUUUCUGGCGAUUUUAAUCUG | |
| | GAUGUAUUACCAUGGACAACGACACAGUGAUGAACACCA | |
| | CCACGAUGACUCCCUCCCGCACCCUCAACAAGCUACCGAU | |
| | GAUUCUGGCCAUGAAUCUGACUCUAACUCCAACGAGGGC | |
| | AGACACCACCUGCUCGUGAGUGGAGCCGGCGACGGACCA | |
| | CCACUCUGCUCUCAGAACCUAGGCGCACCUGGAGGUGGU | |
| | CCUGACAAUGGCCCACAGGACCCUGACAACACGGAUGAU | |
| | AAUGGCCCACAGGACCCUGAUAACACUGAUGACAAUGGC | |
| | CCACAUGACCCGCUGCCUCAAGACCCAGACAAUACUGAUG | |
| | ACAAUGGCCCACAGGACCCUGACAACACUGAUGACAAUG | |
| | GCCCGCACGACCCGUUACCUCAUAGCCCUAGCGACUCUGC | |
| | UGGAAAUGAUGGAGGCCCUCCACAAUUGACGGAAGAGGU | |
| | UGAGAACAAAGGAGGUGACCAGGGCCCGCCUUUGAUGAC | |
| | AGACGGAGGCGGCGGUCAUAGUCAUGAUUCCGGCCAUGG | |
| | CGGCGGUGAUCCACACCUUCCUACGCUGCUGUUGGGUUC | |
| | UUCUGGUUCCGGUGGAGAUGAUGACGACCCGCACGGCCC | |
| | AGUUCAGCUAAGCUACUAUGAC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEHDLERGPPGPRRPPRGPPLSSSLGLALLLLLLALLFWLYIV | 16 |
| amino acid | MSDWTGGALLVLYSFALMLIIIILIIFIFRRDLLCPLGALCILLLM | |
| sequence | ITLLLIALWNLHGQALFLGIVLFIFGCLLVLGIWIYLLEMLWRL | |
| | GATIWQLLAFFLAFFLDLILLIIALYLQQNWWTLLVDLLWLLL | |
| | FLAILIWMYYHGQRHSDEHHHDDSLPHPQQATDDSGHESDSN | |
| | SNEGRHHLLVSGAGDGPPLCSQNLGAPGGGPDNGPQDPDNTD | |
| | DNGPQDPDNTDDNGPHDPLPQDPDNTDDNGPQDPDNTDDNG | |
| | PHDPLPHSPSDSAGNDGGPPQLTEEVENKGGDQGPPLMTDGG | |
| | GGHSHDSGHGGGDPHLPTLLLGSSGSGGDDDDPHGPVQLSYY | |
| | D | |
| PolyA tail | 100 nt | |

EBV LMP2

| SEQ ID NO: 180 consists of from 5′ end to 3′ end: | | 180 |
|---|---|---|
| 5′ UTR SEQ ID NO: 1, mRNA ORF SEQ ID NO: 75, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGGUCCCUAGAAAUGGUGCCAAUGGGCGCGGGUCCU | 75 |
| Construct | CCUAGCCCCGGCGGGGAUCCGGAUGGGUACGAUGGCGGA | |
| (excluding | AACAACUCCCAAUAUCCAUCUGCUUCUGGCUCUUCUGGG | |
| the stop | AACACGCCCACCCCACCGAACGAUGAGGAACGUGAAUCU | |
| codon) | AAUGAAGAGCCACCACCGCCUUAUGAGGACCCAUAUUGG | |
| | GGCAAUGGCGACCGUCACUCGGACUAUCAACCACUAGGA | |
| | ACCCAAGAUCAAAGUCUGUACUUGGGAUUGCAACACGAC | |
| | GGGAAUGACGGGCUCCCUCCACCUCCCUACUCUCCACGGG | |
| | AUGACUCAUCUCAACACAUAUACGAAGAAGCGGGCAGAG | |
| | GAAGUAUGAAUCCAGUAUGCCUGCCUGUAAUUGUUGCGC | |
| | CCUACCUCUUUUGGCUGGCGGCUAUUGCCGCCUCGUGUU | |
| | UCACGGCCUCAGUUAGUACCGUUGUGACCGCCACCGGCU | |
| | UGGCCCUCUCACUUCUACUCUUGGCAGCAGUGGCCAGCUC | |
| | AUAUGCCGCUGCACAAAGGAAACUGCUGACACCGGUGAC | |
| | AGUGCUUACUGCGGUUGUCACUUUCUUUGCAAUUUGCCU | |
| | AACAUGGAGGAUUGAGGACCCACCUUUUAAUUCUCUUCU | |
| | GUUUGCAUUGCUGGCCGCAGCUGGCGGACUACAAGGCAU | |
| | UUACGUUCUGGUGAUGCUUGUGCUCCUGAUACUAGCGUA | |
| | CAGAAGGAGAUGGCGCCGUUUGACUGUUUGUGGCGGCAU | |
| | CAUGUUCUUGGCAUGUGUACUUGUCCUCAUCGUCGACGC | |
| | UGUUUUGCAGCUGAGUCCCCUCCUUGGAGCUGUAACUGU | |
| | GGUUUCCAUGACGCUGCUGCUACUGGCUUUCGUCCUCUG | |
| | GCUCUCUUCGCCAGGUGGCCUAGGUACUCUUGGUGCAGC | |
| | CCUUUUAACAUUGGCAGCAGCUCUGGCACUGCUAGCGUC | |
| | ACUGAUUUUGGGCACACUUAACUUGACUACAAUGUUCCU | |
| | UCUCAUGCUCCUAUGGACACUUGUGGUUCUCCUGAUUUG | |
| | CUCUUCGUGCUCUUCAUGUCCACUGAGCAAGAUCCUUCU | |
| | GGCACGACUGUUCCUAUAUGCUCUCGCACUCUUGUUGCU | |
| | AGCCUCCGCGCUAAUCGCUGGUGGCAGUAUUUUGCAAAC | |
| | AAACUUCAAGAGUUUAAGCAGCACUGAAUUUAUACCCAA | |
| | UUUGUUCUGCAUGUUAUUACUGAUUGUCGCUGGCAUACU | |
| | CUUCAUUCUUGCUAUCCUGACCGAAUGGGGCAGUGGAAA | |
| | UAGAACAUACGGUCCAGUCUUUAUGUGCCUCGGUGGCCU | |
| | GCUCACCAUGGUAGCCGGCGCUGUGUGGCUGACGGUGAU | |
| | GUCUAACACGCUUUUGUCUGCCUGGAUUCUUACAGCAGG | |
| | AUUCCUGAUUUUCCUCAUUGGCUUUGCCCUCUUUGGGGU | |
| | CAUUAGAUGCUGCCGCUACUGCUGCUACUACUGCCUUAC | |
| | ACUGGAAAGUGAGGAGCGCCCACCGACCCCAUAUCGCAA | |
| | CACUGUA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGSLEMVPMGAGPPSPGGDPDGYDGGNNSQYPSASGSSGNT | 22 |
| amino acid | PTPPNDEERESNEEPPPPYEDPYWGNGDRHSDYQPLGTQDQSL | |
| sequence | YLGLQHDGNDGLPPPPYSPRDDSSQHIYEEAGRGSMNPVCLP | |
| | VIVAPYLFWLAAIAASCFTASVSTVVTATGLALSLLLLAAVAS | |
| | SYAAAQRKLLTPVTVLTAVVTFFAICLTWRIEDPPFNSLLFALL | |
| | AAAGGLQGIYVLVMLVLLILAYRRRWRRLTVCGGIMFLACVL | |
| | VLIVDAVLQLSPLLGAVTVVSMTLLLLAFVLWLSSPGGLGTL | |
| | GAALLTLAAALALLASLILGTLNLTTMFLLMLLWTLVVLLICS | |
| | SCSSCPLSKILLARLFLYALALLLLASALIAGGSILQTNFKSLSS | |
| | TEFIPNLFCMLLLIVAGILFILAILTEWGSGNRTYGPVFMCLGG | |
| | LLTMVAGAVWLTVMSNTLLSAWILTAGFLIFLIGFALFGVIRC | |
| | CRYCCYYCLTLESEERPPTPYRNTV | |
| PolyA tail | 100 nt | |

EBV LMP2

| SEQ ID NO: 181 consists of from 5′ end to 3′ end: | | 181 |
|---|---|---|
| 5′ UTR SEQ ID NO: 1, mRNA ORF SEQ ID NO: 76, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGGGUCCCUAGAAAUGGUGCCAAUGGGCGCGGGUCCU | 76 |
| Construct | CCUAGCCCCGGCGGGGAUCCGGAUGGGUACGAUGGCGGA | |
| (excluding | AACAACUCCCAAUAUCCAUCUGCUUCUGGCUCUUCUGGG | |
| the stop | AACACGCCCACCCCACCGAACGAUGAGGAACGUGAAUCU | |
| codon) | AAUGAAGAGCCACCACCGCCUUAUGAGGACCCAUAUUGG | |
| | GGCAAUGGCGACCGUCACUCGGACUAUCAACCACUAGGA | |
| | ACCCAAGAUCAAAGUCUGUACUUGGGAUUGCAACACGAC | |
| | GGGAAUGACGGGCUCCCUCCACCUCCCUACUCUCCACGGG | |
| | AUGACUCAUCUCAACACAUAUACGAAGAAGCGGGCAGAG | |
| | GAAGUAUGAAUCCAGUAUGCCUGCCUGUAAUUGUUGCGC | |
| | CCUACCUCUUUUGGCUGGCGGCUAUUGCCGCCUCGUGUU | |
| | UCACGGCCUCAGUUAGUACCGUUGUGACCGCCACCGGCU | |
| | UGGCCCUCUCACUUCUACUCUUGGCAGCAGUGGCCAGCUC | |
| | AUAUGCCGCUGCACAAAGGAAACUGCUGACACCGGUGAC | |
| | AGUGCUUACUGCGGUUGUCACUUUCUUUGCAAUUUGCCU | |
| | AACAUGGAGGAUUGAGGACCCACCUUUUAAUUCUCUUCU | |
| | GUUUGCAUUGCUGGCCGCAGCUGGCGGACUACAAGGCAU | |
| | UUACGUUCUGGUGAUGCUUGUGCUCCUGAUACUAGCGUA | |
| | CAGAAGGAGAUGGCGCCGUUUGACUGUUUGUGGCGGCAU | |
| | CAUGUUCUUGGCAUGUGUACUUGUCCUCAUCGUCGACGC | |
| | UGUUUUGCAGCUGAGUCCCCUCCUUGGAGCUGUAACUGU | |
| | GGUUUCCAUGACGCUGCUGCUACUGGCUUUCGUCCUCUG | |
| | GCUCUCUUCGCCAGGUGGCCUAGGUACUCUUGGUGCAGC | |
| | CCUUUUAACAUUGGCAGCAGCUCUGGCACUGCUAGCGUC | |
| | ACUGAUUUUGGGCACACUUAACUUGACUACAAUGUUCCU | |
| | UCUCAUGCUCCUAUGGACACUUGUGGUUCUCCUGAUUUG | |
| | CUCUUCGUGCUCUUCAUGUCCACUGAGCAAGAUCCUUCU | |
| | GGCACGACUGUUCCUAUAUGCUCUCGCACUCUUGUUGCU | |
| | AGCCUCCGCGCUAAUCGCUGGUGGCAGUAUUUUGCAAAC | |
| | AAACUUCAAGAGUUUAAGCAGCACUGAAUUUAUACCCAA | |
| | UUUGUUCUGCAUGUUAUUACUGAUUGUCGCUGGCAUACU | |
| | CUUCAUUCUUGCUAUCCUGACCGAAUGGGGCAGUGGAAA | |
| | UAGAACAUACGGUCCAGUCUUUAUGUGCCUCGGUGGCCU | |
| | GCUCACCAUGGUAGCCGGCGCUGUGUGGCUGACGGUGAU | |
| | GUCUAACACGCUUUUGUCUGCCUGGAUUCUUACAGCAGG | |
| | AUUCCUGAUUUUCCUCAUUGGCUUUGCCCUCUUUGGGGU | |
| | CAUUAGAUGCUGCCGCUACUGCUGCUACUACUGCCUUAC | |
| | ACUGGAAAGUGAGGAGCGCCCACCGACCCCAUAUCGCAA | |
| | CACUGUA | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MGSLEMVPMGAGPPSPGGDPDGYDGGNNSQYPSASGSSGNT | 22 |
| amino acid | PTPPNDEERESNEEPPPPYEDPYWGNGDRHSDYQPLGTQDQSL | |
| sequence | YLGLQHDGNDGLPPPPYSPRDDSSQHIYEEAGRGSMNPVCLP | |
| | VIVAPYLFWLAAIAASCFTASVSTVVTATGLALSLLLLAAVAS | |
| | SYAAAQRKLLTPVTVLTAVVTFFAICLTWRIEDPPFNSLLFALL | |
| | AAAGGLQGIYVLVMLVLLILAYRRRWRRLTVCGGIMFLACVL | |
| | VLIVDAVLQLSPLLGAVTVVSMTLLLLAFVLWLSSPGGLGTL | |
| | GAALLTLAAALALLASLILGTLNLTTMFLLMLLWTLVVLLICS | |
| | SCSSCPLSKILLARLFLYALALLLLASALIAGGSILQTNFKSLSS | |
| | TEFIPNLFCMLLLIVAGILFILAILTEWGSGNRTYGPVFMCLGG | |
| | LLTMVAGAVWLTVMSNTLLSAWILTAGFLIFLIGFALFGVIRC | |
| | CRYCCYYCLTLESEERPPTPYRNTV | |
| PolyA tail | 100 nt | |

EBV gp350_003 (modified)

| SEQ ID NO: 182 consists of from 5′ end to 3′ end: | | 182 |
|---|---|---|
| 5′ UTR SEQ ID NO: 1, mRNA ORF SEQ ID NO: 77, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAAGCCGCUCUUCUGGUCUGCCAGUAUACAAUCCAG | 77 |
| Construct | UCGCUGAUUCAUCUCACCGGCGAGGAUCCAGGUUUCUUU | |
| (excluding | AACGUGGAGAUUCCAGAAUUCCCUUUCUAUCCAACUUGU | |
| the stop | AACGUGUGUACUGCCGACGUUAACGUUACUAUUAACUUU | |
| codon) | GACGUGGGCGGAAAGAAGCACCAGCUGGAUCUUGAUUUC | |
| | GGCCAGCUAACACCUCAUACCAAGGCCGUCUACCAACCAC | |
| | GCGGGGCAUUUGGCGGUUCUGAGAAUGCUACAAACCUGU | |
| | UCCUUUUGGAACUCCUCGGAGCAGGGGAACUGGCACUUA | |
| | CAAUGAGGAGUAAGAAGCUGCCCAUCAACGUCACCACGG | |
| | GUGAAGAACAGCAGGUGUCCCUGGAGUCCGUUGAUGUGU | |
| | AUUUCCAGGACGUUUUCGGGACAAUGUGGUGUCAUCAUG | |
| | CCGAAAUGCAGAAUCCGGUGUAUCUCAUUCCUGAGACCG | |
| | UGCCCUAUAUUAAAUGGGAUAACUGUAAUUCGACAAACA | |
| | UAACUGCCGUAGUGCGUGCCCAGGGUCUGGAUGUAACUU | |
| | UGCCUCUCUCCCUUCCCACUUCUGCCCAGGACUCCAAUUU | |
| | CUCAGUGAAGACCGAAAUGCUUGGGAACGAGAUCGACAU | |
| | CGAGUGUAUCAUGGAGGAUGGGGAGAUCAGUCAGGUGUU | |
| | GCCUGGUGACAAUAAGUUUAAUAUAACAUGUUCCGGCUA | |
| | UGAAUCACAUGUUCCAAGCGGAGGUAUUCUCACAAGUAC | |
| | AUCUCCCGUAGCCACGCCGAUUCCGGGAACCGGAUACGCC | |
| | UAUUCACUUCGGCUGACUCCUCGGCCAGUGUCACGAUUC | |
| | CUGGGCAAUAACUCAAUUCUGUACGUCUUUUACAGCGGC | |
| | AACGGCCCAAAGGCCAGUGGAGGCGAUUACUGUAUCCAG | |
| | UCUAACAUCGUAUUUUCAGAUGAGAUUCCAGCAAGCCAA | |
| | GACAUGCCAACUAAUACUACAGACAUUACAUACGUCGGU | |
| | GAUAACGCCACUUAUUCAGUCCCUAUGGUCACAUCCGAG | |
| | GACGCGAACUCACCCAAUGUUACCGUUACCGCAUUCUGG | |
| | GCAUGGCCCAAUAAUACGGAAACAGACUUCAAAUGUAAG | |
| | UGGACCCUGACCAGUGGCACCCCUUCUGGCUGCGAGAAU | |
| | AUAAGCGGGGCAUUUGCUAGCAACAGAACAUUCGAUAUU | |
| | ACUGUGUCUGGCCUGGGGACCGCGCCCAAGACACUCAUU | |
| | AUAACGCGAACAGCUACGAACGCUACCACAACCACACAU | |
| | AAAGUGAUUUUCAGCAAGGCUCCAGAAUCAACUACAACC | |
| | AGUCCGACCCUUAAUACCACAGGCUUCGCAGACCCGAACA | |
| | CAACGACGGGACUGCCCUCCUCAACGCAUGUGCCGACUAA | |
| | UCUGACUGCUCCCGCAAGUACCGGCCCAACAGUCUCCACA | |
| | GCAGACGUCACCUCUCCGACUCCCGCCGGCACUACAUCUG | |
| | GAGCCUCACCGGUGACUCCUAGUCCUUCGCCCUGGGACAA | |
| | UGGGACCGAGUCUAAAGCCCCGGACAUGACGAGUUCUAC | |
| | CUCACCAGUUACAACCCCAACACCUAAUGCCACAAGCCCU | |
| | ACGCCAGCCGUGACCACCCCUACGCCCAAUGCCACCAGCC | |
| | CAACACCCGCUGUUACAACGCCAACGCCCAACGCUACUUC | |
| | UCCAACGCUAGGAAAGACAUCACCCACCUCAGCUGUAACC | |
| | ACGCCCACACCAAACGCUACCGGGCCAACCGUUGGCGAAA | |
| | CCUCGCCCCAGGCCAAUGCGACAAACCACACCCUUGGCGG | |
| | GACUUCUCCCACUCCUGUGGUGACUUCACAACCGAAGAA | |
| | CGCGACGUCGGCCGUUACGACCGGGCAGCACAAUAUCACC | |
| | UCGUCCUCCACGUCGUCCAUGUCCUUGCGGCCUUCCAGUA | |
| | ACCCAGAGACACUGUCUCCUAGUACCAGUGACAACAGCA | |
| | CCUCUCACAUGCCAUUACUGACUUCCGCCCACCCAACCGG | |
| | CGGAGAGAACAUCACUCAGGUGACGCCCGCAUCUAUUUC | |
| | UACCCACCACGUGAGCACUAGCAGCCCGGCACCAAGACCA | |
| | GGGACCACCUCCCAGGCUAGCGGCCCGGGCAACUCCUCCA | |
| | CUAGCACGAAACCAGGAGAGGUGAACGUGACCAAGGGCA | |
| | CACCGCCACAGAAUGCAACUUCCCCACAGGCGCCCUCCGG | |
| | CCAGAAGACUGCUGUACCUACGGUCACUAGCACUGGCGG | |
| | AAAGGCGAAUUCAACCACCGGUGGUAAGCAUACCACCGG | |
| | ACACGGCGCUCGGACUAGCACAGAGCCAACCACUGAUUA | |
| | CGGAGGUGACUCAACGACCCCGAGGCCCAGGUACAACGC | |
| | AACCACCUAUCUCCCACCAUCCACAUCUUCCAAGCUCAGG | |
| | CCUCGUUGGACUUUCACCUCCCCGCCUGUGACUACAGCGC | |
| | AGGCCACAGUGCCAGUGCCUCCAACGUCACAGCCGCGGUU | |
| | UAGCAAUCUCUCCAUGCUGGUCCUGCAGUGGGCAUCUCU | |
| | GGCAGUCCUGACACUGCUUCUUUUGCUCGUGAUGGCCGA | |
| | UUGUGCGUUUAGACGCAACCUUUCAACAUCCCACACAUA | |
| | UACAACUCCACCCUAUGAUGACGCAGAGACUUAUGUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 78 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gH (BXLF2)_RX

| SEQ ID NO: 183 consists of from 5′ end to 3′ end: | | 183 |
|---|---|---|
| 5′ UTR SEQ ID NO: 1, mRNA ORF SEQ ID NO: 79, and | | |
| 3′ UTR SEQ ID NO: 3 | | |
| Chemistry | 1-methylpseudouridine | |
| Cap | 7mG(5′)ppp(5′)NlmpNp | |
| 5′UTR | GGGAAAUAAGAGAGAAAAGAAGAGUAAGAAGAAAUAUA | 1 |
| | AGAGCCACC | |
| ORF of mRNA | AUGCAGUUGUUGUGCGUGUUCUGCCUCGUGUUACUCUGG | 79 |
| Construct | GAGGUGGGCGCCGCCAGCCUUAGCGAGGUGAAGCUCCAC | |
| (excluding | UUGGACAUCGAGGGCCACGCCAGCCACUACACCAUCCCCU | |
| the stop | GGACCGAGCUCAUGGCCAAGGUGCCCGGCCUUAGCCCCGA | |
| codon) | GGCCCUGUGGCGGGAGGCCAACGUGACCGAGGACCUGGC | |
| | CAGCAUGCUGAACCGGUACAAGCUGAUCUACAAGACCAG | |
| | CGGCACCCUGGGCAUCGCCCUGGCCGAGCCCGUGGACAUC | |
| | CCCGCCGUUAGCGAAGGCAGCAUGCAGGUGGACGCCAGC | |
| | AAGGUGCACCCCGGCGUGAUCAGCGGCCUGAACAGCCCCG | |
| | CCUGUAUGUUGAGCGCCCCACUGGAGAAGCAGCUGUUCU | |
| | ACUACAUCGGCACCAUGCUGCCCAACACCCGGCCCCACAG | |
| | CUACGUGUUCUACCAGCUGCGGUGCCACCUGAGCUACGU | |
| | UGCCCUGAGCAUCAACGGCGACAAGUUCCAGUACACCGG | |
| | CGCCAUGACCAGCAAGUUCCUGAUGGGCACCUACAAGCG | |
| | GGUCACCGAGAAGGGCGACGAGCACGUGCUGUCACUGGU | |
| | GUUCGGCAAGACCAAGGACCUGCCCGACCUGCGGGGCCCC | |
| | UUCAGCUACCCUAGUUUGACCAGCGCCCAGAGCGGCGAC | |
| | UACAGCUUGGUGAUCGUGACCACCUUCGUGCACUACGCC | |
| | AACUUCCACAACUACUUCGUGCCCAACCUGAAGGACAUG | |
| | UUCAGCCGGGCCGUGACCAUGACUGCCGCUUCUUACGCCC | |
| | GGUACGUGCUGCAGAAGCUGGUCCUGCUGGAGAUGAAGG | |
| | GCGGCUGCCGGGAGCCCGAGCUGGACACCGAAACACUGA | |
| | CCACCAUGUUCGAGGUGAGCGUGGCCUUCUUCAAGGUGG | |
| | GUCACGCGGUGGGCGAAACCGGCAACGGCUGCGUGGACU | |
| | UACGCUGGCUGGCCAAGAGCUUCUUCGAGCUGACCGUGC | |
| | UGAAGGAUAUCAUCGGCAUCUGCUACGGCGCCACCGUGA | |
| | AGGGCAUGCAGAGCUACGGCCUGGAGCGGCUGGCCGCCA | |
| | UGCUUAUGGCAACAGUGAAGAUGGAGGAGCUGGGACACC | |
| | UGACAACAGAGAAGCAGGAGUACGCCCUGAGACUGGCCA | |
| | CAGUGGGCUACCCAAAGGCCGGCGUGUACAGUGGACUGA | |
| | UCGGCGGCGCAACCAGCGUGCUGCUAUCCGCUUACAACCG | |
| | GCACCCGCUGUUCCAGCCCCUGCACACCGUGAUGCGGGAA | |
| | ACCCUGUUCAUCGGAAGCCACGUCGUGCUGCGGGAGCUG | |
| | AGGCUGAACGUAACCACCCAGGGCCCUAAUCUGGCCCUG | |
| | UAUCAGCUCCUCAGUACCGCCCUGUGCAGCGCCCUUGAGA | |
| | UCGGCGAGGUGCUCAGAGGCCUGGCCCUCGGUACCGAGA | |
| | GCGGCCUCUUCAGCCCAUGCUACUUAAGCCUGCGGUUCG | |
| | ACCUGACCCGGGACAAGUUGCUGAGCAUGGCCCCGCAGG | |
| | AGGCCACACUGGACCAGGCAGCUGUAUCCAACGCCGUGG | |
| | ACGGCUUCCUGGGCAGACUGUCCCUGGAACGGGAGGACC | |
| | GGGACGCCUGGCACCUGCCUGCCUACAAGUGUGUGGAUC | |
| | GGCUGGACAAGGUGCUGAUGAUCAUCCCUCUGAUUAAUG | |
| | UCACCUUCAUCAUCAGCAGCGACCGGGAGGUGCGGGGAU | |
| | CCGCCCUCUACGAGGCCAGCACCACCUAUCUGAGCAGCAG | |
| | CCUGUUCCUGUCUCCUGUGAUCAUGAACAAGUGCAGCCA | |
| | GGGCGCCGUGGCCGGCGAGCCCCGGCAGAUCCCCAAGAUC | |
| | CAGAACUUCACCCGGACCCAGAAGUCUUGCAUCUUCUGC | |
| | GGCUUCGCCCUUUUGUCCUACGACGAGAAGGAGGGCUUG | |
| | GAGACUACAACCUACAUCACCAGCCAGGAGGUGCAGAAC | |
| | AGCAUCCUGUCAUCUAAUUACUUCGACUUCGACAACCUG | |
| | CACGUUCAUUACCUGCUCCUCACCACCAACGGUACCGUCA | |
| | UGGAAAUCGCCGGACUGUACGAGGAGCGGGCCCAUGUUG | |
| | UGCUGGCCAUCAUCCUGUACUUCAUCGCUUUCGCACUUG | |
| | GCAUCUUCCUGGUGCACAAGAUCGUGAUGUUCUUCCUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MQLLCVFCLVLLWEVGAASLSEVKLHLDIEGHASHYTIPWTE | 38 |
| amino acid | LMAKVPGLSPEALWREANVTEDLASMLNRYKLIYKTSGTLGI | |
| sequence | ALAEPVDIPAVSEGSMQVDASKVHPGVISGLNSPACMLSAPLE | |
| | KQLFYYIGTMLPNTRPHSYVFYQLRCHLSYVALSINGDKFQYT | |
| | GAMTSKFLMGTYKRVTEKGDEHVLSLVFGKTKDLPDLRGPFS | |
| | YPSLTSAQSGDYSLVIVTTFVHYANFHNYFVPNLKDMFSRAV | |
| | TMTAASYARYVLQKLVLLEMKGGCREPELDTETLTTMFEVS | |
| | VAFFKVGHAVGETGNGCVDLRWLAKSFFELTVLKDIIGICYG | |
| | ATVKGMQSYGLERLAAMLMATVKMEELGHLTTEKQEYALR | |
| | LATVGYPKAGVYSGLIGGATSVLLSAYNRHPLFQPLHTVMRE | |
| | TLFIGSHVVLRELRLNVTTQGPNLALYQLLSTALCSALEIGEVL | |
| | RGLALGTESGLFSPCYLSLRFDLTRDKLLSMAPQEATLDQAA | |
| | VSNAVDGFLGRLSLEREDRDAWHLPAYKCVDRLDKVLMIIPL | |
| | INVTFIISSDREVRGSALYEASTTYLSSSLFLSPVIMNKCSQGAV | |
| | AGEPRQIPKIQNFTRTQKSCIFCGFALLSYDEKEGLETTTYITSQ | |
| | EVQNSILSSNYFDFDNLHVHYLLLTTNGTVMEIAGLYEERAH | |
| | VVLAIILYFIAFALGIFLVHKIVMFFL | |
| PolyA tail | 100 nt | |

EBV gL (B95-8)

| SEQ ID NO: 184 consists of from 5′ end to 3′ end: | | 184 |
|---|---|---|
| 5′ UTR SEQ ID NO: 1, mRNA ORF SEQ ID NO: 80, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGAGAGCCGUGGGCGUGUUCCUGGCCAUCUGCCUGGUG | 80 |
| Construct | ACCAUCUUCGUGCUGCCCACCUGGGGCAACUGGGCCUACC | |
| (excluding | CCUGCUGCCACGUGACCCAGCUGAGAGCCCAGCACCUGCU | |
| the stop | GGCCCUGGAGAACAUCAGCGACAUCUACCUGGUGAGCAA | |
| codon) | CCAGACCUGCGACGGCUUCAGCCUGGCCAGCCUGAACAGC | |
| | CCCAAGAACGGCAGCAACCAGCUGGUGAUCAGCAGAUGC | |
| | GCCAACGGCCUGAACGUGGUGAGCUUCUUCAUCAGCAUC | |
| | CUGAAGAGAAGCAGCAGCGCCCUGACCGGCCACCUGAGA | |
| | GAGCUGCUGACCACCCUGGAGACCCUGUACGGCAGCUUC | |
| | AGCGUGGAGGACCUGUUCGGCGCCAACCUGAACAGAUAC | |
| | GCCUGGCACAGAGGCGGC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MRAVGVFLAICLVTIFVLPTWGNWAYPCCHVTQLRAQHLLA | 36 |
| amino acid | LENISDIYLVSNQTCDGFSLASLNSPKNGSNQLVISRCANGLNV | |
| sequence | VSFFISILKRSSSALTGHLRELLTTLETLYGSFSVEDLFGANLNR | |
| | YAWHRGG | |
| PolyA tail | 100 nt | |

EBV gp350_001 (modified)

| SEQ ID NO: 185 consists of from 5′ end to 3′ end: | | 185 |
|---|---|---|
| 5′ UTR SEQ ID NO: 1, mRNA ORF SEQ ID NO: 81, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 81 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 28 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

LMP1 del 1-44_RX

| SEQ ID NO: 186 consists of from 5′ end to 3′ end: | | 186 |
|---|---|---|
| 5′ UTR SEQ ID NO: 1, mRNA ORF SEQ ID NO: 82, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGAGUGACUGGACAGGAGGAGCCCUCCUUGUCCUCUAU | 82 |
| Construct | UCCUUUGCUCUCAUGCUUAUAAUUAUCAUCCUGAUAAUC | |
| (excluding | UUUAUCUUCAGACGGGACCUUCUCUGUCCACUUGGAGCC | |
| the stop | CUUUGUAUACUCCUAUUGAUGAUCACCCUCCUCCUCAUC | |
| codon) | GCUCUCUGGAAUUUGCACGGACAGGCAUUGUUCCUUGGA | |
| | AUUGUGCUGUUCAUCUUCGGGUGCUUACUUGUCUUAGGU | |
| | AUCUGGAUCUACUUAUUGGAGAUGCUCUGGCGACUUGGU | |
| | GCCACCAUCUGGCAGCUUUUGGCCUUCUUCCUAGCGUUC | |
| | UUCCUCGACCUCAUUCUACUCAUUAUUGCUCUCUAUCUA | |
| | CAACAGAACUGGUGGACUCUAUUGGUUGAUCUCCUUUGG | |
| | CUCCUCCUGUUUCUGGCGAUUUUAAUCUGGAUGUAUUAC | |
| | CAUGGACAACGACACAGUGAUGAACACCACCACGAUGAC | |
| | UCCCUCCCGCACCCUCAACAAGCUACCGAUGAUUCUGGCC | |
| | AUGAAUCUGACUCUAACUCCAACGAGGGCAGACACCACC | |
| | UGCUCGUGAGUGGAGCCGGCGACGGACCACCACUCUGCU | |
| | CUCAGAACCUAGGCGCACCUGGAGGUGGUCCUGACAAUG | |
| | GCCCACAGGACCCUGACAACACGGAUGAUAACGGUCCAC | |
| | AAGACCCAGAUAACACUGACGACAAUGGACCGCAUGACC | |
| | CGCUGCCUCAGGAUCCUGAUAACACAGACGACAACGGCCC | |
| | ACAAGAUCCGGACAAUACCGACGAUAACGGACCGCACGA | |
| | CCCGUUACCUCAUAGCCCUAGCGACUCUGCUGGAAAUGA | |
| | UGGAGGCCCUCCACAAUUGACGGAAGAGGUUGAGAACAA | |
| | AGGAGGUGACCAGGGCCCGCCUUUGAUGACAGACGGAGG | |
| | CGGCGGUCAUAGUCAUGAUUCCGGCCAUGGCGGAGGAGA | |
| | UCCACACCUUCCUACGCUGCUGUUGGGUUCUUCUGGUUC | |
| | CGGUGGAGAUGAUGACGACCCGCACGGCCCAGUUCAGCU | |
| | AAGCUACUAUGAC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MSDWTGGALLVLYSFALMLIIIILIIFIFRRDLLCPLGALCILLLM | 83 |
| amino acid | ITLLLIALWNLHGQALFLGIVLFIFGCLLVLGIWIYLLEMLWRL | |
| sequence | GATIWQLLAFFLAFFLDLILLIIALYLQQNWWTLLVDLLWLLL | |
| | FLAILIWMYYHGQRHSDEHHHDDSLPHPQQATDDSGHESDSN | |
| | SNEGRHHLLVSGAGDGPPLCSQNLGAPGGGPDNGPQDPDNTD | |
| | DNGPQDPDNTDDNGPHDPLPQDPDNTDDNGPQDPDNTDDNG | |
| | PHDPLPHSPSDSAGNDGGPPQLTEEVENKGGDQGPPLMTDGG | |
| | GGHSHDSGHGGGDPHLPTLLLGSSGSGGDDDDPHGPVQLSYY | |
| | D | |
| PolyA tail | 100 nt | |

EBV gH (BXLF2)_RX

| SEQ ID NO: 187 consists of from 5′ end to 3′ end: | | 187 |
|---|---|---|
| 5′ UTR SEQ ID NO: 1, mRNA ORF SEQ ID NO: 84, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGCAGUUGUUGUGCGUGUUCUGCCUCGUGUUACUCUGG | 84 |
| Construct | GAGGUGGGCGCCGCCAGCCUUAGCGAGGUGAAGCUCCAC | |
| (excluding | UUGGACAUCGAGGGCCACGCCAGCCACUACACCAUCCCCU | |
| the stop | GGACCGAGCUCAUGGCCAAGGUGCCCGGCCUUAGCCCCGA | |
| codon) | GGCCCUGUGGCGGGAGGCCAACGUGACCGAGGACCUGGC | |
| | CAGCAUGCUGAACCGGUACAAGCUGAUCUACAAGACCAG | |
| | CGGCACCCUGGGCAUCGCCCUGGCCGAGCCCGUGGACAUC | |
| | CCCGCCGUUAGCGAAGGCAGCAUGCAGGUGGACGCCAGC | |
| | AAGGUGCACCCCGGCGUGAUCAGCGGCCUGAACAGCCCCG | |
| | CCUGUAUGUUGAGCGCCCCACUGGAGAAGCAGCUGUUCU | |
| | ACUACAUCGGCACCAUGCUGCCCAACACCCGGCCCCACAG | |
| | CUACGUGUUCUACCAGCUGCGGUGCCACCUGAGCUACGU | |
| | UGCCCUGAGCAUCAACGGCGACAAGUUCCAGUACACCGG | |
| | CGCCAUGACCAGCAAGUUCCUGAUGGGCACCUACAAGCG | |
| | GGUCACCGAGAAGGGCGACGAGCACGUGCUGUCACUGGU | |
| | GUUCGGCAAGACCAAGGACCUGCCCGACCUGCGGGGCCCC | |
| | UUCAGCUACCCUAGUUUGACCAGCGCCCAGAGCGGCGAC | |
| | UACAGCUUGGUGAUCGUGACCACCUUCGUGCACUACGCC | |
| | AACUUCCACAACUACUUCGUGCCCAACCUGAAGGACAUG | |
| | UUCAGCCGGGCCGUGACCAUGACUGCCGCUUCUUACGCCC | |
| | GGUACGUGCUGCAGAAGCUGGUCCUGCUGGAGAUGAAGG | |
| | GCGGCUGCCGGGAGCCCGAGCUGGACACCGAAACACUGA | |
| | CCACCAUGUUCGAGGUGAGCGUGGCCUUCUUCAAGGUGG | |
| | GUCACGCGGUGGGCGAAACCGGCAACGGCUGCGUGGACU | |
| | UACGCUGGCUGGCCAAGAGCUUCUUCGAGCUGACCGUGC | |
| | UGAAGGAUAUCAUCGGCAUCUGCUACGGCGCCACCGUGA | |
| | AGGGCAUGCAGAGCUACGGCCUGGAGCGGCUGGCCGCCA | |
| | UGCUUAUGGCAACAGUGAAGAUGGAGGAGCUGGGACACC | |
| | UGACAACAGAGAAGCAGGAGUACGCCCUGAGACUGGCCA | |
| | CAGUGGGCUACCCAAAGGCCGGCGUGUACAGUGGACUGA | |
| | UCGGCGGCGCAACCAGCGUGCUGCUAUCCGCUUACAACCG | |
| | GCACCCGCUGUUCCAGCCCCUGCACACCGUGAUGCGGGAA | |
| | ACCCUGUUCAUCGGAAGCCACGUCGUGCUGCGGGAGCUG | |
| | AGGCUGAACGUAACCACCCAGGGCCCUAAUCUGGCCCUG | |
| | UAUCAGCUCCUCAGUACCGCCCUGUGCAGCGCCCUUGAGA | |
| | UCGGCGAGGUGCUCAGAGGCCUGGCCCUCGGUACCGAGA | |
| | GCGGCCUCUUCAGCCCAUGCUACUUAAGCCUGCGGUUCG | |
| | ACCUGACCCGGGACAAGUUGCUGAGCAUGGCCCCGCAGG | |
| | AGGCCACACUGGACCAGGCAGCUGUAUCCAACGCCGUGG | |
| | ACGGCUUCCUGGGCAGACUGUCCCUGGAACGGGAGGACC | |
| | GGGACGCCUGGCACCUGCCUGCCUACAAGUGUGUGGAUC | |
| | GGCUGGACAAGGUGCUGAUGAUCAUCCCUCUGAUUAAUG | |
| | UCACCUUCAUCAUCAGCAGCGACCGGGAGGUGCGGGGAU | |
| | CCGCCCUCUACGAGGCCAGCACCACCUAUCUGAGCAGCAG | |
| | CCUGUUCCUGUCUCCUGUGAUCAUGAACAAGUGCAGCCA | |
| | GGGCGCCGUGGCCGGCGAGCCCCGGCAGAUCCCCAAGAUC | |
| | CAGAACUUCACCCGGACCCAGAAGUCUUGCAUCUUCUGC | |
| | GGCUUCGCCCUUUUGUCCUACGACGAGAAGGAGGGCUUG | |
| | GAGACUACAACCUACAUCACCAGCCAGGAGGUGCAGAAC | |
| | AGCAUCCUGUCAUCUAAUUACUUCGACUUCGACAACCUG | |
| | CACGUUCAUUACCUGCUCCUCACCACCAACGGUACCGUCA | |
| | UGGAAAUCGCCGGACUGUACGAGGAGCGGGCCCAUGUUG | |
| | UGCUGGCCAUCAUCCUGUACUUCAUCGCUUUCGCACUUG | |
| | GCAUCUUCCUGGUGCACAAGAUCGUGAUGUUCUUCCUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MQLLCVFCLVLLWEVGAASLSEVKLHLDIEGHASHYTIPWTE | 38 |
| amino acid | LMAKVPGLSPEALWREANVTEDLASMLNRYKLIYKTSGTLGI | |
| sequence | ALAEPVDIPAVSEGSMQVDASKVHPGVISGLNSPACMLSAPLE | |
| | KQLFYYIGTMLPNTRPHSYVFYQLRCHLSYVALSINGDKFQYT | |
| | GAMTSKFLMGTYKRVTEKGDEHVLSLVFGKTKDLPDLRGPFS | |
| | YPSLTSAQSGDYSLVIVTTFVHYANFHNYFVPNLKDMFSRAV | |
| | TMTAASYARYVLQKLVLLEMKGGCREPELDTETLTTMFEVS | |
| | VAFFKVGHAVGETGNGCVDLRWLAKSFFELTVLKDIIGICYG | |
| | ATVKGMQSYGLERLAAMLMATVKMEELGHLTTEKQEYALR | |
| | LATVGYPKAGVYSGLIGGATSVLLSAYNRHPLFQPLHTVMRE | |
| | TLFIGSHVVLRELRLNVTTQGPNLALYQLLSTALCSALEIGEVL | |
| | RGLALGTESGLFSPCYLSLRFDLTRDKLLSMAPQEATLDQAA | |
| | VSNAVDGFLGRLSLEREDRDAWHLPAYKCVDRLDKVLMIIPL | |
| | INVTFIISSDREVRGSALYEASTTYLSSSLFLSPVIMNKCSQGAV | |
| | AGEPRQIPKIQNFTRTQKSCIFCGFALLSYDEKEGLETTTYITSQ | |
| | EVQNSILSSNYFDFDNLHVHYLLLTTNGTVMEIAGLYEERAH | |
| | VVLAIILYFIAFALGIFLVHKIVMFFL | |
| PolyA tail | 100 nt | |

EBV gL (B95-8)

| SEQ ID NO: 188 consists of from 5′ end to 3′ end: | | 188 |
|---|---|---|
| 5′ UTR SEQ ID NO: 1, mRNA ORF SEQ ID NO: 85, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGAGAGCCGUGGGCGUGUUCCUGGCCAUCUGCCUGGUG | 85 |
| Construct | ACCAUCUUCGUGCUGCCCACCUGGGGCAACUGGGCCUACC | |
| (excluding | CCUGCUGCCACGUGACCCAGCUGAGAGCCCAGCACCUGCU | |
| the stop | GGCCCUGGAGAACAUCAGCGACAUCUACCUGGUGAGCAA | |
| codon) | CCAGACCUGCGACGGCUUCAGCCUGGCCAGCCUGAACAGC | |
| | CCCAAGAACGGCAGCAACCAGCUGGUGAUCAGCAGAUGC | |
| | GCCAACGGCCUGAACGUGGUGAGCUUCUUCAUCAGCAUC | |
| | CUGAAGAGAAGCAGCAGCGCCCUGACCGGCCACCUGAGA | |
| | GAGCUGCUGACCACCCUGGAGACCCUGUACGGCAGCUUC | |
| | AGCGUGGAGGACCUGUUCGGCGCCAACCUGAACAGAUAC | |
| | GCCUGGCACAGAGGCGGC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MRAVGVFLAICLVTIFVLPTWGNWAYPCCHVTQLRAQHLLA | 36 |
| amino acid | LENISDIYLVSNQTCDGFSLASLNSPKNGSNQLVISRCANGLNV | |
| sequence | VSFFISILKRSSSALTGHLRELLTTLETLYGSFSVEDLFGANLNR | |
| | YAWHRGG | |
| PolyA tail | 100 nt | |

EBV gp42 (B95-8)_DX

| SEQ ID NO: 189 consists of from 5′ end to 3′ end: | | 189 |
|---|---|---|
| 5′ UTR SEQ ID NO: 1, mRNA ORF SEQ ID NO: 86, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGUGAGCUUCAAGCAGGUGAGAGUGCCCCUGUUCACC | 86 |
| Construct | GCCAUCGCCCUGGUGAUCGUGCUGCUGCUGGCCUACUUCC | |
| (excluding | UGCCCCCCAGAGUGAGAGGCGGCGGCAGAGUGGCCGCCG | |
| the stop | CCGCCAUCACCUGGGUGCCCAAGCCCAACGUGGAGGUGU | |
| codon) | GGCCCGUGGACCCCCCACCCCCCGUGAACUUCAACAAGAC | |
| | CGCCGAGCAGGAGUACGGCGACAAGGAGGUGAAGCUGCC | |
| | CCACUGGACCCCCACCCUGCACACCUUCCAGGUGCCCCAG | |
| | AACUACACCAAGGCCAACUGCACCUACUGCAACACCAGAG | |
| | AGUACACCUUCAGCUACAAGGGCUGCUGCUUCUACUUCA | |
| | CCAAGAAGAAGCACACCUGGAACGGCUGCUUCCAGGCCU | |
| | GCGCCGAGCUGUACCCCUGCACCUACUUCUACGGCCCCAC | |
| | CCCCGACAUCCUGCCCGUGGUGACCAGAAACCUGAACGCC | |
| | AUCGAGAGCCUGUGGGUGGGCGUGUACAGAGUGGGCGAG | |
| | GGCAACUGGACCAGCCUGGACGGCGGCACCUUCAAGGUG | |
| | UACCAGAUCUUCGGCAGCCACUGCACCUACGUGAGCAAG | |
| | UUCAGCACCGUGCCCGUGAGCCACCACGAGUGCAGCUUCC | |
| | UGAAGCCCUGCCUGUGCGUGAGCCAGAGAAGCAACAGC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MVSFKQVRVPLFTAIALVIVLLLAYFLPPRVRGGGRVAAAAIT | 34 |
| amino acid | WVPKPNVEVWPVDPPPPVNFNKTAEQEYGDKEVKLPHWTPT | |
| sequence | LHTFQVPQNYTKANCTYCNTREYTFSYKGCCFYFTKKKHTW | |
| | NGCFQACAELYPCTYFYGPTPDILPVVTRNLNAIESLWVGVYR | |
| | VGEGNWTSLDGGTFKVYQIFGSHCTYVSKFSTVPVSHHECSFL | |
| | KPCLCVSQRSNS | |
| PolyA tail | 100 nt | |

gp350 D123_LS_RX

| SEQ ID NO: 190 consists of from 5′ end to 3′ end: | | 190 |
|---|---|---|
| 5′ UTR SEQ ID NO: 1, mRNA ORF SEQ ID NO: 87, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGACAGCAAGGGCAGCAGCCAGAAGGGAAGCAGGUUG | 87 |
| Construct | UUAUUGCUUUUAGUGGUGAGCAACUUACUCUUACCCCAG | |
| (excluding | AUCCAGUCGCUCAUUCAUUUGACAGGCGAGGACCCCGGC | |
| the stop | UUCUUUAACGUGGAAAUCCCAGAGUUCCCUUUCUAUCCA | |
| codon) | ACCUGUAACGUUUGCACAGCCGACGUGAACGUGACAAUC | |
| | AAUUUCGACGUGGGAGGAAAGAAGCAUCAGCUUGAUCUG | |
| | GAUUUCGGCCAACUGACGCCACACACCAAAGCGGUGUAC | |
| | CAACCAAGGGGUGCAUUUGGAGGGAGCGAGAAUGCUACA | |
| | AAUCUGUUCUUACUGGAACUGCUUGGUGCAGGAGAGCUG | |
| | GCACUAACCAUGCGAAGCAAGAAGCUCCCCAUCAACGUU | |
| | ACCACCGGGGAAGAACAGCAAGUCAGCCUUGAAUCCGUG | |
| | GAUGUGUACUUUCAGGAUGUUUUCGGCACCAUGUGGUGU | |
| | CACCACGCUGAGAUGCAGAAUCCCGUGUAUCUCAUACCC | |
| | GAGACUGUACCCUACAUCAAGUGGGAUAAUUGCAAUUCA | |
| | ACUAAUAUUACGGCUGUGGUGAGGGCCCAGGGCUUGGAC | |
| | GUGACUCUGCCCUUAUCUCUACCUACUUCUGCCCAAGACU | |
| | CCAAUUUCUCUGUCAAGACCGAGAUGCUCGGGAAUGAGA | |
| | UCGAUAUCGAGUGCAUCAUGGAGGACGGUGAGAUAAGCC | |
| | AGGUUCUGCCCGGCGACAACAAGUUCAAUAUCACUUGUU | |
| | CUGGCUACGAGUCCCAUGUGCCUAGUGGUGGCAUACUCA | |
| | CAAGUACUUCUCCCGUAGCCACGCCCAUUCCCGGAACCGG | |
| | AUACGCCUACAGUCUGCGUCUGACCCCACGGCCUGUGUCC | |
| | AGAUUCCUGGGUAACAAUAGUAUCUUAUACGUGUUUUAU | |
| | AGCGGAAACGGCCCUAAAGCGUCCGGAGGGGACUAUUGU | |
| | AUUCAGAGUAAUAUCGUUUUCUCUGAUGAGAUCCCUGCC | |
| | AGUCAGGACAUGCCGACAAACACAACUGAUAUUACCUAC | |
| | GUGGGCGACAAUGCCACGUAUUCAGUGCCCAUGGUCACG | |
| | AGCGAGGACGCCAAUUCACCAAAUGUUACUGUAACAGCU | |
| | UUCUGGGCCUGGCCAAAUAACACUGAGACUGACUUCAAA | |
| | UGUAAGUGGACUUUGACCUCUGGAACUCCGUCGGGUUGC | |
| | GAGAAUAUCAGCGGGGCCUUUGCUUCCAACAGGACUUUC | |
| | GACAUCACUGUCUCAGGGCUGGGGACAGCACCGAAGACA | |
| | UUAAUCAUAACACGGACCGCCACCAACGCCACGACUACAA | |
| | CCCAUAAGGUGAUCUUUUCCAAGGCACCUGGCAGCGGCA | |
| | GCGCAGGCUCUGCAAGCGGCGGCGCCUCCAGCGGCUCCGG | |
| | AGGUGCCAGCAUGCAGAUCUACGAGGGCAAGCUGACCGC | |
| | CGAGGGCCUGAGGUUCGGCAUCGUGGCCAGCAGGUUCAA | |
| | CCACGCCCUGGUGGACAGGCUGGUGGAAGGCGCCAUCGA | |
| | CGCCAUCGUGAGGCACGGCGGCAGGGAGGAGGACAUCAC | |
| | CCUGGUGCGGGUGCCCGGCAGCUGGGAGAUCCCCGUGGC | |
| | CGCCGGCGAGCUGGCCAGGAAGGAAGAUAUUGAUGCAGU | |
| | GAUCGCCAUCGGCGUGCUGAUCAGGGGCGCCACCCCACAC | |
| | UUCGACUACAUCGCCAGCGAAGUGAGCAAGGGACUGGCG | |
| | GACCUGAGCCUGGAGCUGAGGAAGCCCAUCACCUUCGGC | |
| | GUGAUCACCGCCGACACCCUGGAGCAGGCCAUCGAGAGG | |
| | GCCGGCACCAAGCACGGCAACAAGGGAUGGGAGGCCGCC | |
| | CUGAGCGCUAUCGAAAUGGCCAACCUCUUCAAGAGCCUG | |
| | AGG | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDSKGSSQKGSRLLLLLVVSNLLLPQGVVEAALLVCQYTIQS | 88 |
| amino acid | LIHLTGEDPGFFNVEIPEFPFYPTCNVCTADVNVTINFDVGGKK | |
| sequence | HQLDLDFGQLTPHTKAVYQPRGAFGGSENATNLFLLELLGAG | |
| | ELALTMRSKKLPINVTTGEEQQVSLESVDVYFQDVFGTMWCH | |
| | HAEMQNPVYLIPETVPYIKWDNCNSTNITAVVRAQGLDVTLP | |
| | LSLPTSAQDSNFSVKTEMLGNEIDIECIMEDGEISQVLPGDNKF | |
| | NITCSGYESHVPSGGILTSTSPVATPIPGTGYAYSLRLTPRPVSR | |
| | FLGNNSILYVFYSGNGPKASGGDYCIQSNIVFSDEIPASQDMPT | |
| | NTTDITYVGDNATYSVPMVTSEDANSPNVTVTAFWAWPNNT | |
| | ETDFKCKWTLTSGTPSGCENISGAFASNRTFDITVSGLGTAPKT | |
| | LIITRTATNATTTTHKVIFSKAPGSGSAGSASGGASSGSGGASM | |
| | QIYEGKLTAEGLRFGIVASRFNHALVDRLVEGAIDAIVRHGGR | |
| | EEDITLVRVPGSWEIPVAAGELARKEDIDAVIAIGVLIRGATPH | |
| | FDYIASEVSKGLADLSLELRKPITFGVITADTLEQAIERAGTKH | |
| | GNKGWEAALSAIEMANLFKSLR | |
| PolyA tail | 100 nt | |

gp350_D123_HBsAg_RX

| SEQ ID NO: 191 consists of from 5′ end to 3′ end: | | 191 |
|---|---|---|
| 5′ UTR SEQ ID NO: 1, mRNA ORF SEQ ID NO: 89, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGACAGCAAGGGCAGCAGCCAGAAGGGCAGUAGGCUC | 89 |
| Construct | CUUUUGCUACUUGUGGUGAGCAACCUCUUGUUACCCCAG | |
| (excluding | GGCGUGGUGGAGGCAGCACUCCUCGUCUGCCAGUAUACA | |
| the stop | AUCCAGUCGCUCAUUCAUUUGACAGGCGAGGACCCCGGC | |
| codon) | UUCUUUAACGUGGAAAUCCCAGAGUUCCCUUUCUAUCCA | |
| | ACCUGUAACGUUUGCACGGCAGACGUGAACGUGACAAUC | |
| | AAUUUCGACGUGGGAGGAAAGAAGCAUCAGCUUGAUCUG | |
| | GAUUUCGGCCAACUGACGCCACACACCAAAGCGGUGUAC | |
| | CAACCAAGGGGUGCAUUUGGAGGGAGCGAGAAUGCUACA | |
| | AAUCUGUUCUUACUGGAACUGCUUGGUGCAGGAGAGCUG | |
| | GCACUAACCAUGCGAAGCAAGAAGCUCCCCAUCAACGUU | |
| | ACCACCGGGGAAGAACAGCAAGUCAGCCUUGAAUCCGUG | |
| | GAUGUGUACUUUCAGGAUGUUUUCGGCACCAUGUGGUGU | |
| | CACCACGCUGAGAUGCAGAAUCCCGUGUAUCUCAUACCC | |
| | GAGACUGUACCCUACAUCAAGUGGGAUAAUUGCAAUUCA | |
| | ACUAAUAUUACGGCUGUAGUGAGGGCCCAGGGCUUGGAC | |
| | GUGACUCUGCCCUUAUCUCUACCUACUUCUGCCCAAGACU | |
| | CCAAUUUCUCUGUCAAGACCGAGAUGCUCGGGAAUGAGA | |
| | UCGAUAUCGAGUGCAUCAUGGAGGACGGUGAGAUAAGCC | |
| | AGGUUCUGCCCGGCGACAACAAGUUCAAUAUCACUUGUU | |
| | CUGGCUACGAGUCCCAUGUGCCUAGUGGUGGCAUACUCA | |
| | CAAGUACUUCUCCCGUAGCCACGCCCAUUCCCGGAACCGG | |
| | AUACGCCUACAGUCUGCGUCUGACCCCACGGCCUGUGUCC | |
| | AGAUUCCUGGGUAACAAUAGUAUCUUAUACGUGUUUUAU | |
| | AGCGGAAACGGCCCUAAAGCGUCCGGAGGGGACUAUUGU | |
| | AUUCAGAGUAAUAUCGUUUUCUCUGAUGAGAUUCCAGCC | |
| | AGUCAGGACAUGCCGACAAACACAACUGAUAUUACCUAC | |
| | GUGGGCGACAAUGCCACGUAUUCAGUGCCCAUGGUCACG | |
| | AGCGAGGACGCCAAUUCACCAAAUGUUACUGUAACAGCU | |
| | UUCUGGGCCUGGCCAAAUAACACUGAGACUGACUUCAAA | |
| | UGUAAGUGGACUUUGACCUCUGGAACUCCGUCGGGUUGC | |
| | GAGAAUAUCAGCGGGGCCUUUGCUUCCAACAGGACUUUC | |
| | GACAUCACUGUCUCAGGGCUGGGGACAGCACCGAAGACA | |
| | UUAAUCAUAACACGGACCGCCACCAACGCCACGACUACAA | |
| | CCCAUAAGGUGAUCUUUUCCAAGGCACCUGGCAGCGGCA | |
| | GCGCUGGCUCCGCUAGCGGCGGCGCCUCUAGCGGAUCAG | |
| | GAGGCGCAUCCAUGGAGAACAUUGCAUCUGGCCUGCUGG | |
| | GACCUCUACUGGUGCUGCAGGCCGGAUUCUUCCUGCUGA | |
| | CCAAGAUCCUGACCAUCCCUCAGAGCCUGGACAGCUGGU | |
| | GGACCAGCCUGAACUUCCUGGGCGGCACCCCAGUGUGCCU | |
| | GGGCCAGAACAGCCAGAGCCAGAUCUCCUCCCACAGCCCC | |
| | ACCUGCUGUCCGCCCAUCUGCCCCGGCUACCGUUGGAUGU | |
| | GCCUGAGGAGGUUCAUCAUCUUCCUGUGCAUACUCCUGU | |
| | UGUGUCUAAUUUUCCUGCUCGUUCUGCUCGACUACCAGG | |
| | GCAUGCUGCCGGUGUGUCCUCUGAUCCCCGGCUCCUCCAC | |
| | CACCAGCACCGGCCCCUGCAGGACCUGCACCACACCAGCA | |
| | CAAGGUACCAGCAUGUUCCCCAGCUGUUGCUGUACCAAG | |
| | CCCACCGACGGCAACUGCACCUGCAUCCCCAUCCCCAGCA | |
| | GCUGGGCCUUCGCCAAGUACCUGUGGGAGUGGGCCAGCG | |
| | UGAGGUUCAGCUGGCUGUCACUUCUUGCGCCAUUCGUGC | |
| | AGUGGUUCGUGGGCCUGAGCCCUACUGUGUGGCUGUCGG | |
| | UAAUCUGGAUGAUGUGGUUCUGGGGCCCCAGCCUGUACA | |
| | ACAUCCUGUCACCGUUCAUGCCUCUCCUACCGCUGUUCUU | |
| | CUGCCUGUGGGUGUACAUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDSKGSSQKGSRLLLLLVVSNLLLPQGVVEAALLVCQYTIQS | 90 |
| amino acid | LIHLTGEDPGFFNVEIPEFPFYPTCNVCTADVNVTINFDVGGKK | |
| sequence | HQLDLDFGQLTPHTKAVYQPRGAFGGSENATNLFLLELLGAG | |
| | ELALTMRSKKLPINVTTGEEQQVSLESVDVYFQDVFGTMWCH | |
| | HAEMQNPVYLIPETVPYIKWDNCNSTNITAVVRAQGLDVTLP | |
| | LSLPTSAQDSNFSVKTEMLGNEIDIECIMEDGEISQVLPGDNKF | |
| | NITCSGYESHVPSGGILTSTSPVATPIPGTGYAYSLRLTPRPVSR | |
| | FLGNNSILYVFYSGNGPKASGGDYCIQSNIVFSDEIPASQDMPT | |
| | NTTDITYVGDNATYSVPMVTSEDANSPNVTVTAFWAWPNNT | |
| | ETDFKCKWTLTSGTPSGCENISGAFASNRTFDITVSGLGTAPKT | |
| | LIITRTATNATTTTHKVIFSKAPGSGSAGSASGGASSGSGGASM | |
| | ENIASGLLGPLLVLQAGFFLLTKILTIPQSLDSWWTSLNFLGGT | |
| | PVCLGQNSQSQISSHSPTCCPPICPGYRWMCLRRFIIFLCILLLC | |
| | LIFLLVLLDYQGMLPVCPLIPGSSTTSTGPCRTCTTPAQGTSMF | |
| | PSCCCTKPTDGNCTCIPIPSSWAFAKYLWEWASVRFSWLSLLA | |
| | PFVQWFVGLSPTVWLSVIWMMWFWGPSLYNILSPFMPLLPLF | |
| | FCLWVYI | |
| PolyA tail | 100 nt | |

gp350 D123_HBcAg_heterotandem_RX

| SEQ ID NO: 192 consists of from 5′ end to 3′ end: | | 192 |
|---|---|---|
| 5′ UTR SEQ ID NO: 1, mRNA ORF SEQ ID NO: 91, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGACAGCAAGGGCAGCAGCCAGAAGGGAUCCAGGCUA | 91 |
| Construct | UUGUUACUAUUAGUGGUGAGCAACCUCCUCUUGCCCCAG | |
| (excluding | GGCGUGGUGGACAUCGACCCCUACAAGGAGUUCGGCGCC | |
| the stop | ACCGUGGAGUUGUUAAGCUUCCUCCCCAGCGACUUCUUC | |
| codon) | CCCAGCGUGAGGGACCUGCUGGACACCGCCGCCGCCCUGU | |
| | ACAGGGAGGCCCUGGAGAGCCCCGAGCACUGCUCGCCUCA | |
| | CCACACCGCCCUGAGGCAGGCCAUCCUGUGCUGGGGCGAG | |
| | CUGAUGACCCUGGCCACCUGGGUGGGCAACAACCUGGAG | |
| | GACCCCGCCAGCCGCGACCUCGUUGUGAACUACGUGAACA | |
| | CCAACAUGGGCCUGAAGAUCAGGCAGCUGCUGUGGUUCC | |
| | ACAUCAGCUGCCUGACCUUCGGCAGGGAGACAGUGCUGG | |
| | AGUACCUGGUGAGCUUCGGCGUGUGGAUCAGGACUCCAC | |
| | CCGCCUACAGGCCUCCCAACGCGCCAAUCCUGAGCACCCU | |
| | GCCCGAGACAACAGUGGUGGGCAGCGGCAGCGCAGGUUC | |
| | CGCCAGCGGCGGCGCAUCCAGUGGCUCUGGAGGAGCUUC | |
| | UGACAUUGACCCAUAUAAGGAAUUCGGUGCUACAGUAGA | |
| | GCUACUGUCUUUCUUGCCUAGUGAUUUCUUCCCGAGUGU | |
| | AAGGGAUCUCCUAGAUACAGCCGCUGCUCUAUAUAGAGA | |
| | GGCACUGGAAUCUCCAGAGCACUGUUCCCCACACCAUACC | |
| | GCUCUCCGGCAGGCAAUCCUGUGUUGGGGUGAACUUAUG | |
| | ACACUGGCAACCUGGGUCGGUAACAACGGAUCAGGUGAG | |
| | GCGGCUCUGCUGGUCUGCCAGUAUACAAUCCAGUCGCUC | |
| | AUUCAUCUGACAGGCGAAGAUCCAGGUUUCUUUAACGUG | |
| | GAAAUCCCAGAGUUCCCUUUCUAUCCAACCUGUAACGUU | |
| | UGUACAGCUGACGUGAACGUGACAAUCAAUUUCGACGUG | |
| | GGAGGAAAGAAGCAUCAGCUUGAUCUGGAUUUCGGCCAA | |
| | CUGACGCCACACACCAAAGCGGUGUACCAACCAAGGGGU | |
| | GCAUUUGGAGGGAGCGAGAAUGCUACAAAUCUGUUCUUA | |
| | CUGGAACUGCUUGGUGCAGGAGAGCUGGCACUAACCAUG | |
| | CGAAGCAAGAAGCUCCCCAUCAACGUUACCACCGGGGAA | |
| | GAACAGCAAGUCAGCCUUGAAUCCGUGGAUGUGUACUUU | |
| | CAGGAUGUUUUCGGCACCAUGUGGUGUCACCACGCUGAG | |
| | AUGCAGAAUCCCGUGUAUCUCAUACCCGAGACUGUACCC | |
| | UACAUCAAGUGGGAUAAUUGCAAUUCAACUAAUAUUACG | |
| | GCUGUGGUGAGGGCCCAGGGCUUGGACGUGACUCUGCCC | |
| | UUAUCUCUACCUACUUCUGCCCAAGACUCCAAUUUCUCU | |
| | GUCAAGACCGAGAUGCUCGGGAAUGAGAUCGAUAUCGAG | |
| | UGCAUCAUGGAGGACGGUGAGAUAAGCCAGGUUCUGCCC | |
| | GGCGACAACAAGUUCAAUAUCACUUGUUCUGGCUACGAG | |
| | UCCCAUGUGCCUUCUGGUGGCAUACUCACAAGUACUUCU | |
| | CCCGUAGCCACGCCCAUUCCCGGAACCGGAUAUGCGUAUA | |
| | GUCUGCGUCUGACCCCACGGCCUGUGUCCAGAUUCCUGG | |
| | GUAACAAUAGUAUCUUAUACGUGUUUUAUAGCGGAAACG | |
| | GCCCUAAAGCGUCCGGAGGGGACUAUUGUAUUCAGAGUA | |
| | AUAUCGUUUUCUCUGAUGAAAUUCCUGCCAGUCAGGACA | |
| | UGCCGACAAACACAACUGAUAUUACCUACGUGGGCGACA | |
| | AUGCCACGUAUUCAGUGCCCAUGGUCACGAGCGAGGACG | |
| | CCAAUUCACCAAAUGUUACUGUAACAGCUUUCUGGGCCU | |
| | GGCCAAAUAACACUGAGACUGACUUCAAAUGUAAGUGGA | |
| | CUUUGACCUCUGGAACUCCGUCGGGUUGCGAGAAUAUCA | |
| | GCGGGGCCUUUGCUUCCAACAGGACUUUCGACAUCACUG | |
| | UCUCAGGGCUGGGGACAGCACCGAAGACAUUAAUCAUAA | |
| | CACGGACCGCCACCAACGCCACGACUACAACCCAUAAGGU | |
| | GAUCUUUUCCAAGGCACCUGGAUCCGGACUGGUGGUCAA | |
| | CUAUGUGAAUACAAAUAUGGGCCUCAAGAUUAGGCAGCU | |
| | CCUAUGGUUCCAUAUUUCCUGCUUAACUUUCGGUAGGGA | |
| | AACUGUCCUGGAAUAUCUCGUUAGCUUCGGAGUGUGGAU | |
| | UCGGACUCCUCCUGCCUACAGACCACCUAACGCUCCAAUU | |
| | CUGUCAACUCUCCCAGAGACGACUGUGGUCAGAAGGAGG | |
| | GGCAGGAGCCCCAGACGGCGGACACCAAGCCCUCGCCGAA | |
| | GAAGAAGCCAAAGCCCGCGGCGCCGGAGAUCUCAGUCCA | |
| | GAGAGAGCCAGUGC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDSKGSSQKGSRLLLLLVVSNLLLPQGVVDIDPYKEFGATVE | 92 |
| amino acid | LLSFLPSDFFPSVRDLLDTAAALYREALESPEHCSPHHTALRQ | |
| sequence | AILCWGELMTLATWVGNNLEDPASRDLVVNYVNTNMGLKIR | |
| | QLLWFHISCLTFGRETVLEYLVSFGVWIRTPPAYRPPNAPILST | |
| | LPETTVVGSGSAGSASGGASSGSGGASDIDPYKEFGATVELLS | |
| | FLPSDFFPSVRDLLDTAAALYREALESPEHCSPHHTALRQAILC | |
| | WGELMTLATWVGNNGSGEAALLVCQYTIQSLIHLTGEDPGFF | |
| | NVEIPEFPFYPTCNVCTADVNVTINFDVGGKKHQLDLDFGQLT | |
| | PHTKAVYQPRGAFGGSENATNLFLLELLGAGELALTMRSKKL | |
| | PINVTTGEEQQVSLESVDVYFQDVFGTMWCHHAEMQNPVYLI | |
| | PETVPYIKWDNCNSTNITAVVRAQGLDVTLPLSLPTSAQDSNF | |
| | SVKTEMLGNEIDIECIMEDGEISQVLPGDNKFNITCSGYESHVP | |
| | SGGILTSTSPVATPIPGTGYAYSLRLTPRPVSRFLGNNSILYVFY | |
| | SGNGPKASGGDYCIQSNIVFSDEIPASQDMPTNTTDITYVGDN | |
| | ATYSVPMVTSEDANSPNVTVTAFWAWPNNTETDFKCKWTLT | |
| | SGTPSGCENISGAFASNRTFDITVSGLGTAPKTLIITRTATNATT | |
| | TTHKVIFSKAPGSGLVVNYVNTNMGLKIRQLLWFHISCLTFGR | |
| | ETVLEYLVSFGVWIRTPPAYRPPNAPILSTLPETTVVRRRGRSP | |
| | RRRTPSPRRRRSQSPRRRRSQSRESQC | |
| PolyA tail | 100 nt | |

EBV gp350 Ecto_RX

| SEQ ID NO: 193 consists of from 5′ end to 3′ end: | | 193 |
|---|---|---|
| 5′ UTR SEQ ID NO: 1, mRNA ORF SEQ ID NO: 93, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORFofmRNA | AUGGACAGCAAGGGCAGCAGCCAGAAGGGUAGCAGGCUA | 93 |
| Construct | CUCUUGCUCCUAGUGGUGAGCAACUUGCUUCUUCCCCAG | |
| (excluding | GGCGUGGUGGAGGCAGCAUUGCUUGUCUGCCAGUAUACA | |
| the stop | AUCCAGUCGCUCAUUCAUCUAACAGGCGAGGACCCCGGC | |
| codon) | UUCUUUAACGUGGAAAUCCCAGAGUUCCCUUUCUAUCCA | |
| | ACCUGUAACGUUUGCACUGCCGACGUGAACGUGACAAUC | |
| | AAUUUCGACGUGGGAGGAAAGAAGCAUCAGCUUGAUCUG | |
| | GAUUUCGGCCAACUGACGCCACACACCAAAGCGGUGUAC | |
| | CAACCAAGGGGUGCAUUUGGAGGGAGCGAGAAUGCUACA | |
| | AAUCUGUUCUUACUGGAACUGCUUGGUGCAGGAGAGCUG | |
| | GCACUAACCAUGCGAAGCAAGAAGCUCCCCAUCAACGUU | |
| | ACCACCGGGGAAGAACAGCAAGUCAGCCUUGAAUCCGUG | |
| | GAUGUGUACUUUCAGGAUGUUUUCGGCACCAUGUGGUGU | |
| | CACCACGCUGAGAUGCAGAAUCCCGUGUAUCUCAUACCC | |
| | GAGACUGUACCCUACAUCAAGUGGGAUAAUUGCAAUUCA | |
| | ACUAAUAUUACGGCUGUGGUGAGGGCCCAGGGCUUGGAC | |
| | GUGACUCUGCCCUUAUCUCUACCUACUUCUGCCCAAGACU | |
| | CCAAUUUCUCUGUCAAGACCGAGAUGCUCGGGAAUGAGA | |
| | UCGAUAUCGAGUGCAUCAUGGAGGACGGUGAGAUAAGCC | |
| | AGGUUCUGCCCGGCGACAACAAGUUCAAUAUCACUUGUU | |
| | CUGGCUACGAGUCCCAUGUGCCUUCCGGCGGCAUACUCAC | |
| | AAGUACUUCUCCCGUAGCCACGCCCAUUCCCGGAACCGGA | |
| | UACGCCUACAGUCUGCGUCUGACCCCACGGCCUGUGUCCA | |
| | GAUUCCUGGGUAACAAUAGUAUCUUAUACGUGUUUUAUA | |
| | GCGGAAACGGCCCUAAAGCGUCCGGAGGGGACUAUUGUA | |
| | UUCAGAGUAAUAUCGUUUUCUCUGAUGAGAUUCCAGCCA | |
| | GUCAGGACAUGCCGACAAACACAACUGAUAUUACCUACG | |
| | UGGGCGACAAUGCCACGUAUUCAGUGCCCAUGGUCACGA | |
| | GCGAGGACGCCAAUUCACCAAAUGUUACUGUAACAGCUU | |
| | UCUGGGCCUGGCCAAAUAACACUGAGACUGACUUCAAAU | |
| | GUAAGUGGACUUUGACCUCUGGAACUCCGUCGGGUUGCG | |
| | AGAAUAUCAGCGGGGCCUUUGCUUCCAACAGGACUUUCG | |
| | ACAUCACUGUCUCAGGGCUGGGGACAGCACCGAAGACAU | |
| | UAAUCAUAACACGGACCGCCACCAACGCCACGACUACAAC | |
| | CCAUAAGGUGAUCUUUUCCAAGGCACCUGAGUCCACCAC | |
| | UACCUCCCCGACUCUUAACACUACGGGCUUCGCUGAUCCC | |
| | AAUACUACUACUGGGUUGCCUAGCUCGACACAUGUGCCG | |
| | ACGAACCUGACUGCCCCUGCAUCGACCGGGCCCACAGUUU | |
| | CGACCGCCGAUGUGACAAGCCCUACGCCCGCAGGUACAAC | |
| | CUCAGGCGCCAGCCCAGUGACCCCUUCCCCAAGCCCCUGG | |
| | GACAACGGAACGGAGUCCAAGGCUCCUGAUAUGACUUCC | |
| | UCUACCAGCCCCGUGACUACACCCACUCCCAACGCAACUA | |
| | GCCCAACCCCAGCUGUGACGACGCCCACCCCGAACGCGAC | |
| | AUCUCCCACACCUGCUGUGACAACCCCUACCCCUAACGCC | |
| | ACUAGCCCUACCCUAGGUAAGACCAGUCCGACUAGCGCCG | |
| | UUACUACACCGACCCCUAAUGCAACCGGCCCGACUGUCGG | |
| | CGAGACUUCCCCGCAAGCCAAUGCGACAAAUCACACAUU | |
| | GGGCGGGACCUCUCCUACACCAGUCGUUACAUCUCAGCCU | |
| | AAGAACGCUACCUCCGCUGUCACUACCGGCCAGCACAACA | |
| | UCACAAGCUCAAGCACGAGUUCCAUGAGCUUGCGGCCGA | |
| | GCUCAAAUCCCGAGACACUAUCACCAUCCACAUCAGACAA | |
| | CAGUACUUCACACAUGCCACUCUUGACGAGCGCUCACCCC | |
| | ACCGGCGGCGAGAACAUUACCCAGGUGACUCCGGCGUCU | |
| | AUUUCCACCCACCACGUCAGCACGUCGUCUCCCGCACCAC | |
| | GGCCAGGGACGACUUCUCAGGCCAGCGGCCCAGGAAACU | |
| | CCUCUACAAGCACAAAGCCAGGCGAAGUUAACGUCACCA | |
| | AGGGAACGCCUCCCCAGAACGCAACCUCCCCACAGGCUCC | |
| | CAGCGGGCAGAAGACUGCGGUGCCAACUGUGACCAGCAC | |
| | CGGUGGCAAGGCCAACUCAACAACUGGAGGCAAGCAUAC | |
| | GACGGGGCACGGCGCCCGGACCUCCACUGAACCCACGACC | |
| | GAUUACGGAGGUGAUAGUACCACCCCGCGGCCACGAUAU | |
| | AAUGCCACCACUUAUCUGCCACCUAGCACUUCCUCCAAGC | |
| | UCCGGCCACGGUGGACCUUCACAAGCCCACCCGUGACGAC | |
| | AGCCCAGGCGACGGUGCCAGUCCCACCUACAAGCCAGCCA | |
| | CGCUUCUCCAACCUUAGU | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDSKGSSQKGSRLLLLLVVSNLLLPQGVVEAALLVCQYTIQS | 94 |
| amino acid | LIHLTGEDPGFFNVEIPEFPFYPTCNVCTADVNVTINFDVGGKK | |
| sequence | HQLDLDFGQLTPHTKAVYQPRGAFGGSENATNLFLLELLGAG | |
| | ELALTMRSKKLPINVTTGEEQQVSLESVDVYFQDVFGTMWCH | |
| | HAEMQNPVYLIPETVPYIKWDNCNSTNITAVVRAQGLDVTLP | |
| | LSLPTSAQDSNFSVKTEMLGNEIDIECIMEDGEISQVLPGDNKF | |
| | NITCSGYESHVPSGGILTSTSPVATPIPGTGYAYSLRLTPRPVSR | |
| | FLGNNSILYVFYSGNGPKASGGDYCIQSNIVFSDEIPASQDMPT | |
| | NTTDITYVGDNATYSVPMVTSEDANSPNVTVTAFWAWPNNT | |
| | ETDFKCKWTLTSGTPSGCENISGAFASNRTFDITVSGLGTAPKT | |
| | LIITRTATNATTTTHKVIFSKAPESTTTSPTLNTTGFADPNTTTG | |
| | LPSSTHVPTNLTAPASTGPTVSTADVTSPTPAGTTSGASPVTPS | |
| | PSPWDNGTESKAPDMTSSTSPVTTPTPNATSPTPAVTTPTPNAT | |
| | SPTPAVTTPTPNATSPTLGKTSPTSAVTTPTPNATGPTVGETSP | |
| | QANATNHTLGGTSPTPVVTSQPKNATSAVTTGQHNITSSSTSS | |
| | MSLRPSSNPETLSPSTSDNSTSHMPLLTSAHPTGGENITQVTPA | |
| | SISTHHVSTSSPAPRPGTTSQASGPGNSSTSTKPGEVNVTKGTP | |
| | PQNATSPQAPSGQKTAVPTVTSTGGKANSTTGGKHTTGHGAR | |
| | TSTEPTTDYGGDSTTPRPRYNATTYLPPSTSSKLRPRWTFTSPP | |
| | VTTAQATVPVPPTSQPRFSNLS | |
| PolyA tail | 100 nt | |

EBV gL-gH_LS_RX

| SEQ ID NO: 194 consists of from 5′ end to 3′ end: | | 194 |
|---|---|---|
| 5′ UTR SEQ ID NO: 1, mRNA ORF SEQ ID NO: 95, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGACUCGAAGGGAUCUAGUCAGAAAGGCUCUAGGCUC | 95 |
| Construct | CUACUCCUCUUGGUGGUGAGCAACUUACUCCUUCCCCAG | |
| (excluding | GGCGUGGUGAGAGCAGUGGGCGUUUUCUUAGCAAUCUGC | |
| the stop | CUCGUGACAAUCUUUGUGUUGCCGACCUGGGGCAACUGG | |
| codon) | GCCUACCCCUGCUGCCACGUGACCCAGCUGAGAGCUCAAC | |
| | ACCUGCUUGCCCUGGAGAACAUCAGCGACAUCUACCUUG | |
| | UAAGCAACCAGACAUGCGACGGCUUCAGUCUGGCCAGCC | |
| | UGAACAGCCCCAAGAACGGCUCAAACCAGCUAGUGAUCA | |
| | GCCGGUGCGCUAACGGUCUGAACGUUGUGAGCUUCUUCA | |
| | UCAGCAUCUUGAAGAGAAGUAGUAGCGCGCUGACCGGCC | |
| | ACCUGAGAGAGCUGCUGACCACUCUCGAGACACUCUAUG | |
| | GCAGCUUCAGCGUGGAGGACCUGUUCGGCGCAAACCUGA | |
| | ACCGCUACGCCUGGCACAGAGGCGGCGGAAGCGGUAGCG | |
| | CUGGCAGCGCCAGCGGAGGCGCUUCAUCCGGAUCCGGCG | |
| | GCGCAAGCAGCCUUUCUGAGGUGAAGCUCCACUUGGACA | |
| | UCGAAGGCCACGCCAGCCAUUACACCAUACCAUGGACCGA | |
| | GCUGAUGGCAAAGGUGCCCGGCCUUAGCCCAGAAGCCCU | |
| | GUGGCGAGAGGCCAACGUUACCGAAGACUUGGCUAGUAU | |
| | GUUAAACAGGUACAAACUUAUCUACAAGACUAGCGGCAC | |
| | CCUGGGCAUCGCAUUAGCCGAGCCCGUGGACAUCCCCGCC | |
| | GUUAGCGAGGGCAGCAUGCAGGUGGACGCCAGCAAGGUU | |
| | CACCCUGGUGUAAUUAGUGGCCUCAACAGCCCAGCCUGU | |
| | AUGUUGAGCGCCCCACUGGAGAAGCAGCUGUUUUACUAU | |
| | AUCGGGACCAUGUUGCCCAAUACCCGGCCACACAGUUAC | |
| | GUGUUCUACCAGCUGCGGUGUCACCUGAGCUACGUGGCC | |
| | CUGUCUAUCAACGGCGACAAGUUCCAGUACACCGGCGCC | |
| | AUGACCAGCAAGUUCCUGAUGGGCACCUACAAGCGGGUC | |
| | ACCGAGAAGGGCGACGAGCACGUGCUGUCACUUGUGUUC | |
| | GGUAAGACCAAGGACCUGCCCGACCUUCGGGGCCCCUUU | |
| | UCAUACCCUAGUUUGACCAGCGCCCAAAGUGGCGACUAC | |
| | AGCCUUGUGAUAGUGACCACCUUCGUGCAUUACGCCAAC | |
| | UUCCACAACUACUUCGUGCCCAACCUGAAGGACAUGUUC | |
| | AGCCGGGCGGUAACCAUGACGGCCGCUUCUUAUGCCCGG | |
| | UAUGUGCUGCAGAAGCUGGUCCUGCUGGAGAUGAAGGGC | |
| | GGGUGCCGGGAGCCCGAGUUGGACACCGAAACCCUGACC | |
| | ACCAUGUUCGAGGUUAGCGUGGCCUUCUUCAAGGUGGGU | |
| | CACGCGGUGGGCGAAACCGGCAAUGGAUGCGUGGACUUA | |
| | CGCUGGCUGGCCAAGAGUUUCUUUGAGCUUACCGUGCUG | |
| | AAGGAUAUCAUCGGCAUCUGCUACGGCGCCACCGUAAAG | |
| | GGAAUGCAGAGCUAUGGGCUGGAGAGACUUGCCGCCAUG | |
| | CUCAUGGCUACCGUGAAGAUGGAGGAGCUGGGACACCUG | |
| | ACAACAGAGAAGCAGGAGUACGCCCUGAGACUGGCCACC | |
| | GUGGGCUACCCUAAGGCCGGCGUGUACAGUGGACUCAUA | |
| | GGCGGAGCUACCAGCGUGCUCCUAUCCGCUUAUAACAGA | |
| | CACCCCUUAUUUCAGCCCCUGCACACAGUGAUGCGGGAG | |
| | ACUUUAUUCAUCGGAUCUCACGUCGUACUGCGGGAGUUG | |
| | CGGCUGAACGUAACCACCCAGGGCCCUAAUUUAGCACUU | |
| | UAUCAGCUGCUCAGUACUGCCCUGUGCAGCGCACUUGAG | |
| | AUCGGCGAGGUACUCAGAGGCCUGGCCCUCGGUACGGAG | |
| | AGUGGCCUGUUUUCUCCAUGCUACCUUAGCCUGCGGUUU | |
| | GAUCUGACCAGAGACAAGUUGCUGAGCAUGGCUCCUCAA | |
| | GAGGCGACCCUUGACCAGGCAGCCGUGUCAAACGCCGUG | |
| | GACGGAUUCCUGGGCAGACUGUCUCUGGAACGGGAAGAC | |
| | CGGGAUGCUUGGCACCUUCCUGCCUAUAAGUGUGUGGAU | |
| | CGGCUGGAUAAGGUCCUGAUGAUAAUCCCUCUGAUUAAC | |
| | GUUACUUUCAUCAUCUCUAGCGAUCGGGAGGUCCGAGGG | |
| | UCCGCCCUCUACGAGGCCAGCACCACCUAUCUGAGCAGCA | |
| | GCCUCUUCUUGUCUCCUGUGAUUAUGAACAAGUGCAGCC | |
| | AGGGUGCAGUAGCCGGCGAGCCCCGGCAGAUCCCGAAGA | |
| | GCGGCUUCGCCCUUCUGAGUUACGACGAGAAGGAGGGCC | |
| | UGGAGACUACAACUUACAUCACAAGCCAGGAGGUGCAGA | |
| | ACAGCAUCCUGUCAUCUAAUUACUUUGACUUCGACAACC | |
| | UGCACGUUCAUUAUCUACUGCUAACAACUAACGGUACCG | |
| | UCAUGGAAAUCGCAGGACUGUAUGAGGAGCGGGCCGGCA | |
| | GCGGGAGCGCGGGCUCUGCCAGCGGUGGUGCCAGCAGCG | |
| | GCAGUGGAGGUGCCUCCAUGCAGAUUUACGAAGGAAAGC | |
| | UGACCGCCGAAGGCCUGAGGUUCGGCAUCGUGGCUUCCA | |
| | GAUUCAACCACGCACUGGUGGACAGGCUGGUGGAGGGCG | |
| | CCAUCGACGCAAUCGUGCGCCACGGCGGCAGGGAGGAGG | |
| | ACAUCACCCUCGUAAGAGUCCCUGGUAGCUGGGAAAUUC | |
| | CCGUGGCCGCGGGCGAGCUGGCUAGGAAGGAGGAUAUCG | |
| | ACGCCGUCAUCGCCAUCGGCGUUCUCAUCCGGGGUGCCAC | |
| | ACCACAUUUCGACUACAUCGCAAGCGAGGUUUCAAAGGG | |
| | ACUCGCCGACUUAAGUCUGGAGCUGAGGAAGCCCAUCAC | |
| | CUUUGGCGUGAUCACCGCCGACACCCUGGAGCAGGCCAUC | |
| | GAGCGCGCCGGCACCAAGCACGGGAACAAGGGCUGGGAG | |
| | GCAGCCUUAUCAGCCAUCGAAAUGGCGAAUCUGUUCAAG | |
| | AGCCUGCGG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDSKGSSQKGSRLLLLLVVSNLLLPQGVVRAVGVFLAICLVTI | 96 |
| amino acid | FVLPTWGNWAYPCCHVTQLRAQHLLALENISDIYLVSNQTCD | |
| sequence | GFSLASLNSPKNGSNQLVISRCANGLNVVSFFISILKRSSSALTG | |
| | HLRELLTTLETLYGSFSVEDLFGANLNRYAWHRGGGSGSAGS | |
| | ASGGASSGSGGASSLSEVKLHLDIEGHASHYTIPWTELMAKVP | |
| | GLSPEALWREANVTEDLASMLNRYKLIYKTSGTLGIALAEPV | |
| | DIPAVSEGSMQVDASKVHPGVISGLNSPACMLSAPLEKQLFYY | |
| | IGTMLPNTRPHSYVFYQLRCHLSYVALSINGDKFQYTGAMTS | |
| | KFLMGTYKRVTEKGDEHVLSLVFGKTKDLPDLRGPFSYPSLT | |
| | SAQSGDYSLVIVTTFVHYANFHNYFVPNLKDMFSRAVTMTAA | |
| | SYARYVLQKLVLLEMKGGCREPELDTETLTTMFEVSVAFFKV | |
| | GHAVGETGNGCVDLRWLAKSFFELTVLKDIIGICYGATVKGM | |
| | QSYGLERLAAMLMATVKMEELGHLTTEKQEYALRLATVGYP | |
| | KAGVYSGLIGGATSVLLSAYNRHPLFQPLHTVMRETLFIGSHV | |
| | VLRELRLNVTTQGPNLALYQLLSTALCSALEIGEVLRGLALGT | |
| | ESGLFSPCYLSLRFDLTRDKLLSMAPQEATLDQAAVSNAVDG | |
| | FLGRLSLEREDRDAWHLPAYKCVDRLDKVLMIIPLINVTFIISS | |
| | DREVRGSALYEASTTYLSSSLFLSPVIMNKCSQGAVAGEPRQI | |
| | PKIQNFTRTQKSCIFCGFALLSYDEKEGLETTTYITSQEVQNSIL | |
| | SSNYFDFDNLHVHYLLLTTNGTVMEIAGLYEERAGSGSAGSA | |
| | SGGASSGSGGASMQIYEGKLTAEGLRFGIVASRFNHALVDRL | |
| | VEGAIDAIVRHGGREEDITLVRVPGSWEIPVAAGELARKEDID | |
| | AVIAIGVLIRGATPHFDYIASEVSKGLADLSLELRKPITFGVITA | |
| | DTLEQAIERAGTKHGNKGWEAALSAIEMANLFKSLR | |
| PolyA tail | 100 nt | |

EBV gBEcto_LS_RX

| SEQ ID NO: 195 consists of from 5′ end to 3′ end: | | 195 |
|---|---|---|
| 5′ UTR SEQ ID NO: 1, mRNA ORF SEQ ID NO: 97, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGACUCUAAGGGCAGCAGCCAGAAGGGCAGUAGGCUC | 97 |
| Construct | CUACUUCUCCUCGUGGUGAGCAACUUAUUGCUUCCCCAG | |
| (excluding | GGAGUGGUGCAGACUCCGGAACAGCCCGCUCCGCCCGCUA | |
| the stop | CCACCGUCCAGCCUACCGCCACGCGCCAGCAGACUUCCUU | |
| codon) | CCCGUUUCGCGUCUGCGAGCUCUCCUCUCACGGCGAUCUC | |
| | UUCCGCUUCUCCUCCGAUAUUCAGUGCCCCUCCUUCGGCA | |
| | CCCGCGAGAACCAUACCGAGGGCCUCCUCAUGGUCUUUA | |
| | AGGACAACAUCAUCCCUUACUCCUUCAAGGUACGCUCCU | |
| | ACACAAAGAUAGUCACCAACAUCCUCAUCUAUAAUGGAU | |
| | GGUACGCUGACUCUGUUACCAAUCGCCACGAGGAGAAGU | |
| | UCUCCGUUGAUUCCUACGAGACUGACCAGAUGGAUACCA | |
| | UCUACCAGUGCUACAAUGCCGUCAAGAUGACCAAGGACG | |
| | GCCUGACCCGCGUCUAUGUCGACCGCGACGGCGUUAACA | |
| | UAACUGUCAACCUCAAGCCUACAGGCGGCCUGGCCAAUG | |
| | GCGUCAGACGCUACGCCUCCCAGACCGAGCUCUACGACGC | |
| | UCCUGGCUGGCUGAUCUGGACCUACCGGACCCGCACCACU | |
| | GUCAAUUGCCUCAUUACCGACAUGAUGGCCAAGUCCAAU | |
| | AGCCCAUUCGAUUUCUUCGUAACCACCACCGGCCAGACCG | |
| | UCGAGAUGUCGCCAUUCUAUGACGGCAAGAAUAAGGAAA | |
| | CUUUCCACGAGAGAGCCGAUAGCUUCCACGUCCGCACGA | |
| | ACUACAAGAUUGUCGACUACGACAAUCGCGGCACCAAUC | |
| | CACAGGGCGAGCGCCGCGCCUUCCUGGACAAGGGCACCUA | |
| | CACCCUCUCCUGGAAACUCGAGAACAGAACCGCCUACUGU | |
| | CCCCUCCAGCACUGGCAGACUUUUGAUUCCACCAUCGCCA | |
| | CUGAGACUGGCAAGAGUAUCCAUUUCGUCACCGACGAAG | |
| | GAACAUCUUCAUUCGUCACAAAUACCACUGUGGGCAUUG | |
| | AGCUCCCCGACGCCUUCAAGUGCAUCGAGGAACAGGUCA | |
| | ACAAGACAAUGCACGAGAAGUACGAGGCCGUGCAGGACC | |
| | GGUAUACCAAGGGCCAGGAGGCCAUUACAUACUUCAUCA | |
| | CCAGCGGUGGAUUGCUUCUCGCCUGGCUUCCACUCACACC | |
| | UCGCAGUCUGGCCACAGUUAAGAACCUCACUGAGUUGAC | |
| | CACUCCAACAUCGUCACCACCUUCUUCACCUAGCCCUCCU | |
| | GCUCCUUCCGCCGCCCGCGGCUCCACUCCUGCCGCUGUCU | |
| | UAAGGGGCUCGGGCAGCGCCGGCAGCGCAUCGGGAGGGG | |
| | CGAGUUCAGGGUCAGGCGGUGCCUCCGCAGGCAAUGCUA | |
| | CCACACCAGUGCCACCAACCGCCCCUGGCAAGUCCUUAGG | |
| | UACCCUGAACAAUCCAGCGACAGUUCAGAUCCAGUUCGC | |
| | UUACGAUUCACUCAGGCGCCAGAUCAACCGCAUGCUUGG | |
| | UGACCUUGCCCGGGCCUGGUGCCUCGAACAGAAGCGCCA | |
| | GAACAUGGUACUUAGGGAACUUACCAAGAUUAAUCCUAC | |
| | UACAGUCAUGUCCUCUAUCUACGGCAAGGCCGUUGCUGC | |
| | CAAGCGCCUGGGCGACGUCAUCAGCGUGUCGCAGUGCGU | |
| | CCCAGUCAACCAGGCUACCGUUACCCUCAGGAAGUCCAUG | |
| | AGAGUGCCAGGCUCCGAAACAAUGUGCUAUUCCCGCCCU | |
| | CUGGUUUCAUUUUCCUUCAUCAACGACACAAAGACCUAC | |
| | GAGGGCCAGUUAGGAACUGACAAUGAGAUAUUCUUGACU | |
| | AAGAAGAUGACUGAGGUAUGCCAGGCAACUUCUCAAUAC | |
| | UACUUCCAGUCUGGAAAUGAAAUCCACGUAUAUAACGAC | |
| | UAUCAUCACUUUAAGACUAUCGAACUCGACGGAAUUGCU | |
| | ACACUCCAAACAUUCAUCUCACUGAAUACCUCCCUUAUCG | |
| | AGAACAUCGACUUCGCAUCCCUCGAGCUGUAUAGCAGAG | |
| | ACGAGCAGAGAGCCAGCAACGUGUUCGACCUGGAGGGUA | |
| | UCUUUCGCGAGUACAACUUCCAGGCACAGAAUAUAGCCG | |
| | GCCUCCGUAAGGACCUGGAUAACGCCGUGUCCAAUGGCC | |
| | GCAACCAGUUCGUCGACGGUCUUGGCGAACUCAUGGACU | |
| | CUCUGGGAUCCGUGGGGCAAUCCAUCACAAACUUAGUCU | |
| | CUACUGUCGGUGGACUAUUCAGCAGCCUGGUUAGCGGAU | |
| | UUAUCUCUUUCUUUAAGAACCCAGGCAGUGGUUCUGCCG | |
| | GCUCAGCCAGCGGCGGCGCUAGUAGCGGCAGCGGAGGCG | |
| | CCUCAAUGCAGAUCUAUGAGGGUAAGUUGACAGCCGAAG | |
| | GCUUGAGGUUUGGCAUCGUAGCCAGCCGGUUCAACCACG | |
| | CCCUGGUGGACAGGCUGGUUGAGGGUGCCAUUGACGCCA | |
| | UUGUGAGGCACGGCGGCAGGGAGGAGGACAUUACCCUGG | |
| | UCAGAGUCCCCGGAUCAUGGGAGAUCCCCGUGGCCGCCG | |
| | GGGAGCUGGCCAGGAAGGAAGACAUCGACGCUGUGAUCG | |
| | CCAUCGGCGUGCUGAUCCGAGGAGCCACACCCCAUUUCGA | |
| | CUACAUCGCCAGUGAGGUUAGCAAAGGCCUAGCGGACCU | |
| | GAGCCUGGAGCUGCGCAAGCCCAUCACCUUCGGCGUCAU | |
| | UACUGCCGACACCUUGGAACAGGCCAUCGAAAGGGCCGG | |
| | UACAAAGCACGGCAACAAGGGCUGGGAGGCAGCCCUUAG | |
| | CGCCAUCGAGAUGGCAAACCUGUUUAAGUCCUUGAGG | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDSKGSSQKGSRLLLLLVVSNLLLPQGVVQTPEQPAPPATTV | 98 |
| amino acid | QPTATRQQTSFPFRVCELSSHGDLFRFSSDIQCPSFGTRENHTE | |
| sequence | GLLMVFKDNIIPYSFKVRSYTKIVTNILIYNGWYADSVTNRHE | |
| | EKFSVDSYETDQMDTIYQCYNAVKMTKDGLTRVYVDRDGV | |
| | NITVNLKPTGGLANGVRRYASQTELYDAPGWLIWTYRTRTTV | |
| | NCLITDMMAKSNSPFDFFVTTTGQTVEMSPFYDGKNKETFHE | |
| | RADSFHVRTNYKIVDYDNRGTNPQGERRAFLDKGTYTLSWK | |
| | LENRTAYCPLQHWQTFDSTIATETGKSIHFVTDEGTSSFVTNT | |
| | TVGIELPDAFKCIEEQVNKTMHEKYEAVQDRYTKGQEAITYFI | |
| | TSGGLLLAWLPLTPRSLATVKNLTELTTPTSSPPSSPSPPAPSAA | |
| | RGSTPAAVLRGSGSAGSASGGASSGSGGASAGNATTPVPPTAP | |
| | GKSLGTLNNPATVQIQFAYDSLRRQINRMLGDLARAWCLEQK | |
| | RQNMVLRELTKINPTTVMSSIYGKAVAAKRLGDVISVSQCVP | |
| | VNQATVTLRKSMRVPGSETMCYSRPLVSFSFINDTKTYEGQL | |
| | GTDNEIFLTKKMTEVCQATSQYYFQSGNEIHVYNDYHHFKTIE | |
| | LDGIATLQTFISLNTSLIENIDFASLELYSRDEQRASNVFDLEGI | |
| | FREYNFQAQNIAGLRKDLDNAVSNGRNQFVDGLGELMDSLG | |
| | SVGQSITNLVSTVGGLFSSLVSGFISFFKNPGSGSAGSASGGAS | |
| | SGSGGASMQIYEGKLTAEGLRFGIVASRFNHALVDRLVEGAID | |
| | AIVRHGGREEDITLVRVPGSWEIPVAAGELARKEDIDAVIAIGV | |
| | LIRGATPHFDYIASEVSKGLADLSLELRKPITFGVITADTLEQAI | |
| | ERAGTKHGNKGWEAALSAIEMANLFKSLR | |
| PolyA tail | 100 nt | |

EBV gp350 A44Y V10 Stab

| SEQ ID NO: 196 consists of from 5′ end to 3′ end: | | 196 |
|---|---|---|
| 5′ UTR SEQ ID NO: 1, mRNA ORF SEQ ID NO: 99, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 99 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUUACGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTY | 45 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gp350 V15 Stab4_NGM6

| SEQ ID NO: 197 consists of from 5′ end to 3′ end: | | 197 |
|---|---|---|
| 5′ UTR SEQ ID NO: 1, mRNA ORF SEQ ID NO: 100, and | | |
| 3′ UTR SEQ ID NO: 3 | | |
| Chemistry | 1-methylpseudouridine | |
| Cap | 7mG(5′)ppp(5′)NlmpNp | |
| 5′UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 100 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUUACGACGUGAACGUGACAUUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUGCGAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAGCGAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | GCGGUCAAGACCGAGAUACUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCGCGUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUGCGGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCGCGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCUGG | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTY | 55 |
| amino acid | DVNVTFNFDVGGKKHQLDLDFGQLTPHTKAVYQPRGAFGGS | |
| sequence | ENAANLFLLELLGAGELALTMRSKKLPINVTTGEEQQVSLESV | |
| | DVYFQDVFGTMWCHHAEMQNPVYLIPETVPYIKWDNCNSAN | |
| | ITAVVRAQGLDVTLPLSLPTSAQDSNFAVKTEILGNEIDIECIM | |
| | EDGEISQVLPGDNKFNIACSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVAVTAFWAWPNNTETDFKCKWTLTSGTPSGCENISGAFASN | |
| | RTFDITVSGLGTAPKTLIITRTATNAATTTHKVIFSKAPESTTTS | |
| | PTLNTTGFADPNTTTGLPSSTHVPTNLTAPASTGPTVSTWDVT | |
| | SPTPAGTTSGASPVTPSPSPWDNGTESKAPDMTSSTSPVTTPTP | |
| | NATSPTPAVTTPTPNATSPTPAVTTPTPNATSPTLGKTSPTSAV | |
| | TTPTPNATGPTVGETSPQANATNHTLGGTSPTPVVTSQPKNAT | |
| | SAVTTGQHNITSSSTSSMSLRPSSNPETLSPSTSDNSTSHMPLLT | |
| | SAHPTGGENITQVTPASISTHHVSTSSPAPRPGTTSQASGPGNS | |
| | STSTKPGEVNVTKGTPPQNATSPQAPSGQKTAVPTVTSTGGK | |
| | ANSTTGGKHTTGHGARTSTEPTTDYGGDSTTPRPRYNATTYL | |
| | PPSTSSKLRPRWTFTSPPVTTAQATVPVPPTSQPRFSNLSMLVL | |
| | QWASLAVLTLLLLLVMADCAFRRNLSTSHTYTTPPYDDAETY | |
| | V | |
| PolyA tail | 100 nt | |

V6_NGM N195_EBV_gp350_001

| SEQ ID NO: 198 consists of from 5′ end to 3′ end: | | 198 |
|---|---|---|
| 5′ UTR SEQ ID NO: 1, mRNA ORF SEQ ID NO: 101, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 101 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | GCGGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 65 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFAVKTEMLGNEIDIECI | |
| | MEDGEISQVLPGDNKFNITCSGYESHVPSGGILTSTSPVATPIPG | |
| | TGYAYSLRLTPRPVSRFLGNNSILYVFYSGNGPKASGGDYCIQ | |
| | SNIVFSDEIPASQDMPTNTTDITYVGDNATYSVPMVTSEDANS | |
| | PNVTVTAFWAWPNNTETDFKCKWTLTSGTPSGCENISGAFAS | |
| | NRTFDITVSGLGTAPKTLIITRTATNATTTTHKVIFSKAPESTTT | |
| | SPTLNTTGFADPNTTTGLPSSTHVPTNLTAPASTGPTVSTADVT | |
| | SPTPAGTTSGASPVTPSPSPWDNGTESKAPDMTSSTSPVTTPTP | |
| | NATSPTPAVTTPTPNATSPTPAVTTPTPNATSPTLGKTSPTSAV | |
| | TTPTPNATGPTVGETSPQANATNHTLGGTSPTPVVTSQPKNAT | |
| | SAVTTGQHNITSSSTSSMSLRPSSNPETLSPSTSDNSTSHMPLLT | |
| | SAHPTGGENITQVTPASISTHHVSTSSPAPRPGTTSQASGPGNS | |
| | STSTKPGEVNVTKGTPPQNATSPQAPSGQKTAVPTVTSTGGK | |
| | ANSTTGGKHTTGHGARTSTEPTTDYGGDSTTPRPRYNATTYL | |
| | PPSTSSKLRPRWTFTSPPVTTAQATVPVPPTSQPRFSNLSMLVL | |
| | QWASLAVLTLLLLLVMADCAFRRNLSTSHTYTTPPYDDAETY | |
| | V | |
| PolyA tail | 100 nt | |

V8_NGM N345_EBV_gp350_001

| SEQ ID NO: 199 consists of from 5′ end to 3′ end: | | 199 |
|---|---|---|
| 5′ UTR SEQ ID NO: 1, mRNA ORF SEQ ID NO: 102, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 102 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUGCGGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 114 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVAVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

V9_NGM N411_EBV_gp350_001

| SEQ ID NO: 200 consists of from 5′ end to 3′ end: | | 200 |
|---|---|---|
| 5′ UTR SEQ ID NO: 1, mRNA ORF SEQ ID NO: 103, and | | |
| 3′ UTR SEQ ID NO: 3 | | |
| Chemistry | 1-methylpseudouridine | |
| Cap | 7mG(5′)ppp(5′)NlmpNp | |
| 5′UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 103 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCGCGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 70 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNAATTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gH (BXLF2)

| SEQ ID NO: 201 consists of from 5′ end to 3′ end: | | 201 |
|---|---|---|
| 5′ UTR SEQ ID NO: 104, mRNA ORF SEQ ID NO: 106, and | | |
| 3′ UTR SEQ ID NO: 106 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGCAGUUGUUGUGCGUGUUCUGCCUCGUGUUACUCUGG | 105 |
| Construct | GAGGUGGGCGCCGCCAGCCUUAGCGAGGUGAAGCUCCAC | |
| (excluding | UUGGACAUCGAGGGCCACGCCAGCCACUACACCAUCCCCU | |
| the stop | GGACCGAGCUCAUGGCCAAGGUGCCCGGCCUUAGCCCCGA | |
| codon) | GGCCCUGUGGCGGGAGGCCAACGUGACCGAGGACCUGGC | |
| | CAGCAUGCUGAACCGGUACAAGCUGAUCUACAAGACCAG | |
| | CGGCACCCUGGGCAUCGCCCUGGCCGAGCCCGUGGACAUC | |
| | CCCGCCGUUAGCGAAGGCAGCAUGCAGGUGGACGCCAGC | |
| | AAGGUGCACCCCGGCGUGAUCAGCGGCCUGAACAGCCCCG | |
| | CCUGUAUGUUGAGCGCCCCACUGGAGAAGCAGCUGUUCU | |
| | ACUACAUCGGCACCAUGCUGCCCAACACCCGGCCCCACAG | |
| | CUACGUGUUCUACCAGCUGCGGUGCCACCUGAGCUACGU | |
| | UGCCCUGAGCAUCAACGGCGACAAGUUCCAGUACACCGG | |
| | CGCCAUGACCAGCAAGUUCCUGAUGGGCACCUACAAGCG | |
| | GGUCACCGAGAAGGGCGACGAGCACGUGCUGUCACUGGU | |
| | GUUCGGCAAGACCAAGGACCUGCCCGACCUGCGGGGCCCC | |
| | UUCAGCUACCCUAGUUUGACCAGCGCCCAGAGCGGCGAC | |
| | UACAGCUUGGUGAUCGUGACCACCUUCGUGCACUACGCC | |
| | AACUUCCACAACUACUUCGUGCCCAACCUGAAGGACAUG | |
| | UUCAGCCGGGCCGUGACCAUGACUGCCGCUUCUUACGCCC | |
| | GGUACGUGCUGCAGAAGCUGGUCCUGCUGGAGAUGAAGG | |
| | GCGGCUGCCGGGAGCCCGAGCUGGACACCGAAACACUGA | |
| | CCACCAUGUUCGAGGUGAGCGUGGCCUUCUUCAAGGUGG | |
| | GUCACGCGGUGGGCGAAACCGGCAACGGCUGCGUGGACU | |
| | UACGCUGGCUGGCCAAGAGCUUCUUCGAGCUGACCGUGC | |
| | UGAAGGAUAUCAUCGGCAUCUGCUACGGCGCCACCGUGA | |
| | AGGGCAUGCAGAGCUACGGCCUGGAGCGGCUGGCCGCCA | |
| | UGCUUAUGGCAACAGUGAAGAUGGAGGAGCUGGGACACC | |
| | UGACAACAGAGAAGCAGGAGUACGCCCUGAGACUGGCCA | |
| | CAGUGGGCUACCCAAAGGCCGGCGUGUACAGUGGACUGA | |
| | UCGGCGGCGCAACCAGCGUGCUGCUAUCCGCUUACAACCG | |
| | GCACCCGCUGUUCCAGCCCCUGCACACCGUGAUGCGGGAA | |
| | ACCCUGUUCAUCGGAAGCCACGUCGUGCUGCGGGAGCUG | |
| | AGGCUGAACGUAACCACCCAGGGCCCUAAUCUGGCCCUG | |
| | UAUCAGCUCCUCAGUACCGCCCUGUGCAGCGCCCUUGAGA | |
| | UCGGCGAGGUGCUCAGAGGCCUGGCCCUCGGUACCGAGA | |
| | GCGGCCUCUUCAGCCCAUGCUACUUAAGCCUGCGGUUCG | |
| | ACCUGACCCGGGACAAGUUGCUGAGCAUGGCCCCGCAGG | |
| | AGGCCACACUGGACCAGGCAGCUGUAUCCAACGCCGUGG | |
| | ACGGCUUCCUGGGCAGACUGUCCCUGGAACGGGAGGACC | |
| | GGGACGCCUGGCACCUGCCUGCCUACAAGUGUGUGGAUC | |
| | GGCUGGACAAGGUGCUGAUGAUCAUCCCUCUGAUUAAUG | |
| | UCACCUUCAUCAUCAGCAGCGACCGGGAGGUGCGGGGAU | |
| | CCGCCCUCUACGAGGCCAGCACCACCUAUCUGAGCAGCAG | |
| | CCUGUUCCUGUCUCCUGUGAUCAUGAACAAGUGCAGCCA | |
| | GGGCGCCGUGGCCGGCGAGCCCCGGCAGAUCCCCAAGAUC | |
| | CAGAACUUCACCCGGACCCAGAAGUCUUGCAUCUUCUGC | |
| | GGCUUCGCCCUUUUGUCCUACGACGAGAAGGAGGGCUUG | |
| | GAGACUACAACCUACAUCACCAGCCAGGAGGUGCAGAAC | |
| | AGCAUCCUGUCAUCUAAUUACUUCGACUUCGACAACCUG | |
| | CACGUUCAUUACCUGCUCCUCACCACCAACGGUACCGUCA | |
| | UGGAAAUCGCCGGACUGUACGAGGAGCGGGCCCAUGUUG | |
| | UGCUGGCCAUCAUCCUGUACUUCAUCGCUUUCGCACUUG | |
| | GCAUCUUCCUGGUGCACAAGAUCGUGAUGUUCUUCCUG | |
| 3′ UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MQLLCVFCLVLLWEVGAASLSEVKLHLDIEGHASHYTIPWTE | 38 |
| amino acid | LMAKVPGLSPEALWREANVTEDLASMLNRYKLIYKTSGTLGI | |
| sequence | ALAEPVDIPAVSEGSMQVDASKVHPGVISGLNSPACMLSAPLE | |
| | KQLFYYIGTMLPNTRPHSYVFYQLRCHLSYVALSINGDKFQYT | |
| | GAMTSKFLMGTYKRVTEKGDEHVLSLVFGKTKDLPDLRGPFS | |
| | YPSLTSAQSGDYSLVIVTTFVHYANFHNYFVPNLKDMFSRAV | |
| | TMTAASYARYVLQKLVLLEMKGGCREPELDTETLTTMFEVS | |
| | VAFFKVGHAVGETGNGCVDLRWLAKSFFELTVLKDIIGICYG | |
| | ATVKGMQSYGLERLAAMLMATVKMEELGHLTTEKQEYALR | |
| | LATVGYPKAGVYSGLIGGATSVLLSAYNRHPLFQPLHTVMRE | |
| | TLFIGSHVVLRELRLNVTTQGPNLALYQLLSTALCSALEIGEVL | |
| | RGLALGTESGLFSPCYLSLRFDLTRDKLLSMAPQEATLDQAA | |
| | VSNAVDGFLGRLSLEREDRDAWHLPAYKCVDRLDKVLMIIPL | |
| | INVTFIISSDREVRGSALYEASTTYLSSSLFLSPVIMNKCSQGAV | |
| | AGEPRQIPKIQNFTRTQKSCIFCGFALLSYDEKEGLETTTYITSQ | |
| | EVQNSILSSNYFDFDNLHVHYLLLTTNGTVMEIAGLYEERAH | |
| | VVLAIILYFIAFALGIFLVHKIVMFFL | |
| PolyA tail | 100 nt | |

EBV gL (B95-8)

| SEQ ID NO: 202 consists of from 5′ end to 3′ end: | | 202 |
|---|---|---|
| 5′ UTR SEQ ID NO: 104, mRNA ORF SEQ ID NO: 107, and | | |
| 3′ UTR SEQ ID NO: 106 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGAGAGCCGUGGGCGUGUUCCUGGCCAUCUGCCUGGUG | 107 |
| Construct | ACCAUCUUCGUGCUGCCCACCUGGGGCAACUGGGCCUACC | |
| (excluding | CCUGCUGCCACGUGACCCAGCUGAGAGCCCAGCACCUGCU | |
| the stop | GGCCCUGGAGAACAUCAGCGACAUCUACCUGGUGAGCAA | |
| codon) | CCAGACCUGCGACGGCUUCAGCCUGGCCAGCCUGAACAGC | |
| | CCCAAGAACGGCAGCAACCAGCUGGUGAUCAGCAGAUGC | |
| | GCCAACGGCCUGAACGUGGUGAGCUUCUUCAUCAGCAUC | |
| | CUGAAGAGAAGCAGCAGCGCCCUGACCGGCCACCUGAGA | |
| | GAGCUGCUGACCACCCUGGAGACCCUGUACGGCAGCUUC | |
| | AGCGUGGAGGACCUGUUCGGCGCCAACCUGAACAGAUAC | |
| | GCCUGGCACAGAGGCGGC | |
| 3′ UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MRAVGVFLAICLVTIFVLPTWGNWAYPCCHVTQLRAQHLLA | 36 |
| amino acid | LENISDIYLVSNQTCDGFSLASLNSPKNGSNQLVISRCANGLNV | |
| sequence | VSFFISILKRSSSALTGHLRELLTTLETLYGSFSVEDLFGANLNR | |
| | YAWHRGG | |
| PolyA tail | 100 nt | |

EBV gp42 (B95-8)

| SEQ ID NO: 203 consists of from 5′ end to 3′ end: | | 203 |
|---|---|---|
| 5′ UTR SEQ ID NO: 104, mRNA ORF SEQ ID NO: 108, and | | |
| 3′ UTR SEQ ID NO: 1-6 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGGUGAGCUUCAAGCAGGUGAGAGUGCCCCUGUUCACC | 108 |
| Construct | GCCAUCGCCCUGGUGAUCGUGCUGCUGCUGGCCUACUUCC | |
| (excluding | UGCCACCCAGAGUGAGAGGCGGCGGCAGAGUGGCCGCCG | |
| the stop | CCGCCAUCACCUGGGUGCCCAAGCCCAACGUGGAGGUGU | |
| codon) | GGCCCGUGGACCCGCCACCUCCCGUGAACUUCAACAAGAC | |
| | CGCCGAGCAGGAGUACGGCGACAAGGAGGUGAAGCUGCC | |
| | CCACUGGACGCCGACCCUGCACACCUUCCAGGUGCCCCAG | |
| | AACUACACCAAGGCCAACUGCACCUACUGCAACACCAGAG | |
| | AGUACACCUUCAGCUACAAGGGCUGCUGCUUCUACUUCA | |
| | CCAAGAAGAAGCACACCUGGAACGGCUGCUUCCAGGCCU | |
| | GCGCCGAGCUGUACCCCUGCACCUACUUCUACGGCCCCAC | |
| | CCCAGACAUCCUGCCCGUGGUGACCAGAAACCUGAACGCC | |
| | AUCGAGAGCCUGUGGGUGGGCGUGUACAGAGUGGGCGAG | |
| | GGCAACUGGACCAGCCUGGACGGCGGCACCUUCAAGGUG | |
| | UACCAGAUCUUCGGCAGCCACUGCACCUACGUGAGCAAG | |
| | UUCAGCACCGUGCCCGUGAGCCACCACGAGUGCAGCUUCC | |
| | UGAAGCCCUGCCUGUGCGUGAGCCAGAGAAGCAACAGC | |
| 3′ UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MVSFKQVRVPLFTAIALVIVLLLAYFLPPRVRGGGRVAAAAIT | 34 |
| amino acid | WVPKPNVEVWPVDPPPPVNFNKTAEQEYGDKEVKLPHWTPT | |
| sequence | LHTFQVPQNYTKANCTYCNTREYTFSYKGCCFYFTKKKHTW | |
| | NGCFQACAELYPCTYFYGPTPDILPVVTRNLNAIESLWVGVYR | |
| | VGEGNWTSLDGGTFKVYQIFGSHCTYVSKFSTVPVSHHECSFL | |
| | KPCLCVSQRSNS | |
| PolyA tail | 100 nt | |

EBV gp350_001 (modified)

| SEQ ID NO: 204 consists of from 5′ end to 3′ end: | | 204 |
|---|---|---|
| 5′ UTR SEQ ID NO: 104, mRNA ORF SEQ ID NO: 109, and | | |
| 3′ UTR SEQ ID NO: 106 | | |
| Chemistry | 1-methylpseudouridine | |
| Cap | 7mG(5′)ppp(5′)NlmpNp | |
| 5′UTR | GGGAAAUAAGAGAGAAAAGAAGAGUAAGAAGAAAUAUA | 104 |
| | AGACCCCGGCGCCGCCACC | |
| ORF of mRNA | AUGGAGGCAGCACUGCUGGUCUGCCAGUAUACAAUCCAG | 109 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUACAAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAACUAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | UCUGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCACUUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUACUGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCACGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 28 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV D123_Ferritin_RX

| SEQ ID NO: 205 consists of from 5′ end to 3′ end: | | 205 |
|---|---|---|
| 5′ UTR SEQ ID NO: 104, mRNA ORF SEQ ID NO: 110, and | | |
| 3′ UTR SEQ ID NO: 106 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGGACAGCAAGGGCAGCAGCCAGAAGGGAUCGAGGCUA | 110 |
| Construct | UUAUUGUUACUUGUGGUGAGCAACUUGCUCCUGCCUCAG | |
| (excluding | GGCGUGGUGGGCGAGGCCGCCUUGUUGGUGUGCCAGUAC | |
| the stop | ACCAUCCAGAGCCUCAUCCACCUCACCGGCGAGGACCCCG | |
| codon) | GCUUCUUCAACGUGGAGAUCCCCGAGUUCCCCUUCUACCC | |
| | CACCUGCAACGUGUGCACCGCCGACGUGAACGUGACCAUC | |
| | AACUUCGACGUGGGCGGCAAGAAGCACCAGCUGGACCUG | |
| | GACUUCGGCCAGCUGACCCCGCACACCAAGGCCGUGUACC | |
| | AGCCCAGGGGCGCCUUCGGCGGCAGCGAGAACGCCACCAA | |
| | CCUGUUCCUGCUGGAGCUGCUGGGCGCCGGCGAGCUGGC | |
| | CCUGACCAUGAGGUCCAAGAAGCUGCCCAUCAAUGUGAC | |
| | CACAGGAGAGGAGCAGCAGGUGAGCCUGGAGAGCGUGGA | |
| | CGUGUACUUCCAGGACGUGUUCGGCACCAUGUGGUGCCA | |
| | CCACGCCGAGAUGCAGAACCCAGUUUACUUGAUUCCUGA | |
| | GACAGUGCCCUACAUCAAGUGGGACAACUGCAACAGCAC | |
| | CAACAUCACCGCCGUGGUGAGGGCCCAGGGCCUGGACGU | |
| | GACCCUGCCCCUGAGCCUGCCCACCAGCGCCCAGGAUAGC | |
| | AACUUCAGCGUGAAGACCGAAAUGCUGGGCAACGAGAUC | |
| | GACAUCGAGUGCAUCAUGGAGGACGGCGAGAUCAGCCAG | |
| | GUGCUGCCCGGCGACAACAAGUUCAACAUUACCUGCAGC | |
| | GGCUACGAGAGCCACGUGCCCAGCGGCGGCAUCCUGACCU | |
| | CAACUUCACCCGUGGCCACUCCGAUCCCCGGCACCGGCUA | |
| | CGCCUACAGCCUGCGUCUGACACCUAGGCCCGUGAGCAGG | |
| | UUCCUGGGCAAUAACAGCAUCCUGUACGUGUUCUACAGC | |
| | GGCAACGGCCCCAAGGCUUCCGGCGGCGACUACUGUAUU | |
| | CAAUCAAACAUCGUGUUCAGCGACGAGAUACCAGCCAGC | |
| | CAGGACAUGCCUACUAACACCACCGACAUCACCUAUGUG | |
| | GGAGACAAUGCUACUUACAGCGUGCCCAUGGUGACCAGC | |
| | GAGGACGCCAACAGCCCAAAUGUCACCGUGACCGCCUUCU | |
| | GGGCCUGGCCCAACAACACUGAGACAGACUUCAAGUGCA | |
| | AGUGGACCCUCACAUCCGGCACCCCUAGCGGAUGCGAGA | |
| | ACAUCAGCGGAGCGUUCGCCAGCAACAGGACCUUCGAUA | |
| | UAACAGUGAGCGGCCUGGGCACCGCUCCUAAGACCCUGA | |
| | UCAUCACCAGGACGGCCACAAAUGCAACUACUACUACAC | |
| | ACAAGGUGAUCUUCAGCAAGGCUCCGGAAUCACAAGUGA | |
| | GGCAGCAGUUCUCAAAGGAUAUCGAGAAGCUUCUGAACG | |
| | AGCAAGUUAACAAGGAAAUGCAGAGCAGUAAUCUCUACA | |
| | UGAGCAUGAGCAGCUGGUGCUACACCCACUCCCUGGACG | |
| | GAGCAGGCCUCUUCCUGUUCGACCACGCAGCCGAGGAGU | |
| | ACGAGCACGCUAAGAAGUUGAUCAUUUUCUUGAACGAGA | |
| | ACAACGUGCCCGUGCAGCUAACGUCAAUCAGCGCACCUG | |
| | AGCACAAGUUCGAGGGCCUGACCCAGAUCUUCCAGAAGG | |
| | CCUACGAACACGAACAGCACAUCUCCGAGAGCAUCAACA | |
| | AUAUUGUGGAUCACGCUAUCAAGUCCAAGGACCACGCUA | |
| | CCUUCAACUUCCUGCAGUGGUACGUGGCCGAGCAACAUG | |
| | AGGAGGAGGUGCUGUUCAAGGACAUCCUGGACAAGAUCG | |
| | AGCUGAUCGGUAAUGAGAAUCACGGCCUGUACCUGGCCG | |
| | ACCAGUACGUGAAGGGCAUCGCCAAGAGCCGGAAGUCA | |
| 3′ UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDSKGSSQKGSRLLLLLVVSNLLLPQGVVGEAALLVCQYTIQ | 111 |
| amino acid | SLIHLTGEDPGFFNVEIPEFPFYPTCNVCTADVNVTINFDVGGK | |
| sequence | KHQLDLDFGQLTPHTKAVYQPRGAFGGSENATNLFLLELLGA | |
| | GELALTMRSKKLPINVTTGEEQQVSLESVDVYFQDVFGTMWC | |
| | HHAEMQNPVYLIPETVPYIKWDNCNSTNITAVVRAQGLDVTL | |
| | PLSLPTSAQDSNFSVKTEMLGNEIDIECIMEDGEISQVLPGDNK | |
| | FNITCSGYESHVPSGGILTSTSPVATPIPGTGYAYSLRLTPRPVS | |
| | RFLGNNSILYVFYSGNGPKASGGDYCIQSNIVFSDEIPASQDMP | |
| | TNTTDITYVGDNATYSVPMVTSEDANSPNVTVTAFWAWPNN | |
| | TETDFKCKWTLTSGTPSGCENISGAFASNRTFDITVSGLGTAPK | |
| | TLIITRTATNATTTTHKVIFSKAPESQVRQQFSKDIEKLLNEQV | |
| | NKEMQSSNLYMSMSSWCYTHSLDGAGLFLFDHAAEEYEHAK | |
| | KLIIFLNENNVPVQLTSISAPEHKFEGLTQIFQKAYEHEQHISESI | |
| | NNIVDHAIKSKDHATFNFLQWYVAEQHEEEVLFKDILDKIELI | |
| | GNENHGLYLADQYVKGIAKSRKS | |
| PolyA tail | 100 nt | |

EBV D123_LS

| SEQ ID NO: 206 consists of from 5′ end to 3′ end: | | 206 |
|---|---|---|
| 5′ UTR SEQ ID NO: 104, mRNA ORF SEQ ID NO: 112, and | | |
| 3′ UTR SEQ ID NO: 106 | | |
| Chemistry | 1-methylpseudouridine | |
| Cap | 7mG(5′)ppp(5′)NlmpNp | |
| 5′UTR | GGGAAAUAAGAGAGAAAAGAAGAGUAAGAAGAAAUAUA | 104 |
| | AGACCCCGGCGCCGCCACC | |
| ORF of mRNA | AUGGACAGCAAGGGCAGCAGCCAGAAGGGCUCCCGGUUG | 112 |
| Construct | UUGCUCUUGCUCGUGGUGAGCAACUUGUUGUUGCCUCAG | |
| (excluding | GGCGUGGUGGGCGAGGCCGCCUUGUUGGUGUGCCAGUAC | |
| the stop | ACCAUCCAGAGCCUCAUCCACCUCACCGGCGAGGACCCUG | |
| codon) | GCUUCUUCAACGUGGAGAUCCCUGAGUUCCCUUUCUACC | |
| | CUACCUGCAACGUGUGCACCGCCGACGUGAACGUGACCA | |
| | UCAACUUCGACGUGGGCGGCAAGAAGCACCAGCUGGACC | |
| | UGGACUUCGGCCAGCUGACCCCUCACACCAAGGCCGUGUA | |
| | CCAGCCUAGAGGCGCCUUCGGCGGCAGCGAGAACGCCACC | |
| | AACCUGUUCCUGCUGGAGCUGCUGGGCGCCGGCGAGCUG | |
| | GCCCUGACCAUGAGAAGUAAGAAGCUGCCUAUCAACGUC | |
| | ACUACUGGCGAGGAGCAGCAGGUGAGCCUGGAGAGCGUG | |
| | GACGUGUACUUCCAGGACGUGUUCGGCACCAUGUGGUGC | |
| | CACCACGCCGAGAUGCAGAACCCUGUGUACCUGAUCCCAG | |
| | AAACCGUGCCUUACAUCAAGUGGGACAACUGCAACAGCA | |
| | CCAACAUCACCGCUGUGGUGAGAGCCCAGGGCCUGGACG | |
| | UGACCCUGCCUCUGAGCCUGCCUACCAGCGCCCAAGACAG | |
| | UAACUUCAGCGUGAAGACGGAGAUGCUGGGCAACGAGAU | |
| | CGACAUCGAGUGCAUCAUGGAGGACGGCGAGAUCAGCCA | |
| | GGUGCUGCCUGGCGACAACAAGUUCAACAUAACAUGCAG | |
| | CGGCUACGAGAGCCACGUGCCUAGCGGCGGCAUCCUGACC | |
| | UCCACCAGCCCUGUGGCCACCCCUAUCCCUGGCACCGGCU | |
| | ACGCCUACAGCCUGAGACUCACGCCAAGACCUGUGAGCA | |
| | GAUUCUUAGGAAACAACAGCAUCCUGUACGUGUUCUACA | |
| | GCGGCAACGGCCCUAAGGCCUCCGGCGGUGACUACUGUA | |
| | UCCAGUCUAACAUCGUGUUCAGCGACGAAAUCCCAGCCA | |
| | GCCAGGACAUGCCUACAAACACCACCGACAUCACUUAUG | |
| | UCGGCGACAAUGCGACAUACAGCGUGCCUAUGGUGACCA | |
| | GCGAGGACGCCAACAGCCCUAACGUAACCGUGACCGCCUU | |
| | CUGGGCCUGGCCUAACAACACCGAAACCGACUUCAAGUG | |
| | CAAGUGGACUUUAACUAGUGGCACCCCUUCUGGCUGCGA | |
| | GAACAUCAGCGGCGCAUUCGCCAGCAACAGAACCUUCGA | |
| | UAUCACCGUGAGCGGCCUGGGCACCGCCCCUAAGACCCUG | |
| | AUCAUCACCAGAACCGCAACUAAUGCAACUACAACAACCC | |
| | ACAAGGUGAUCUUCAGCAAGGCCCCUGGCUCCGGCUCUG | |
| | CCGGCAGUGGCAGCGCAGGCAGUGGAUCCGCCGGUUCAG | |
| | GUUCCGCCAUGCAGAUCUACGAGGGCAAGCUGACCGCCG | |
| | AGGGCCUGAGAUUCGGCAUCGUGGCCAGCAGGUUCAACC | |
| | ACGCCCUGGUGGACAGACUGGUGGAGGGCGCCAUCGACG | |
| | CCAUCGUGAGACACGGAGGAAGAGAGGAGGAUAUAACAC | |
| | UGGUCAGAGUGCCUGGAUCAUGGGAAAUCCCUGUCGCCG | |
| | CUGGUGAGCUGGCAAGAAAGGAGGACAUCGAUGCGGUGA | |
| | UCGCCAUCGGCGUGCUGAUCCGCGGUGCAACUCCACACUU | |
| | CGACUACAUCGCCUCUGAAGUGUCAAAGGGCCUUGCGGA | |
| | CCUGUCCCUCGAACUCAGAAAGCCUAUCACAUUCGGAGU | |
| | GAUUACUGCAGAUACCCUGGAGCAGGCCAUCGAGAGAGC | |
| | CGGCACCAAGCACGGCAACAAGGGCUGGGAAGCCGCGCU | |
| | GUCUGCCAUAGAAAUGGCAAACCUUUUCAAGUCCCUUAG | |
| | A | |
| 3′UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MDSKGSSQKGSRLLLLLVVSNLLLPQGVVGEAALLVCQYTIQ | 113 |
| amino acid | SLIHLTGEDPGFFNVEIPEFPFYPTCNVCTADVNVTINFDVGGK | |
| sequence | KHQLDLDFGQLTPHTKAVYQPRGAFGGSENATNLFLLELLGA | |
| | GELALTMRSKKLPINVTTGEEQQVSLESVDVYFQDVFGTMWC | |
| | HHAEMQNPVYLIPETVPYIKWDNCNSTNITAVVRAQGLDVTL | |
| | PLSLPTSAQDSNFSVKTEMLGNEIDIECIMEDGEISQVLPGDNK | |
| | FNITCSGYESHVPSGGILTSTSPVATPIPGTGYAYSLRLTPRPVS | |
| | RFLGNNSILYVFYSGNGPKASGGDYCIQSNIVFSDEIPASQDMP | |
| | TNTTDITYVGDNATYSVPMVTSEDANSPNVTVTAFWAWPNN | |
| | TETDFKCKWTLTSGTPSGCENISGAFASNRTFDITVSGLGTAPK | |
| | TLIITRTATNATTTTHKVIFSKAPGSGSAGSGSAGSGSAGSGSA | |
| | MQIYEGKLTAEGLRFGIVASRFNHALVDRLVEGAIDAIVRHGG | |
| | REEDITLVRVPGSWEIPVAAGELARKEDIDAVIAIGVLIRGATP | |
| | HFDYIASEVSKGLADLSLELRKPITFGVITADTLEQAIERAGTK | |
| | HGNKGWEAALSAIEMANLFKSLR | |
| PolyA tail | 100 nt | |

EBV gp350_004 (modified)

| SEQ ID NO: 207 consists of from 5′ end to 3′ end: | | 207 |
|---|---|---|
| 5′ UTR SEQ ID NO: 1, mRNA ORF SEQ ID NO: 124, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAAGCCGCCCUAUUAGUCUGCCAGUACACCAUUCAA | 124 |
| Construct | AGCCUCAUCCACUUGACUGGGGAGGAUCCUGGGUUCUUU | |
| (excluding | AACGUGGAAAUCCCGGAAUUCCCUUUCUACCCAACUUGC | |
| the stop | AACGUGUGCACGGCUGACGUCAACGUGACUAUCAACUUC | |
| codon) | GAUGUGGGAGGGAAGAAACAUCAACUUGAUCUGGACUUU | |
| | GGCCAGCUUACUCCCCACACCAAGGCCGUGUACCAGCCCC | |
| | GCGGCGCAUUCGGUGGGAGCGAGAACGCGACAAAUCUAU | |
| | UCUUGCUGGAACUGCUGGGCGCAGGAGAGCUCGCCCUGA | |
| | CGAUGCGAUCUAAGAAGCUUCCCAUCAACGUUACCACUG | |
| | GCGAGGAGCAGCAGGUGUCCCUCGAAAGCGUCGACGUCU | |
| | AUUUCCAAGACGUCUUCGGAACAAUGUGGUGCCACCACG | |
| | CUGAGAUGCAGAACCCCGUAUAUCUUAUUCCCGAAACCG | |
| | UGCCAUACAUCAAGUGGGACAACUGCAACUCAACUAACA | |
| | UCACUGCAGUAGUGCGCGCCCAGGGACUGGACGUCACUU | |
| | UGCCACUGUCACUGCCCACAUCAGCCCAAGACAGCAACUU | |
| | CAGCGUCAAGACAGAAAUGCUGGGCAACGAAAUCGACAU | |
| | CGAAUGCAUAAUGGAGGAUGGGGAAAUUUCCCAGGUCCU | |
| | CCCCGGUGAUAACAAGUUUAAUAUUACUUGUUCUGGAUA | |
| | UGAAUCCCAUGUGCCGUCUGGUGGUAUACUGACGAGUAC | |
| | AUCGCCAGUGGCUACCCCAAUCCCCGGGACAGGCUAUGCC | |
| | UACAGCCUGAGACUGACGCCAAGACCAGUUUCCAGGUUU | |
| | CUGGGGAAUAACUCUAUCCUGUAUGUGUUUUAUUCUGGU | |
| | AACGGACCCAAGGCAUCCGGCGGUGAUUAUUGUAUCCAG | |
| | AGCAAUAUAGUGUUCUCUGAUGAGAUUCCGGCCUCCCAG | |
| | GAUAUGCCAACCAACACGACAGAUAUCACCUAUGUCGGG | |
| | GACAACGCCACAUAUAGCGUGCCAAUGGUGACCAGCGAA | |
| | GAUGCCAAUUCUCCCAACGUCACAGUGACAGCCUUUUGG | |
| | GCGUGGCCUAAUAACACAGAAACUGACUUUAAAUGCAAA | |
| | UGGACUCUAACCUCAGGCACCCCAUCUGGCUGCGAGAAC | |
| | AUAAGCGGCGCGUUUGCAUCGAACAGAACUUUUGAUAUU | |
| | ACAGUAUCUGGGUUGGGCACUGCGCCCAAGACUCUAAUA | |
| | AUUACACGGACGGCCACUAACGCCACCACGACGACUCACA | |
| | AGGUGAUCUUCAGCAAAGCUCCCGAAUCUACUACUACCU | |
| | CGCCGACACUGAAUACGACAGGAUUCGCCGACCCAAAUA | |
| | CUACGACCGGCCUUCCCAGCUCGACACAUGUGCCUACAAA | |
| | CUUAACGGCGCCCGCCAGUACCGGCCCCACCGUGAGCACC | |
| | GCUGACGUCACCUCUCCUACCCCUGCCGGGACUACAAGUG | |
| | GUGCUUCCCCAGUUACUCCCAGCCCUAGCCCUUGGGACAA | |
| | CGGCACUGAGAGCAAAGCUCCGGACAUGACCAGCAGUAC | |
| | CAGUCCAGUGACAACUCCGACACCCAAUGCGACGUCCCCA | |
| | ACUCCCGCCGUGACCACGCCAACACCAAAUGCCACGUCAC | |
| | CUACUCCAGCGGUCACCACCCCAACGCCAAACGCUACUAG | |
| | UCCCACUCUAGGCAAGACAUCUCCCACCUCCGCUGUCACC | |
| | ACCCCUACACCCAAUGCUACCGGUCCCACGGUGGGUGAGA | |
| | CUAGCCCACAGGCCAACGCAACUAAUCAUACAUUGGGUG | |
| | GCACAAGCCCAACGCCCGUCGUCACUAGCCAGCCUAAGAA | |
| | CGCCACAAGCGCUGUAACUACCGGCCAGCACAAUAUAAC | |
| | AAGUUCCAGUACUAGCAGUAUGUCUCUGAGACCCAGCUC | |
| | AAAUCCUGAAACCCUCUCGCCCUCUACGUCGGAUAACUCC | |
| | ACGUCCCACAUGCCUCUACUCACAAGCGCGCACCCUACCG | |
| | GAGGAGAGAAUAUCACUCAGGUUACCCCAGCUUCUAUCU | |
| | CGACUCACCAUGUCUCUACUUCUUCCCCAGCGCCACGGCC | |
| | CGGCACUACCAGCCAGGCUAGCGGGCCAGGUAAUUCCUCC | |
| | ACUAGCACUAAACCUGGCGAAGUCAACGUUACCAAGGGA | |
| | ACACCUCCCCAGAAUGCAACCUCGCCCCAAGCUCCCUCUG | |
| | GACAGAAGACAGCCGUCCCUACCGUCACAAGCACCGGUG | |
| | GGAAGGCGAAUUCUACGACAGGCGGGAAGCACACUACCG | |
| | GACACGGAGCAAGGACAUCCACUGAGCCCACGACCGACU | |
| | ACGGUGGCGACUCCACCACGCCCAGACCCAGGUACAAUGC | |
| | GACUACCUACCUGCCGCCUUCCACUUCCAGCAAACUGAGG | |
| | CCUCGUUGGACUUUCACUUCUCCACCAGUCACCACCGCUC | |
| | AGGCUACCGUGCCUGUCCCGCCGACCUCUCAGCCCCGGUU | |
| | UUCAAAUUUGAGUAUGCUCGUGCUGCAGUGGGCCAGCCU | |
| | GGCAGUGCUUACCCUCCUGCUCUUGCUGGUUAUGGCUGA | |
| | CUGCGCCUUCCGACGUAACUUAUCCACCAGUCAUACUUAC | |
| | ACUACACCUCCUUAUGACGACGCGGAGACCUACGUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 125 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

EBV gp350_005 (modified)

| SEQ ID NO: 208 consists of from 5′ end to 3′ end: | | 208 |
|---|---|---|
| 5′ UTR SEQ ID NO: 1, mRNA ORF SEQ ID NO: 126, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAAGCCGCCUUACUGGUCUGCCAGUACACUAUUCAG | 126 |
| Construct | UCCCUCAUCCAUCUCACAGGCGAGGACCCGGGGUUCUUCA | |
| (excluding | ACGUCGAAAUCCCCGAAUUCCCAUUCUAUCCCACCUGUAA | |
| the stop | CGUCUGCACAGCCGACGUUAACGUUACAAUCAAUUUCGA | |
| codon) | CGUGGGCGGGAAGAAACACCAGCUGGACCUCGACUUUGG | |
| | GCAGCUGACGCCCCACACUAAAGCCGUUUACCAGCCCAGG | |
| | GGUGCAUUCGGUGGAUCUGAGAAUGCUACAAACCUCUUU | |
| | CUGCUUGAGCUGCUUGGUGCUGGAGAGCUGGCCCUGACA | |
| | AUGCGCAGCAAGAAGUUGCCAAUCAAUGUGACCACAGGU | |
| | GAAGAACAGCAGGUGUCCCUAGAAUCCGUGGAUGUGUAC | |
| | UUCCAGGACGUUUUCGGCACAAUGUGGUGCCAUCACGCA | |
| | GAGAUGCAGAAUCCGGUGUACCUUAUCCCCGAAACAGUG | |
| | CCGUACAUUAAAUGGGAUAAUUGUAACUCCACAAACAUC | |
| | ACGGCAGUGGUGCGCGCUCAGGGGCUGGACGUGACACUC | |
| | CCGCUUUCCUUACCAACAAGUGCUCAAGAUUCCAACUUU | |
| | UCUGUGAAGACCGAGAUGCUGGGCAACGAGAUUGAUAUU | |
| | GAAUGCAUCAUGGAGGAUGGCGAGAUUAGCCAGGUGUUG | |
| | CCCGGCGACAAUAAAUUUAAUAUUACCUGUUCCGGCUAU | |
| | GAGUCACACGUGCCAAGUGGAGGCAUUCUCACGAGCACC | |
| | UCUCCCGUUGCAACACCCAUUCCUGGGACAGGGUACGCA | |
| | UAUAGUCUCAGGCUGACACCGAGACCGGUAUCGCGCUUC | |
| | UUAGGUAACAAUUCCAUCUUGUAUGUGUUUUACAGCGGA | |
| | AACGGGCCAAAGGCCUCAGGAGGAGACUACUGCAUCCAG | |
| | AGCAAUAUAGUCUUUAGCGACGAGAUCCCUGCCUCACAA | |
| | GACAUGCCCACCAACACCACCGACAUCACGUACGUCGGUG | |
| | AUAAUGCCACUUACAGCGUGCCUAUGGUGACCUCUGAAG | |
| | ACGCAAAUUCACCCAAUGUCACGGUUACCGCAUUCUGGG | |
| | CUUGGCCAAAUAACACUGAAACCGACUUCAAGUGUAAAU | |
| | GGACCCUAACCAGCGGCACCCCAUCAGGGUGCGAGAAUA | |
| | UCAGCGGGGCCUUCGCCAGCAACCGGACAUUCGACAUCAC | |
| | CGUGUCAGGACUAGGGACUGCUCCAAAGACAUUAAUUAU | |
| | UACACGGACAGCAACUAACGCUACGACUACGACUCACAA | |
| | AGUGAUAUUUAGCAAAGCGCCCGAAUCGACUACUACGAG | |
| | CCCGACCCUAAACACCACCGGUUUCGCCGAUCCCAACACA | |
| | ACUACUGGUCUCCCAUCUUCGACCCAUGUUCCAACGAAUC | |
| | UGACAGCUCCAGCAAGUACGGGGCCCACUGUGAGUACAG | |
| | CCGACGUUACUAGCCCAACUCCCGCGGGUACGACUAGUG | |
| | GCGCGUCUCCAGUGACCCCUUCGCCUUCUCCGUGGGAUAA | |
| | UGGAACCGAGUCAAAGGCUCCUGAUAUGACCAGCUCCAC | |
| | CAGUCCCGUUACAACGCCAACGCCCAAUGCAACGAGUCCA | |
| | ACUCCGGCGGUUACCACUCCGACACCAAACGCCACCUCGC | |
| | CCACGCCUGCAGUUACUACGCCCACCCCUAAUGCUACGUC | |
| | ACCCACUCUGGGAAAGACCUCACCAACCUCUGCCGUGACU | |
| | ACUCCUACACCUAAUGCCACAGGACCCACAGUUGGGGAA | |
| | ACCAGCCCACAGGCUAACGCAACUAAUCAUACGCUUGGC | |
| | GGGACCUCUCCCACGCCCGUGGUCACUUCACAGCCUAAGA | |
| | AUGCCACAAGCGCUGUGACUACAGGCCAGCAUAAUAUAA | |
| | CCAGCAGUUCAACCAGUAGCAUGUCCCUGCGUCCUUCAUC | |
| | CAAUCCUGAGACUCUGAGCCCCAGCACUAGUGACAACUC | |
| | AACUUCUCACAUGCCGUUGCUGACCUCCGCGCAUCCCACG | |
| | GGCGGUGAGAAUAUCACACAGGUGACACCCGCGUCUAUU | |
| | AGCACACAUCACGUGAGUACGUCCUCGCCCGCACCCCGCC | |
| | CAGGCACAACCUCACAAGCAUCAGGUCCGGGGAAUAGCA | |
| | GCACAAGUACCAAGCCUGGCGAAGUGAAUGUAACCAAGG | |
| | GCACGCCACCGCAGAACGCCACAUCCCCUCAAGCCCCAUC | |
| | CGGCCAGAAGACCGCUGUACCCACCGUGACAUCAACUGG | |
| | GGGGAAAGCGAACUCAACUACCGGUGGGAAGCAUACAAC | |
| | AGGGCACGGUGCCAGAACUUCUACAGAGCCAACCACGGA | |
| | CUACGGAGGGGACAGCACAACGCCCAGACCGCGGUACAA | |
| | UGCGACCACAUACCUGCCUCCCUCAACUUCGAGUAAGCUG | |
| | CGACCCAGAUGGACCUUUACUUCUCCUCCUGUCACCACCG | |
| | CUCAAGCCACCGUCCCGGUUCCACCUACUUCCCAGCCUAG | |
| | AUUCUCCAAUCUGAGCAUGCUUGUCCUACAGUGGGCAUC | |
| | ACUCGCGGUACUAACCUUGCUGCUACUGUUGGUGAUGGC | |
| | GGACUGCGCGUUUCGCCGCAACUUAUCCACCUCCCACACG | |
| | UACACAACACCUCCCUAUGAUGACGCUGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 127 |
| amino acid | DVNVTINFDVGGKKHQLDLDFGQLTPHTKAVYQPRGAFGGS | |
| sequence | ENATNLFLLELLGAGELALTMRSKKLPINVTTGEEQQVSLESV | |
| | DVYFQDVFGTMWCHHAEMQNPVYLIPETVPYIKWDNCNSTN | |
| | ITAVVRAQGLDVTLPLSLPTSAQDSNFSVKTEMLGNEIDIECIM | |
| | EDGEISQVLPGDNKFNITCSGYESHVPSGGILTSTSPVATPIPGT | |
| | GYAYSLRLTPRPVSRFLGNNSILYVFYSGNGPKASGGDYCIQS | |
| | NIVFSDEIPASQDMPTNTTDITYVGDNATYSVPMVTSEDANSP | |
| | NVTVTAFWAWPNNTETDFKCKWTLTSGTPSGCENISGAFASN | |
| | RTFDITVSGLGTAPKTLIITRTATNATTTTHKVIFSKAPESTTTS | |
| | PTLNTTGFADPNTTTGLPSSTHVPTNLTAPASTGPTVSTADVTS | |
| | PTPAGTTSGASPVTPSPSPWDNGTESKAPDMTSSTSPVTTPTPN | |
| | ATSPTPAVTTPTPNATSPTPAVTTPTPNATSPTLGKTSPTSAVT | |
| | TPTPNATGPTVGETSPQANATNHTLGGTSPTPVVTSQPKNATS | |
| | AVTTGQHNITSSSTSSMSLRPSSNPETLSPSTSDNSTSHMPLLTS | |
| | AHPTGGENITQVTPASISTHHVSTSSPAPRPGTTSQASGPGNSS | |
| | TSTKPGEVNVTKGTPPQNATSPQAPSGQKTAVPTVTSTGGKA | |
| | NSTTGGKHTTGHGARTSTEPTTDYGGDSTTPRPRYNATTYLPP | |
| | STSSKLRPRWTFTSPPVTTAQATVPVPPTSQPRFSNLSMLVLQ | |
| | WASLAVLTLLLLLVMADCAFRRNLSTSHTYTTPPYDDAETYV | |
| PolyA tail | 100 nt | |

SE_EBV gB_019

| SEQ ID NO: 209 consists of from 5′ end to 3′ end: | | 209 |
|---|---|---|
| 5′ UTR SEQ ID NO: 1, mRNA ORF SEQ ID NO: 128, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGACCCGGCGGCGGGUGUUAAGCGUGGUGGUGCUCUUA | 128 |
| Construct | GCCGCCUUAGCCUGCCGGCUUGGCGCCCAGACUCCGGAGC | |
| (excluding | AGCCCGCUCCUCCCGCCACCACCGUCCAGCCCACCGCCAC | |
| the stop | CCGCCAGCAGACCUCCUUCCCCUUCCGCGUCUGCGAGCUC | |
| codon) | UCCUCCCACGGCGACCUCUUCCGCUUCUCCUCCGACAUCC | |
| | AGUGCCCCUCCUUCGGCACCCGCGAGAACCACACCGAGGG | |
| | CCUCCUCAUGGUCUUCAAGGACAACAUCAUCCCCUACUCC | |
| | UUCAAGGUCCGCUCCUACACCAAGAUCGUCACCAACAUCC | |
| | UCAUCUACAACGGCUGGUACGCCGACUCUGUGACUAAUC | |
| | GCCACGAGGAGAAGUUCUCCGUCGACUCCUACGAGACUG | |
| | ACCAGAUGGACACCAUCUACCAGUGCUACAACGCCGUCA | |
| | AGAUGACCAAGGACGGCCUCACCCGCGUCUACGUCGACCG | |
| | CGACGGCGUCAACAUCACCGUCAACCUCAAGCCUACAGGC | |
| | GGCCUCGCCAACGGCGUCCGCCGCUACGCCUCCCAGACCG | |
| | AGCUCUACGACGCCCCUGGCUGGCUCAUCUGGACCUACCG | |
| | CACCAGGACCACUGUCAAUUGCCUCAUCACCGACAUGAU | |
| | GGCCAAGUCCAACUCCCCUUUCGACUUCUUCGUGACCACC | |
| | ACAGGCCAGACCGUCGAGAUGUCGCCAUUCUAUGACGGC | |
| | AAGAACAAGGAGACUUUCCACGAGAGAGCGGACUCUUUC | |
| | CAUGUCCGCACCAACUACAAGAUUGUCGACUACGACAAC | |
| | CGCGGCACCAAUCCACAGGGCGAGCGCCGCGCCUUCCUCG | |
| | ACAAGGGCACCUACACCCUCUCCUGGAAGCUCGAGAACCG | |
| | CACCGCCUACUGUCCUCUCCAGCACUGGCAGACCUUCGAU | |
| | UCCACCAUCGCCACCGAGACUGGCAAGUCCAUCCAUUUCG | |
| | UCACCGACGAAGGAACAUCUUCAUUCGUCACAAAUACAA | |
| | CCGUGGGCAUCGAGCUCCCCGACGCCUUCAAGUGCAUCGA | |
| | GGAGCAGGUCAACAAGACCAUGCACGAGAAGUACGAGGC | |
| | CGUCCAGGACCGGUAUACAAAGGGCCAGGAGGCCAUCAC | |
| | CUACUUCAUCACCAGCGGAGGUCUGCUUCUCGCCUGGCU | |
| | UCCACUCACACCUCGCUCCCUGGCCACAGUGAAGAACCUC | |
| | ACUGAGUUGACCACCCCAACAAGUUCCCCACCUAGUAGUC | |
| | CUAGCCCUCCUGCUCCUUCCGCCGCCCGCGGCUCCACGCC | |
| | UGCCGCCGUCCUGCGGAGACGGAGAAGGGACGCCGGCAA | |
| | CGCUACCACUCCUGUGCCGCCGACAGCCCCUGGAAAGUCC | |
| | CUAGGUACCCUCAACAACCCAGCGACAGUUCAGAUCCAG | |
| | UUCGCCUACGACUCUCUCAGGCGCCAGAUCAACCGCAUGC | |
| | UUGGUGACCUUGCCCGCGCCUGGUGCCUCGAGCAGAAGC | |
| | GCCAGAACAUGGUGCUUCGUGAACUAACAAAGAUUAAUC | |
| | CUACUACAGUGAUGUCCUCCAUCUACGGCAAGGCCGUCG | |
| | CCGCCAAGCGCCUCGGCGACGUCAUCUCCGUCUCCCAGUG | |
| | CGUCCCAGUGAACCAGGCUACCGUGACCCUCAGGAAGUCC | |
| | AUGAGAGUGCCAGGCUCCGAGACAAUGUGCUACUCCCGC | |
| | CCACUCGUCUCCUUCUCCUUCAUCAACGACACAAAGACCU | |
| | ACGAGGGCCAGUUAGGCACUGACAACGAGAUCUUCUUGA | |
| | CUAAGAAGAUGACUGAGGUAUGCCAGGCAACUUCUCAAU | |
| | ACUACUUCCAGUCUGGAAAUGAGAUCCACGUAUAUAACG | |
| | ACUACCACCACUUCAAGACUAUUGAACUCGACGGAAUUG | |
| | CCACCCUCCAAACAUUCAUCUCACUGAAUACCUCCCUCAU | |
| | CGAGAACAUCGACUUCGCCUCCCUCGAGCUGUAUAGCAG | |
| | AGACGAGCAGCGCGCCUCCAACGUCUUCGACCUCGAGGGC | |
| | AUCUUCCGCGAGUACAACUUCCAGGCACAGAACAUAGCC | |
| | GGCCUCCGUAAGGAUCUGGACAAUGCCGUGUCCAACGGC | |
| | CGCAACCAGUUCGUCGACGGUUUGGGUGAACUCAUGGAC | |
| | UCUCUGGGCUCCGUCGGCCAGUCCAUAACUAACUUAGUC | |
| | UCUACGGUGGGAGGCCUAUUCAGCAGCCUGGUGAGCGGC | |
| | UUCAUCUCUUUCUUCAAGAACCCCUUCGGCGGCAUGCUC | |
| | AUCCUCGUCCUCGUCGCCGGCGUCGUCAUACUGGUGAUC | |
| | UCACUCACAAGGAGGACGCGCCAAAUGUCCCAGCAGCCA | |
| | GUGCAGAUGCUCUACCCAGGCAUAGACGAGCUCGCUCAG | |
| | CAGCACGCGUCGGGUGAGGGACCAGGCAUCAAUCCUAUC | |
| | UCCAAGACUGAGCUGCAAGCCAUUAUGCUCGCCCUCCACG | |
| | AGCAGAACCAGGAACAGAAGCGGGCCGCCCAGCGAGCUG | |
| | CCGGCCCCUCCGUCGCCAGUAGGGCACUACAAGCCGCCCG | |
| | GGACCGCUUCCCAGGCCUGAGAAGGAGGAGAUACCACGA | |
| | CCCAGAGACAGCCGCUGCCCUCCUUGGCGAAGCAGAAACC | |
| | GAGUUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MTRRRVLSVVVLLAALACRLGAQTPEQPAPPATTVQPTATRQ | 129 |
| amino acid | QTSFPFRVCELSSHGDLFRFSSDIQCPSFGTRENHTEGLLMVFK | |
| sequence | DNIIPYSFKVRSYTKIVTNILIYNGWYADSVTNRHEEKFSVDSY | |
| | ETDQMDTIYQCYNAVKMTKDGLTRVYVDRDGVNITVNLKPT | |
| | GGLANGVRRYASQTELYDAPGWLIWTYRTRTTVNCLITDMM | |
| | AKSNSPFDFFVTTTGQTVEMSPFYDGKNKETFHERADSFHVR | |
| | TNYKIVDYDNRGTNPQGERRAFLDKGTYTLSWKLENRTAYC | |
| | PLQHWQTFDSTIATETGKSIHFVTDEGTSSFVTNTTVGIELPDA | |
| | FKCIEEQVNKTMHEKYEAVQDRYTKGQEAITYFITSGGLLLA | |
| | WLPLTPRSLATVKNLTELTTPTSSPPSSPSPPAPSAARGSTPAA | |
| | VLRRRRRDAGNATTPVPPTAPGKSLGTLNNPATVQIQFAYDSL | |
| | RRQINRMLGDLARAWCLEQKRQNMVLRELTKINPTTVMSSIY | |
| | GKAVAAKRLGDVISVSQCVPVNQATVTLRKSMRVPGSETMC | |
| | YSRPLVSFSFINDTKTYEGQLGTDNEIFLTKKMTEVCQATSQY | |
| | YFQSGNEIHVYNDYHHFKTIELDGIATLQTFISLNTSLIENIDFA | |
| | SLELYSRDEQRASNVFDLEGIFREYNFQAQNIAGLRKDLDNAV | |
| | SNGRNQFVDGLGELMDSLGSVGQSITNLVSTVGGLFSSLVSGF | |
| | ISFFKNPFGGMLILVLVAGVVILVISLTRRTRQMSQQPVQMLY | |
| | PGIDELAQQHASGEGPGINPISKTELQAIMLALHEQNQEQKRA | |
| | AQRAAGPSVASRALQAARDRFPGLRRRRYHDPETAAALLGE | |
| | AETEF | |
| PolyA tail | 100 nt | |

V3_NGM 6mut CR2_EBV_gp350_001

| SEQ ID NO: 210 consists of from 5′ end to 3′ end: | | 210 |
|---|---|---|
| 5′ UTR SEQ ID NO: 1, mRNA ORF SEQ ID NO: 211, and | | |
| 3′ UTR SEQ ID NO: 3. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 1 |
| | AGAGCCACC | |
| ORF of mRNA | AUGGAGGCAGCACUGCUGGUCUGCCAGUAUACAAUCCAG | 211 |
| Construct | UCGCUCAUUCAUCUGACAGGCGAGGACCCCGGCUUCUUU | |
| (excluding | AACGUGGAAAUCCCAGAGUUCCCUUUCUAUCCAACCUGU | |
| the stop | AACGUUUGCACUGCUGACGUGAACGUGACAAUCAAUUUC | |
| codon) | GACGUGGGAGGAAAGAAGCAUCAGCUUGAUCUGGAUUUC | |
| | GGCCAACUGACGCCACACACCAAAGCGGUGUACCAACCAA | |
| | GGGGUGCAUUUGGAGGGAGCGAGAAUGCUGCGAAUCUGU | |
| | UCUUACUGGAACUGCUUGGUGCAGGAGAGCUGGCACUAA | |
| | CCAUGCGAAGCAAGAAGCUCCCCAUCAACGUUACCACCGG | |
| | GGAAGAACAGCAAGUCAGCCUUGAAUCCGUGGAUGUGUA | |
| | CUUUCAGGAUGUUUUCGGCACCAUGUGGUGUCACCACGC | |
| | UGAGAUGCAGAAUCCCGUGUAUCUCAUACCCGAGACUGU | |
| | ACCCUACAUCAAGUGGGAUAAUUGCAAUUCAGCGAAUAU | |
| | UACGGCUGUGGUGAGGGCCCAGGGCUUGGACGUGACUCU | |
| | GCCCUUAUCUCUACCUACUUCUGCCCAAGACUCCAAUUUC | |
| | GCGGUCAAGACCGAGAUGCUCGGGAAUGAGAUCGAUAUC | |
| | GAGUGCAUCAUGGAGGACGGUGAGAUAAGCCAGGUUCUG | |
| | CCCGGCGACAACAAGUUCAAUAUCGCGUGUUCUGGCUAC | |
| | GAGUCCCAUGUGCCUAGUGGUGGCAUACUCACAAGUACU | |
| | UCUCCCGUAGCCACGCCCAUUCCCGGAACCGGAUACGCCU | |
| | ACAGUCUGCGUCUGACCCCACGGCCUGUGUCCAGAUUCCU | |
| | GGGUAACAAUAGUAUCUUAUACGUGUUUUAUAGCGGAAA | |
| | CGGCCCUAAAGCGUCCGGAGGGGACUAUUGUAUUCAGAG | |
| | UAAUAUCGUUUUCUCUGAUGAGAUUCCUGCCAGUCAGGA | |
| | CAUGCCGACAAACACAACUGAUAUUACCUACGUGGGCGA | |
| | CAAUGCCACGUAUUCAGUGCCCAUGGUCACGAGCGAGGA | |
| | CGCCAAUUCACCAAAUGUUGCGGUAACAGCUUUCUGGGC | |
| | CUGGCCAAAUAACACUGAGACUGACUUCAAAUGUAAGUG | |
| | GACUUUGACCUCUGGAACUCCGUCGGGUUGCGAGAAUAU | |
| | CAGCGGGGCCUUUGCUUCCAACAGGACUUUCGACAUCAC | |
| | UGUCUCAGGGCUGGGGACAGCACCGAAGACAUUAAUCAU | |
| | AACACGGACCGCCACCAACGCCGCGACUACAACCCAUAAG | |
| | GUGAUCUUUUCCAAGGCACCUGAGUCCACCACUACCUCCC | |
| | CGACUCUUAACACUACGGGCUUCGCUGAUCCCAACACCAC | |
| | UACGGGGUUGCCUAGCUCGACACAUGUGCCGACGAACCU | |
| | GACUGCCCCUGCAUCGACCGGGCCCACAGUUUCGACCGCC | |
| | GAUGUGACAUCACCAACGCCCGCAGGUACAACCUCAGGC | |
| | GCCAGCCCAGUGACCCCUUCCCCAAGCCCCUGGGAUAAUG | |
| | GAACGGAGUCCAAGGCUCCUGAUAUGACUUCCUCUACCA | |
| | GCCCCGUGACUACACCCACUCCCAACGCAACUAGCCCAAC | |
| | CCCAGCUGUGACGACGCCCACCCCGAACGCGACAUCUCCC | |
| | ACACCUGCUGUGACAACCCCAACCCCUAACGCCACUAGCC | |
| | CUACCCUAGGUAAGACCAGUCCGACUAGCGCCGUUACAA | |
| | CCCCUACCCCUAACGCAACCGGCCCGACCGUGGGCGAGAC | |
| | UUCCCCGCAAGCCAAUGCGACAAAUCACACAUUGGGCGG | |
| | GACCUCUCCUACACCAGUCGUUACAUCUCAGCCUAAGAAC | |
| | GCUACCUCCGCUGUCACUACCGGACAGCACAACAUCACCA | |
| | GCUCAAGCACGAGUUCCAUGAGCUUGCGGCCGAGCUCAA | |
| | AUCCCGAGACCCUAUCACCAUCCACAUCAGACAACAGUAC | |
| | UUCACACAUGCCACUCUUGACGAGCGCUCACCCCACCGGC | |
| | GGCGAGAACAUCACCCAGGUGACUCCGGCGUCUAUUUCC | |
| | ACCCACCACGUCAGCACGUCGUCUCCCGCACCGAGACCAG | |
| | GGACGACUUCUCAGGCCAGUGGGCCUGGCAACUCCUCUA | |
| | CAAGCACAAAGCCAGGCGAAGUUAACGUGACAAAGGGAA | |
| | CGCCUCCCCAGAACGCAACCAGUCCUCAGGCGCCCAGCGG | |
| | GCAGAAGACUGCGGUGCCAACUGUGACCAGCACCGGUGG | |
| | CAAGGCCAACUCAACAACUGGAGGCAAGCAUACGACGGG | |
| | GCACGGCGCCCGGACCUCCACUGAACCCACGACCGAUUAC | |
| | GGAGGUGACAGCACAACACCGCGGCCACGAUAUAAUGCC | |
| | ACCACUUAUCUGCCACCAUCCACAAGCUCCAAGCUGCGGC | |
| | CACGGUGGACCUUCACAAGCCCACCCGUGACGACUGCCCA | |
| | AGCGACGGUGCCAGUGCCACCUACAAGCCAGCCACGCUUC | |
| | UCCAACCUUAGUAUGCUCGUUCUCCAGUGGGCCAGCCUU | |
| | GCUGUUCUGACCCUCCUCCUGCUGCUCGUGAUGGCCGAU | |
| | UGCGCCUUUAGGAGAAACCUCAGCACUAGCCACACGUAC | |
| | ACCACGCCGCCCUACGAUGACGCCGAGACUUACGUC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MEAALLVCQYTIQSLIHLTGEDPGFFNVEIPEFPFYPTCNVCTA | 70 |
| amino acid | DVNVTINFDVGGKKHQLDLDFGQLTPHTKAVYQPRGAFGGS | |
| sequence | ENAANLFLLELLGAGELALTMRSKKLPINVTTGEEQQVSLESV | |
| | DVYFQDVFGTMWCHHAEMQNPVYLIPETVPYIKWDNCNSAN | |
| | ITAVVRAQGLDVTLPLSLPTSAQDSNFAVKTEMLGNEIDIECI | |
| | MEDGEISQVLPGDNKFNIACSGYESHVPSGGILTSTSPVATPIP | |
| | GTGYAYSLRLTPRPVSRFLGNNSILYVFYSGNGPKASGGDYCI | |
| | QSNIVFSDEIPASQDMPTNTTDITYVGDNATYSVPMVTSEDAN | |
| | SPNVAVTAFWAWPNNTETDFKCKWTLTSGTPSGCENISGAFA | |
| | SNRTFDITVSGLGTAPKTLIITRTATNAATTTHKVIFSKAPESTT | |
| | TSPTLNTTGFADPNTTTGLPSSTHVPTNLTAPASTGPTVSTADV | |
| | TSPTPAGTTSGASPVTPSPSPWDNGTESKAPDMTSSTSPVTTPT | |
| | PNATSPTPAVTTPTPNATSPTPAVTTPTPNATSPTLGKTSPTSA | |
| | VTTPTPNATGPTVGETSPQANATNHTLGGTSPTPVVTSQPKNA | |
| | TSAVTTGQHNITSSSTSSMSLRPSSNPETLSPSTSDNSTSHMPLL | |
| | TSAHPTGGENITQVTPASISTHHVSTSSPAPRPGTTSQASGPGN | |
| | SSTSTKPGEVNVTKGTPPQNATSPQAPSGQKTAVPTVTSTGGK | |
| | ANSTTGGKHTTGHGARTSTEPTTDYGGDSTTPRPRYNATTYL | |
| | PPSTSSKLRPRWTFTSPPVTTAQATVPVPPTSQPRFSNLSMLVL | |
| | QWASLAVLTLLLLLVMADCAFRRNLSTSHTYTTPPYDDAETY | |
| | V | |
| PolyA tail | 100 nt | |

EBV Linked Construct 1 (1723942)

| SEQ ID NO: 212 consists of from 5′ end to 3′ end: | | 212 |
|---|---|---|
| 5′ UTR SEQ ID NO: 104, mRNA ORF SEQ ID NO: 213, and | | |
| 3′ UTR SEQ ID NO: 106. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGGUGAGCUUCAAGCAGGUGAGAGUGCCCCUGUUCACC | 213 |
| Construct | GCCAUCGCCCUGGUGAUCGUGCUGCUGCUGGCCUACUUCC | |
| (excluding | UGCCACCCAGAGUGAGAGGCGGCGGCAGAGUGGCCGCCG | |
| the stop | CCGCCAUCACCUGGGUGCCCAAGCCCAACGUGGAGGUGU | |
| codon) | GGCCCGUGGACCCGCCACCUCCCGUGAACUUCAACAAGAC | |
| | CGCCGAGCAGGAGUACGGCGACAAGGAGGUGAAGCUGCC | |
| | CCACUGGACGCCGACCCUGCACACCUUCCAGGUGCCCCAG | |
| | AACUACACCAAGGCCAACUGCACCUACUGCAACACCAGAG | |
| | AGUACACCUUCAGCUACAAGGGCUGCUGCUUCUACUUCA | |
| | CCAAGAAGAAGCACACCUGGAACGGCUGCUUCCAGGCCU | |
| | GCGCCGAGCUGUACCCCUGCACCUACUUCUACGGCCCCAC | |
| | CCCAGACAUCCUGCCCGUGGUGACCAGAAACCUGAACGCC | |
| | AUCGAGAGCCUGUGGGUGGGCGUGUACAGAGUGGGCGAG | |
| | GGCAACUGGACCAGCCUGGACGGCGGCACCUUCAAGGUG | |
| | UACCAGAUCUUCGGCAGCCACUGCACCUACGUGAGCAAG | |
| | UUCAGCACCGUGCCCGUGAGCCACCACGAGUGCAGCUUCC | |
| | UGAAGCCCUGCCUGUGCGUGAGCCAGAGAAGCAACAGCG | |
| | GCACUAGCGGCUCUAGCGGAUCAGGCAGCGGAGGGAGUG | |
| | GAGGCUCAGGAGGGACCAGCGGCAGCAGUGGCUCCGGAA | |
| | GCGGAGGCAGCCCCUGCUGCCACGUGACCCAGCUGAGAGC | |
| | CCAGCACCUGCUGGCCCUGGAGAACAUCAGCGACAUCUAC | |
| | CUGGUGAGCAACCAGACCUGCGACGGCUUCAGCCUGGCC | |
| | AGCCUGAACAGCCCCAAGAACGGCAGCAACCAGCUGGUG | |
| | AUCAGCAGAUGCGCCAACGGCCUGAACGUGGUGAGCUUC | |
| | UUCAUCAGCAUCCUGAAGAGAAGCAGCAGCGCCCUGACC | |
| | GGCCACCUGAGAGAGCUGCUGACCACCCUGGAGACCCUG | |
| | UACGGCAGCUUCAGCGUGGAGGACCUGUUCGGCGCCAAC | |
| | CUGAACAGAUACGCCUGGCACAGAGGCGGCGGAGGAGGU | |
| | GGCUCCGGUGGAGGUGGCAGCGGAGGAGGAGGCAGUGGU | |
| | GGCGGAGGCAGCCUUAGCGAGGUGAAGCUCCACUUGGAC | |
| | AUCGAGGGCCACGCCAGCCACUACACCAUCCCCUGGACCG | |
| | AGCUCAUGGCCAAGGUGCCCGGCCUUAGCCCCGAGGCCCU | |
| | GUGGCGGGAGGCCAACGUGACCGAGGACCUGGCCAGCAU | |
| | GCUGAACCGGUACAAGCUGAUCUACAAGACCAGCGGCAC | |
| | CCUGGGCAUCGCCCUGGCCGAGCCCGUGGACAUCCCCGCC | |
| | GUUAGCGAAGGCAGCAUGCAGGUGGACGCCAGCAAGGUG | |
| | CACCCCGGCGUGAUCAGCGGCCUGAACAGCCCCGCCUGUA | |
| | UGUUGAGCGCCCCACUGGAGAAGCAGCUGUUCUACUACA | |
| | UCGGCACCAUGCUGCCCAACACCCGGCCCCACAGCUACGU | |
| | GUUCUACCAGCUGCGGUGCCACCUGAGCUACGUUGCCCU | |
| | GAGCAUCAACGGCGACAAGUUCCAGUACACCGGCGCCAU | |
| | GACCAGCAAGUUCCUGAUGGGCACCUACAAGCGGGUCAC | |
| | CGAGAAGGGCGACGAGCACGUGCUGUCACUGGUGUUCGG | |
| | CAAGACCAAGGACCUGCCCGACCUGCGGGGCCCCUUCAGC | |
| | UACCCUAGUUUGACCAGCGCCCAGAGCGGCGACUACAGC | |
| | UUGGUGAUCGUGACCACCUUCGUGCACUACGCCAACUUC | |
| | CACAACUACUUCGUGCCCAACCUGAAGGACAUGUUCAGC | |
| | CGGGCCGUGACCAUGACUGCCGCUUCUUACGCCCGGUACG | |
| | UGCUGCAGAAGCUGGUCCUGCUGGAGAUGAAGGGCGGCU | |
| | GCCGGGAGCCCGAGCUGGACACCGAAACACUGACCACCAU | |
| | GUUCGAGGUGAGCGUGGCCUUCUUCAAGGUGGGUCACGC | |
| | GGUGGGCGAAACCGGCAACGGCUGCGUGGACUUACGCUG | |
| | GCUGGCCAAGAGCUUCUUCGAGCUGACCGUGCUGAAGGA | |
| | UAUCAUCGGCAUCUGCUACGGCGCCACCGUGAAGGGCAU | |
| | GCAGAGCUACGGCCUGGAGCGGCUGGCCGCCAUGCUUAU | |
| | GGCAACAGUGAAGAUGGAGGAGCUGGGACACCUGACAAC | |
| | AGAGAAGCAGGAGUACGCCCUGAGACUGGCCACAGUGGG | |
| | CUACCCAAAGGCCGGCGUGUACAGUGGACUGAUCGGCGG | |
| | CGCAACCAGCGUGCUGCUAUCCGCUUACAACCGGCACCCG | |
| | CUGUUCCAGCCCCUGCACACCGUGAUGCGGGAAACCCUGU | |
| | UCAUCGGAAGCCACGUCGUGCUGCGGGAGCUGAGGCUGA | |
| | ACGUAACCACCCAGGGCCCUAAUCUGGCCCUGUAUCAGCU | |
| | CCUCAGUACCGCCCUGUGCAGCGCCCUUGAGAUCGGCGAG | |
| | GUGCUCAGAGGCCUGGCCCUCGGUACCGAGAGCGGCCUC | |
| | UUCAGCCCAUGCUACUUAAGCCUGCGGUUCGACCUGACCC | |
| | GGGACAAGUUGCUGAGCAUGGCCCCGCAGGAGGCCACAC | |
| | UGGACCAGGCAGCUGUAUCCAACGCCGUGGACGGCUUCC | |
| | UGGGCAGACUGUCCCUGGAACGGGAGGACCGGGACGCCU | |
| | GGCACCUGCCUGCCUACAAGUGUGUGGAUCGGCUGGACA | |
| | AGGUGCUGAUGAUCAUCCCUCUGAUUAAUGUCACCUUCA | |
| | UCAUCAGCAGCGACCGGGAGGUGCGGGGAUCCGCCCUCU | |
| | ACGAGGCCAGCACCACCUAUCUGAGCAGCAGCCUGUUCCU | |
| | GUCUCCUGUGAUCAUGAACAAGUGCAGCCAGGGCGCCGU | |
| | GGCCGGCGAGCCCCGGCAGAUCCCCAAGAUCCAGAACUUC | |
| | ACCCGGACCCAGAAGUCUUGCAUCUUCUGCGGCUUCGCCC | |
| | UUUUGUCCUACGACGAGAAGGAGGGCUUGGAGACUACAA | |
| | CCUACAUCACCAGCCAGGAGGUGCAGAACAGCAUCCUGU | |
| | CAUCUAAUUACUUCGACUUCGACAACCUGCACGUUCAUU | |
| | ACCUGCUCCUCACCACCAACGGUACCGUCAUGGAAAUCGC | |
| | CGGACUGUACGAGGAGCGGGCCCAUGUUGUGCUGGCCAU | |
| | CAUCCUGUACUUCAUCGCUUUCGCACUUGGCAUCUUCCU | |
| | GGUGCACAAGAUCGUGAUGUUCUUCCUG | |
| 3′ UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MVSFKQVRVPLFTAIALVIVLLLAYFLPPRVRGGGRVAAAAIT | 214 |
| amino acid | WVPKPNVEVWPVDPPPPVNFNKTAEQEYGDKEVKLPHWTPT | |
| sequence | LHTFQVPQNYTKANCTYCNTREYTFSYKGCCFYFTKKKHTW | |
| | NGCFQACAELYPCTYFYGPTPDILPVVTRNLNAIESLWVGVYR | |
| | VGEGNWTSLDGGTFKVYQIFGSHCTYVSKFSTVPVSHHECSFL | |
| | KPCLCVSQRSNSGTSGSSGSGSGGSGGSGGTSGSSGSGSGGSP | |
| | CCHVTQLRAQHLLALENISDIYLVSNQTCDGFSLASLNSPKNG | |
| | SNQLVISRCANGLNVVSFFISILKRSSSALTGHLRELLTTLETLY | |
| | GSFSVEDLFGANLNRYAWHRGGGGGGSGGGGSGGGGSGGG | |
| | GSLSEVKLHLDIEGHASHYTIPWTELMAKVPGLSPEALWREA | |
| | NVTEDLASMLNRYKLIYKTSGTLGIALAEPVDIPAVSEGSMQV | |
| | DASKVHPGVISGLNSPACMLSAPLEKQLFYYIGTMLPNTRPHS | |
| | YVFYQLRCHLSYVALSINGDKFQYTGAMTSKFLMGTYKRVT | |
| | EKGDEHVLSLVFGKTKDLPDLRGPFSYPSLTSAQSGDYSLVIV | |
| | TTFVHYANFHNYFVPNLKDMFSRAVTMTAASYARYVLQKLV | |
| | LLEMKGGCREPELDTETLTTMFEVSVAFFKVGHAVGETGNGC | |
| | VDLRWLAKSFFELTVLKDIIGICYGATVKGMQSYGLERLAAM | |
| | LMATVKMEELGHLTTEKQEYALRLATVGYPKAGVYSGLIGG | |
| | ATSVLLSAYNRHPLFQPLHTVMRETLFIGSHVVLRELRLNVTT | |
| | QGPNLALYQLLSTALCSALEIGEVLRGLALGTESGLFSPCYLSL | |
| | RFDLTRDKLLSMAPQEATLDQAAVSNAVDGFLGRLSLEREDR | |
| | DAWHLPAYKCVDRLDKVLMIIPLINVTFIISSDREVRGSALYE | |
| | ASTTYLSSSLFLSPVIMNKCSQGAVAGEPRQIPKIQNFTRTQKS | |
| | CIFCGFALLSYDEKEGLETTTYITSQEVQNSILSSNYFDFDNLH | |
| | VHYLLLTTNGTVMEIAGLYEERAHVVLAIILYFIAFALGIFLVH | |
| | KIVMFFL | |
| PolyA tail | 100 nt | |

EBV Linked Construct 2 (1723941)

| SEQ ID NO: 215 consists of from 5′ end to 3′ end: | | 215 |
|---|---|---|
| 5′ UTR SEQ ID NO: 104, mRNA ORF SEQ ID NO: 216, and | | |
| 3′ UTR SEQ ID NO: 106. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGGUGAGCUUCAAGCAGGUGAGAGUGCCCCUGUUCACC | 216 |
| Construct | GCCAUCGCCCUGGUGAUCGUGCUGCUGCUGGCCUACUUCC | |
| (excluding | UGCCACCCAGAGUGAGAGGCGGCGGCAGAGUGGCCGCCG | |
| the stop | CCGCCAUCACCUGGGUGCCCAAGCCCAACGUGGAGGUGU | |
| codon) | GGCCCGUGGACCCGCCACCUCCCGUGAACUUCAACAAGAC | |
| | CGCCGAGCAGGAGUACGGCGACAAGGAGGUGAAGCUGCC | |
| | CCACUGGACGCCGACCCUGCACACCUUCCAGGUGCCCCAG | |
| | AACUACACCAAGGCCAACUGCACCUACUGCAACACCAGAG | |
| | AGUACACCUUCAGCUACAAGGGCUGCUGCUUCUACUUCA | |
| | CCAAGAAGAAGCACACCUGGAACGGCUGCUUCCAGGCCU | |
| | GCGCCGAGCUGUACCCCUGCACCUACUUCUACGGCCCCAC | |
| | CCCAGACAUCCUGCCCGUGGUGACCAGAAACCUGAACGCC | |
| | AUCGAGAGCCUGUGGGUGGGCGUGUACAGAGUGGGCGAG | |
| | GGCAACUGGACCAGCCUGGACGGCGGCACCUUCAAGGUG | |
| | UACCAGAUCUUCGGCAGCCACUGCACCUACGUGAGCAAG | |
| | UUCAGCACCGUGCCCGUGAGCCACCACGAGUGCAGCUUCC | |
| | UGAAGCCCUGCCUGUGCGUGAGCCAGAGAAGCAACAGCG | |
| | GAGGAGGAGGAUCUGGAGGCGGCGGAAGUGGUGGAGGAG | |
| | GAAGCGGAGGAGGUGGCAGCGGAGGUGGUGGAAGUGGCG | |
| | GCGGAGGAAGCCCCUGCUGCCACGUGACCCAGCUGAGAG | |
| | CCCAGCACCUGCUGGCCCUGGAGAACAUCAGCGACAUCUA | |
| | CCUGGUGAGCAACCAGACCUGCGACGGCUUCAGCCUGGCC | |
| | AGCCUGAACAGCCCCAAGAACGGCAGCAACCAGCUGGUG | |
| | AUCAGCAGAUGCGCCAACGGCCUGAACGUGGUGAGCUUC | |
| | UUCAUCAGCAUCCUGAAGAGAAGCAGCAGCGCCCUGACC | |
| | GGCCACCUGAGAGAGCUGCUGACCACCCUGGAGACCCUG | |
| | UACGGCAGCUUCAGCGUGGAGGACCUGUUCGGCGCCAAC | |
| | CUGAACAGAUACGCCUGGCACAGAGGCGGCGGAGGAGGU | |
| | GGCUCCGGUGGAGGUGGCAGCGGAGGAGGAGGCAGUGGU | |
| | GGCGGAGGCAGCCUUAGCGAGGUGAAGCUCCACUUGGAC | |
| | AUCGAGGGCCACGCCAGCCACUACACCAUCCCCUGGACCG | |
| | AGCUCAUGGCCAAGGUGCCCGGCCUUAGCCCCGAGGCCCU | |
| | GUGGCGGGAGGCCAACGUGACCGAGGACCUGGCCAGCAU | |
| | GCUGAACCGGUACAAGCUGAUCUACAAGACCAGCGGCAC | |
| | CCUGGGCAUCGCCCUGGCCGAGCCCGUGGACAUCCCCGCC | |
| | GUUAGCGAAGGCAGCAUGCAGGUGGACGCCAGCAAGGUG | |
| | CACCCCGGCGUGAUCAGCGGCCUGAACAGCCCCGCCUGUA | |
| | UGUUGAGCGCCCCACUGGAGAAGCAGCUGUUCUACUACA | |
| | UCGGCACCAUGCUGCCCAACACCCGGCCCCACAGCUACGU | |
| | GUUCUACCAGCUGCGGUGCCACCUGAGCUACGUUGCCCU | |
| | GAGCAUCAACGGCGACAAGUUCCAGUACACCGGCGCCAU | |
| | GACCAGCAAGUUCCUGAUGGGCACCUACAAGCGGGUCAC | |
| | CGAGAAGGGCGACGAGCACGUGCUGUCACUGGUGUUCGG | |
| | CAAGACCAAGGACCUGCCCGACCUGCGGGGCCCCUUCAGC | |
| | UACCCUAGUUUGACCAGCGCCCAGAGCGGCGACUACAGC | |
| | UUGGUGAUCGUGACCACCUUCGUGCACUACGCCAACUUC | |
| | CACAACUACUUCGUGCCCAACCUGAAGGACAUGUUCAGC | |
| | CGGGCCGUGACCAUGACUGCCGCUUCUUACGCCCGGUACG | |
| | UGCUGCAGAAGCUGGUCCUGCUGGAGAUGAAGGGCGGCU | |
| | GCCGGGAGCCCGAGCUGGACACCGAAACACUGACCACCAU | |
| | GUUCGAGGUGAGCGUGGCCUUCUUCAAGGUGGGUCACGC | |
| | GGUGGGCGAAACCGGCAACGGCUGCGUGGACUUACGCUG | |
| | GCUGGCCAAGAGCUUCUUCGAGCUGACCGUGCUGAAGGA | |
| | UAUCAUCGGCAUCUGCUACGGCGCCACCGUGAAGGGCAU | |
| | GCAGAGCUACGGCCUGGAGCGGCUGGCCGCCAUGCUUAU | |
| | GGCAACAGUGAAGAUGGAGGAGCUGGGACACCUGACAAC | |
| | AGAGAAGCAGGAGUACGCCCUGAGACUGGCCACAGUGGG | |
| | CUACCCAAAGGCCGGCGUGUACAGUGGACUGAUCGGCGG | |
| | CGCAACCAGCGUGCUGCUAUCCGCUUACAACCGGCACCCG | |
| | CUGUUCCAGCCCCUGCACACCGUGAUGCGGGAAACCCUGU | |
| | UCAUCGGAAGCCACGUCGUGCUGCGGGAGCUGAGGCUGA | |
| | ACGUAACCACCCAGGGCCCUAAUCUGGCCCUGUAUCAGCU | |
| | CCUCAGUACCGCCCUGUGCAGCGCCCUUGAGAUCGGCGAG | |
| | GUGCUCAGAGGCCUGGCCCUCGGUACCGAGAGCGGCCUC | |
| | UUCAGCCCAUGCUACUUAAGCCUGCGGUUCGACCUGACCC | |
| | GGGACAAGUUGCUGAGCAUGGCCCCGCAGGAGGCCACAC | |
| | UGGACCAGGCAGCUGUAUCCAACGCCGUGGACGGCUUCC | |
| | UGGGCAGACUGUCCCUGGAACGGGAGGACCGGGACGCCU | |
| | GGCACCUGCCUGCCUACAAGUGUGUGGAUCGGCUGGACA | |
| | AGGUGCUGAUGAUCAUCCCUCUGAUUAAUGUCACCUUCA | |
| | UCAUCAGCAGCGACCGGGAGGUGCGGGGAUCCGCCCUCU | |
| | ACGAGGCCAGCACCACCUAUCUGAGCAGCAGCCUGUUCCU | |
| | GUCUCCUGUGAUCAUGAACAAGUGCAGCCAGGGCGCCGU | |
| | GGCCGGCGAGCCCCGGCAGAUCCCCAAGAUCCAGAACUUC | |
| | ACCCGGACCCAGAAGUCUUGCAUCUUCUGCGGCUUCGCCC | |
| | UUUUGUCCUACGACGAGAAGGAGGGCUUGGAGACUACAA | |
| | CCUACAUCACCAGCCAGGAGGUGCAGAACAGCAUCCUGU | |
| | CAUCUAAUUACUUCGACUUCGACAACCUGCACGUUCAUU | |
| | ACCUGCUCCUCACCACCAACGGUACCGUCAUGGAAAUCGC | |
| | CGGACUGUACGAGGAGCGGGCCCAUGUUGUGCUGGCCAU | |
| | CAUCCUGUACUUCAUCGCUUUCGCACUUGGCAUCUUCCU | |
| | GGUGCACAAGAUCGUGAUGUUCUUCCUG | |
| 3′ UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MVSFKQVRVPLFTAIALVIVLLLAYFLPPRVRGGGRVAAAAIT | 217 |
| amino acid | WVPKPNVEVWPVDPPPPVNFNKTAEQEYGDKEVKLPHWTPT | |
| sequence | LHTFQVPQNYTKANCTYCNTREYTFSYKGCCFYFTKKKHTW | |
| | NGCFQACAELYPCTYFYGPTPDILPVVTRNLNAIESLWVGVYR | |
| | VGEGNWTSLDGGTFKVYQIFGSHCTYVSKFSTVPVSHHECSFL | |
| | KPCLCVSQRSNSGGGGSGGGGSGGGGSGGGGSGGGGSGGGG | |
| | SPCCHVTQLRAQHLLALENISDIYLVSNQTCDGFSLASLNSPK | |
| | NGSNQLVISRCANGLNVVSFFISILKRSSSALTGHLRELLTTLET | |
| | LYGSFSVEDLFGANLNRYAWHRGGGGGGSGGGGSGGGGSG | |
| | GGGSLSEVKLHLDIEGHASHYTIPWTELMAKVPGLSPEALWR | |
| | EANVTEDLASMLNRYKLIYKTSGTLGIALAEPVDIPAVSEGSM | |
| | QVDASKVHPGVISGLNSPACMLSAPLEKQLFYYIGTMLPNTRP | |
| | HSYVFYQLRCHLSYVALSINGDKFQYTGAMTSKFLMGTYKR | |
| | VTEKGDEHVLSLVFGKTKDLPDLRGPFSYPSLTSAQSGDYSLV | |
| | IVTTFVHYANFHNYFVPNLKDMFSRAVTMTAASYARYVLQK | |
| | LVLLEMKGGCREPELDTETLTTMFEVSVAFFKVGHAVGETGN | |
| | GCVDLRWLAKSFFELTVLKDIIGICYGATVKGMQSYGLERLA | |
| | AMLMATVKMEELGHLTTEKQEYALRLATVGYPKAGVYSGLI | |
| | GGATSVLLSAYNRHPLFQPLHTVMRETLFIGSHVVLRELRLNV | |
| | TTQGPNLALYQLLSTALCSALEIGEVLRGLALGTESGLFSPCYL | |
| | SLRFDLTRDKLLSMAPQEATLDQAAVSNAVDGFLGRLSLERE | |
| | DRDAWHLPAYKCVDRLDKVLMIIPLINVTFIISSDREVRGSAL | |
| | YEASTTYLSSSLFLSPVIMNKCSQGAVAGEPRQIPKIQNFTRTQ | |
| | KSCIFCGFALLSYDEKEGLETTTYITSQEVQNSILSSNYFDFDN | |
| | LHVHYLLLTTNGTVMEIAGLYEERAHVVLAIILYFIAFALGIFL | |
| | VHKIVMFFL | |
| PolyA tail | 100 nt | |

EBV Linked Construct 3 (1723940)

| SEQ ID NO: 218 consists of from 5′ end to 3′ end: | | 218 |
|---|---|---|
| 5′ UTR SEQ ID NO: 104, mRNA ORF SEQ ID NO: 219, and | | |
| 3′ UTR SEQ ID NO: 106. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGAGAGCCGUGGGCGUGUUCCUGGCCAUCUGCCUGGUG | 219 |
| Construct | ACCAUCUUCGUGCUGCCCACCUGGGGCAACUGGGCCUACC | |
| (excluding | CCUGCUGCCACGUGACCCAGCUGAGAGCCCAGCACCUGCU | |
| the stop | GGCCCUGGAGAACAUCAGCGACAUCUACCUGGUGAGCAA | |
| codon) | CCAGACCUGCGACGGCUUCAGCCUGGCCAGCCUGAACAGC | |
| | CCCAAGAACGGCAGCAACCAGCUGGUGAUCAGCAGAUGC | |
| | GCCAACGGCCUGAACGUGGUGAGCUUCUUCAUCAGCAUC | |
| | CUGAAGAGAAGCAGCAGCGCCCUGACCGGCCACCUGAGA | |
| | GAGCUGCUGACCACCCUGGAGACCCUGUACGGCAGCUUC | |
| | AGCGUGGAGGACCUGUUCGGCGCCAACCUGAACAGAUAC | |
| | GCCUGGCACAGAGGCGGCGGAGGAGGUGGCAGCGGAGGA | |
| | GGAGGGUCUGGAGGUGGCGGCAGCCUUAGCGAGGUGAAG | |
| | CUCCACUUGGACAUCGAGGGCCACGCCAGCCACUACACCA | |
| | UCCCCUGGACCGAGCUCAUGGCCAAGGUGCCCGGCCUUAG | |
| | CCCCGAGGCCCUGUGGCGGGAGGCCAACGUGACCGAGGA | |
| | CCUGGCCAGCAUGCUGAACCGGUACAAGCUGAUCUACAA | |
| | GACCAGCGGCACCCUGGGCAUCGCCCUGGCCGAGCCCGUG | |
| | GACAUCCCCGCCGUUAGCGAAGGCAGCAUGCAGGUGGAC | |
| | GCCAGCAAGGUGCACCCCGGCGUGAUCAGCGGCCUGAAC | |
| | AGCCCCGCCUGUAUGUUGAGCGCCCCACUGGAGAAGCAG | |
| | CUGUUCUACUACAUCGGCACCAUGCUGCCCAACACCCGGC | |
| | CCCACAGCUACGUGUUCUACCAGCUGCGGUGCCACCUGAG | |
| | CUACGUUGCCCUGAGCAUCAACGGCGACAAGUUCCAGUA | |
| | CACCGGCGCCAUGACCAGCAAGUUCCUGAUGGGCACCUAC | |
| | AAGCGGGUCACCGAGAAGGGCGACGAGCACGUGCUGUCA | |
| | CUGGUGUUCGGCAAGACCAAGGACCUGCCCGACCUGCGG | |
| | GGCCCCUUCAGCUACCCUAGUUUGACCAGCGCCCAGAGCG | |
| | GCGACUACAGCUUGGUGAUCGUGACCACCUUCGUGCACU | |
| | ACGCCAACUUCCACAACUACUUCGUGCCCAACCUGAAGGA | |
| | CAUGUUCAGCCGGGCCGUGACCAUGACUGCCGCUUCUUA | |
| | CGCCCGGUACGUGCUGCAGAAGCUGGUCCUGCUGGAGAU | |
| | GAAGGGCGGCUGCCGGGAGCCCGAGCUGGACACCGAAAC | |
| | ACUGACCACCAUGUUCGAGGUGAGCGUGGCCUUCUUCAA | |
| | GGUGGGUCACGCGGUGGGCGAAACCGGCAACGGCUGCGU | |
| | GGACUUACGCUGGCUGGCCAAGAGCUUCUUCGAGCUGAC | |
| | CGUGCUGAAGGAUAUCAUCGGCAUCUGCUACGGCGCCAC | |
| | CGUGAAGGGCAUGCAGAGCUACGGCCUGGAGCGGCUGGC | |
| | CGCCAUGCUUAUGGCAACAGUGAAGAUGGAGGAGCUGGG | |
| | ACACCUGACAACAGAGAAGCAGGAGUACGCCCUGAGACU | |
| | GGCCACAGUGGGCUACCCAAAGGCCGGCGUGUACAGUGG | |
| | ACUGAUCGGCGGCGCAACCAGCGUGCUGCUAUCCGCUUA | |
| | CAACCGGCACCCGCUGUUCCAGCCCCUGCACACCGUGAUG | |
| | CGGGAAACCCUGUUCAUCGGAAGCCACGUCGUGCUGCGG | |
| | GAGCUGAGGCUGAACGUAACCACCCAGGGCCCUAAUCUG | |
| | GCCCUGUAUCAGCUCCUCAGUACCGCCCUGUGCAGCGCCC | |
| | UUGAGAUCGGCGAGGUGCUCAGAGGCCUGGCCCUCGGUA | |
| | CCGAGAGCGGCCUCUUCAGCCCAUGCUACUUAAGCCUGCG | |
| | GUUCGACCUGACCCGGGACAAGUUGCUGAGCAUGGCCCC | |
| | GCAGGAGGCCACACUGGACCAGGCAGCUGUAUCCAACGC | |
| | CGUGGACGGCUUCCUGGGCAGACUGUCCCUGGAACGGGA | |
| | GGACCGGGACGCCUGGCACCUGCCUGCCUACAAGUGUGU | |
| | GGAUCGGCUGGACAAGGUGCUGAUGAUCAUCCCUCUGAU | |
| | UAAUGUCACCUUCAUCAUCAGCAGCGACCGGGAGGUGCG | |
| | GGGAUCCGCCCUCUACGAGGCCAGCACCACCUAUCUGAGC | |
| | AGCAGCCUGUUCCUGUCUCCUGUGAUCAUGAACAAGUGC | |
| | AGCCAGGGCGCCGUGGCCGGCGAGCCCCGGCAGAUCCCCA | |
| | AGAUCCAGAACUUCACCCGGACCCAGAAGUCUUGCAUCU | |
| | UCUGCGGCUUCGCCCUUUUGUCCUACGACGAGAAGGAGG | |
| | GCUUGGAGACUACAACCUACAUCACCAGCCAGGAGGUGC | |
| | AGAACAGCAUCCUGUCAUCUAAUUACUUCGACUUCGACA | |
| | ACCUGCACGUUCAUUACCUGCUCCUCACCACCAACGGUAC | |
| | CGUCAUGGAAAUCGCCGGACUGUACGAGGAGCGGGCCCA | |
| | UGUUGUGCUGGCCAUCAUCCUGUACUUCAUCGCUUUCGC | |
| | ACUUGGCAUCUUCCUGGUGCACAAGAUCGUGAUGUUCUU | |
| | CCUG | |
| 3′UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MRAVGVFLAICLVTIFVLPTWGNWAYPCCHVTQLRAQHLLA | 220 |
| amino acid | LENISDIYLVSNQTCDGFSLASLNSPKNGSNQLVISRCANGLNV | |
| sequence | VSFFISILKRSSSALTGHLRELLTTLETLYGSFSVEDLFGANLNR | |
| | YAWHRGGGGGGSGGGGSGGGGSLSEVKLHLDIEGHASHYTIP | |
| | WTELMAKVPGLSPEALWREANVTEDLASMLNRYKLIYKTSG | |
| | TLGIALAEPVDIPAVSEGSMQVDASKVHPGVISGLNSPACMLS | |
| | APLEKQLFYYIGTMLPNTRPHSYVFYQLRCHLSYVALSINGDK | |
| | FQYTGAMTSKFLMGTYKRVTEKGDEHVLSLVFGKTKDLPDL | |
| | RGPFSYPSLTSAQSGDYSLVIVTTFVHYANFHNYFVPNLKDMF | |
| | SRAVTMTAASYARYVLQKLVLLEMKGGCREPELDTETLTTM | |
| | FEVSVAFFKVGHAVGETGNGCVDLRWLAKSFFELTVLKDIIGI | |
| | CYGATVKGMQSYGLERLAAMLMATVKMEELGHLTTEKQEY | |
| | ALRLATVGYPKAGVYSGLIGGATSVLLSAYNRHPLFQPLHTV | |
| | MRETLFIGSHVVLRELRLNVTTQGPNLALYQLLSTALCSALEI | |
| | GEVLRGLALGTESGLFSPCYLSLRFDLTRDKLLSMAPQEATLD | |
| | QAAVSNAVDGFLGRLSLEREDRDAWHLPAYKCVDRLDKVL | |
| | MIIPLINVTFIISSDREVRGSALYEASTTYLSSSLFLSPVIMNKCS | |
| | QGAVAGEPRQIPKIQNFTRTQKSCIFCGFALLSYDEKEGLETTT | |
| | YITSQEVQNSILSSNYFDFDNLHVHYLLLTTNGTVMEIAGLYE | |
| | ERAHVVLAIILYFIAFALGIFLVHKIVMFFL | |
| PolyA tail | 100 nt | |

EBV Linked Construct 4 (1723939)

| SEQ ID NO: 221 consists of from 5′ end to 3′ end: | | 221 |
|---|---|---|
| 5′ UTR SEQ ID NO: 104, mRNA ORF SEQ ID NO: 222, and | | |
| 3′ UTR SEQ ID NO: 106. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGAGAGCCGUGGGCGUGUUCCUGGCCAUCUGCCUGGUG | 222 |
| Construct | ACCAUCUUCGUGCUGCCCACCUGGGGCAACUGGGCCUACC | |
| (excluding | CCUGCUGCCACGUGACCCAGCUGAGAGCCCAGCACCUGCU | |
| the stop | GGCCCUGGAGAACAUCAGCGACAUCUACCUGGUGAGCAA | |
| codon) | CCAGACCUGCGACGGCUUCAGCCUGGCCAGCCUGAACAGC | |
| | CCCAAGAACGGCAGCAACCAGCUGGUGAUCAGCAGAUGC | |
| | GCCAACGGCCUGAACGUGGUGAGCUUCUUCAUCAGCAUC | |
| | CUGAAGAGAAGCAGCAGCGCCCUGACCGGCCACCUGAGA | |
| | GAGCUGCUGACCACCCUGGAGACCCUGUACGGCAGCUUC | |
| | AGCGUGGAGGACCUGUUCGGCGCCAACCUGAACAGAUAC | |
| | GCCUGGCACAGAGGCGGCGGAGGAGGUGGCUCCGGUGGA | |
| | GGUGGCAGCGGAGGAGGAGGCAGUGGUGGCGGAGGCAGC | |
| | CUUAGCGAGGUGAAGCUCCACUUGGACAUCGAGGGCCAC | |
| | GCCAGCCACUACACCAUCCCCUGGACCGAGCUCAUGGCCA | |
| | AGGUGCCCGGCCUUAGCCCCGAGGCCCUGUGGCGGGAGG | |
| | CCAACGUGACCGAGGACCUGGCCAGCAUGCUGAACCGGU | |
| | ACAAGCUGAUCUACAAGACCAGCGGCACCCUGGGCAUCG | |
| | CCCUGGCCGAGCCCGUGGACAUCCCCGCCGUUAGCGAAGG | |
| | CAGCAUGCAGGUGGACGCCAGCAAGGUGCACCCCGGCGU | |
| | GAUCAGCGGCCUGAACAGCCCCGCCUGUAUGUUGAGCGC | |
| | CCCACUGGAGAAGCAGCUGUUCUACUACAUCGGCACCAU | |
| | GCUGCCCAACACCCGGCCCCACAGCUACGUGUUCUACCAG | |
| | CUGCGGUGCCACCUGAGCUACGUUGCCCUGAGCAUCAAC | |
| | GGCGACAAGUUCCAGUACACCGGCGCCAUGACCAGCAAG | |
| | UUCCUGAUGGGCACCUACAAGCGGGUCACCGAGAAGGGC | |
| | GACGAGCACGUGCUGUCACUGGUGUUCGGCAAGACCAAG | |
| | GACCUGCCCGACCUGCGGGGCCCCUUCAGCUACCCUAGUU | |
| | UGACCAGCGCCCAGAGCGGCGACUACAGCUUGGUGAUCG | |
| | UGACCACCUUCGUGCACUACGCCAACUUCCACAACUACUU | |
| | CGUGCCCAACCUGAAGGACAUGUUCAGCCGGGCCGUGAC | |
| | CAUGACUGCCGCUUCUUACGCCCGGUACGUGCUGCAGAA | |
| | GCUGGUCCUGCUGGAGAUGAAGGGCGGCUGCCGGGAGCC | |
| | CGAGCUGGACACCGAAACACUGACCACCAUGUUCGAGGU | |
| | GAGCGUGGCCUUCUUCAAGGUGGGUCACGCGGUGGGCGA | |
| | AACCGGCAACGGCUGCGUGGACUUACGCUGGCUGGCCAA | |
| | GAGCUUCUUCGAGCUGACCGUGCUGAAGGAUAUCAUCGG | |
| | CAUCUGCUACGGCGCCACCGUGAAGGGCAUGCAGAGCUA | |
| | CGGCCUGGAGCGGCUGGCCGCCAUGCUUAUGGCAACAGU | |
| | GAAGAUGGAGGAGCUGGGACACCUGACAACAGAGAAGCA | |
| | GGAGUACGCCCUGAGACUGGCCACAGUGGGCUACCCAAA | |
| | GGCCGGCGUGUACAGUGGACUGAUCGGCGGCGCAACCAG | |
| | CGUGCUGCUAUCCGCUUACAACCGGCACCCGCUGUUCCAG | |
| | CCCCUGCACACCGUGAUGCGGGAAACCCUGUUCAUCGGA | |
| | AGCCACGUCGUGCUGCGGGAGCUGAGGCUGAACGUAACC | |
| | ACCCAGGGCCCUAAUCUGGCCCUGUAUCAGCUCCUCAGUA | |
| | CCGCCCUGUGCAGCGCCCUUGAGAUCGGCGAGGUGCUCA | |
| | GAGGCCUGGCCCUCGGUACCGAGAGCGGCCUCUUCAGCCC | |
| | AUGCUACUUAAGCCUGCGGUUCGACCUGACCCGGGACAA | |
| | GUUGCUGAGCAUGGCCCCGCAGGAGGCCACACUGGACCA | |
| | GGCAGCUGUAUCCAACGCCGUGGACGGCUUCCUGGGCAG | |
| | ACUGUCCCUGGAACGGGAGGACCGGGACGCCUGGCACCU | |
| | GCCUGCCUACAAGUGUGUGGAUCGGCUGGACAAGGUGCU | |
| | GAUGAUCAUCCCUCUGAUUAAUGUCACCUUCAUCAUCAG | |
| | CAGCGACCGGGAGGUGCGGGGAUCCGCCCUCUACGAGGC | |
| | CAGCACCACCUAUCUGAGCAGCAGCCUGUUCCUGUCUCCU | |
| | GUGAUCAUGAACAAGUGCAGCCAGGGCGCCGUGGCCGGC | |
| | GAGCCCCGGCAGAUCCCCAAGAUCCAGAACUUCACCCGGA | |
| | CCCAGAAGUCUUGCAUCUUCUGCGGCUUCGCCCUUUUGU | |
| | CCUACGACGAGAAGGAGGGCUUGGAGACUACAACCUACA | |
| | UCACCAGCCAGGAGGUGCAGAACAGCAUCCUGUCAUCUA | |
| | AUUACUUCGACUUCGACAACCUGCACGUUCAUUACCUGC | |
| | UCCUCACCACCAACGGUACCGUCAUGGAAAUCGCCGGACU | |
| | GUACGAGGAGCGGGCCCAUGUUGUGCUGGCCAUCAUCCU | |
| | GUACUUCAUCGCUUUCGCACUUGGCAUCUUCCUGGUGCA | |
| | CAAGAUCGUGAUGUUCUUCCUG | |
| 3′ UTR | UGAUAAUAGGCUGGAGCCUCGGUGGCCUAGCUUCUUGCC | 106 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MRAVGVFLAICLVTIFVLPTWGNWAYPCCHVTQLRAQHLLA | 223 |
| amino acid | LENISDIYLVSNQTCDGFSLASLNSPKNGSNQLVISRCANGLNV | |
| sequence | VSFFISILKRSSSALTGHLRELLTTLETLYGSFSVEDLFGANLNR | |
| | YAWHRGGGGGGSGGGGSGGGGSGGGGSLSEVKLHLDIEGHA | |
| | SHYTIPWTELMAKVPGLSPEALWREANVTEDLASMLNRYKLI | |
| | YKTSGTLGIALAEPVDIPAVSEGSMQVDASKVHPGVISGLNSP | |
| | ACMLSAPLEKQLFYYIGTMLPNTRPHSYVFYQLRCHLSYVAL | |
| | SINGDKFQYTGAMTSKFLMGTYKRVTEKGDEHVLSLVFGKT | |
| | KDLPDLRGPFSYPSLTSAQSGDYSLVIVTTFVHYANFHNYFVP | |
| | NLKDMFSRAVTMTAASYARYVLQKLVLLEMKGGCREPELDT | |
| | ETLTTMFEVSVAFFKVGHAVGETGNGCVDLRWLAKSFFELTV | |
| | LKDIIGICYGATVKGMQSYGLERLAAMLMATVKMEELGHLT | |
| | TEKQEYALRLATVGYPKAGVYSGLIGGATSVLLSAYNRHPLF | |
| | QPLHTVMRETLFIGSHVVLRELRLNVTTQGPNLALYQLLSTAL | |
| | CSALEIGEVLRGLALGTESGLFSPCYLSLRFDLTRDKLLSMAP | |
| | QEATLDQAAVSNAVDGFLGRLSLEREDRDAWHLPAYKCVDR | |
| | LDKVLMIIPLINVTFIISSDREVRGSALYEASTTYLSSSLFLSPVI | |
| | MNKCSQGAVAGEPRQIPKIQNFTRTQKSCIFCGFALLSYDEKE | |
| | GLETTTYITSQEVQNSILSSNYFDFDNLHVHYLLLTTNGTVMEI | |
| | AGLYEERAHVVLAIILYFIAFALGIFLVHKIVMFFL | |
| PolyA tail | 100 nt | |

EBV gH (BXLF2)_RX-UTRB

| SEQ ID NO: 228 consists of from 5′ end to 3′ end: | | 228 |
|---|---|---|
| 5′ UTR SEQ ID NO: 104, mRNA ORF SEQ ID NO: 84, and | | |
| 3′ UTR SEQ ID NO: 3 | | |
| Chemistry | 1-methylpseudouridine | |
| Cap | 7mG(5′)ppp(5′)NlmpNp | |
| 5′UTR | GGGAAAUAAGAGAGAAAAGAAGAGUAAGAAGAAAUAUA | 104 |
| | AGACCCCGGCGCCGCCACC | |
| ORF of mRNA | AUGCAGUUGUUGUGCGUGUUCUGCCUCGUGUUACUCUGG | 84 |
| Construct | GAGGUGGGCGCCGCCAGCCUUAGCGAGGUGAAGCUCCAC | |
| (excluding | UUGGACAUCGAGGGCCACGCCAGCCACUACACCAUCCCCU | |
| the stop | GGACCGAGCUCAUGGCCAAGGUGCCCGGCCUUAGCCCCGA | |
| codon) | GGCCCUGUGGCGGGAGGCCAACGUGACCGAGGACCUGGC | |
| | CAGCAUGCUGAACCGGUACAAGCUGAUCUACAAGACCAG | |
| | CGGCACCCUGGGCAUCGCCCUGGCCGAGCCCGUGGACAUC | |
| | CCCGCCGUUAGCGAAGGCAGCAUGCAGGUGGACGCCAGC | |
| | AAGGUGCACCCCGGCGUGAUCAGCGGCCUGAACAGCCCCG | |
| | CCUGUAUGUUGAGCGCCCCACUGGAGAAGCAGCUGUUCU | |
| | ACUACAUCGGCACCAUGCUGCCCAACACCCGGCCCCACAG | |
| | CUACGUGUUCUACCAGCUGCGGUGCCACCUGAGCUACGU | |
| | UGCCCUGAGCAUCAACGGCGACAAGUUCCAGUACACCGG | |
| | CGCCAUGACCAGCAAGUUCCUGAUGGGCACCUACAAGCG | |
| | GGUCACCGAGAAGGGCGACGAGCACGUGCUGUCACUGGU | |
| | GUUCGGCAAGACCAAGGACCUGCCCGACCUGCGGGGCCCC | |
| | UUCAGCUACCCUAGUUUGACCAGCGCCCAGAGCGGCGAC | |
| | UACAGCUUGGUGAUCGUGACCACCUUCGUGCACUACGCC | |
| | AACUUCCACAACUACUUCGUGCCCAACCUGAAGGACAUG | |
| | UUCAGCCGGGCCGUGACCAUGACUGCCGCUUCUUACGCCC | |
| | GGUACGUGCUGCAGAAGCUGGUCCUGCUGGAGAUGAAGG | |
| | GCGGCUGCCGGGAGCCCGAGCUGGACACCGAAACACUGA | |
| | CCACCAUGUUCGAGGUGAGCGUGGCCUUCUUCAAGGUGG | |
| | GUCACGCGGUGGGCGAAACCGGCAACGGCUGCGUGGACU | |
| | UACGCUGGCUGGCCAAGAGCUUCUUCGAGCUGACCGUGC | |
| | UGAAGGAUAUCAUCGGCAUCUGCUACGGCGCCACCGUGA | |
| | AGGGCAUGCAGAGCUACGGCCUGGAGCGGCUGGCCGCCA | |
| | UGCUUAUGGCAACAGUGAAGAUGGAGGAGCUGGGACACC | |
| | UGACAACAGAGAAGCAGGAGUACGCCCUGAGACUGGCCA | |
| | CAGUGGGCUACCCAAAGGCCGGCGUGUACAGUGGACUGA | |
| | UCGGCGGCGCAACCAGCGUGCUGCUAUCCGCUUACAACCG | |
| | GCACCCGCUGUUCCAGCCCCUGCACACCGUGAUGCGGGAA | |
| | ACCCUGUUCAUCGGAAGCCACGUCGUGCUGCGGGAGCUG | |
| | AGGCUGAACGUAACCACCCAGGGCCCUAAUCUGGCCCUG | |
| | UAUCAGCUCCUCAGUACCGCCCUGUGCAGCGCCCUUGAGA | |
| | UCGGCGAGGUGCUCAGAGGCCUGGCCCUCGGUACCGAGA | |
| | GCGGCCUCUUCAGCCCAUGCUACUUAAGCCUGCGGUUCG | |
| | ACCUGACCCGGGACAAGUUGCUGAGCAUGGCCCCGCAGG | |
| | AGGCCACACUGGACCAGGCAGCUGUAUCCAACGCCGUGG | |
| | ACGGCUUCCUGGGCAGACUGUCCCUGGAACGGGAGGACC | |
| | GGGACGCCUGGCACCUGCCUGCCUACAAGUGUGUGGAUC | |
| | GGCUGGACAAGGUGCUGAUGAUCAUCCCUCUGAUUAAUG | |
| | UCACCUUCAUCAUCAGCAGCGACCGGGAGGUGCGGGGAU | |
| | CCGCCCUCUACGAGGCCAGCACCACCUAUCUGAGCAGCAG | |
| | CCUGUUCCUGUCUCCUGUGAUCAUGAACAAGUGCAGCCA | |
| | GGGCGCCGUGGCCGGCGAGCCCCGGCAGAUCCCCAAGAUC | |
| | CAGAACUUCACCCGGACCCAGAAGUCUUGCAUCUUCUGC | |
| | GGCUUCGCCCUUUUGUCCUACGACGAGAAGGAGGGCUUG | |
| | GAGACUACAACCUACAUCACCAGCCAGGAGGUGCAGAAC | |
| | AGCAUCCUGUCAUCUAAUUACUUCGACUUCGACAACCUG | |
| | CACGUUCAUUACCUGCUCCUCACCACCAACGGUACCGUCA | |
| | UGGAAAUCGCCGGACUGUACGAGGAGCGGGCCCAUGUUG | |
| | UGCUGGCCAUCAUCCUGUACUUCAUCGCUUUCGCACUUG | |
| | GCAUCUUCCUGGUGCACAAGAUCGUGAUGUUCUUCCUG | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MQLLCVFCLVLLWEVGAASLSEVKLHLDIEGHASHYTIPWTE | 38 |
| amino acid | LMAKVPGLSPEALWREANVTEDLASMLNRYKLIYKTSGTLGI | |
| sequence | ALAEPVDIPAVSEGSMQVDASKVHPGVISGLNSPACMLSAPLE | |
| | KQLFYYIGTMLPNTRPHSYVFYQLRCHLSYVALSINGDKFQYT | |
| | GAMTSKFLMGTYKRVTEKGDEHVLSLVFGKTKDLPDLRGPFS | |
| | YPSLTSAQSGDYSLVIVTTFVHYANFHNYFVPNLKDMFSRAV | |
| | TMTAASYARYVLQKLVLLEMKGGCREPELDTETLTTMFEVS | |
| | VAFFKVGHAVGETGNGCVDLRWLAKSFFELTVLKDIIGICYG | |
| | ATVKGMQSYGLERLAAMLMATVKMEELGHLTTEKQEYALR | |
| | LATVGYPKAGVYSGLIGGATSVLLSAYNRHPLFQPLHTVMRE | |
| | TLFIGSHVVLRELRLNVTTQGPNLALYQLLSTALCSALEIGEVL | |
| | RGLALGTESGLFSPCYLSLRFDLTRDKLLSMAPQEATLDQAA | |
| | VSNAVDGFLGRLSLEREDRDAWHLPAYKCVDRLDKVLMIIPL | |
| | INVTFIISSDREVRGSALYEASTTYLSSSLFLSPVIMNKCSQGAV | |
| | AGEPRQIPKIQNFTRTQKSCIFCGFALLSYDEKEGLETTTYITSQ | |
| | EVQNSILSSNYFDFDNLHVHYLLLTTNGTVMEIAGLYEERAH | |
| | VVLAIILYFIAFALGIFLVHKIVMFFL | |
| PolyA tail | 100 nt | |

EBV gL (B95-8)-UTRB

| SEQ ID NO: 229 consists of from 5′ end to 3′ end: | | 229 |
|---|---|---|
| 5′ UTR SEQ ID NO: 104, mRNA ORF SEQ ID NO: 85, and | | |
| 3′ UTR SEQ ID NO: 3 | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 104 |
| | AGACCCCGGCGCCGCCACC | |
| ORF of mRNA | AUGAGAGCCGUGGGCGUGUUCCUGGCCAUCUGCCUGGUG | 85 |
| Construct | ACCAUCUUCGUGCUGCCCACCUGGGGCAACUGGGCCUACC | |
| (excluding | CCUGCUGCCACGUGACCCAGCUGAGAGCCCAGCACCUGCU | |
| the stop | GGCCCUGGAGAACAUCAGCGACAUCUACCUGGUGAGCAA | |
| codon) | CCAGACCUGCGACGGCUUCAGCCUGGCCAGCCUGAACAGC | |
| | CCCAAGAACGGCAGCAACCAGCUGGUGAUCAGCAGAUGC | |
| | GCCAACGGCCUGAACGUGGUGAGCUUCUUCAUCAGCAUC | |
| | CUGAAGAGAAGCAGCAGCGCCCUGACCGGCCACCUGAGA | |
| | GAGCUGCUGACCACCCUGGAGACCCUGUACGGCAGCUUC | |
| | AGCGUGGAGGACCUGUUCGGCGCCAACCUGAACAGAUAC | |
| | GCCUGGCACAGAGGCGGC | |
| 3′ UTR | UGAUAAUAGGCUGGAGCCUCGGUGGCCAUGCUUCUUGCC | 3 |
| | CCUUGGGCCUCCCCCCAGCCCCUCCUCCCCUUCCUGCAC | |
| | CCGUACCCCCGUGGUCUUUGAAUAAAGUCUGAGUGGGCG | |
| | GC | |
| Corresponding | MRAVGVFLAICLVTIFVLPTWGNWAYPCCHVTQLRAQHLLA | 36 |
| amino acid | LENISDIYLVSNQTCDGFSLASLNSPKNGSNQLVISRCANG | |
| sequence | LNVVSFFISILKRSSSALTGHLRELLTTLETLYGSFSVED | |
| | LFGANLNRYAWHRGG | |
| PolyA tail | 100 nt | |

1.-49. (canceled)

50. An Epstein-Barr virus (EBV) vaccine comprising a lipid nanoparticle, a first mRNA, and a second mRNA, wherein the first mRNA comprises a first open reading frame (ORF) encoding an EBV glycoprotein L (gL) protein consisting of the amino acid sequence set forth in SEQ ID NO:36, and wherein the second mRNA comprises a second ORF encoding an EBV glycoprotein H (gH) protein consisting of the amino acid sequence set forth in SEQ ID NO:38.

51. The EBV vaccine of claim 50, further comprising a third mRNA, wherein the third mRNA comprises a third ORF encoding an EBV glycoprotein 42 (gp42) protein.

52. The EBV vaccine of claim 50, wherein all uridine residues in the mRNAs are 1-methyl-pseudouridines.

53. The EBV vaccine of claim 51, wherein all uridine residues in the mRNAs are 1-methyl-pseudouridines.

54. The EBV vaccine of claim 50, wherein the lipid nanoparticle comprises an ionizable cationic lipid, a neutral lipid, a sterol, and a polyethylene glycol (PEG)-modified lipid.

55. The EBV vaccine of claim 51, wherein the lipid nanoparticle comprises an ionizable cationic lipid, a neutral lipid, a sterol, and a PEG-modified lipid.

56. The EBV vaccine of claim 52, wherein the lipid nanoparticle comprises an ionizable cationic lipid, a neutral lipid, a sterol, and a PEG-modified lipid.

57. The EBV vaccine of claim 53, wherein the lipid nanoparticle comprises an ionizable cationic lipid, a neutral lipid, a sterol, and a PEG-modified lipid.

58. A method of EBV vaccination comprising administering to a human subject the EBV vaccine of claim 50 in an amount effective to induce in the human subject an EBV neutralizing antibody titer and/or a T cell immune response.

59. The method of claim 58, wherein the vaccine is administered intramuscularly.
